(19) United States
(12) Patent Application Publication
(10) Pub. No.: US 2007/0202515 A1
(43) Pub. Date: Aug. 30, 2007

(54) Promac signature application

(75) Inventors: Kenneth G. Hadlock, San Francisco, CA (US); Hien Kim Do, Palo Alto, CA (US); Stephanie Yu, San Francisco, CA (US); Hope Lancero, Palo Alto, CA (US)

Correspondence Address: COOLEY GODWARD KRONISH LLP;ATTN: Patent Group, Suite 500, 1200 - 19th Street, NW, WASHINGTON, DC, 20036-2402, US

(73) Assignee: PATHOLOGICA, LLC., Burlingame, CA (US)

(21) Appl. No.: 11/549,019

(22) Filed: Oct. 12, 2006

Related U.S. Application Data
(60) Provisional application No. 60/725,275, filed on Oct. 12, 2005.

(51) Int. Cl. C12Q 1/68; C12M 3/00

(52) U.S. Cl. 435/6; 435/287.2

(57) ABSTRACT

The present invention is directed to ProMac signature genes and methods and kits for using the ProMac signature genes for diagnostic, prognostic, or monitoring ProMac associated diseases.

This application claims priority to U.S. provisional application Ser. No. 60/725,275 filed on Oct. 12, 2005, the content of which is incorporated herein in its entirety.

### TECHNICAL FIELD

This invention relates generally to proliferating macrophages and disorders associated therewith. More specifically, it relates to the gene expression signatures of proliferating macrophages and their associated disorders, and methods and kits of using these signatures to determine the presence and relative levels of ProMacs in peripheral blood and condition of the disorders associated with proliferating macrophages.

### BACKGROUND OF THE INVENTION

Human macrophages serve as a first line of defense against a wide range of pathogenic organisms. Depending on the various cytokines, chemokines, and other proteins that macrophages and neighboring cells secrete, these terminally-differentiated immune cells will ingest or phagocytose foreign bacteria or proteins, initiate immune responses, or inhibit existing immune responses.

When functioning properly, macrophages act solely for the benefit of their host individual. At times, however, these cells can have deleterious effects, promoting the formation and spread of tumors (Zenger et al., 62 CANCER RESEARCH 5336 (2002)). Pathogenic roles for macrophages have been described for a wide range of chronic inflammatory conditions, including, but not limited to, amyotrophic lateral sclerosis (ALS), Alzheimer's Disease (AD), HIV-associated dementia (HAD), macular degeneration (MacDgn), scleroderma, and arteriosclerosis (Zhang et al., 159 J. NEUROIMMUNOL. 215 (2005); Pulliam et al., 87 J. CLINICAL INVESTIGATION 503 (1991); Atamas & White, 14 CYTOKINE GROWTH FACTOR REV. 537 (2003); Ambati et al., 9 NAT. MED. 1390 (2003); Boyle, 3 CURR. VASC. PHARMACOL. 63 (2005)). Often pathogenic macrophages undergo proliferation, and are therefore referred to as proliferating macrophages or ProMacs. See, for example, U.S. Pat. No. 6,924,095

A healthy macrophage can convert into a ProMac through integration of a retroviral transcriptional controlling sequence into the macrophage genome at a position relative to a cell proliferation gene such that the gene becomes activated. (U.S. Pat. Nos. 6,537,523; 5,744,122.) Identification of HIV or other retroviral integration in macrophage DNA can serve as a diagnostic factor of ProMac involvement in a patient's lymphoma or ALS. (U.S. Pub. No. 2003/0104009; U.S. Pat. Nos. 5,639,600; 5,580,715; Pub. No. WO 2004/069174.) Identifying macrophages with elevated levels of histocompatibility antigen HLA-DR has also been disclosed as a means of aiding diagnoses of ALS. (U.S. Pub. No. 2003/0175832.)

To reduce macrophage proliferation, polyamine analogs can be administered. (U.S. Pub. No. 2005/0159493.) Polyamine analogs modulate macrophage proliferation by blocking replication, and thereby effectively killing the cells.

Though methods exist for identifying ProMacs with a specific retroviral integration or elevated protein level, there is a need in the field for characterization of the ProMac gene expression signature. Knowing the ProMac signature is advantageous because it allows for straightforward and unambiguous means of diagnosing, prognosing, determining a predisposition for, tracking the remission of, and screening for treatments of ProMac-associated diseases. Knowing the genetic fingerprint of a ProMac additionally allows clinicians to easily determine whether a condition is ProMac-associated, thereby enabling them to treat the condition appropriately.

### SUMMARY OF THE INVENTION

The present invention features the molecular signature of ProMacs. The transcripts within the signature share the properties of: (1) being expressed primarily in macrophages; (2) having expression that is highly correlated with other transcripts in the signature; and (3) having expression that is relatively poorly correlated with transcripts from other cell populations in the peripheral blood cell or from T cells. Through detecting expression levels of transcripts in the ProMac signature, the presence and relative levels of ProMacs in biological samples can be determined. Consequently, the ProMac signature enables one to predict, diagnose, monitor, and identify therapeutics for ProMac associated diseases. Diseases associated with ProMacs include, but are not limited to, neurodegenerative disorders, vascular disorders, inflammatory disorders, immunological disorders, etc.

In one embodiment, the present invention provides a method for diagnosing a neurodegenerative disorder in a subject. The method comprises detecting the expression of a panel of ProMac signature genes in a biological sample of the subject, wherein a higher than normal level of expression of the panel of ProMac signature genes is indicative of a neurodegenerative disorder in the subject.

In another embodiment, the present invention provides a kit comprising one or more probes useful for detecting the expression of a panel of ProMac signature genes in a sample from a subject.

In yet another embodiment, the present invention provides a method for distinguishing a first neurodegenerative disorder from a second neurodegenerative disorder. The method comprises evaluating the expression of a panel of ProMac secondary signature genes associated with the first and the second neurodegenerative disorder in a biological sample from the subject, and correlating the expression of the panel of ProMac secondary signature genes with the determination of the first neurodegenerative disorder or the second neurodegenerative disorder, wherein the first neurodegenerative disorder is cerebral neuron degeneration and the second neurodegenerative disorder is motor neuron degeneration.

In yet another embodiment, the present invention provides a method for monitoring the treatment of a neurodegenerative disease in a subject. The method comprises monitoring the expression of a panel of ProMac signature genes in a biological sample from the subject, wherein the level of expression of the panel of ProMac signature genes positively correlates with the progress of the neurodegenerative disease in the subject.

In yet another embodiment, the present invention provides a method for monitoring the treatment of a ProMac associated disease in a subject. The method comprises monitoring the expression of a panel of ProMac signature genes in a biological sample from the subject, wherein the level of expression of the panel of ProMac signature genes positively correlates with the progress of the ProMac associated disease in the subject.

In yet another embodiment, the present invention provides a method for monitoring the level of disease associated macrophages in a subject. The method comprises monitoring the expression of a panel of ProMac signature genes in a biological sample from the subject, wherein the level of expression of the panel of ProMac signature genes positively correlates with the level of disease associated macrophages in the subject.

In yet another embodiment, the present invention provides a method for evaluating an agent comprising contacting the agent with a macrophage and evaluating the expression of a panel of ProMac signature genes in the presence and absence of the agent, wherein a change caused by the agent is indicative of the agent as a modulator of ProMac.

In yet another embodiment, the present invention provides a method for providing a prognosis of a ProMac associated disease in a subject. The present invention comprises detecting the expression of a panel of ProMac signature genes in a biological sample from the subject, wherein the expression of the panel of ProMac signature genes is negatively associated with a positive outcome of the ProMac associated disease.

In yet another embodiment, the present invention provides a method for providing a prognosis of a ProMac associated disease in a subject. The present invention comprises detecting the expression of a panel of ProMac secondary signature genes in a biological sample from the subject, wherein the expression of the panel of ProMac secondary signature genes is negatively associated with a positive outcome of the ProMac associated disease.

### BRIEF DESCRIPTION OF THE DRAWINGS

FIG. 1: Classification of ALS and control samples by LC5-CI. The LC5-CI scores obtained for ALS and control patient samples from both the training (open symbols) and test (shaded symbols) sets. Each symbol represents the LC5-CI score from a single sample. The lines indicate the mean LC5-CI score for each category.

FIG. 2: LC5—Classification Index vs time or severity of disease—(A) The LC5-CI scores obtained with ALS patients (Δ) classified by the time since ALS was diagnosed (x axis), Each symbol represents the LC5-CI score from a single sample. The lines indicate the mean LC5-CI score for each category and the error bars indicate one standard deviation. (B) The LC5-CI scores obtained with ALS patients (Δ) classified by the ALSFRS of the patient at the time the sample was drawn x-axis). The median ALSFRS of the population was 31. Each symbol represents the LC5-CI score from a single sample. The lines indicate the mean LC5-CI for each category. p>0.05 for the comparison.

FIG. 3: LC5 Classification Index and use of various medications—(A) The LC5-CI scores obtained with ALS patients (Δ) classified by whether or not the patient was taking anti-inflammatory medication (x axis). Each symbol represents the LC5-CI score from a single sample. The lines indicate the mean LC5-CI score for each category. p=ns for the comparison. (B) The LC5-CI scores obtained with ALS patients (A) classified by whether or not the patient was taking SSRIs (x axis). Each symbol represents the LC5-CI score from a single sample. The lines indicate the mean LC5-CI score for each category. p=ns for the comparison.

FIG. 4: Correlation analysis of LC5-CI—(A) Plot of the age of the ALS patients (x axis) versus the LC5-CI scores (y axis) obtained with the ALS patients (Δ). Multiple samples were assayed for 9 of the patients and the mean LC5 CI value is plotted. Error bars indicate one standard deviation from the mean for those samples. The Pearson correlation coefficient (R) and the p value of the correlation are indicated. (B) Plot of the percentage of CD14 positive cells with staining for FC gamma receptor (CD16, x axis) versus the LC5-CI scores (y axis) obtained with the ALS patients. CD14/CD16++ percentages were determined by flow cytometry as described (1). Labeling is as for A above. (C) Plot of the rate of change in the ALSFRS per month for individual patients versus the LC5-CI scores obtained. Negative values indicate a decrease; positive values indicate an increase in ALSFRS score. Between 2-3 separate ALSFRS determinations spaced 2-6 months apart were used to calculate the rate of change. Labeling is as for A, above. (D) Plot of the rate of change in forced vital capacity (FVC) for individual patients versus the LC5-CI scores obtained. Determination of the rate of change in FVC was performed as described in C, above.

FIG. 5: LC5-CI values obtained from various patient populations—The LC5-CI scores obtained with ALS patients (Δ), healthy controls (∇), patients with macular degeneration (Mac Dgn, □), Alzheimer's disease (◯), otherwise healthy HIV infected individuals (HIV-H, ♦), and HIV infected individuals with neurocognitive impairment (HIV neuro, ♦). Each symbol represents the LC5-CI score from a single sample. The lines indicate the mean LC5-CI score for each category. ALS vs controls, macular degeneration, and HIV-H p<0.001. AD vs controls, macular degeneration, and HIV-H p<0.001. ALS or AD v HIV-Neuro p=ns. Controls vs Mac Dgn, or HIV H p=ns. HIV neuro vs controls, p<0.001. HIV-H vs HIV neuro p=ns. All comparisons by one way ANOVA with Bonferroni post test.

FIG. 6: AD5 comparison of ALS, AD, and Control Patients—Plot of the AD5 scores obtained from ALS patients (n=25), AD patients (n=12) and age-matched controls (n=6). The line indicates the mean value for each group.

FIG. 7: Correlation of G1P3 signals with IFIT2 and IL16 signals. (A) Plot of the actin normalized signal obtained by quantitative RT-PCR analysis of G1P3 (x axis) versus the actin normalized signal obtained with primers for the gene IFIT-2 (y axis). Each triangle represents the signals obtained from one ALS PBMC sample. The best fit linear regression line is indicated, as are the Pearson correction coefficient (R) and the significance of the relationship. (B) Plot of actin normalized signal of G1P3 vs that obtained for the gene IL16 (y-axis). Labeling is as for 7A, above.

FIG. 8: Distance map of gene correlation in ALS PBMCs—Interconnectivity map of correlation between genes evaluated by quantitatitve light cycler PCR in ALS PBMCs. A line is a drawn between 2 genes if it has a distance of <0.3. The distances between 2 genes corresponds in a general way to the distance value but the line lengths are not to scale. Genes of the LC-5 are outlined in purple. Genes primarily expressed in macrophages are shaded yellow. Interferon induced genes are shaded blue. Human endogenous retroviral sequences are shaded red. The 17 signature genes are circled.

FIG. 9: Signature values obtained with changes in blood collection or incubation. (A) Comparison of the LC5-CI values obtained from PBMCs from 6 ALS patients and one AD patient collected into either heparin anticoagulant blood collection tubes (green bars) or acid-citrate-dextrose anti coagulant tubes (yellow) bars. (B) Comparison of the LC5-CI signals obtained from PBMCs from ALS (A) and AD (O) patients whose PBMCs were incubated for 3 or 16 hours (x axis) after Percoll gradient purification. Each symbol represents the LC5-CI signal (y axis) from a single PBMC sample. The line indicates the mean LC5-CI for each group. P>0.05 for all comparisons using one way ANOVA with Bonferroni post test.

FIG. 10: LC5-CI and Signature values in cutaneous T-cell lymphoma patient—the percent of CD14 cells also staining with antibodies to CD16 by flow cytometry (filled triangles) is plotted (left y axis) along with the LC5-CI (open triangles) determined for the same samples (right y axis). The samples include the baseline sample obtained prior to the administration of the first cycle of CG-47 (baseline, x axis), the samples obtained prior to the patient receiving the 2nd, 3rd, and 4th cycle of CG-47 (cy2, cy3, and cy4, respectively) and the final sample obtained when the patient experienced a relapse (ReLps).

FIG. 11: Transcriptional profiles of ALS and AD patient PBMCs—plot of the fold change for all expressed probe sets (N=28,935) for ALS patients/healthy controls (x axis) versus AD patients/healthy controls (y axis). The Pearson correlation for the entire data set is given.

FIG. 12: QRT-PCR analysis of upregulated genes. The bars indicate mean signal obtained from QRT-PCR of total RNA samples from ALS patients (black bars), AD patients (grey bars) or age matched healthy controls (white bars) for the indicated genes (above graphs). Error bars indicate one standard deviation from the mean. Values are expressed relative to values obtained from β-actin from the same samples. P values for the comparisons are given.

FIG. 13: Correlation in expression of upregulated genes. (A) Comparison of the β-actin normalized signals obtained from individual ALS (Δ) and AD patients (●) between the indicated genes (x and y axes). The best fit linear regression line for the ALS patients (black line) and AD patients (grey line) are also indicated as are the Pearson correlation coefficients for the comparisons. All correlations were significant at a level of at least p<0.01. (B) Histogram of number of Probe sets that have a Pearson correlations coefficient of greater or equal to 0.70 with the indicated number of survey probes. The 12 survey probe sets were 41469_at (PI3), 205041_s_at (ORM1), 217502_at (IFIT2), 209396_s_at (CHI3L1), 220005_at (P2RY13), 1559573_at (AK096134), 203021_at (SLPI), 221345_at (GPR43), 220000_at (SIGLEC5), 203591_S_at (CSF3R), 202905_x_at (BN), and 217897_at (FXYD6). The black bar numbers were derived using actual data. White bars indicate mean number of probe sets with R>=0.7 from 10 random permutations of the actual data set. Error bars indicate one standard deviation from the mean.

FIG. 14: Levels of MIFN signature gene RNA expression compared with levels of macrophage activation—the β-actin normalized signals (y axis) for the MIFN signature genes CHI3L1 (top graphs) and ORM1 (bottom graphs) in mononuclear cells from ALS patients (▴), AD patients (●), and healthy individuals (▾) compared to the mean HLA-DR staining of CD14+ monocytes (left column) or the percentage of CD14+ monocytes that also expressed CD16 (right column). HLA-DR and CD16 staining was determined by flow cytometry. Mononuclear cells were incubated overnight in culture media prior to isolation of total RNA. The best fit linear regression line for the ALS patients (grey line) are indicated, as are the Pearson correlation coefficients and p values for the correlations.

FIG. 15: MIFN signature genes are induced within 3 hours of isolation—RNA expression for 4 of the MIFN signature genes (indicated above graphs) in mononuclear cells from 6 ALS patients (top panels, black lines) and 6 controls (bottom panels, grey lines) after increasing amounts of time in culture at 37° C. Mononuclear cells were prepared by ammonium chloride mediated-lysis of red blood cells. Cells were grown under non-adherent conditions as described in materials and methods. Values are expressed as the fold change observed relative to the time 0 (immediately after red cell lysis) time point.

FIG. 16: Induction of MIFN signature proteins in ALS patients—mean levels of elafin (PI3, top panel) and interleukin 1 receptor antagonist (IL1RN, bottom panel) from cultures of mononuclear cells from 5 ALS patients (black bars) and 6 healthy individuals (white bars) after the indicated amount of time in culture at 37° C. Error bars indicate one standard deviation from the mean. Mononuclear cells were prepared by ammonium chloride mediated-lysis of red blood cells. Cells were grown under non-adherent conditions as described in materials and methods.

FIG. 17: High degree of correlation in expression of MIFN signature genes. The left Panel shows a comparison of the β-actin normalized signals obtained from individuals with the indicated diseases (see key at right of graphs) between the MIFN signature gene GPR43 and the MIFN signature gene IFIT2 (left graph) and GPR43 and the gene osteopontin (SPP1, right graph). Each symbol represents the signals obtained with the indicated genes (x and y axis) from an individual sample. The best fit linear regression lines obtained for the two comparisons are also indicated as are the Pearson correlation coefficients for the comparisons and the p values of the correlations.

FIG. 18: Distance map of 72 genes evaluated by QRT-PCR—a representation of the distance (defined as 1-Pearson correlation coefficient) between the indicated genes (identified by their Gene Symbol) using results obtained by QRT-PCR with gene specific primers and all available samples (range 30-211 median=84). Genes that have a distance value of 0.2 or less (e.g. an R>=0.8) are connected by Bold lines. Genes that have a distance of between 0.2 to 0.3 (R 0.8-0.8) are connected by a solid line and genes with a distance of greater than 0.3 are connected with dashed lines. Each gene is connected to the two other genes that it is closest to and any additional genes with which it has a distance of 0.3 or less. The genes of the MIFN-signature are located within the confines of the indicated ellipse. Gene symbols indicate whether a gene is for a secreted protein (diamond), membrane protein (hexagon) or intercellular protein (ellipse). Genes are also classified as myeloid associated, Interferon associated, induced by NFκB, or some combination of the foregoing using the color-code at upper right.

FIG. 19: Time course of MIFN signature RNA expression—RNA expression for 2 MIFN signature genes (ORM1 and NBS1) in mononuclear cells from 6 ALS patients (top panels, red/purple lines) and 6 controls (bottom panels, grey/green lines) after increasing amounts of time in culture at 37° C. Mononuclear cells were prepared by ammonium chloride mediated-lysis of red blood cells. Cells were grown under non-adherent conditions as described above. Values are expressed as the fold change observed relative to the time 0 (immediately after red cell lysis) time point.

FIG. 20: Time course of RNA expression of myeloid genes not in MIFN-signature—RNA expression for 2 myeloid associated genes GPR86 (aka P2RY13) and TNFRSF10c (aka TRAIL decoy receptor) in mononuclear cells from 6 ALS patients (top panels, red/purple lines) and 6 controls (bottom panels, grey/green lines) after increasing amounts of time in culture at 37° C. Mononuclear cells were prepared by ammonium chloride mediated-lysis of red blood cells. Cells were grown under non-adherent conditions as described above. Values are expressed as the fold change observed relative to the time 0 (immediately after red cell lysis) time point. Note that neither GPR86 nor TNFRSF10c demonstrate the 10-100 fold increase in signal at 3 hours seen in MIFN-signature genes.

FIG. 21: LC5 vs CD14/16% Top Panel: Indicates the LC5 score of samples from individuals with diverse diseases (x axis). Each symbol represents the LC5 score of an individual sample. The line indicates the mean value of the entire population. Values above 0 are expected from individuals with neurodegenerative diseases. Values of less than 0 are expected from healthy individuals. LC5 values were determined as described in Example 2. Bottom panel. Indicates the percentage of CD14+ monocytes that co-stain for CD16 (Fcgamma receptor III). Each symbol represents the CD14/16 percentage of an individual sample. The line indicates the mean value of the entire population. Values above 40% are considered elevated. CD14/16% s were determined by flow cytometry as described in Example 18.

FIG. 22: Macrophage vs Interferon score plots—graphs indicate the macrophage index (x axis) and interferon index (y axis) of samples from individuals with neurodegenerative disease (upper left panel), HIV infection who failed HAART (upper right panel), Age-related-macular-degeneration (lower right panel), and healthy individuals (lower left panel). Each symbol represents the scores obtained from an individual sample. Macrophage and interferon indexes were calculated using signals obtained by QRT-PCR of the appropriate genes.

FIG. 23: Comparisons of RNA signals from 6 genes in ALS and AD patients-graphs showing the β-actin normalized signals obtained from ALS and AD patients (x axis) with the indicated 6 genes (top of graphs). Each symbol represents the signal obtained from an individual sample. The line indicates the mean value of the entire population. The p value (unpaired t test) for the comparison is given.

FIG. 24: AD-10 assay—the graph indicates the AD10 score of samples from individuals with ALS and AD (x axis). Each symbol represents the AD10 score of an individual sample. The line indicates the mean value of the entire population. Values above 0 are expected from individuals with ALS. Values of less than 0 are expected from individuals with AD.

FIG. 25: MIFN-signature expression in different cell types—Percoll purified mononuclear cells were incubated overnight at 37 C and fractionated into CD16+ and negative fractions using magnetic separation. CD16-positive and negative fractions were then fractionated into CD14 and positive and sub-fractions. The upper left graph indicates the mean number of cells obtained in each cell fraction (x axis) from mononuclear cells obtained from 6 different healthy individuals. The bottom left graph indicates the mean threshold cycle values obtained from the indicated fractions when RNA purified from the fractions was amplified with primers to β-actin. The remaining four graphs indicate the actin-normalized signals obtained with primers to the MIFN signature genes GPR43, CLEC4E, ORM1, and PI3 from each cell fraction. The error bars indicate one standard deviation from the mean for all graphs. Significance testing was by exact T test with Bonferroni's correction for multiple comparisons.

FIG. 26: Flow cytometric analysis of CD14 expression—percoll purified mononuclear cells from a patient with Alzheimer's disease and a healthy individual were prepared and either immediately stained with antibodies to the pan-monocyte antigen CD14 or incubated overnight in RPMI media prior to staining. Results obtained with anti CD14 antibody (black lines) are compared to those obtained with an isotype-matched control (grey line).

FIG. 27: Flow cytometric analysis of CD16 expression—percoll purified mononuclear cells from a patient with Alzheimer's disease and a healthy individual were prepared and either immediately stained with antibodies to the human Fcγ III receptor CD16 or incubated overnight in RPMI media prior to staining. Results obtained with anti CD16 antibody (black lines) are compared to those obtained with an isotype-matched control (grey line).

FIG. 28: Increased CD16 expression on CD14 monocytes after overnight incubation—percoll purified mononuclear cells from a patient with Alzheimer's disease and a healthy individual were prepared and either immediately stained with antibodies to CD14 and CD16, or were incubated overnight in RPMI media prior to staining. Results obtained with PerCP congugated CD14 antibody are plotted on the x-axis. Results obtained with FITC conjugated CD16 antibody are plotted on the y axis. Staining obtained with isotype control antibodies (not shown) was restricted to the lower left quadrant.

FIG. 29: MIFN-signature proteins are expressed on CD14/16++monocytes-percoll purified mononuclear cells from a patient with Alzheimer's disease and a healthy individual were prepared and either immediately stained with antibodies to CD14 (PerCP), CD16 (PE), or the indicated MIFN-signature protein (FITC, above panels) or cells were incubated overnight in RPMI media prior to staining. Cells were then gated according to their staining with CD14 and/or CD16 antibodies with CD14 monocytes colored blue, CD16 singly positive cells colored red, and double staining cells colored green. Results obtained with PerCP congugated CD14 antibody are plotted on the x-axis. Results obtained with FITC conjugated GPR109B antibody or rabbit antisera to GPR43 and FITC conjugated anti rabbit antibody are plotted on the y axis.

FIG. 30: MIFN-signature proteins are expressed on CD14/16++monocytes-percoll purified mononuclear cells from a patient with Alzheimer's disease and a healthy individual were prepared and either immediately stained with antibodies to CD14 (PerCP), CD16 (PE), or the indicated MIFN-signature protein (FITC, above panels) or cells were incubated overnight in RPMI media prior to staining. Cells were then gated according to their staining with CD14 and/or CD16 antibodies with CD14 monocytes colored blue, CD16 singly positive cells colored red, and double staining cells colored green. Results obtained with PerCP congugated CD14 antibody are plotted on the x-axis. Results obtained with FITC conjugated NBS1/NBN antibody or FITC conjugated isotype-matched control are plotted on the y-axis.

FIG. 31: Expression of MIFN-signature proteins in CD14/16++monocytes in individuals with neurodegenerative disease—percoll purified mononuclear cells from 4 patients with Alzheimer's disease, 2 patients with ALS (black bars), and 5 healthy individuals (light grey bars) were prepared as described in FIGS. 5 and 6 and the mean fluorescent staining with antibodies to the MIFN-signature proteins GPR43 and FPRL1 were determined for CD16 positive cells (left graphs), CD14+ monocytes (center graphs), and CD14/CD16 double positive cells (right graphs) at isolation and after an overnight incubation. Error bars indicate one standard deviation from the mean. Significance testing of the difference between patients with neurodegenerative disease and healthy controls was by exact T test.

FIG. 32: Prediction of survival in ALS patients with 6 gene assay. The relationship between survival index score (y-axis) and survival in days x-axis) from the time the sample was drawn is shown. Higher signal with indicated genes is associated with shorter survival. GPR43 and MX2 are MIFN-signature genes.

FIG. 33: Table 1—Clinical and demographic features of the patients and controls in this study.

FIG. 34: Table 2—Results of microarray studies of neurodegenerative disease.

FIG. 35: Table 3—Primers employed for quantitative real-time RT-PCR.

FIG. 36: Table 4—Light cycler analysis of genes upregulated in ALS and AD patients.

FIG. 37: Table 5—Comparison of ten genes at discriminating AD from ALS.

FIG. 38: Table 6—Intercorrelation of signature vs. other genes in ALS PBMCs.

FIG. 39: Table 7—Information on light-cycler defined signature genes.

FIG. 40: Table 8—Intercorrelation of signature genes Affymetrix vs. RT-PCR.

FIG. 41: Table 9—Intercorrelation of signature vs. other genes in AD PBMCs.

FIG. 42: Table 10—Intercorrelation of signature vs. other genes in control PBMCs.

FIG. 43: Table 11—PCR primer sequences for genes tested by light cycler.

FIG. 44: Table 12—Patients employed.

FIG. 45: Table 13-QRT-PCR primers employed.

FIG. 46: Tables 14A and 14B—Probe sets significantly changed in ALS and AD patients (both transcripts and known genes).

FIG. 47: Table 15—All genes and probe sets upregulated 4 or more fold. The genes are identified by their HUGO Gene Nomenclature Committee official symbol. The Probe Set identifiers and the associated “Representative Public ID” for all Probe sets of the gene that have a mean increase in fold signal of greater than or equal to 4.0 are provided as are the mean increase in fold signal and associated p value (unpaired T test with Welch's correction for unequal variance) for both ALS and AD patient PBMCs. Gene IDs are coded in different fonts by association as follows: underlined: myeloid-associated; italics: α/β interferon-stimulated; bold: bound to or induced by NFκB; bold and underlined: both myeloid and NFκB associated; bold and italics: IFN stimulated and NFκB associated; italics and underlined: myeloid-associated and interferon-stimulated; bold, italics and underlined: in all three lists.

FIG. 48: Table 16—Upregulated genes associated with myeloid cells.

FIG. 49: Table 17—Upregulated genes stimulated by Type I interferon.

FIG. 50: Table 18—Upregulated genes associated with NFκB-mediated transcription.

FIG. 51: Table 19A and 19B—MIFN-signature genes (both transcripts and known genes).

FIG. 52: Table 20—Samples evaluated by QRT-PCR.

FIG. 53: Table 21—Genes confirmed to be in the MIFN signature.

FIG. 54: Table 22—Correlation analysis of MIFN signature in ALS/AD patients and controls.

FIG. 55: Table 23—Diagnostic utility of 24 genes (17 MIFN signature and 7 others)

FIG. 56: Table 24—Use of CLEC4E, GPR43 and IFIT2 for discrimination of neurodegenerative disease.

FIG. 57: Table 25—Results with two different four gene combinations.

FIG. 58: Table 26—LC5 vs, LC8.

FIG. 59: Table 27—Weighted voting parameters for cerebral vs motor neuron degeneration.

FIG. 60: Table 28—Representative ProMac signature genes.

FIG. 61: Table 29—Subgroup of ProMac signature genes.

FIG. 62: Table 30—Representative ProMac secondary signature genes.

FIG. 63: Table 31—Correlation of multiple genes with ALS rating scales and survival.

### DETAILED DESCRIPTION OF THE PREFERRED EMBODIMENTS

The following description outlines the invention summarized above. The invention, however, is not limited to the particular methodologies, protocols, genera, and reagents described herein and consequently may vary. Likewise, the terminology used herein describes particular embodiments only and is not intended to limit the scope of the invention.

All publications and patents mentioned herein are hereby incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### Definitions

Unless otherwise defined, all technical and scientific terms used herein have the same meaning as commonly understood to one of ordinary skill in the relevant art.

The singular forms “a,” “an,” and “the” include plural reference unless the context clearly dictates otherwise.

“Biological activity” refers to the biological behavior, function, and effects of a gene product or group of gene products, and can manifest on molecular and macromolecular levels. The biological activity, for example, of a protein may be affected at the molecular level (e.g., through preventing proper folding or binding) and may influence cellular level biological activities (e.g., signal transduction, cell proliferation, cell cycle regulation, apoptosis).

As used herein, “biological sample” encompasses a variety of sample types obtained from an organism that can be used in a diagnostic or monitoring assay. The definition encompasses blood and other liquid samples of biological origin, solid tissue samples, such as a biopsy specimen, or derived tissue cultures or cells, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples. Generally, the sample will be, or be derived from, peripheral (or circulating) blood. In some cases, the blood will have been enriched for a macrophage fraction, by using, for example, glass or plastic adherence. Alternatively, mononuclear cells may also be purified using Percoll gradients.

“Correlation” generally refers to the statistical association between variables. This term is not limited to any specific statistical method. Common statistical methods include the mathematical Pearson correlation, Kendall's tau, and the modifications seen in analysis of high-volume, high-density genotyping data, such as GeneSpring.

“Diagnosis” generally includes a determination of a subject's susceptibility to a disease or disorder, a determination as to whether a subject is presently affected by a disease or disorder, a prognosis of a subject affected by a disease or disorder, and therametrics (e.g., monitoring a patient's condition to provide information as to the effect or efficacy of therapy).

“Expression” generally refers to transcriptional or translational activity of a partial or entire gene, post-transcriptional or translational activities, e.g., activation or stabilization of a partial or entire gene, or the presence of any detectable level of one or more partial or entire transcription or translation products of a gene.

“Gene” refers to a polynucleotide sequence that comprises coding sequences, and optionally control sequences necessary for the production of a polypeptide or precursor. The polypeptide can be encoded by a full length coding sequence or by any portion of the coding sequence. A gene may constitute an uninterrupted coding sequence or it may include one or more introns, bound by the appropriate splice junctions. Moreover, a gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides.

“Gene product” refers to a biomolecule, such as a protein or mRNA, that is produced when a gene in an organism is transcribed or translated or post-translationally modified.

“Hybridization” refers to any process by which a polynucleotide sequence binds to a complementary sequence through base pairing. Hybridization conditions can be defined by, for example, the concentrations of salt or formamide in the prehybridization and hybridization solutions, or by the hybridization temperature, and are well known in the art. Hybridization can occur under conditions of various stringency.

“Kit” refers to a combination of physical elements, e.g., probes, including without limitation specific primers, labeled nucleotic acid probes, antibodies, protein-capture agent(s), reagent(s), instruction sheet(s) and other elements useful to practice the invention. These physical elements can be arranged in any way suitable for carrying out the invention. For example, probes can be provided in one or more containers or in an array or microarray device.

“Macrophage” refers to a mononuclear cell in tissue that expresses CD14, or a monocyte in circulation.

“Microarray,” as used herein, comprises a surface with an array, preferably ordered array, of putative binding (e.g., by hybridization) sites for a biochemical sample which often has undetermined characteristics. The term “microarray” generally refers to the type of genes or proteins represented on a microarray by polynucleotide sequences or protein-capture agents, and where the type of genes or proteins represented on the microarray is dependent on the intended purpose of the microarray (e.g., to monitor expression of human genes or proteins). The oligonucleotides or protein-capture agents on a given microarray may correspond to the same type, category, or group of genes or proteins. Genes or proteins may be considered to be of the same type if they share some common characteristics such as species of origin (e.g., human, mouse, rat); disease state (e.g., cancer); functions (e.g., protein kinases, tumor suppressors); same biological process (e.g., apoptosis, signal transduction, cell cycle regulation, proliferation, differentiation).

“Modulation” refers to the increasing or decreasing of an indicated phenomenon. “Modulation of ProMac biological activity,” therefore, refers to increasing or decreasing the biological activity of proliferating macrophages. Modulation of ProMac biological activity includes, but is not limited to, modulation of the rate of macrophage proliferation. Preferably, modulating macrophage proliferation refers to changing the rate of proliferation by at least 25%, preferably by at least 50%, more preferably by at least 75%, and even more preferably by at least 90%. For purposes of this invention, modulation of macrophage proliferation generally refers to decreasing the proliferative rate when compared to the rate of proliferation without administration of a modulator. However, because it may at times be desirable to increase the rate of proliferation from a previously measured level (e.g., during the course of therapy), increasing the rate of ProMac proliferation is also included within “modulation.”

“Modulator” and “agent that modulates” are used interchangeably herein and refer to a biological or chemical compound, natural or synthesized, that induces modulation, either directly or indirectly. A ProMac modulator, for example, either increases or decreases a biological activity of ProMacs (e.g., proliferation).

As used interchangeably herein, “candidate modulator” and “candidate agent,” refer to a compound that may have modulating effects, although the actuality or extent of the modulating effects has yet to be definitively determined. Included within this definition are known modulators whose effects on a particular set of circumstances is not certain (e.g., patient, type of disease, severity of disease, etc.).

“Oligonucleotide” refers to a polynucleotide sequence comprising, for example, from about 10 nucleotides to about 20, 30, 40, 50, 60, 70, 80, 90, 100, 200, 300, 400, 500, 600, 700, 800, 900, or 1000 nucleotides. The term “oligonucleotide” encompasses naturally occurring, synthetic, and modified oligonucleotides.

“Patient or Subject” as used herein, refers to any mammalian subject for whom diagnosis, treatment, or therapy is desired. In one preferred embodiment, the patient or subject is human. Other subjects may include, but are not limited to, cattle, horses, dogs, cats, guinea pigs, rabbits, rats, primates, and mice.

“Polynucleotide” refers to a polymeric form of nucleotides of any length, either ribonucleotides or deoxyribonucleotides. Thus, the term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The polynucleotide can contain intronic sequences. Polynucleotides that have been modified in order to introduce a means for attachment (e.g., to a support for use as a microarray) are included in this definition.

“Predisposition” to a disease refers to an individual's susceptibility to such disease. Individuals who are susceptible are statistically more likely to have a particular disease than normal/wildtype individuals.

“Prognosis” refers to the art or act of foretelling the course of a disease or disorder. Additionally, the term refers to the prospect of survival and recovery from a disease or disorder as anticipated from the usual course or indicated by special features of the individual's case. Further, the term refers to the art or act of identifying a disease or disorder from its signs and symptoms.

“Prognostic indicator” refers to anything that may serve as, or relate to, a ground or basis for prognosis. The term further refers to any grounds or basis of a differential diagnosis, including the results of testing and characterization of gene expression as described herein, and the distinguishing of a disease or condition from others presenting similar symptoms. Additionally, “prognostic indicator” refers to any grounds or basis, including the results of testing and characterization of gene expression as described herein, which may be used to distinguish the probable course of a malignant disease.

“Proliferating macrophage” and “ProMac” are interchangeable terms understood in the art and used herein to denote a macrophage which is capable of dividing. Normally, a macrophage is a terminally differentiated cell incapable of further division. For purposes of this invention, a “proliferating macrophage” is capable of further division or is in a portion of the cell cycle not considered to be terminal or end stage. Proliferation may be clonal, i.e., is derived from a single cell.

As used herein, identifying “the presence of ProMacs” refers to observing or detecting proliferating macrophages. An absolute or relative level of proliferating macrophages need not be determined.

A “ProMac associated disease,” a “disease associated with ProMacs,” a “disease characterized by ProMacs,” or any like term, refers to a disease, disorder or indication, that is associated with an elevated, or abnormal, level of macrophage proliferation as compared to a control sample.

“Polypeptide” and “protein” refer to a polymeric form of amino acids of any length, which can include coded and non-coded amino acids, chemically or biochemically modified (e.g., post-translational modification such as glycosylation) or derivatized amino acids, polymeric polypeptides, and polypeptides having modified peptide backbones. The term includes fusion proteins, immunologically tagged proteins; and the like. Proteins can also be modified to, for example, facilitate attachment to a support (e.g., to a support for use as a microarray).

“Protein-capture agent” refers to a molecule or a multi-molecular complex that can bind a protein to itself. The protein-capture agent may comprise a biomolecule such as a protein or a polynucleotide. Examples of protein-capture agents include immunoglobulins, antigens, receptors, or other proteins, or portions or fragments thereof. Furthermore, protein-capture agents are understood not to be limited to agents that only interact with their binding partners through noncovalent interactions. Protein-capture agents may also become covalently attached to the proteins with which they bind.

The terms “signature,” “gene expression signature,” “molecular signature,” and “genetic fingerprint,” all used interchangeably herein, refer to a group of genes or gene products which represent a particular cell or tissue type (e.g., ProMacs).

The terms “ProMac gene,” “ProMac signature,” “ProMac signature gene,” “MIFN signature gene” and “MIFN signature” are used interchangeably herein, and refer to a group of genes that are upregulated in ProMacs and have certain statistically significant association with the presence of ProMacs. They can be characterized by: (1) an increased expression in individuals with ALS and AD; (2) a high degree of correlation of signals with each other; (3) a similar time course of expression; and (4) expression that is relatively poorly correlated with transcripts from other cell populations in the peripheral blood cell or from T cells.

“ProMac secondary signature genes” refer to a group of genes that have an association with the presence of ProMacs that is secondary to the association held by ProMac signature genes. For example, ProMac secondary genes can be genes that are not ProMac signature genes, but their expression is upregulated in AD patients for at least 4-fold over the normal level.

“Transcript” refers to an RNA product transcribed from DNA. The category of “transcripts” includes, but is not limited to, pre-mRNA nascent transcripts, transcript processing intermediates, mature mRNAs and degradation products thereof. When detecting transcripts to practice the invention, it is sufficient to detect only one type of transcript, such as mature mRNA.

A “transcript primarily expressed in ProMacs,” as used herein, refers to a transcript whose expression is highly correlated with the expression of other transcripts in the ProMac signature and relatively poorly correlated with the expression of transcripts from other cell populations in the peripheral blood and from T cells.

Throughout this specification, the word “comprise,” or variations thereof, will be understood to imply the inclusion of a stated element, integer or step, or group of elements, integers or steps, but not the exclusion of any other element, integer or step, or group of elements, integers or steps.

### MODES FOR CARRYING OUT THE INVENTION

1. Detecting Levels of ProMac Signature Genes or ProMac Secondary Signature Genes

For methods of the invention that involve detecting the expression or expression level of a ProMac signature gene, any method for observing gene expression can be used, without limitation. For example, these methods can include traditional nucleic acid hybridization techniques, microarrays, polymerase chain reaction (PCR) methodologies, and protein determination. In one embodiment, it includes detection methods that use solid support-based assay formats (e.g., microarrays) as well as those that use solution-based assay formats (e.g., quantitative PCR). The gene expression assays can involve as many different transcripts from the ProMac signature as is necessary or useful. Preferred assays for detecting ProMacs through transcript expression include microarrays and quantitative PCRs.

Absolute measurements of the expression levels need not be made, although they can be made. The invention includes methods comprising comparisons of differences in expression levels between samples, and thus determining relative levels. Comparison of expression levels can be done visually or manually, or can be automated and done by a machine, using, for example, optical detection means. Subrahmanyam et al., 97 BLOOD 2457 (2001); Prashar et al., 303 METHODS ENZYMOL. 258 (1999). Hardware and software for analyzing differential expression of genes are available, and can be used in practicing the present invention. See, e.g., GenStat Software and GeneExpress® GX Explorer™ Training Manual; Baxevanis et al., 7 CURR. OPIN. BIOTECHNOL. 102 (1996).

a. Quantitative PCR-Based Methodologies

In one specific embodiment, quantitative PCR-based methodologies are used to detect expression of transcripts from the ProMac signature. These methods are well known to those of ordinary skill in the art, and may include, but are not limited to: real time quantitative PCR, quantitative competitive PCR, relative quantitation methods, branched DNA metholodologies, RRR, Gen-Probe-like and associated methods, and ligase chain amplification. Methods of quantitative PCR may be carried out using kits and methods that are commercially available from, for example, Applied BioSystems and Stratagene®. Preferred real-time PCR systems include the ABI PRISM® Sequence Detection System and the LightCycler® System. See also Kochanowski, QUANTITATIVE PCR PROTOCOLS (Humana Press, 1999); Innis et al., supra.; Vandesompele et al., 3 GENOME BIOL. RESEARCH0034 (2002); Stein, 59 CELL MOL. LIFE SCI. 1235 (2002).

Quantitative real time PCR, particularly quantitative real time reverse transcriptase PCR (Q-RTPCR), provides a method for detecting expression of ProMac signature genes in solution (in contrast to a microarray or other hybridization solid support). Q-RTPCR relies on detection of a fluorescent signal produced proportionally during amplification of a PCR product. See Innis et al., supra. Like traditional PCR method, Q-RTPCR employs PCR oligonucleotide primers, typically 15-30 bases long, that hybridize to opposite strands and regions flanking the DNA region of interest. Additionally, a probe (e.g., TaqMan®, Applied Biosystems) is designed to hybridize to the target sequence between the forward and reverse primers traditionally used in the PCR technique. The probe is labeled at the 5′ end with a reporter fluorophore, such as 6-carboxyfluorescein (6-FAM) and a quencher fluorophore like 6-carboxy-tetramethyl-rhodamine (TAMRA). As long as the probe is intact, fluorescent energy transfer occurs which results in the absorbance of the fluorescence emission of the reporter fluorophore by the quenching fluorophore. As Taq polymerase extends the primer, however, the intrinsic 5′ to 3′ nuclease activity of Taq degrades the probe, releasing the reporter fluorophore. The increase in the fluorescence signal detected during the amplification cycle is proportional to the amount of product generated in each cycle.

The forward and reverse amplification primers and internal hybridization probe is designed to hybridize specifically and uniquely with one nucleotide derived from the transcript of a target gene. In one embodiment, the selection criteria for primer and probe sequences incorporates constraints regarding nucleotide content and size to accommodate TaqMan® requirements.

Probe-less Q-RTPCR alternatives to the Taqman-type assay discussed above can be used. These probe-less systems, including the ABI PRISM® and the Lightcycler®, use labels such as SYBR Green®. See ABI PRISM® 7900 SEQUENCE DETECTION SYSTEM USER GUIDE APPLIED BIOSYSTEMS, chap. 1-8, App. A-F. (2002). The probe-less Q-RTPCR systems detect and measure the fluorescence emitted by the binding of the SYBR Green® or equivalent label to double-stranded DNA molecules.

Fluorescence in real time quantitative PCR, which is directly proportional to the amount of PCR amplified product in a well, is measured during the course of each amplification cycle. The measurements therefore occur in “real time,” as the amplification product accumulates in the reaction. As is well known in the art, following the amplifications in real time makes possible the quantification of transcripts as they are amplified. In parallel with the ProMac signature samples, a standard, corresponding to a target sequence dilution range, can be used which will make it possible to establish a standard curve, which in turn can be used to deduce the amount of the target in each sample. Levels of a macrophage housekeeping gene can also be measured and used as a standard to account for any experiment variations.

The quantitative PCR methods described herein are designed to be descriptive, not limiting. Modifications and/or substitutions that may be made to these methodologies, for example, using a different polymerase, fall within the scope of the invention.

b. Polynucleotide Hybridization

Detection of expression of a transcript from the ProMac signature can be accomplished through well known hybridization techniques for polynucleotides, including, but not limited to: Northern blotting, Southern blotting, solution hybridization, and S1 nuclease protection assays.

Nucleic acid hybridization typically involves contacting an oligonucleotide probe and a sample comprising nucleic acids under conditions where the probe can form stable hybrid duplexes with its complementary nucleic acid through complementary base pairing. See, e.g., Berger & Kimmel, 152 METHODS ENZYMOL. 1 (1987). The polynucleotides that do not form hybrid duplexes are then washed away leaving the hybridized polynucleotides to be detected, typically through detection of an attached detectable label. The detectable label can be present on the probe or on the sample. Detectable labels are commonly radioactive or fluorescent labels, but any label capable of detection can be used. Labels can be incorporated by several approaches well known in the art. In one aspect RNA can be amplified using the MessageAmp kit (Ambion) with the addition of aminoallyl-UTP as well as free UTP. The aminoallyl groups incorporated into the amplified RNA can be reacted with a fluorescent chromophore, such as CyDye (Amersham Biosciences)

Duplexes of nucleic acids are destabilized by increasing the temperature or decreasing the salt concentration of the buffer containing the nucleic acids. Under low stringency conditions (e.g., low temperature and/or high salt) hybrid duplexes (e.g., DNA:DNA, RNA:RNA or RNA:DNA) will form even where the annealed sequences are not perfectly complementary. Thus, specificity of hybridization is reduced at lower stringency. Conversely, at higher stringency (e.g., higher temperature and/or lower salt and/or in the presence of destabilizing reagents) hybridization tolerates fewer mismatches.

Typically, stringent conditions for short probes (e.g., 10 to 50 nucleotide bases) will be those in which the salt concentration is at least about 0.01 to 1.0 M at pH 7.0 to 8.3 and the temperature is at least about 30° C. Stringent conditions can also be achieved with the addition of destabilizing agents such as formamide.

Under some circumstances, it can be desirable to perform hybridization at conditions of low stringency, e.g., 6×SSPE-T (0.9 M NaCl, 60 mM NaH₂PO₄, pH 7.6, 6 mM EDTA, 0.005% Triton) at 37° C., to ensure hybridization. Subsequent washes can then be performed at higher stringency (e.g., 1×SSPE-T at 37° C.) to eliminate mismatched hybrid duplexes. Successive washes can be performed at increasingly higher stringency (e.g., down to as low as 0.25×SSPE-T at 37° C. to 50° C.) until a desired level of hybridization specificity is obtained.

In general, standard conditions for hybridization is a compromise between stringency (hybridization specificity) and signal intensity. Thus, the hybridized polynucleotides may be washed at successively higher stringency conditions and read between each wash. Analysis of the data sets produced in this manner will reveal a wash stringency above which the hybridization pattern is not appreciably altered and which provides adequate signal for the particular oligonucleotide probes of interest. For example, the final wash may be selected as that of the highest stringency that produces consistent results and that provides a signal intensity greater than approximately 10% of the background intensity.

Probes useful in polynucleotide hybridization techniques are oligonucleotides capable of binding to a polynucleotide of complementary sequence through one or more types of chemical bonds, usually through complementary base pairing via hydrogen bond formation. A probe can include natural bases (i.e., A, G, U, C or T) or modified bases (7-deazaguanosine, inosine, etc.). In addition, the nucleotide bases in the probes can be joined by a linkage other than a phosphodiester bond, so long as it does not interfere with hybridization (e.g., peptide bonds).

Oligonucleotide probes can be designed by any means known in the art. See, e.g., Li and Stormo, 17 BIOINFORMATICS 1067 (2001). Oligonucleotide probe design can be effected using software. Exemplary software includes ArrayDesigner, GeneScan, and ProbeSelect. Probes complementary to a defined nucleic acid sequence can be synthesized chemically, generated from longer nucleotides using restriction enzymes, or can be obtained using techniques such as polymerase chain reaction (PCR). PCR methods are well known and are described, for example, in Innis et al. eds., PCR PROTOCOLS: A GUIDE TO METHODS AND APPLICATIONS, Academic Press Inc. San Diego, Calif. (1990). The probes can be labeled, for example, with a radioactive, biotinylated, or fluorescent tag. Optimally, the ProMac transcripts in the sample are labeled and the probes are not labeled. Oligonucleotide probes generated by the above methods can be used in solution or solid support-based methods.

c. Microarrays

The invention also provides microarrays, microarray kits, and the use of microarrays to determine the expression of ProMac signature gene(s), and optionally detecting, prognosing, and monitoring the treatment of ProMac associated diseases. In one specific embodiment, microarray technology can be used to assess the level of at least one transcript primarily expressed in ProMacs in a biological sample, and through comparison with a control level of the transcript, diagnose whether a patient is afflicted with, or likely to become afflicted with, a ProMac associated disease. The claimed polynucleotide and protein-capture microarrays may also be used to identify drug compounds that modulate expression of ProMac signature genes or gene products. Additionally, microarrays may be created that model various ProMac associated diseases, and in turn, novel drug compounds may be analyzed as potential therapeutics or treatments.

Microarrays may also be used to identify compounds that bind ProMacs. Modulating compounds that increase transcription rate of a ProMac signature gene or stimulate the biological activity of ProMacs are considered activating, and compounds that decrease rates or inhibit ProMac biological activity are non-activating. Thus, the microarrays of the invention may be used to analyze and characterize the transcriptional state of a signature gene following exposure to an activating or, preferably, a non-activating compound.

Microarray technology further provides the opportunity to analyze a large number of genes and gene products. This technology may therefore be utilized for comparative gene expression analysis, drug discovery, and characterization of molecular interactions. With respect to expression analysis, the expression pattern of a ProMac gene may be used to characterize its function. In addition, microarrays may be utilized to analyze both the static expression of a ProMac gene or gene product (e.g., expression in a specific tissue) and the dynamic expression of a gene or gene product (e.g., expression relative to the expression of a known ProMac signature gene/gene product). See Duggan et al., 21 NATURE GENET. 10 (1999). The described microarrays therefore enable identification of additional genes and gene products that may be useful prognostic indicators of ProMac associated diseases.

i. Polynucleotide Arrays

An advantage of the polynucleotide microarray technology is the use of an impermeable, rigid support as compared to the porous membranes used in the traditional blotting methods (e.g., Northern and Southern analyses). Hybridization buffers do not penetrate the support resulting in greater access to the oligonucleotide probes, enhanced rates of hybridization, and improved reproducibility. In addition, the microarray technology provides better image acquisition and image processing. See Southern et al., 21 NATURE GENET. 5 (1999). For microarray analysis, polynucleotides (e.g., RNA transcripts) may be isolated from a biological sample. Polynucleotide samples include, but are not limited to, DNA, mRNA transcripts of the gene or genes, cDNA reverse transcribed from the mRNA, cRNA transcribed from the cDNA, DNA amplified from the genes, RNA transcribed from amplified DNA, and the like.

(1) DNA Microarrays

In one specific embodiment, gene chip systems, or high density DNA microarrays, are a preferable technology to use for detecting the presence and/or level of ProMacs in a biological sample through identifying expression of at least one gene identified to be in the ProMac signature. Variations of any number of ProMac signature genes can be printed onto a gene chip and then, through hybridizing DNA from a patient's biological sample, it can be determined whether the sample contains ProMacs. Gene chips may be custom-designed and are also commercially available (Affymetrix, Agilent, Quantum Dot, Celera, etc.). Gene chip arrays and the probes for use thereon can be produced according to the same methods employed for all polynucleotide microarrays.

(2) Methods for Producing Polynucleotide Microarrays

The microarrays may be produced through spatially-directed oligonucleotide synthesis. Methods for spatially-directed oligonucleotide synthesis include, without limitation, light-directed oligonucleotide synthesis, microlithography, application by ink jet, microchannel deposition to specific locations and sequestration with physical barriers. In general, these methods involve generating active sites, usually by removing protective groups, and coupling to the active site a nucleotide that, itself, optionally has a protected active site if further nucleotide coupling is desired.

A microarray may be configured, for example, by in situ synthesis or by direct deposition (“spotting” or “printing”) of synthesized oligonucleotide probes onto the support. The oligonucleotide probes are used to detect complementary polynucleotide sequences in a target sample of interest. In situ synthesis has several advantages over direct placement, such as higher yields, consistency, efficiency, cost, and potential use of combinatorial strategies. See Southern et al., 21 NATURE GENET. 5 (1999). However, for longer polynucleotide sequences such as PCR products, deposition may be the preferred method. Generation of microarrays by in situ synthesis may be accomplished by a number of methods including photochemical deprotection, ink-jet delivery, and flooding channels. See Lipshutz et al., 21 NATURE GENET. 20 (1999); Blanchard et al., 11 BIOSENSORS AND BIOELECTRONICS, 687 (1996); Maskos et al., 21 NUCL. ACIDS RES. 4663 (1993).

The microarrays of the invention may be constructed by the in situ synthesis method using solid-phase DNA synthesis and photolithography. See Lipshutz et al., 21 NATURE GENET. 20 (1999). Linkers with photolabile protecting groups may be covalently or non-covalently attached to a support (e.g., glass). Light is then directed through a photolithographic screen to specific areas on the support resulting in localized photodeprotection and yielding reactive hydroxyl groups in the illuminated regions. A 3′-O-phosphoramidite-activated deoxynucleoside (protected at the 5′-hydroxyl with a photolabile group) is then incubated with the support and coupling occurs at deprotected sites that were exposed to light. Following the optional capping of unreacted active sites and oxidation, the support is rinsed and the surface is illuminated through a second screen, to expose additional hydroxyl groups for coupling to the linker. A second 5′-protected, 3′-O-phosphoramidite-activated deoxynucleoside is presented to the support. The selective photodeprotection and coupling cycles are repeated until the desired products are obtained. Photolabile groups may then be removed and the sequence may be capped. Side chain protective groups may also be removed. Because photolithography is used, the process may be miniaturized to generate high-density microarrays of oligonucleotide probes. Thus, thousands to hundreds of thousands of oligonucleotide probes may be generated on a single microarray support using this technology.

To produce a microarray by the spotting (or printing) method, oligonucleotide probes are prepared, generally by PCR, for printing onto the microarray support. As described for the in situ technique, the probes may be selected from a number of sources including polynucleotide databases such as GenBank, Unigen, HomoloGene, RefSeq, dbEST, and dbSNP. See Wheeler et al., 33 NUCL. ACIDS RES. 39 (2005). Alternatively or in addition, oligonucleotide probes may be randomly selected from cDNA libraries reflecting, for example, a tissue type (e.g., lymphoid or neuronal tissue), or a genomic library representing a species of interest (e.g., Drosophila melanogaster). If PCR is used to generate the probes, for example, approximately 100-500 μg of the purified PCR product (about 0.6-2.4 kb) may be spotted onto the support. Duggan et al., 21 NATURE GENET. 10 (1999). The spotting (or printing) may be performed by a robotic arrayer. See, e.g., U.S. Pat. Nos. 6,150,147; 5,968,740; 5,856,101; 5,474,796; and 5,445,934.

Polynucleotide microarrays may also be prepared via a solid phase synthesis method that utilizes electrochemical placement of monomers or nucleic acids. See, e.g., U.S. Pat. Nos. 6,280,595 and 6,093,302.

A number of different microarray configurations and methods for their production are known to those of skill in the art and are disclosed in U.S. Pat. Nos. 6,156,501; 6,077,674; 6,022,963; 5,919,523; 5,885,837; 5,874,219; 5,856,101; 5,837,832; 5,770,722; 5,770,456; 5,744,305; 5,700,637; 5,624,711; 5,593,839; 5,571,639; 5,556,752; 5,561,071; 5,554,501; 5,545,531; 5,529,756; 5,527,681; 5,472,672; 5,445,934; 5,436,327; 5,429,807; 5,424,186; 5,412,087; 5,405,783; 5,384,261; 5,242,974; and the disclosures of which are herein incorporated by reference. Patents describing methods of using microarrays in various applications include: U.S. Pat. Nos. 5,874,219; 5,848,659; 5,661,028; 5,580,732; 5,547,839; 5,525,464; 5,510,270; 5,503,980; 5,492,806; 5,470,710; 5,432,049; 5,324,633; 5,288,644; 5,143,854; and the disclosures of which are incorporated herein by reference.

(a) Microarray Supports

A microarray support may comprise a flexible or rigid support. A flexible support is capable of being bent, folded, or similarly manipulated without breakage. Examples of solid materials that are flexible supports with respect to the invention include membranes, such as nylon and flexible plastic films. The rigid supports of microarrays are sufficient to provide physical support and structure to the associated oligonucleotides under the appropriate assay conditions.

The support may be biological, nonbiological, organic, inorganic, or a combination of any of these, existing as particles, strands, precipitates, gels, sheets, tubing, spheres, containers, capillaries, pads, slices, films, plates, or slides. In addition, the support may have any convenient shape, such as a disc, square, sphere, or circle. In one embodiment, the support is flat but may take on a variety of alternative surface configurations. For example, the support may contain raised or depressed regions on which the synthesis takes place. The support and its surface may form a rigid support on which the reactions described herein may be carried out. The support and its surface may also be chosen to provide appropriate light-absorbing characteristics. For example, the support may be a polymerized Langmuir Blodgett film, functionalized glass, Si, Ge, GaAs, GaP, SiO₂, SIN₄, modified silicon, or any one of a wide variety of gels or polymers such as (poly)tetrafluoroethylene, (poly)vinylidenedifluoride, polystyrene, polycarbonate, or combinations thereof. The surface of the support may also contain reactive groups, such as carboxyl, amino, hydroxyl, and thiol groups. The surface may be transparent and contain SiOH functional groups, such as found on silica surfaces.

The support may be composed of a number of materials, including glass. There are several advantages for utilizing glass supports in constructing a microarray. For example, microarrays prepared using a glass support generally utilize microscope slides due to the low inherent fluorescence, thus minimizing background noise. Moreover, hundreds to thousands of oligonucleotide probes may be attached to a slide. The glass slides may be coated with polylysine, amino silanes, or amino-reactive silanes that enhance the hydrophobicity of the slide and improve the adherence of the oligonucleotides. Duggan et al., 21 NATURE GENET. 10 (1999). Ultraviolet irradiation may be used to crosslink the oligonucleotide probes to the glass support. Following irradiation, the support may be treated with succinic anhydride to reduce the positive charge of the amines. For double-stranded oligonucleotides, the support may be subjected to heat (e.g., 95° C.) or alkali treatment to generate single-stranded probes. An additional advantage of using glass is its nonporous nature, thus requiring a minimal volume of hybridization buffer and resulting in enhanced binding of target samples to probes.

In another embodiment, the support may be flat glass or single-crystal silicon with surface relief features of less than about 10 angstroms. The surface of the support may be etched using well-known techniques to provide desired surface features. For example, trenches, v-grooves, or mesa structures allow the synthesis regions to be more closely placed within the focus point of impinging light.

The invention also relates to polynucleotide microarray supports comprising beads. These beads may have a wide variety of shapes and may be composed of numerous materials. Generally, the beads used as supports may have a homogenous size between about 1 and about 100 microns, and may include microparticles made of controlled pore glass (CPG), highly crosslinked polystyrene, acrylic copolymers, cellulose, nylon, dextran, latex, and polyacrolein. See, e.g., U.S. Pat. Nos. 6,060,240; 4,678,814; and 4,413,070.

Several factors may be considered when selecting a bead for a support including material, porosity, size, shape, and linking moiety. Other important factors to be considered in selecting the appropriate support include uniformity, efficiency as a synthesis support, surface area, and optical properties (e.g., autofluoresence). Typically, a population of uniform oligonucleotide or polynucleotide fragments may be employed. However, beads with spatially discrete regions each containing a uniform population of the same oligonucleotide or polynucleotide fragment (and no other), may also be employed. In one embodiment, such regions are spatially discrete so that signals generated by fluorescent emissions at adjacent regions can be resolved by the detection system being employed.

In general, the support beads may be composed of glass (silica), plastic (synthetic organic polymer), or carbohydrate (sugar polymer). A variety of materials and shapes may be used, including beads, pellets, disks, capillaries, cellulose beads, pore-glass beads, silica gels, polystyrene beads optionally crosslinked with divinylbenzene, grafted co-poly beads, polyacrylamide beads, latex beads, dimethylacrylamide beads optionally cross-linked with N,N-1-bis-acryloyl ethylene diamine, and glass particles coated with a hydrophobic polymer (e.g., a material having a rigid or semirigid surface). The beads may also be chemically derivatized so that they support the initial attachment and extension of nucleotides on their surface.

Oligonucleotide probes, including probes specific for polynucleotides primarily expressed in ProMacs, may be synthesized either directly on the bead or separately synthesized and attached to the bead. See, e.g., Albretsen et al., 189 ANAL. BIOCHEM. 40 (1990); Lund et al., 16 NUCL. ACIDS RES. 10861 (1988); Ghosh et al., 15 NUCL. ACIDS RES. 5353 (1987); Wolf et al., 15 NUCL. ACIDS RES. 2911 (1987). Attachment to the bead may be permanent, or a cleavable linker between the bead and the probe may be used. The link should not interfere with the probe-target binding during screening. Linking moieties for attaching and synthesizing tags on microparticle surfaces are disclosed in U.S. Pat. No. 4,569,774; Beattie et al., 39 CLIN. CHEM. 719 (1993); Maskos and Southern, 20 NUCL. ACIDS RES. 1679 (1992); Damba et al., 18 NUCL. ACIDS RES. 3813 (1990); and Pon et al., 6 BIOTECHNIQUES 768 (1988). Various links may include polyethyleneoxy, saccharide, polyol, esters, amides, saturated or unsaturated alkyl, aryl, and combinations thereof.

If the oligonucleotide probes are chemically synthesized on the bead, the bead-oligo linkage may be stable during the deprotection step of photolithography. During standard phosphoramidite chemical synthesis of oligonucleotides, a succinyl ester linkage may be used to bridge the 3′ nucleotide to the resin. This linkage may be readily hydrolyzed by NH₃ prior to and during deprotection of the bases. The finished oligonucleotides may be released from the resin in the process of deprotection. The probes may be linked to the beads by a siloxane linkage to Si atoms on the surface of glass beads; a phosphodiester linkage to the phosphate of the 3′-terminal nucleotide via nucleophilic attack by a hydroxyl (typically an alcohol) on the bead surface; or a phosphoramidate linkage between the 3′-terminal nucleotide and a primary amine conjugated to the bead surface.

Numerous functional groups and reactants may be used to detach the oligonucleotide probes. For example, functional groups present on the bead may include hydroxy, carboxy, iminohalide, amino, thio, active halogen (Cl or Br) or pseudohalogen (e.g., CF₃, CN), carbonyl, silyl, tosyl, mesylates, brosylates, and triflates. In some instances, the bead may have protected functional groups that may be partially or wholly deprotected.

(b) Microarray Support Surface

The support of the microarrays may comprise at least one surface on which a pattern of oligonucleotide probes is present, where the surface may be smooth or substantially planar, or have irregularities, such as depressions or elevations. The surface on which the probes are located may be modified with one or more different layers of compounds that serve to modulate the properties of the surface. Such modification layers may generally range in thickness from a monomolecular thickness of about 1 mm, preferably from a monomolecular thickness of about 0.1 mm, and more preferably from a monomolecular thickness of about 0.001 mm. Modification layers include, for example, inorganic and organic layers such as metals, metal oxides, polymers, small organic molecules and the like. Polymeric layers include peptides, proteins, polynucleotides or mimetics thereof (e.g., peptide nucleic acids), polysaccharides, phospholipids, polyurethanes, polyesters, polycarbonates, polyureas, polyamides, polyethyleneamines, polyarylene sulfides, polysiloxanes, polyimides, and polyacetates. The polymers may be hetero- or homopolymeric, and may or may not have separate functional moieties attached.

The oligonucleotide probes of a microarray may be arranged on the surface of the support based on size. With respect to the arrangement according to size, the probes may be arranged in a continuous or discontinuous size format. In a continuous size format, each successive position in the microarray, for example, a successive position in a lane of probes, comprises oligonucleotide probes of the same molecular weight. In a discontinuous size format, each position in the pattern (e.g., band in a lane) represents a fraction of target molecules derived from the original source, where the probes in each fraction will have a molecular weight within a determined range.

The probe pattern may take on a variety of configurations as long as each position in the microarray represents a unique size (e.g., molecular weight or range of molecular weights), depending on whether the microarray has a continuous or discontinuous format. The microarrays may comprise a single lane or a plurality of lanes on the surface of the support. Where a plurality of lanes are present, the number of lanes will usually be at least about 2 but less than about 200 lanes, preferably more than about 5 but less than about 100 lanes, and most preferred more than about 8 but less than about 80 lanes.

Each microarray may contain oligonucleotide probes isolated from the same source (e.g., the same tissue), or contain probes from different sources (e.g., different tissues, different species, disease and normal tissue). As such, probes isolated from the same source may be represented by one or more lanes; whereas probes from different sources may be represented by individual patterns on the microarray where probes from the same source are similarly located. Therefore, the surface of the support may represent a plurality of patterns of oligonucleotide probes derived from different sources (e.g., tissues), where the probes in each lane are arranged according to size, either continuously or discontinuously.

Surfaces of the support are usually, though not always, composed of the same material as the support. Alternatively, the surface may be composed of any of a wide variety of materials, for example, polymers, plastics, resins, polysaccharides, silica or silica-based materials, carbon, metals, inorganic glasses, membranes, or any of the above-listed support materials. The surface may contain reactive groups, such as carboxyl, amino, or hydroxyl groups. The surface may be optically transparent and may have surface SiOH functionalities, such as are found on silica surfaces.

(c) Attachment of Oligonucleotide Probes

The surface of the support may possess a layer of linker molecules (or spacers). The linker molecules may be of sufficient length to permit oligonucleotide probes (polynucleotide sequences) on the support to hybridize to polynucleotide molecules and to interact freely with molecules exposed to the support. The linker molecules may be about 6-50 molecules long to provide sufficient exposure. The linker molecules may also be, for example, aryl acetylene, ethylene glycol oligomers containing about 2-10 monomer units, diamines, diacids, amino acids, or combinations thereof.

The linker molecules may be attached to the support via carbon-carbon bonds using, for example, (poly)trifluorochloroethylene surfaces, or preferably, by siloxane bonds (using, for example, glass or silicon oxide surfaces). Siloxane bonds may be formed via reactions of linker molecules containing trichlorosilyl or trialkoxysilyl groups. The linker molecules may also have a site for attachment of a longer chain portion. For example, groups that are suitable for attachment to a longer chain portion may include amines, hydroxyl, thiol, and carboxyl groups. The surface attaching portions may include aminoalkylsilanes, hydroxyalkylsilanes, bis(2-hydroxyethyl)-aminopropyltriethoxysilane, 2-hydroxyethylaminopropyltriethoxysilane, aminopropyltriethoxysilane, and hydroxypropyltriethoxysilane. The linker molecules may be attached in an ordered array (e.g., as parts of the head groups in a polymerized Langinuir Blodgett film). Alternatively, the linker molecules may be adsorbed to the surface of the support.

The linker may be a length that is at least the length spanned by, for example, two to four nucleotide monomers. The linking group may be an alkylene group (from about 6 to about 24 carbons in length), a polyethyleneglycol group (from about 2 to about 24 monomers in a linear configuration), a polyalcohol group, a polyamine group (e.g., spermine, spermidine, or polymeric derivatives thereof), a polyester group (e.g., poly(ethylacrylate) from 3 to 15 ethyl acrylate monomers in a linear configuration), a polyphosphodiester group, or a polynucleotide (from about 2 to about 12 polynucleotides). For in situ synthesis, the linking group may be provided with functional groups that can be suitably protected or activated. The linking group may be covalently attached to the oligonucleotide probes by an ether, ester, carbamate, phosphate ester, or amine linkage. In one embodiment, linkages are phosphate ester linkages, which can be formed in the same manner as the oligonucleotide linkages. For example, hexaethyleneglycol may be protected on one terminus with a photolabile protecting group (e.g., NVOC or MeNPOC) and activated on the other terminus with 2-cyanoethyl-N,N-diisopropylamino-chlorophosphite to form a phosphoramidite. This linking group may then be used for construction of oligonucleotide probes in the same manner as the photolabile-protected, phosphoramidite-activated nucleotides.

Furthermore, the linker molecules and oligonucleotide probes may contain a functional group with a bound protective group. In one embodiment, the protective group is on the distal or terminal end of the linker molecule opposite the support. The protective group may be either a negative protective group (e.g., the protective group renders the linker molecules less reactive with a monomer upon exposure) or a positive protective group (e.g., the protective group renders the linker molecules more reactive with a monomer upon exposure). In the case of negative protective groups, an additional reactivation step may be required, for example, through heating. The protective group on the linker molecules may be selected from a wide variety of positive light-reactive groups preferably including nitro aromatic compounds, such as o-nitrobenzyl derivatives or benzylsulfonyl. Other protective groups include 6-nitroveratryloxycarbonyl (NVOC), 2-nitrobenzyloxycarbonyl (BOC) or α,α-dimethyl-dimethoxybenzyloxycarbonyl (DDZ). Photoremovable protective groups are described in, for example, Patchomik, 92 J. AM. CHEM. SOC. 6333 (1970) and Amit et al., 39 J. ORG. CHEM. 192 (1974).

(3) Oligonucleotide Probes

To detect gene expression, preferably of genes in the ProMac signature, oligonucleotide probes (polynucleotide sequences) may be designed and synthesized based on known sequence information. For example, 20- to 30-mer oligonucleotides may be selected to monitor expression of deleterious ProMac genes. See Lipshutz et al., 21 NATURE GENET. 20 (1999). In addition to the sequences provided herein, probes for ProMac signature genes, as well as oligonucleotide probes specific for other potentially relevant genes, may be selected from a number of sources including the aforementioned polynucleotide databases. Generally, the probe is complementary to the reference sequence, preferably unique to the cell type of interest (e.g., macrophages), and preferably hybridizes with high affinity and specificity. Lockhart et al., 14 NATURE BIOTECHNOL. 1675 (1996). In addition, the oligonucleotide probe may represent non-overlapping sequences of the reference sequence, which improves probe redundancy resulting in a reduction in false positive rate and an increased accuracy in target quantification. Lipshutz et al., 21 NATURE GENET. 20 (1999).

The oligonucleotide probes may comprise sequences derived from a ProMac signature gene or fragments of a ProMac signature gene. These sequences include, but are not limited to, the ProMac signature genes shown in sequences 1-1474 and 1478. Alternatively, modified oligonucleotides about 80-300 nucleotides in length or about 100-200 nucleotides in length are used on the microarrays. Such microarrays may comprise one or more of such probes. These are especially useful in place of cDNAs for determining the presence of mRNA in a sample, as the modified oligonucleotides have the advantage of rapid synthesis and purification and analysis prior to attachments to the support surface. In particular, oligonucleotides with 2′-modified sugar groups demonstrate increased binding affinity with RNA, and these oligonucleotides are particularly advantageous in identifying mRNA in a sample exposed to a microarray. These probes therefore may be particularly useful for efficient methods of determining the presence and/or level of ProMacs in a samples through transcript expression.

Generally, the oligonucleotide probes are generated by standard synthesis chemistries such as phosphoramidite chemistry (U.S. Pat. Nos. 4,980,460; 4,973,679; 4,725,677; 4,458,066; and 4,415,732; Beaucage and Iyer, 48 TETRAHEDRON 2223 (1992)). Alternative chemistries that create non-natural backbone groups, such as phosphorothionate and phosphoroamidate may also be employed.

Using the “flow channel” method, oligonucleotide probes are synthesized at selected regions on the support by forming flow channels on the surface of the support through which appropriate reagents flow or in which appropriate reagents are placed. For example, if a monomer is to be bound to the support in a selected region, all or part of the surface of the selected region may be activated for binding by flowing appropriate reagents through all or some of the channels, or by washing the entire support with appropriate reagents. After placing a channel block on the surface of the support, a reagent containing the monomer may flow through or may be placed in all or some of the channels. The channels provide fluid contact to the first selected region, thereby binding the monomer on the support directly or indirectly (via a spacer) in the first selected region.

If a second monomer is coupled to a second selected region, some of which may be included among the first selected region, the second selected region may be in fluid contact with second flow channels through translation, rotation, or replacement of the channel block on the surface of the support; through opening or closing a selected valve; or through deposition. The second region may then be activated. Thereafter, the second monomer may then flow through or may be placed in the second flow channels, binding the second monomer to the second selected region. Thus, the resulting oligonucleotides bound to the support are, for example, A, B, and AB. The process is repeated to form a microarray of oligonucleotide probes of desired length at known locations on the support.

Microarrays may have a plurality of modified oligonucleotides or polynucleotides stably associated with the surface of a support, e.g., covalently attached to the surface with or without a linker molecule. Each oligonucleotide on the microarray comprises a modified oligonucleotide composition of known identity and usually of known sequence. By stable association, the associated modified oligonucleotides maintain their position relative to the support under hybridization and washing conditions.

The oligonucleotides may be non-covalently or covalently associated with the support surface. Examples of non-covalent association include non-specific adsorption, binding based on electrostatic interactions (e.g., ion pair interactions), hydrophobic interactions, hydrogen bonding interactions, and specific binding through a specific binding pair member covalently attached to the support surface. Examples of covalent binding include covalent bonds formed between the oligonucleotides and a functional group present on the surface of the rigid support (e.g., —OH), where the functional group may be naturally occurring or present as a member of an introduced linking group.

ii. Polypeptide Arrays

Although attempts to evaluate gene activity and to decipher biological processes have traditionally focused on genomics, proteomics offers a promising look at the biological activities of a cell. Proteomics involves the qualitative and quantitative measurement of gene activity by detecting and quantitating expression at the protein level, rather than at the mRNA level. Proteomic-directed assays are also beneficial for their inclusion of non-genome encoded events such as post-translational modification of proteins, protein-protein interactions, and protein localization within the cell.

The study of ProMac signature gene expression at the protein level is important because many of the important cellular processes are regulated by the protein status of the cell, not by the nucleic acid status of gene expression. Indeed, a disparity may exist between ProMac gene expression and protein expression. See, e.g., Ma et al., 98(17) PROC. NATL. ACAD. SCI. USA 9778 (2001). In addition, the protein content of a cell is highly relevant to developing therapeutics because many drugs are designed to be active against protein targets.

(1) Microarray Supports

The support of the microarray may be either organic or inorganic, biological or non-biological, or any combination of these materials. In addition, the support may be transparent or translucent. The portion of the surface of the support on which the regions of protein-capture agents reside may, for example, be flat and either firm or semi-firm. It is not necessary, however, that the protein microarrays of the invention be flat or entirely two-dimensional. Indeed, significant topological features may be present on the surface of the support surrounding the regions, between the regions or beneath the regions. Walls or other barriers, for example, may separate the regions of the microarray.

Numerous materials are suitable for use as a support in the protein microarrays of the invention. The support may comprise a material selected from the group consisting of silicon, silica, quartz, glass, controlled pore glass, carbon, alumina, titania, tantalum oxide, germanium, silicon nitride, zeolites, and gallium arsenide. Many metals such as gold, platinum, aluminum, copper, titanium, and their alloys may be useful as supports of the microarray. Alternatively, many ceramics and polymers may also be used as supports. Polymers that may be used as supports include, but are not limited to: polystyrene; poly(tetra)fluoroethylene (PTFE); polyvinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyalkenesulfone (PAS); polypropylethylene, polyethylene; polyhydroxyethylmethacrylate (HEMA); polydimethylsiloxane; polyacrylamide; polyimide; and block-copolymers. The support on which the regions of protein-capture agents reside may also be a combination of any of the aforementioned support materials.

(a) Microarray Support Surface

The support surfaces comprise the surfaces on which each of the protein-capture agents is immobilized. The support surfaces may comprise a support surface, an altered support surface, a coating applied to or formed on the support surface, or an organic thinfilm applied to or formed on the support surface or coating surface. Support surfaces comprise materials suitable for immobilization of the protein-capture agents to the microarrays. Suitable support surfaces include membranes, such as nitrocellulose membranes, polyvinylidenedifluoride (PVDF) membranes, and the like. In another embodiment, the support surfaces may comprise a hydrogel such as dextran. Alternatively, the support surfaces may comprise an organic thinfilm including lipids, charged peptides (e.g., polylysine or poly-arginine), or a neutral amino acid (e.g., polyglycine).

The support surfaces may also comprise a compound that has the ability to interact with both the support and the protein-capture agent. For example, functionalities enabling interaction with the support may include hydrocarbons having functional groups, which may interact with functional groups on the support (e.g., —O—, —CONH—, CONHCO—, —NH—, —CO—, —S—, —SO—). Functionalities enabling interaction with the protein-capture agent comprise antibodies, antigens, receptor ligands, compounds comprising binding sites for affinity tags, and the like.

In another embodiment, the support surfaces may include a coating. The coating may be formed on, or applied to, the support surfaces. The support may be modified with a coating by using thinfilm technology based, for example, on physical vapor deposition (PVD), plasma-enhanced chemical vapor deposition (PECVD), or thermal processing.

Alternatively, plasma exposure may be used to directly activate or alter the support and create a coating. For example, plasma etch procedures can be used to oxidize a polymeric surface (for example, polystyrene or polyethylene to expose polar functionalities such as hydroxyls, carboxylic acids, aldehydes and the like) which then act as a coating.

The coating may further comprise a component to reduce non-specific binding. For example, a polypropylene support may be coated with a compound, such as bovine serum albumin, to reduce non-specific binding.

Alternatively, the coating may comprise an antibody. More particularly, antibodies that recognize epitope tags engineered into recombinant proteins may be employed. Alternatively, recombinant proteins may comprise a poly-histidine affinity tag, in which case an anti-histidine antibody chemically linked to the support provides a support surfaces for immobilization of the protein-capture agents.

In yet another embodiment, the coating may comprise a metal film. The metal film may range from about 50 nm to about 500 nm in thickness. Alternatively, the metal film may range from about 1 nm to about 1 μm in thickness. Examples of metal films that may be used as support coatings include aluminum, chromium, titanium, tantalum, nickel, stainless steel, zinc, lead, iron, copper, magnesium, manganese, cadmium, tungsten, cobalt, and alloys or oxides thereof. In one embodiment, the metal film is a noble metal film. Noble metals that may be used for a coating include, but are not limited to, gold, platinum, silver, and copper. The coating may also comprise a gold alloy. Electron-beam evaporation may be used to provide a thin coating of gold on the surface of the support. Additionally, commercial metal-like substances may be employed, such as TALON metal affinity resin and the like.

In alternative embodiments, the coating may comprise a composition selected from the group consisting of silicon, silicon oxide, titania, tantalum oxide, silicon nitride, silicon hydride, indium tin oxide, magnesium oxide, alumina, glass, hydroxylated surfaces, and polymers.

It is contemplated that the coatings of the microarrays may require the addition of at least one adhesion layer between the coating and the support. The adhesion layer may be at least about 6 angstroms thick but may be much thicker. For example, a layer of titanium or chromium may be desirable between a silicon wafer and a gold coating. Alternatively, an epoxy glue such as Epo-tek 377® or Epo-tek 301-2® (Epoxy Technology Inc., Billerica, Mass.) may be used to aid adherence of the coating to the support. Determinations as to what material should be used for the adhesion layer would be obvious to one skilled in the art once materials are chosen for both the support and coating. In other embodiments, additional adhesion mediators or interlayers may be necessary to improve the optical properties of the microarray, for example, waveguides for detection purposes.

In one embodiment of the invention, the surface of the coating is atomically flat. The mean roughness of the surface of the coating may be less than about 5 angstroms for areas of at least about 25 μm². In another embodiment, the mean roughness of the surface of the coating is less than about 3 angstroms for areas of at least about 25 μm². In yet another embodiment, the coating may be a template-stripped surface. See, e.g., Hegner et al., 291 SURFACE SCIENCE 39 (1993); Wagner et al., 11 LANGMUIR 3867 (1995).

Several different types of coating may be combined on the surface. The coating may cover the whole surface of the support or only parts of it. In one embodiment, the coating covers the support surface only at the site of the regions of protein-capture agents. Techniques useful for the formation of coated regions on the surface of the support are well known to those of ordinary skill in the art. For example, the regions of coatings on the support may be fabricated by photolithography, micromolding (U.S. Pat. No. 6,180,239), wet chemical or dry etching, or any combination of these.

(i) Organic Thinfilms

The support surface of the array may comprise an organic thinfilm layer. The organic thinfilm on which each of the regions of protein-capture agents reside forms a layer either on the support itself or on a coating covering the support. In one embodiment, the organic thinfilm on which the protein-capture agents of the regions are immobilized is less than about 20 nm thick. In another embodiment, the organic thinfilm of each of the regions is less than about 10 nm thick.

A variety of different organic thinfilms are suitable for use in the invention. For example, a hydrogel composed of a material such as dextran may serve as a suitable organic thinfilm on the regions of the microarray. A lipid bilayer may also serve as a suitable thinfilm material.

In another embodiment, the organic thinfilm of each of the regions of the microarray is a monolayer. A monolayer of polyarginine or polylysine adsorbed on a negatively charged support or coating may comprise the organic thinfilm. Another option is a disordered monolayer of tethered polymer chains. The organic thinfilm may be a self-assembled monolayer. Specifically, the self-assembled monolayer may comprise molecules of the formula X—R—Y, wherein R is a spacer, X is a functional group that binds R to the surface, and Y is a functional group for binding protein-capture agents onto the monolayer. Alternatively, the self-assembled monolayer may be comprised of molecules of the formula (X)ₐR(Y)_{b} where a and b are, independently, integers greater than or equal to 1 and X, R, and Y are as previously defined.

In another embodiment, the organic thinfilm comprises a combination of organic thinfilms such as a combination of a lipid bilayer immobilized on top of a self-assembled monolayer of molecules of the formula X—R—Y. As another example, a monolayer of polylysine may be combined with a self-assembled monolayer of molecules of the formula X—R—Y. See U.S. Pat. No. 5,629,213.

In all cases, the coating, or the support itself if no coating is present, must be compatible with the chemical or physical adsorption of the organic thinfilm on its surface. For example, if the microarray comprises a coating between the support and a monolayer of molecules of the formula X—R—Y, then it is understood that the coating must be composed of a material for which a suitable functional group X is available. If no such coating is present, then it is understood that the support must be composed of a material for which a suitable functional group X is available.

In one embodiment of the invention, the area of the support surface, or coating surface, which separates the regions of protein-capture agents are free of organic thinfilm. In an alternative embodiment, the organic thinfilm may extend beyond the area of the support surface, or coating surface if present, covered by the regions of protein-capture agents. For example, the entire surface of the microarray may be covered by an organic thinfilm on which the plurality of spatially distinct regions of protein-capture agents reside. An organic thinfilm that covers the entire surface of the microarray may be homogenous or may comprise regions of differing exposed functionalities useful in the immobilization of regions of different protein-capture agents.

In yet another embodiment, the areas of the support surface or coating surface between the regions of protein-capture agents are covered by an organic thinfilm, but an organic thinfilm of a different type than that of the regions of protein-capture agents. For example, the surfaces between the regions of protein-capture agents may be coated with an organic thinfilm characterized by low non-specific binding properties for proteins and other analytes.

A variety of techniques may be used to generate regions of organic thinfilm on the surface of the support or on the surface of a coating on the support. These techniques are well known to those skilled in the art and will vary depending upon the nature of the organic thinfilm, the support, and the coating, if present. The techniques will also vary depending on the structure of the underlying support and the pattern of any coating present on the support. For example, regions of a coating that are highly reactive with an organic thinfilm may have already been produced on the support surface. Areas of organic thinfilm may be created by microfluidics printing, microstamping (U.S. Pat. Nos. 5,731,152 and 5,512,131), or microcontact printing (U.S. Pat. No. 6,180,239). Subsequent immobilization of protein-capture agents to the reactive monolayer regions result in two-dimensional microarrays of the agents. Inkjet printer heads provide another option for patterning monolayer X—R—Y molecules, or components thereof, or other organic thinfilm components to nanometer or micrometer scale sites on the surface of the support or coating. See, e.g., Lemmo et al., 69 ANAL CHEM. 543 (1997); U.S. Pat. Nos. 5,843,767 and 5,837,860. In some cases, commercially available arrayers based on capillary dispensing may also be of use in directing components of organic thinfilms to spatially distinct regions of the microarray (OmniGrid® from Genemachines, Inc, San Carlos, Calif., and High-Throughput Microarrayer from Intelligent Bio-Instruments, Cambridge, Mass.). Other methods for the formation of organic thinfilms include in situ growth from the surface, deposition by physisorption, spin-coating, chemisorption, self-assembly, or plasma-initiated polymerization from gas phase.

Diffusion boundaries between the regions of protein-capture agents immobilized on organic thinfilms such as self-assembled monolayers may be integrated as topographic patterns (physical barriers) or surface functionalities with orthogonal wetting behavior (chemical barriers). For example, walls of support material may be used to separate some of the regions of protein-capture agents from some of the others or all of the regions from each other. Alternatively, non-bioreactive organic thinfilms, such as monolayers, with different wettability may be used to separate regions of protein-capture agents from one another.

(2) Protein-Capture Agents

A protein microarray contemplated by the invention may contain any number of different proteins, amino acid sequences, polynucleotide sequences, or small molecules. In one specific embodiment, the microarrays comprise at least one protein-capture agent that binds a polypeptide encoded by a ProMac signature gene.

The target proteins bound by the protein-capture agents immobilized on the microarray may be members of the same family. Such families include, but are not limited to, families of mucins, growth factor receptors, hormone receptors, neurotransmitter receptors, catecholamine receptors, amino acid derivative receptors, cytokine receptors, extracellular matrix receptors, antibodies, lectins, cytokines, serpins, proteinases, kinases, phosphatases, ras-like GTPases, hydrolases, steroid hormone receptors, transcription factors, DNA binding proteins, zinc finger proteins, leucine-zipper proteins, homeodomain proteins, intracellular signal transduction modulators and effectors, apoptosis-related factors, DNA synthesis factors, DNA repair factors, DNA recombination factors, cell-surface antigens, Hepatitis C virus (HCV) proteases, HIV proteases, viral integrases, and proteins from pathogenic bacteria.

A protein-capture agent on the microarray may be any molecule or complex of molecules that has the ability to bind a target ProMac protein and immobilize it to the site of the protein-capture agent on the microarray. In one aspect, the protein-capture agent binds its target protein in a substantially specific manner. For example, the protein-capture agent may be a protein whose natural function in a cell is to specifically bind another protein, such as an antibody, a receptor, an antibody fragment, or a receptor fragment. Alternatively, the protein-capture agent may be a partially or wholly synthetic or recombinant protein that specifically binds a target protein.

Moreover, the protein-capture agent may be a protein that has been selected in vitro from a mutagenized, randomized, or completely random and synthetic library by its binding affinity to a specific target protein or peptide target. The selection method used may be a display method such as ribosome display or phage display. Alternatively, the protein-capture agent obtained via in vitro selection may be a DNA or RNA aptamer that specifically binds a protein target. See, e.g., Potyrailo et al., 70 ANAL. CHEM. 3419 (1998); Cohen, et al., 94 PROC. NATL. ACAD. SCI. USA 14272 (1998); Fukuda, et al., 37 NUCL. ACIDS SYMP. SER., 237 (1997). Alternatively, the in vitro selected protein-capture agent may be a polypeptide. Roberts and Szostak, 94 PROC. NATL. ACAD. SCI. USA 12297 (1997). In yet another embodiment, the protein-capture agent may be a small molecule that has been selected from a combinatorial chemistry library or is isolated from an organism.

(a) Attachment of Protein-Capture Agents

It is necessary to immobilize proteins-capture agents on a solid support in a way that preserves their folded conformations. Methods of arraying functionally active proteins using microfabricated polyacrylamide gel pads to preserve samples and microelectrophoresis to accelerate diffusion have been described elsewhere. Arenkov et al., 278 ANAL. BIOCHEM. 123 (2000). The method of attachment will vary with the support and protein-capture agent selected.

In one embodiment, the protein-immobilizing regions of the microarray comprise an affinity tag that enhances immobilization of the protein-capture agent onto the organic thinfilm. The use of an affinity tag on the protein-capture agent of the microarray provides several advantages. An affinity tag can confer enhanced binding or reaction of the protein-capture agent with the functionalities on the organic thinfilm, such as Y if the organic thinfilm is an X—R—Y monolayer as previously described. This enhancement effect may be either kinetic or thermodynamic. The affinity tag/organic thinfilm combination used in the regions of protein-capture agents residing on the microarray allows for immobilization of the protein-capture agents in a manner that does not require harsh reaction conditions which are adverse to protein stability or function. In most embodiments, the protein-capture agents are immobilized to the organic thinfilm in aqueous, biological buffers.

An affinity tag also offers immobilization on the organic thinfilm that is specific to a designated site or location on the protein-capture agent (site-specific immobilization). For this to occur, attachment of the affinity tag to the protein-capture agent must be site-specific. Site-specific immobilization helps ensure that the protein-binding site of the agent, such as the antigen-binding site of the antibody moiety, remains accessible to ligands in solution. Another advantage of immobilization through affinity tags is that it allows for a common immobilization strategy to be used with multiple, different protein-capture agents.

The affinity tag may be attached directly, either covalently or noncovalently, to the protein-capture agent. In an alternative embodiment, however, the affinity tag is either covalently or noncovalently attached to an adaptor that is either covalently or noncovalently attached to the protein-capture agent.

In one embodiment, the affinity tag comprises at least one amino acid. The affinity tag may be a polypeptide comprising at least two amino acids which are reactive with the functionalities of the organic thinfilm. Alternatively, the affinity tag may be a single amino acid that is reactive with the organic thinfilm. Examples of possible amino acids that could be reactive with an organic thinfilm include cysteine, lysine, histidine, arginine, tyrosine, aspartic acid, glutamic acid, tryptophan, serine, threonine, and glutamine. A polypeptide or amino acid affinity tag may be expressed as a fusion protein with the protein-capture agent when the protein-capture agent is a protein, such as an antibody or antibody fragment. Amino acid affinity tags provide either a single amino acid or a series of amino acids that may interact with the functionality of the organic thinfilm, such as the Y-functional group of the self-assembled monolayer molecules. Amino acid affinity tags may be readily introduced into recombinant proteins to facilitate oriented immobilization by covalent binding to the Y-functional group of a monolayer or to a functional group on an alternative organic thinfilm.

The affinity tag may comprise a poly-amino acid tag. A poly-amino acid tag is a polypeptide that comprises from about 2 to about 100 residues of a single amino acid, optionally interrupted by residues of other amino acids. For example, the affinity tag may comprise a poly-cysteine, poly-lysine, poly-arginine, or poly-histidine. Amino acid tags may comprise about two to about twenty residues of a single amino acid, such as, for example, histidines, lysines, arginines, cysteines, glutamines, tyrosines, or any combination of these. For example, an amino acid tag of one to twenty amino acids includes at least one to ten cysteines for thioether linkage; or one to ten lysines for amide linkage; or one to ten arginines for coupling to vicinal dicarbonyl groups. One of ordinary skill in the art can readily pair suitable affinity tags with a given functionality on an organic thinfilm.

The position of the amino acid tag may be at the N- or C-terminus of the protein-capture agent which is a protein, or anywhere in-between, provided that the protein-binding region of the protein-capture agent remains accessible for protein binding. Affinity tags introduced for protein purification may be located at the C-terminus of the recombinant protein to ensure that only full-length proteins are isolated during protein purification. If intact antibodies are used on the microarrays, then the attachment point of the affinity tag on the antibody may be at a C-terminus of the effector (Fc) region of the antibody. If scFvs are used on the microarrays, then the attachment point of the affinity tag may also be located at the C-terminus of the molecules.

Affinity tags may also contain one or more unnatural amino acids. Unnatural amino acids may be introduced using suppressor tRNAs that recognize stop codons (i.e., amber) See, e.g., Cload et al., 3 CHEM. BIOL. 1033 (1996); Ellman et al., 202 METHODS ENZYM. 301 (1991); and Noren et al., 244 SCIENCE 182 (1989). The tRNAs are chemically amino-acylated to contain chemically altered (“unnatural”) amino acids for use with specific coupling chemistries (i.e., ketone modifications, photoreactive groups).

In an alternative embodiment, the affinity tag comprises an intact protein, such as, but not limited to, glutathione S-transferase, an antibody, avidin, or streptavidin.

In embodiments where the protein-capture agent is a protein and the affinity tag is a protein, such as a poly-amino acid tag or a single amino acid tag, the affinity tag may be attached to the protein-capture agent by generating a fusion protein. Alternatively, protein synthesis or protein ligation techniques known to those skilled in the art may be used. See, e.g., Mathys, et al., 231 GENE 1 (1999); Evans, et al., 7 PROTEIN SCIENCE 2256 (1998).

Other conjugation and immobilization techniques known in the art may be adapted for the purpose of attaching affinity tags to the protein-capture agent. For example, the affinity tag may be an organic bioconjugate that may be chemically coupled to the protein-capture agent. Biotin or antigens may be chemically cross-linked to the protein. Alternatively, a chemical crosslinker may be used that attaches a simple functional moiety such as a thiol or an amine to the surface of a protein serving as a protein-capture agent on the microarray.

In one embodiment of the invention, the organic thinfilm of each of the regions may comprise, at least in part, a lipid monolayer or bilayer, and the affinity tag may comprise a membrane anchor.

In an alternative embodiment, no affinity tag is used to immobilize the protein-capture agents onto the organic thinfilm. An amino acid or other moiety (such as a carbohydrate moiety) inherent to the protein-capture agent itself may instead be used to tether the protein-capture agent to the reactive group of the organic thinfilm. In one embodiment, the immobilization is site-specific with respect to the location of the site of immobilization on the protein-capture agent. For example, the sulfhydryl group on the C-terminal region of the heavy chain portion of a Fab′ fragment generated by pepsin digestion of an antibody, followed by selective reduction of the disulfide bond between monovalent Fab′ fragments, may be used as the affinity tag. Alternatively, a carbohydrate moiety on the Fc portion of an intact antibody may be oxidized under mild conditions to an aldehyde group suitable for immobilizing the antibody on a monolayer via reaction with a hydrazide-activated Y group on the monolayer. See, e.g., U.S. Pat. No. 6,329,209; Dammer et al., 70 BIOPHYS J. 2437 (1996).

Because the protein-capture agents of at least some of the different regions on the microarray are different from each other, different solutions, each containing a different protein-capture agent, must be delivered to the individual regions. Solutions of protein-capture agents may be transferred to the appropriate regions via arrayers, which are well-known in the art and commercially available. For example, microcapillary-based dispensing systems may be used. These dispensing systems may be automated and computer-aided. A description of and building instructions for an example of a microarrayer comprising an automated capillary system can be found on the internet at http://cmgm.stanford.edu/pbrown/microarray.html and http://cmgm.stanford.edu/pbrown/mguide/index.html. The use of other microprinting techniques for transferring solutions containing the protein-capture agents to the agent-reactive regions is also possible. Ink-jet printer heads may also be used for precise delivery of the protein-capture agents to the agent-reactive regions. Representative, non-limiting disclosures of techniques useful for depositing the protein-capture agents on the appropriate regions of the support may be found, for example, in U.S. Pat. Nos. 5,843,767 (ink-jet printing technique, Hamilton 2200 robotic pipetting delivery system); 5,837,860 (ink-jet printing technique, Hamilton 2200 robotic pipetting delivery system); 5,807,522 (capillary dispensing device); and 5,731,152 (stamping apparatus). Other methods of arraying functionally active proteins include attaching proteins to the surfaces of chemically derivatized microscope slides. See MacBeath & Schreiber, 289 SCIENCE 1760 (2000).

(i) Adaptors

Another embodiment of the protein microarrays of the invention comprises an adaptor that links the affinity tag to the protein-capture agent on the regions of the microarray. The additional spacing of the protein-capture agent from the surface of the support (or coating) that is afforded by the use of an adaptor is particularly advantageous if the protein-capture agent is a protein, because proteins are prone to surface inactivation. The adaptor may afford some additional advantages as well. For example, the adaptor may help facilitate the attachment of the protein-capture agent to the affinity tag. In another embodiment, the adaptor may help facilitate the use of a particular detection technique with the microarray. One of ordinary skill in the art will be able to choose an adaptor which is appropriate for a given affinity tag. For example, if the affinity tag is streptavidin, then the adaptor could be biotin that is chemically conjugated to the protein-capture agent which is to be immobilized.

The adaptor may comprise a protein. Alternatively, the affinity tag, adaptor, and protein-capture agent together may comprise a fusion protein. Such a fusion protein may be readily constructed using standard recombinant DNA technology. Protein adaptors are especially useful to increase the solubility of the protein-capture agent of interest and to increase the distance between the surface of the support or coating and the protein-capture agent. A protein adaptor can also be useful in facilitating the preparative steps of protein purification by affinity binding prior to immobilization on the microarray. Examples of possible adaptor proteins include glutathione-5-transferase (GST), maltose-binding protein, chitin-binding protein, thioredoxin, and green-fluorescent protein (GFP). GFP may also be used for quantification of surface binding. In an embodiment in which the protein-capture agent is an antibody moiety comprising the Fc region, the adaptor may be a polypeptide, such as protein G, protein A, or recombinant protein A/G (a gene fusion product secreted from a non-pathogenic form of Bacillus which contains four Fc binding domains from protein A and two from protein G).

(b) Preparation of the Protein-Capture Agents

The protein-capture agents used on the microarray may be produced by any of the variety of means known to those of ordinary skill in the art. The protein-capture agents may comprise proteins, specifically, antibodies or fragments thereof, ligands, receptor proteins, aptamers, or small molecules.

In preparation for immobilization to the microarrays of the invention, the antibody moiety, or any other protein-capture agent that is a protein or polypeptide, may be expressed from recombinant DNA either in vivo or in vitro. The cDNA encoding the protein-capture agent may be cloned into an expression vector (many examples of which are commercially available) and introduced into cells of the appropriate organism for expression. Expression in vivo may be accomplished in bacteria (e.g., E. coli), plants (e.g., N. tabacum), lower eukaryotes (e.g., S. cerevisiae, S. pombe, P. pastoris), or higher eukaryotes (e.g., bacculovirus-infected insect cells, insect cells, mammalian cells). For in vitro expression, PCR-amplified DNA sequences may be directly used in coupled in vitro transcription/translation systems (e.g., E. coli S30 lysates from T7 RNA polymerase expressing, preferably protease-deficient strains; wheat germ lysates; reticulocyte lysates). The choice of organism for optimal expression depends on the extent of post-translational modifications (e.g., glycosylation, lipid-modifications) desired. The choice of protein-capture agent also depends on other issues, for example, whether an intact antibody is to be produced or only a fragment thereof (and which fragment), because disulfide bond formation will be affected by the choice of a host cell. One of ordinary skill in the art will be able to readily choose which host cell type is most suitable for the protein-capture agent and application desired.

DNA sequences encoding affinity tags and adaptors may be engineered into the expression vectors such that the protein-capture agent genes of interest can be cloned in frame either 5′ or 3′ of the DNA sequence encoding the affinity tag and adaptor protein. In most aspects, the expressed protein-capture agents may purified by affinity chromatography using commercially available resins.

Production of a plurality of protein-capture agents may involve parallel processing from cloning to protein expression and protein purification. cDNAs encoding the protein-capture agent of interest may be amplified by PCR using cDNA libraries or expressed sequence tag (EST) clones as templates. For in vivo expression of the proteins, cDNAs may be cloned into commercial expression vectors and introduced into an appropriate organism for expression. For in vitro expression PCR-amplified DNA sequences may be directly used in coupled transcription/translation systems.

E. coli-based protein expression is generally the method of choice for soluble proteins that do not require extensive post-translational modifications for activity. Extracellular or intracellular domains of membrane proteins may be fused to protein adaptors for expression and purification.

The entire approach may be performed using 96-well plates. PCR reactions may be carried out under standard conditions. Oligonucleotide primers may contain unique restriction sites for facile cloning into the expression vectors. Alternatively, the TA cloning system may be used. The expression vectors may further contain the sequences for affinity tags and the protein adaptors. PCR products may be ligated into the expression vectors (under inducible promoters) and introduced into the appropriate competent E. coli strain by a method such as calcium-dependent transformation. Transformed E. coli cells are plated and individual colonies are transferred into 96-microarray blocks. Cultures are grown to mid-log phase, induced for expression, and cells collected by centrifugation. Cells are resuspended in solutions containing lysozyme and the membranes are broken by rapid freeze/thaw cycles or sonication. Cell debris is removed by centrifugation and the supernatants transferred to 96-well arrays. The appropriate affinity matrix is added, the protein-capture agent of interest is bound and nonspecifically bound proteins are removed by repeated washing and other steps using centrifugation devices. Alternatively, magnetic affinity beads and filtration devices may be used. The proteins are eluted and transferred to a new 96-well microarray. Protein concentrations are determined and an aliquot of each protein-capture agent is spotted onto a nitrocellulose filter and verified by Western analysis using an antibody directed against the affinity tag on the protein-capture agent. The purity of each sample is assessed by SDS-PAGE and Silver staining or mass spectrometry. The protein-capture agents are then snap-frozen and stored at −80° C.

S. cerevisiae allows for the production of glycosylated protein-capture agents such as antibodies or antibody fragments. For production in S. cerevisiae, the approach described above for E. coli may be used with the appropriate and obvious modifications for transformation and cell lysis procedures. Variations of post-translational modifications may be obtained by using different yeast strains (i.e., S. pombe, P. pastoris).

One aspect of the bacculovirus system is the microarray of post-translational modifications that can be obtained, although antibodies and other proteins produced in bacculovirus contain carbohydrate structures very different from those produced by mammalian cells. The bacculovirus-infected insect cell system requires cloning of viruses, obtaining high titer stocks and infection of liquid insect cell suspensions (cells such as SF9, SF21).

Mammalian cell-based expression requires transfection and cloning of cell lines. Either lymphoid or non-lymphoid cell may be used in the preparation of antibodies and antibody fragments. Soluble proteins such as antibodies are collected from the medium while intracellular or membrane bound proteins require cell lysis (either detergent solubilization or freeze-thaw). The protein-capture agents may then be purified by a procedure analogous to that described for E. coli.

For in vitro translation, the system of choice is E. coli lysates obtained from protease-deficient and T7 RNA polymerase overexpressing strains. E. coli lysates provide efficient protein expression (30-50 μg/ml lysate). The entire process may be carried out in 96-well arrays. Antibody genes or other protein-capture agent genes of interest may be amplified by PCR using oligonucleotides that contain the gene-specific sequences containing a T7 RNA polymerase promoter and binding site and a sequence encoding the affinity tag. Alternatively, an adaptor protein may be fused to the gene of interest by PCR. Amplified DNAs may be directly transcribed and translated in the E. coli lysates without prior cloning for fast analysis. The antibody fragments or other proteins may then be isolated by binding to an affinity matrix and processed as described above.

Alternative in vitro translation systems that may be used include wheat germ extracts and reticulocyte extracts. In vitro synthesis of membrane proteins or post-translationally modified proteins will require reticulocyte lysates in combination with microsomes.

In one embodiment of the invention, the protein-capture agents on the microarray comprise monoclonal antibodies. The production of monoclonal antibodies against specific protein targets is routine using standard hybridoma technology. In fact, numerous monoclonal antibodies are available commercially.

As an alternative to obtaining antibodies or antibody fragments by cell fusion or from continuous cell lines, the antibody moieties may be expressed in bacteriophage. Such antibody phage display technologies are well known to those skilled in the art. The bacteriophage protein-capture agents allow for the random recombination of heavy- and light-chain sequences, thereby creating a library of antibody sequences that may be selected against the desired antigen. The protein-capture agent may be based on bacteriophage lambda or on filamentous phage. The bacteriophage protein-capture agent may be used to express Fab fragments, Fv's with an engineered intermolecular disulfide bond to stabilize the V_{H}-V_{L} pair (dsFv's), scFvs, or diabody fragments.

The antibody genes of the phage display libraries may be derived from pre-immunized donors. For example, the phage display library could be a display library prepared from the spleens of mice previously immunized with a mixture of proteins, such as a lysate of human T-cells. Immunization may be used to bias the library to contain a greater number of recombinant antibodies reactive towards a specific set of proteins, such as proteins found in human T-cells. Alternatively, the library antibodies may be derived from native or synthetic libraries. The native libraries may be constructed from spleens of mice that have not been contacted by external antigen. In a synthetic library, portions of the antibody sequence, typically those regions corresponding to the complementarity determining regions (CDR) loops, have been mutagenized or randomized.

iii. Target Samples

Biological samples may be isolated from several sources including, but not limited to, a patient or a cell line. Patient samples may include blood, urine, amniotic fluid, plasma, semen, bone marrow, and tissues. Once isolated, total RNA or protein may be extracted using methods well known in the art. For example, target samples may be generated from total RNA by dT-primed reverse transcription producing cDNA. See, e.g., SAMBROOK ET AL., MOLECULAR CLONING: A LAB. MANUAL (2001); and AUSUBEL ET AL., CURRENT PROTOCOLS IN MOLECULAR BIOLOGY, John Wiley & Sons, Inc. (1995). The cDNA may then be transcribed to cRNA by in vitro transcription resulting in a linear amplification of the RNA. The target samples may be labeled with, for example, a fluorescent dye (e.g., Cy3-dUTP) or biotin. The labeled targets may be hybridized to the microarray. Laser excitation of the target samples produces fluorescence emissions, which are captured by a detector. This information may then be used to generate a quantitative two-dimensional fluorescence image of the hybridized targets.

Gene expression profiles of a particular tissue or cell type (e.g., ProMacs) may be generated from RNA (i.e., total RNA or mRNA). Reverse transcription with an oligo-dT primer may be used to isolate and generate mRNA from cellular RNA. To maximize the amount of sample or signal, labeled total RNA may also be used. The RNA may be fluorescently labeled or labeled with a radioactive isotope. For radioactive detection, a low energy emitter, such as ³³P-dCTP, is preferred due to close proximity of the oligonucleotide probes on the support. The fluorophores, Cy3-dUTP or Cy5-dUTP, may used for fluorescent labeling. These fluorophores demonstrate efficient incorporation with reverse transcriptase and better yields. Furthermore, these fluorophores possess distinguishable excitation and emission spectra. Thus, two samples, each labeled with a different fluorophore, may be simultaneously hybridized to a microarray.

Typically, the polynucleotide sample may be amplified prior to hybridization. Amplification methods include, but are not limited to PCR (INNIS ET AL., PCR PROTOCOLS: A GUIDE TO METHODS & APPLICATION (1990)), ligase chain reaction (LCR) (Wu & Wallace, 4(4) GENOMICS 560 (1989); Landegren et al., 241(4869) SCIENCE 1077 (1988); Barringer et al., 89(1) GENE 117 (1990)), transcription amplification (Kwoh et al., 86(4) PNAS 1173 (1989)), and self-sustained sequence replication (Guatelli et al., 87(5) PNAS 1874 (1990)). The labeled RNA targets are then hybridized to the microarray. See, e.g., Cheung et al., 21 NATURE GENET. 15 (1999).

The target polynucleotides may be labeled at one or more nucleotides during or after amplification. Labels suitable for use with microarray technology include labels detectable by spectroscopic, photochemical, biochemical, immunochemical, electrical, optical, or chemical means. The detectable label may be a luminescent label, such as a fluorescent label, a chemiluminescent label, a bioluminescent label, or a colorimetric label. A fluorescent label may be fluorescein, rhodamine, lissamine, phycoerythrin, polymethine dye derivative, phosphor, or Cy2, Cy3, Cy3.5, Cy5, Cy5.5, Cy7. Commercially available fluorescent labels include fluorescein phosphoramidites such as Fluoreprime (Pharmacia, Piscataway, N.J.), Fluoredite (Millipore, Bedford, Mass.), and FAM (ABI, Foster City, Calif.). Other labels include biotin for staining with labeled streptavidin conjugate, magnetic beads (e.g., Dynabeads), fluorescent dyes (e.g., texas red, rhodamine, green fluorescent protein), radiolabels (e.g., ³H, ¹²⁵I, ³⁵S, ¹⁴C, or ³²P), enzymes (e.g., horseradish peroxidase, alkaline phosphatase), and colorimetric labels such as colloidal gold or colored glass or plastic (e.g., polystyrene, polypropylene, latex) beads (see, e.g., U.S. Pat. Nos. 4,366,241; 4,277,437; 4,275,149; 3,996,345; 3,939,350; 3,850,752; and 3,817,837).

The labeled polynucleotide targets are then hybridized to the microarray. A number of buffers may be used for hybridization assays. By way of example, but not limitation, the buffers can be any of the following: 5 M betaine, 1 M NaCl, pH 7.5; 4.5 M betaine, 0.5 M LiCl, pH 8.0; 3 M TMACl, 50 mM Tris-HCl, 1 mM EDTA, 0.1% N-lauroyl-sarkosine (LS); 2.4 M TEAC1, 50 mM Tris-HCl, pH 8.0, 0.1% NLS; 1 M LiCl, 10 mM Tris-HCl, pH 8.0, 10% formamide; 2 M GuSCN, 30 mM NaCitrate, pH 7.5; 1 M LiCl, 10 mM Tris-HCl, pH 8.0, 1 mM CTAB; 0.3 mM spermine, 10 mM Tris-HCl, pH 7.5; and 2 M N₄OAc with 2 volumes absolute ethanol. Addition volumes of ionic detergents (such as N-lauroyl-sarkosine) may be added to the buffer. Hybridization may be performed at about 20-65° C. See, e.g., U.S. Pat. No. 6,045,996. Additional examples of hybridization conditions are disclosed in SAMBROOK ET AL., MOLECULAR CLONING: A LAB. MANUAL (2001); Berger and Kimmel, GUIDE TO MOLECULAR CLONING TECHNIQUES, METHODS IN ENZYMOLOGY, (1987), Volume 152, Academic Press, Inc., San Diego, Calif.; Young and Davis, 80 PROC. NATL. ACAD. SCI. U.S.A. 1194 (1983).

The hybridization buffer may be a formamide-based buffer or an aqueous buffer containing dextran sulfate or polyethylene glycol. See, e.g., Cheung et al., 21 NATURE GENET. 15-19 (1999); SAMBROOK ET AL., MOLECULAR CLONING: A LAB. MANUAL (2001). In addition, the hybridization buffer may contain blocking agents such as sheared salmon sperm DNA or Denhardt's reagent to minimize nonspecific binding or background noise. Approximately 50-200 μg labeled total RNA or 2-5 μg labeled mRNA per hybridization is required for a sufficient fluorescent signal and detection. Typically, the amount of oligonucleotide probes attached to the support is in excess of the labeled target RNA.

Following hybridization, the polynucleotides may be analyzed by detecting one or more labels attached to the target polynucleotides. The labels may be incorporated by any of a number of methods well known in the art. In one embodiment, the label may be simultaneously incorporated during the amplification step in the preparation of the target polynucleotides. For example, a labeled amplification product may be generated by PCR using labeled primers or labeled nucleotides. Transcription amplification using a labeled nucleotide (e.g., fluorescein-labeled UTP or CTP) incorporates a label into the transcribed polynucleotides. Alternatively, a label may be added directly to the original polynucleotide sample or to the amplification product following amplification. Methods for labeling polynucleotides are well-known in the art and include, for example, nick translation or end-labeling.

The hybridized microarray is then subjected to laser excitation, which produces an emission with a unique spectra. The spectra are scanned, for example, with a scanning confocal laser microscope generating monochrome images of the microarray. These images are digitally processed and normalized based on a threshold value (e.g., background) using mathematical algorithms. For example, a threshold value of 0 may be assigned when no change in the level of fluorescence is observed; an increase in fluorescence may be assigned a value of +1 and a decrease in fluorescence may be assigned a value of −1. Normalization may be based on a designated subgroup of genes where variations in this subgroup are utilized to generate statistics applicable for evaluating the complete gene microarray. Chen et al., 2 J. BIOMED. OPTICS 364 (1997).

Use of one of the protein microarrays of the invention may involve placing the two-dimensional microarray in a flowchamber with approximately 1-10 μl of fluid volume per 25 mm² overall surface area. The cover over the microarray in the flowchamber is preferably transparent or translucent. In one embodiment, the cover may comprise Pyrex or quartz glass. In other embodiments, the cover may be part of a detection system that monitors interaction between the protein-capture agents immobilized on the microarray and protein in a solution such as a cellular extract from a biological sample. The flowchambers should remain filled with appropriate aqueous solutions to preserve protein activity. Salt, temperature, and other conditions are preferably kept similar to those of normal physiological conditions. Target proteins in a fluid solution may be flushed into the flow chamber as desired and their interaction with the immobilized protein-capture agents determined. Sufficient time must be given to allow for binding between the protein-capture agent and its target protein to occur. The amount of time required for this will vary depending upon the nature and tightness of the affinity of the protein-capture agent for its target protein. No specialized microfluidic pumps, valves, or mixing techniques are required for fluid delivery to the microarray.

Alternatively, target protein-containing fluid may be delivered to each of the regions of protein-capture agents individually. For example, in one embodiment, the regions of the support surface where the protein-capture agents reside may be microfabricated in such a way as to allow integration of the microarray with a number of fluid delivery channels oriented perpendicular to the microarray surface, each one of the delivery channels terminating at the site of an individual protein-capture agent-coated region.

The sample, which is delivered to the microarray, will typically be a fluid. In a one embodiment, the sample is a cellular extract or a biological sample. The sample to be assayed may comprise a complex mixture of proteins, including a multitude of proteins which are not target proteins of the protein-capture agents of the microarray. If the proteins to be analyzed in the sample are membrane proteins, then those proteins will typically need to be solubilized prior to administration of the sample to the microarray. If the proteins to be assayed in the sample are proteins secreted by a population of cells in an organism, the sample may be a biological sample. If the proteins to be assayed in the sample are intracellular, a sample may be a cellular extract. In another embodiment, the microarray may comprise protein-capture agents that bind fragments of the expression products of a cell or population of cells in an organism. In such a case, the proteins in the sample to be assayed may have been prepared by performing a digest of the protein in a cellular extract or a biological sample. In an alternative application, the proteins from only specific fractions of a cell are collected for analysis in the sample.

In general, delivery of solutions containing target proteins to be bound by the protein-capture agents of the microarray may be preceded, followed, or accompanied by delivery of a blocking solution. A blocking solution contains protein or another moiety that will adhere to sites of non-specific binding on the microarray. For example, solutions of bovine serum albumin or milk may be used as blocking solutions.

The target proteins of the plurality of protein-capture agents on the microarray are proteins that are all expression products, or fragments thereof, of a cell or population of cells of a single organism. The expression products may be proteins, including peptides, of any size or function. They may be intracellular proteins or extracellular proteins. The expression products may be from a one-celled or multicellular organism. The organism may be a plant or an animal. In a specific embodiment of the invention, the target proteins are human expression products, or fragments thereof.

In another embodiment of the invention, the target proteins of the protein-capture agents of the microarray may be a randomly chosen subset of all the proteins, including peptides, which are expressed by a cell or population of cells in a given organism or a subset of all the fragments of those proteins. Thus, the target proteins of the protein-capture agents of the microarray may represent a wide distribution of different proteins from a single organism.

The target proteins of some or all of the protein-capture agents on the microarray need not necessarily be known. Indeed, the target protein of a protein-capture agent of the microarray may be a protein or peptide of unknown function. For example, the different protein-capture agents of the microarray may together bind a wide range of cellular proteins from a single cell type, many of which are of unknown identity and/or function.

In one embodiment of the invention, the target proteins of the protein-capture agents on the microarray are related proteins. The different proteins bound by the protein-capture agents may be members of the same protein family. The different target proteins of the protein-capture agents of the microarray may be either functionally related or simply suspected of being functionally related. The different proteins bound by the protein-capture agents of the microarray may also be proteins that share a similarity in structure or sequence or are simply suspected of sharing a similarity in structure or sequence. For example, the target proteins of the protein-capture agents on the microarray may be mucins, growth factor receptors, hormone receptors, neurotransmitter receptors, catecholamine receptors, amino acid derivative receptors, cytokine receptors, extracellular matrix receptors, antibodies, lectins, cytokines, serpins, proteases, kinases, phosphatases, ras-like GTPases, hydrolases, steroid hormone receptors, transcription factors, heat-shock transcription factors, DNA-binding proteins, zinc-finger proteins, leucine-zipper proteins, homeodomain proteins, intracellular signal transduction modulators and effectors, apoptosis-related factors, DNA synthesis factors, DNA repair factors, DNA recombination factors, cell-surface antigens, hepatitis C virus (HCV) proteases or HIV proteases and may correspond to all or part of the proteins encoded by the genes of the gene expression profiles of the invention.

iv. Control Oligonucleotides and Protein-Capture Agents

Control oligonucleotides corresponding to genomic DNA, housekeeping genes, or negative and positive control genes may also be present on the microarray. Similarly, protein-capture agents that bind housekeeping proteins, or negative and positive control proteins, such as beta actin protein, may also be present on the microarray. These controls are used to calibrate background or basal levels of expression, and to provide other useful information.

Normalization controls may be oligonucleotide probes that are perfectly complementary to labeled reference oligonucleotides that are added to the polynucleotide sample. Normalization controls may be protein-capture agents that bind specifically and consistently to a labeled reference protein that is added to the protein sample. For example, a protein-capture agent/normalization control pair may comprise avidin/biotin or a well-known antibody/antigen combination with a known binding coefficient. The signals obtained from the normalization controls after hybridization provide a control for variations in hybridization conditions, label intensity, efficiency, and other factors that may cause the hybridization signal to vary between microarrays. To normalize fluorescence intensity measurements, for example, signals from all probes of the microarray may be divided by the signal from the control probes.

Expression level controls are oligonucleotide probes or protein-capture agents that hybridize/bind specifically with constitutively expressed genes in the biological sample and are designed to control the overall metabolic activity of a cell. Analysis of the variations in the levels of the expression control as compared to the expression level of the target polynucleotide or target protein indicates whether variations in the expression level of a gene or protein is due specifically to changes in the transcription rate of that gene or to general variations in the health of the cell. Thus, if the expression levels of both the expression control and the target gene decrease or increase, these alterations may be attributed to changes in the metabolic activity of the cell as a whole, not to differential expression of the target gene or protein in question. If only the expression of the target gene or protein varies, however, then the variation in the expression may be attributed to differences in regulation of that gene or protein and not to overall variations in the metabolic activity of the cell. Constitutively expressed genes such as housekeeping genes (e.g., β-actin gene, transferrin receptor gene, GAPDH gene) may serve as expression level controls.

Mismatch controls may also be used for expression level controls or for normalization controls. These oligonucleotide probes and protein-capture agents provide a control for non-specific binding or cross-hybridization to a polynucleotide in the sample other than the target to which the probe is directed. Mismatch controls are oligonucleotide probes identical to the corresponding test or control probes except for the presence of one or more mismatched bases. One or more mismatches (e.g., substituting guanine, cytidine, or thymine for adenine) are selected such that under appropriate hybridization conditions (e.g., stringent conditions), the test or control probe would be expected to hybridize with its target sequence, but the mismatch probe would not hybridize or would hybridize to a significantly lesser extent. Similarly, an antibody may be used as a mismatch control protein-capture agent. For example, an antibody may be used that has a base pair mismatch in the binding domain that affects binding as compared to the normal antibody.

v. Detection Methods and Analysis of Hybridization Results

Methods for signal detection of labeled target polynucleotides hybridized to microarray probes are well-known in the art. For example, a radioactive labeled probe may be detected by radiation emission using photographic film or a gamma counter. For fluorescently labeled target polynucleotides, the localization of the label on the probe microarray may be accomplished with fluorescent microscopy. The hybridized microarray is excited with a light source at the excitation wavelength of the particular fluorescent label and the resulting fluorescence is detected. The excitation light source may be a laser appropriate for the excitation of the fluorescent label.

Confocal microscopy may be automated with a computer-controlled stage to automatically scan the entire microarray. Similarly, a microscope may be equipped with a phototransducer (e.g., a photomultiplier) attached to an automated data acquisition system to automatically record the fluorescence signal produced by hybridization to oligonucleotide probes. See, e.g., U.S. Pat. No. 5,143,854.

The invention also relates to methods for evaluating the hybridization results. These methods may vary with the nature of the specific oligonucleotide probes or protein-capture agent used as well as the controls provided. For example, quantification of the fluorescence intensity for each probe may be accomplished by measuring the probe signal strength at each location (representing a different probe) on the microarray (e.g., detection of the amount of florescence intensity produced by a fixed excitation illumination at each location on the microarray). The fluorescent intensity for each protein-capture agent and target protein may be accomplished using similar methods. The absolute intensities of the target polynucleotides or target proteins hybridized to the microarray may then be compared with the intensities produced by the controls, providing a measure of the relative expression of the target polynucleotides or target proteins that hybridize to each of the probes or protein-capture agents.

Normalization of the signal derived from the target polynucleotides to the normalization controls may provide a control for variations in hybridization conditions. Typically, normalization may be accomplished by dividing the measured signal from the other probes or protein-capture agents in the microarray by the average signal produced by the normalization controls. Normalization may also include correction for variations due to sample preparation and amplification. Such normalization may be accomplished by dividing the measured signal by the average signal from the sample preparation/amplification control probes or protein-capture agents. The resulting values may be multiplied by a constant value to scale the results. Other methods for analyzing microarray data are well-known in the art including coupled two-way clustering analysis, clustering algorithms (hierarchical clustering, self-organizing maps), and support vector machines. See, e.g., Brown et al., 97 PROC. NATL. ACAD. SCI. USA 262 (2000); Getz et al., 97 PROC. NATL. ACAD. SCI. USA 12079 (2000); Holter et al., 97 PROC. NATL. ACAD. SCI. USA 8409 (2000); Tamayo et al., 96 PROC. NATL. ACAD. SCI. USA 2907 (1999); Eisen et al., 95 PROC. NATL. ACAD. SCI. USA 14863 (1998); and Ermolaeva et al., 20 NATURE GENET. 19 (1998).

Indeed, the methodologies useful in analyzing gene expression profiles and gene expression data are equally applicable in the context of the study of protein expression. In general, for a variety of applications including proteomics and diagnostics, the methods of the invention involve the delivery of the sample containing the proteins to be analyzed to the microarrays. After the proteins of the sample have been allowed to interact with and become immobilized on the regions comprising protein-capture agents with the appropriate biological specificity, the presence and/or amount of protein bound at each region is then determined. The detection methods, analysis tools, and algorithms described for the polynucleotide microarrays are equally applicable in the context of protein microarrays.

In addition to the methods described above, a wide range of detection methods are available to analyze the results of protein microarray experiments. Detection may be quantitative and/or qualitative. The protein microarray may be interfaced with optical detection methods such as absorption in the visible or infrared range, chemiluminescence, and fluorescence (including lifetime, polarization, fluorescence correlation spectroscopy (FCS), and fluorescence-resonance energy transfer (FRET)). Other modes of detection such as those based on optical waveguides (WO 96/26432 and U.S. Pat. No. 5,677,196), surface plasmon resonance, surface charge sensors, and surface force sensors are compatible with many embodiments of the invention. Alternatively, technologies such as those based on Brewster Angle microscopy (BAM) (Schaaf et al., 3 LANGMUIR 1131 (1987)) and ellipsometry (U.S. Pat. Nos. 5,141,311 and 5,116,121; Kim, 22 MACROMOLECULES 2682 (1984)) may be utilized. Quartz crystal microbalances and desorption processes provide still other alternative detection means suitable for at least some embodiments of the invention microarray. See, e.g., U.S. Pat. No. 5,719,060. An example of an optical biosensor system compatible both with some microarrays of the invention and a variety of non-label detection principles including surface plasmon resonance, total internal reflection fluorescence (TIRF), Brewster Angle microscopy, optical waveguide lightmode spectroscopy (OWLS), surface charge measurements, and ellipsometry are discussed in U.S. Pat. No. 5,313,264.

Other different types of detection systems suitable to assay the protein expression microarrays of the invention include, but are not limited to, fluorescence, measurement of electronic effects upon exposure to a compound or analyte, luminescence, ultraviolet visible light, and laser induced fluorescence (LIF) detection methods, collision induced dissociation (CID), mass spectroscopy (MS), CCD cameras, electron and three dimensional microscopy. Other techniques are known to those of skill in the art. For example, analyses of combinatorial microarrays and biochip formats have been conducted using LIF techniques that are relatively sensitive. See, e.g., Ideue et al., 337 CHEM. PHYSICS LETTERS 79 (2000).

One detection system of particular interest is time-of-flight mass spectrometry (TOF-MS). Using parallel sampling techniques, time-of-flight mass spectrometry may be used for the detailed characterization of hundreds of molecules in a sample mixture at each discreet location within the microarray. Time-of-flight mass spectrometry based systems enable extremely rapid analysis (microseconds to milliseconds instead of seconds for scanning MS devises) high levels of selectivity compared to other techniques with good sensitivity (better than one part per million, as opposed to one part per ten thousand for scanning MS), As a mass spectroscopic technique, time-of-flight mass spectrometry provides molecular weight and structural information for identification of unknown samples.

Additional levels of sensitivity are added by coupling time-of-flight mass spectrometry to another separation system. Thus, in an embodiment, the invention comprises using ion mobility in combination with time-of-flight mass spectrometry for the analysis of microarrays. The combination of ion mobility and time-of-flight mass spectrometry is referred to as multi-dimensional spectroscopy (MDS). Ions are electro-sprayed into the front of the MDS device. Electrospray is a method for ionizing relatively large molecules and having them form a gas phase. The solution containing the sample is sprayed at high voltage, forming charged droplets. These droplets evaporate, leaving the sample's ionized molecules in the gas phase. These ions continue into the ion mobility chamber where the ions travel under the influence of a uniform electric field through a buffer gas. The principle underlying ion mobility separation techniques is that compact ions undergo fewer collisions than ions having extended shapes and thus, have increased mobility. As the separated components (comprising ions/molecules of different mobility) exit the drift tube, they are pulsed into a time-of-flight mass spectrometer.

Although non-label detection methods are generally preferred, some of the types of detection methods commonly used for traditional immunoassays that require the use of labels may be applied to the microarrays of the invention. These techniques include noncompetitive immunoassays, competitive immunoassays, and dual label, radiometric immunoassays. These techniques are primarily suitable for use with the microarrays of protein-capture agents when the number of different protein-capture agents with different specificity is small (less than about 100). In the competitive method, binding-site occupancy is determined indirectly. In this method, the protein-capture agents of the microarray are exposed to a labeled developing agent, which is typically a labeled version of the analyte or an analyte analog. The developing agent competes for the binding sites on the protein-capture agent with the analyte. The fractional occupancy of the protein-capture agents on different regions can be determined by the binding of the developing agent to the protein-capture agents of the individual regions.

In the noncompetitive method, binding site occupancy is determined directly. In this method, the regions of the microarray are exposed to a labeled developing agent capable of binding to either the bound analyte or the occupied binding sites on the protein-capture agent. For example, the developing agent may be a labeled antibody directed against occupied sites (i.e., a “sandwich assay”). Alternatively, a dual label, radiometric, approach may be taken where the protein-capture agent is labeled with one label and the second, developing agent is labeled with a second label. See Ekins, et al., 194 CLINICA CHIMICA ACTA. 91 (1990). Many different labeling methods may be used in the aforementioned techniques, including radioisotopic, enzymatic, chemiluminescent, and fluorescent methods.

vi. Types of Microarrays

The invention contemplates a variety of microarrays that may be used to study and monitor ProMac signature gene expression. For example, the microarrays of the invention may be derived from or representative of a specific organism, tissue type, or cell type, including human microarrays, cancer microarrays, apoptosis microarrays, oncogene and tumor suppressor microarrays, cell-cell interaction microarrays, cytokine and cytokine receptor microarrays, blood microarrays, cell cycle microarrays, neuroarrays, mouse microarrays, and rat microarrays, or combinations thereof. Preferable, the invention contemplates microarrays comprising a gene or protein expression profile generated from ProMacs.

In further embodiments, the microarrays may represent diseases, including cardiovascular diseases, neurological diseases, immunological diseases, various cancers, infectious diseases, endocrine disorders, and genetic diseases. In some embodiments, the disease may be a known ProMac associated disease, such as ALS, AD, HAD, or MacDgn, while in other embodiments the microarray may represent a disease that is not yet determined to be ProMac associated.

In a specific embodiment, the invention provides a microarray comprising one or more polynucleotide sequences substantially homologous to or complementary to a polynucleotide sequence, or portions thereof, selected from the group consisting of sequences 1-1474 and 1478.

In another embodiment, the invention provides a microarray comprising one or more protein-capture agents that bind one or more amino acid sequences encoded by all or a portion of one or more amino acid sequences selected from the group consisting of sequences 1-147 and 1478.

vii. Expression Profiles and Microarray Methods of Use

In one aspect, the invention provides methods for the reproducible measurement and assessment of the expression of specific transcripts or proteins, specifically those in the signature for ProMacs. One method combines and utilizes the techniques of laser capture microdissection, T7-based RNA amplification, production of cDNA from amplified RNA, and DNA microarrays containing immobilized DNA molecules for a wide variety of specific genes to produce a profile of gene expression analysis for very small numbers of specific cells. The desired cells are individually identified and attached to a substrate by the laser capture technique, and the captured cells are then separated from the remaining cells. RNA is then extracted from the captured cells and amplified about one million-fold using the T7-based amplification technique, and cDNA may be prepared from the amplified RNA. A wide variety of specific DNA molecules are prepared that hybridize with specific polynucleotides of the microarray, and the DNA molecules are immobilized on a suitable substrate. The cDNA made from the captured cells is applied to the microarray under conditions that allow hybridization of the cDNA to the immobilized DNA on the microarray. The expression profile of the captured cells is obtained from the analysis of the hybridization results using the amplified RNA or cDNA made from the amplified RNA of the captured cells, and the specific immobilized DNA molecules on the microarray. The hybridization results demonstrate, for example, which genes of those represented on the microarray as probes are hybridized to cDNA from the captured cells, and/or the amount of specific gene expression. The hybridization results represent the gene expression profile of the captured cells. The gene expression profile of the captured cells can be used to compare the gene expression profile of a different set of captured cells. The similarities and differences provide useful information for determining the differences in gene expression between different cell types, and differences between the same cell type under different conditions.

The techniques used for gene expression analysis are likewise applicable in the context of protein expression profiles. Total protein may be isolated from a cell sample and hybridized to a microarray comprising a plurality of protein-capture agents, which may include antibodies, receptor proteins, small molecules, and the like. Using any of several assays known in the art, hybridization may be detected and analyzed as described above. In the case of fluorescent detection, algorithms may be used to extract a protein expression profile representative of the particular cell type.

The microarrays of the invention may be used to distinguish normal tissue from disease tissue resulting from ProMac associated diseases. In addition, the invention may be used to diagnosis diseases associated with ProMacs. A patient sample may be hybridized to a microarray consisting of probes representing signature ProMac genes. The resulting expression pattern of the patient sample may then be compared to the expression profile of a normal tissue sample to determine the disease status. For example, increased levels of expression of genes within the ProMac signature may be indicative of a ProMac associated disease or a predisposition to a disease, for example, ALS, AD, HAD, or MacDgn.

In another embodiment of the invention, a microarray consisting of probes representing the ProMac signature is used to detect modulation of gene transcription levels of at least one ProMac signature gene that results from exposing the selected tissue or cells to a candidate drug. In this embodiment, a biological sample derived from an organism, or an established cell line, may be exposed to the candidate drug in vivo or ex vivo. Thereafter, the gene transcripts, primarily mRNA, of the tissue or cells are isolated by methods well-known in the art. SAMBROOK ET AL., MOLECULAR CLONING: A LAB. MANUAL (2001). The isolated transcripts are then contacted with a microarray under conditions where the transcripts hybridize with a corresponding probe to form hybridization pairs. Thus, the microarray provides a model of the transcriptional responsiveness, or modulation, of the ProMac signature genes following exposure to a particular drug candidate.

Gene and/or protein expression profiles and microarrays may also be used to identify ProMac modulators. The biological effects of a compound may be reflected in the biological state of a cell, which can be characterized by the cell's transcriptional state. The gene expression profiles, microarrays, and algorithms of the invention may be used to analyze and characterize the transcriptional state of a given cell or tissue following exposure to candidate modulator.

viii. Microarray Kits

The microarray methods for detecting the presence and/or relative levels of ProMacs in, for example, a biological sample may be provided as part of a kit. The kit may comprise either a fixed array of nucleotide sequences for detecting a transcript primarily expressed in ProMacs or a fixed array of protein-capture agents that binds a polypeptide sequence primarily expressed in ProMacs. Thus, the invention further provides kits for detecting and/or prognosing ProMac associated diseases in patients. Procedures using these kits may be performed by clinical laboratories, experimental laboratories, medical practitioners, or private individuals. The kit may provide additional components that are useful in procedures, including, but not limited to, buffers, developing reagents, labels, reacting surfaces, means for detection, control samples, standards, instructions, and interpretive information.

d. Non-Microarray Methods of Polypeptide Detection

Polypeptide observations to detect the expression of ProMac genes in a sample can be accomplished through methods other than microarray. The expression of a polypeptide translated from a signature transcript can be determined by a variety of methods familiar to those of ordinary skill in the art. These include, but are not limited to, immunological assays, enzyme assays, e.g., ELISAs, radioimmunoassay (RIA), and the like. Functional assays for biological activity or flow cytometry for ProMac-specific cell surface markers may also be used to detect the expression of a ProMac signature encoded polypeptide.

In another embodiment, these detection methodologies may be used in identifying ProMac polypeptides that can serve as additional prognostic indicators of a ProMac associated disease. Further, the method mentioned may be used to determine of any modulation in ProMac biological activity that may occur as a result of modulator administration.

2. Detecting, Prognosing, and Monitoring ProMac Associated Diseases

In other aspects, the invention provides methods for diagnosing, prognosing, and monitoring ProMac associated diseases. In one embodiment, the methods of the present invention include using a panel of ProMac signature genes and/or ProMac secondary signature genes for diagnosing neurodegenerative disorders, e.g., amyotrophic lateral sclerosis (ALS), Charcot-Marie Tooth syndrome, Alzheimer's disease (AD), HIV-associated dementia (HAD), HIV associated neurological disorders, peripheral sensory neuropathy, diabetic neuropathy, autism, Parkinson's disease, schizophrenia. In another embodiment, the methods of the present invention include using a panel of ProMac signature genes and/or ProMac secondary signature genes for diagnosing, prognosing, and/or monitoring ProMac associated diseases, e.g., the diseases discussed under the section of “ProMacs and Their Implications”. In yet another embodiment, the methods provided comprise taking a biological sample from a patient, determining the expression level in that sample of at least one ProMac signature gene, e.g., one gene or gene product primarily expressed in ProMacs according to the methods described herein, and comparing the determined level to the expression level of that gene or gene product in a wildtype (control) sample. In specific non-limiting embodiments, the methods are useful for diagnosing a ProMac associated disease, predicting the onset or relapse of a ProMac associated disease, determining the progression or remission of the disease, facilitating determination of the prognosis of a patient, and assessing the responsiveness of a patient to therapy (e.g., by determining whether a relative decrease in the number of ProMacs has occurred, based on a decrease in gene expression).

In one embodiment, a higher than normal expression level of the genes or gene products from the ProMac signature set indicates an elevated presence of ProMacs. If a patient with an elevated level of ProMacs is already afflicted with a disease, then the disease can be ProMac associated. If the patient is not yet suffering from a ProMac associated disease, elevated ProMac levels suggest predisposition to such a disease.

Determining the expression of a ProMac signature gene or gene product and comparing a patient's expression level to a known wildtype expression level may allow for a more effective determination of the best possible treatment for the a patient, particularly in terms of the specificity of the treatment. Additionally, monitoring of expression levels enables detection and treatment of ProMac associated diseases, even independent of knowledge regarding the classification of the disease. See Golub, et al., 286 SCIENCE 531 (1999). Such monitoring can also provide patients with the benefit of detecting potentially malignant or pathological events at a molecular level before that become detectable at a gross morphological level.

a. ProMac Genes

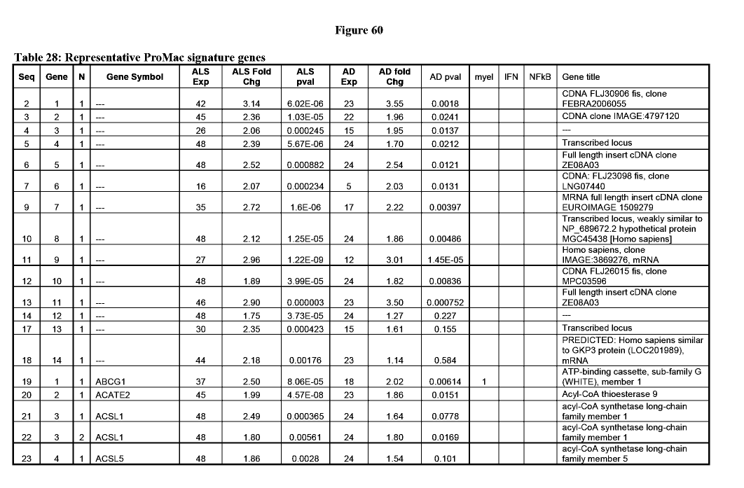

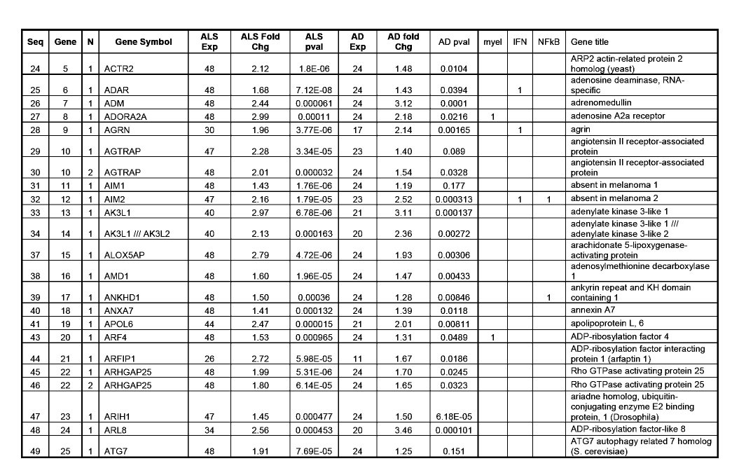

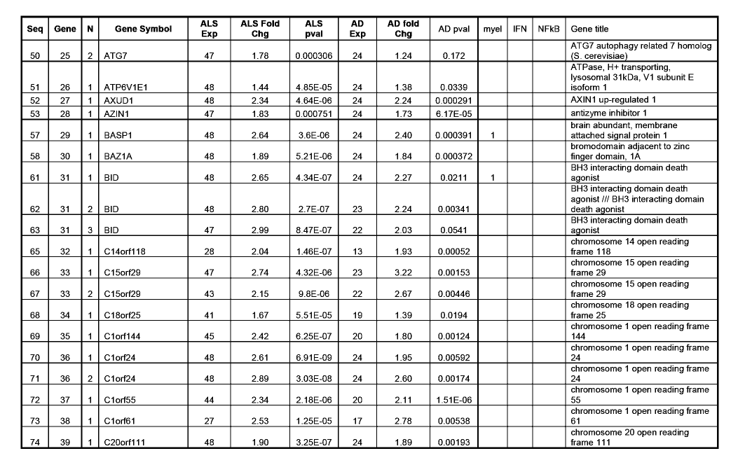

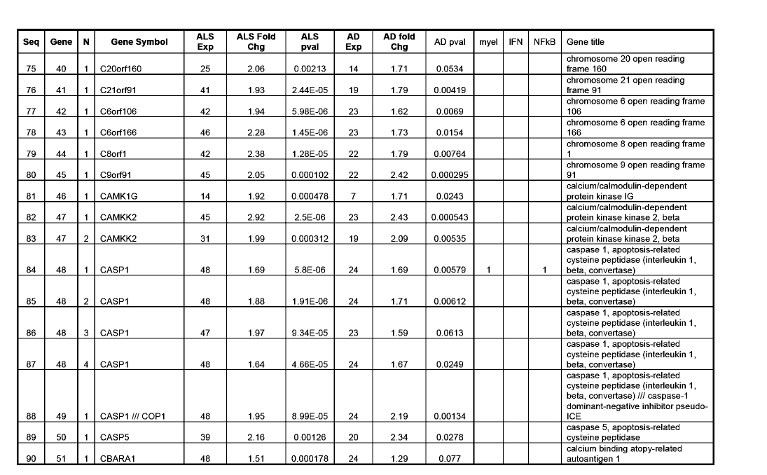

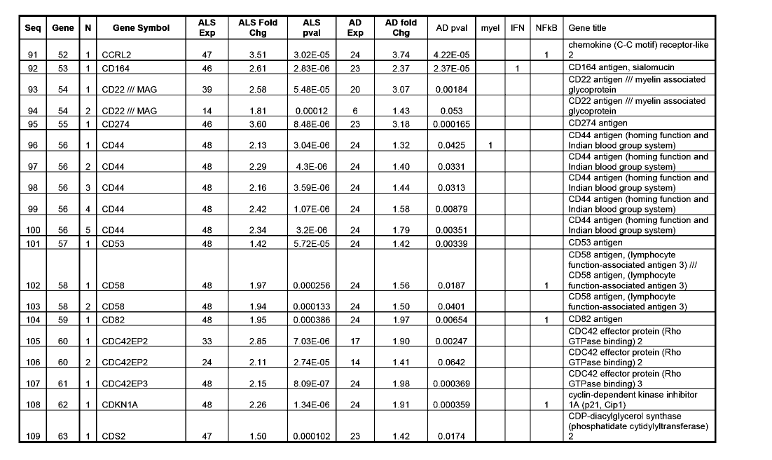

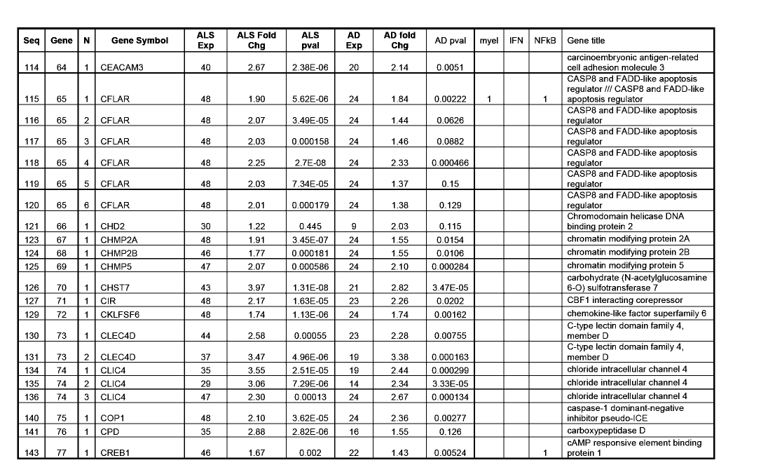

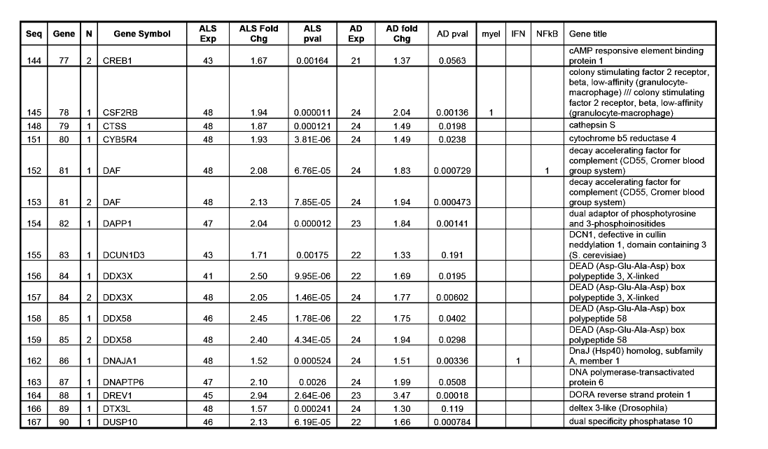

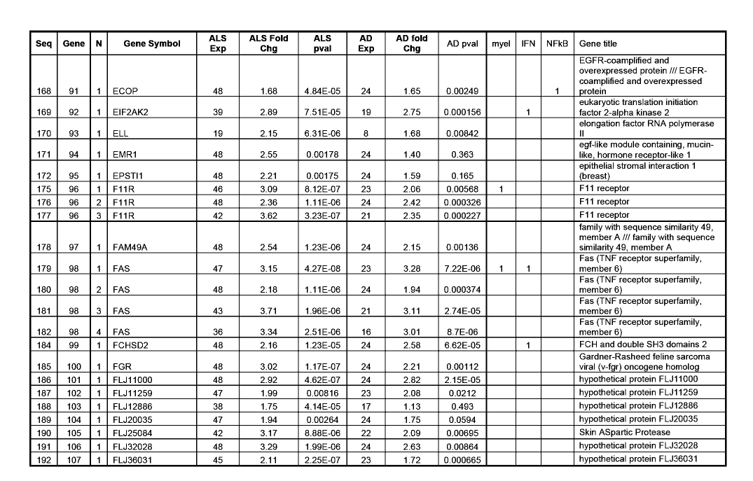

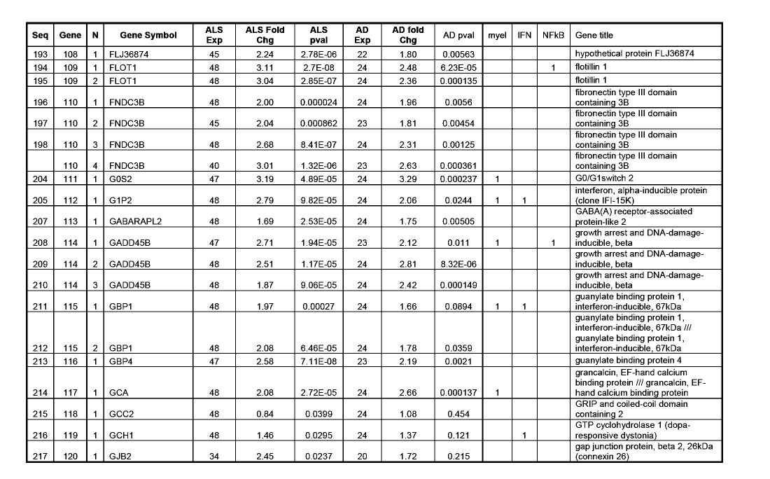

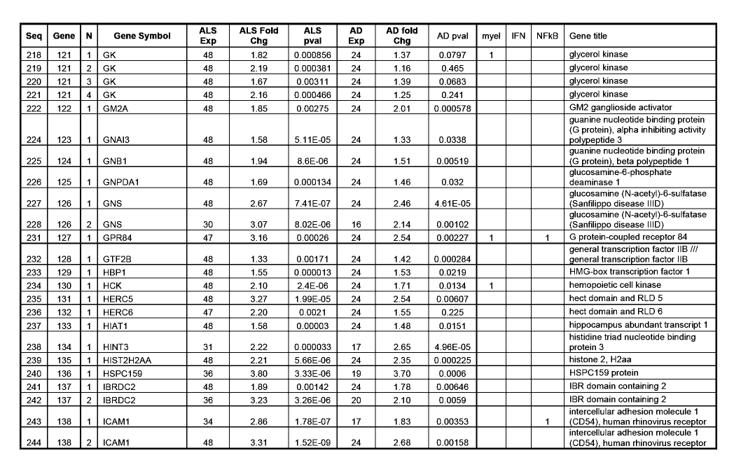

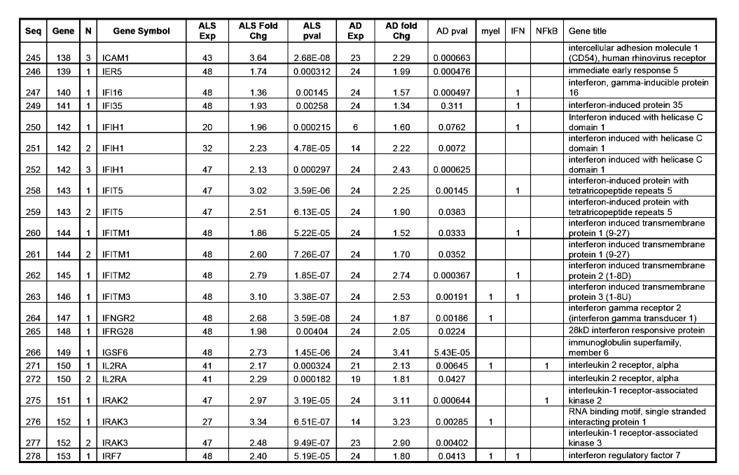

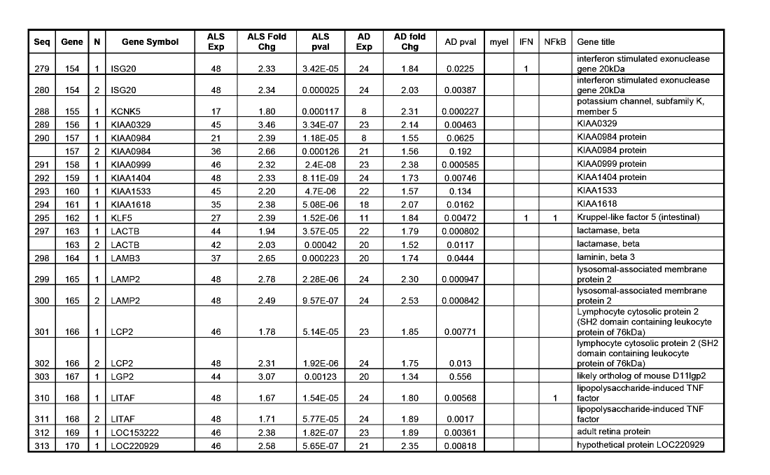

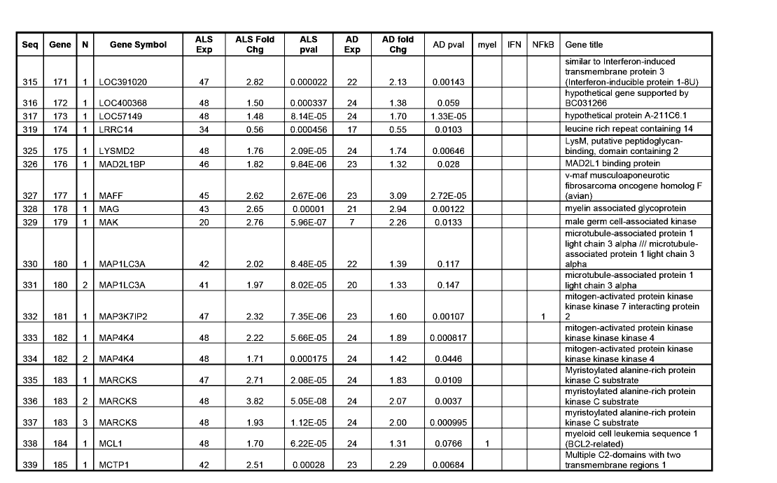

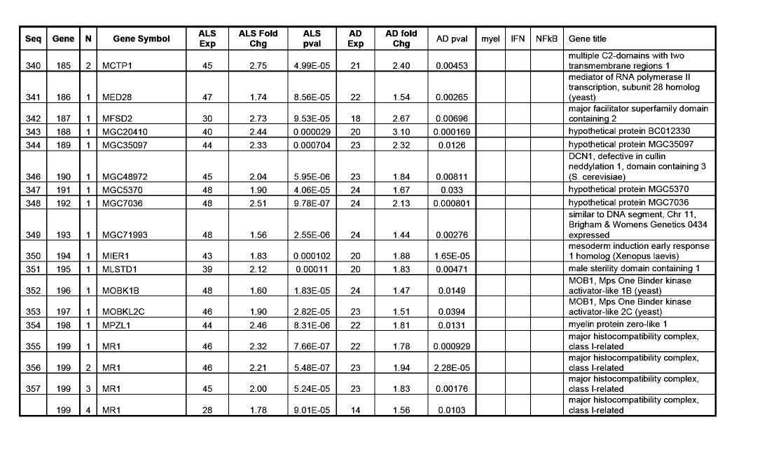

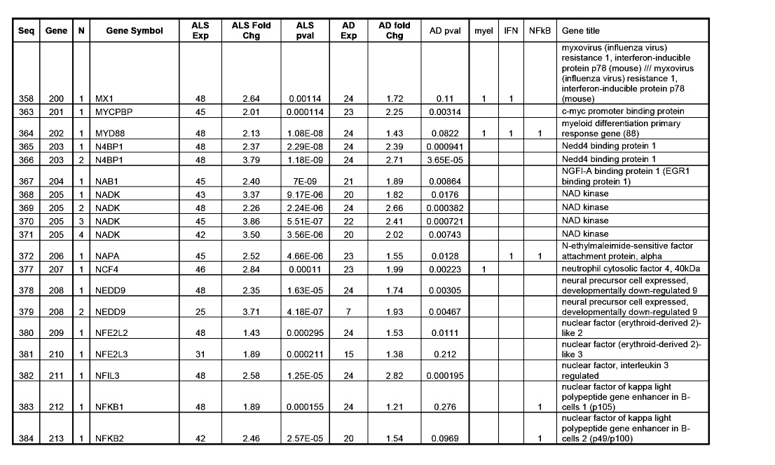

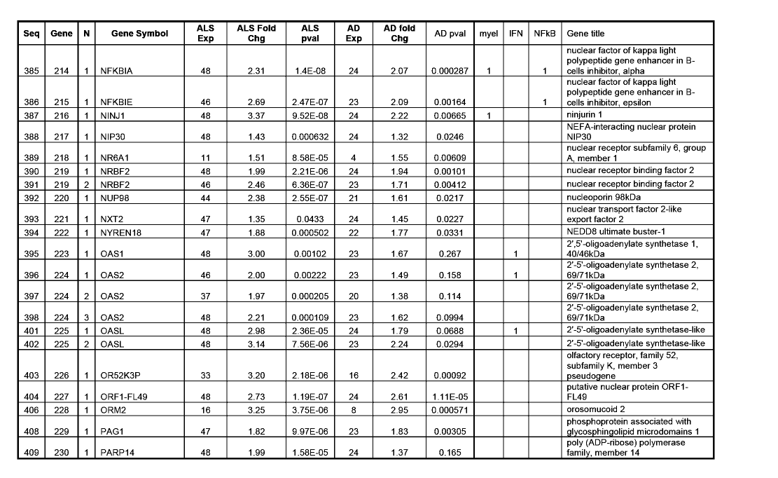

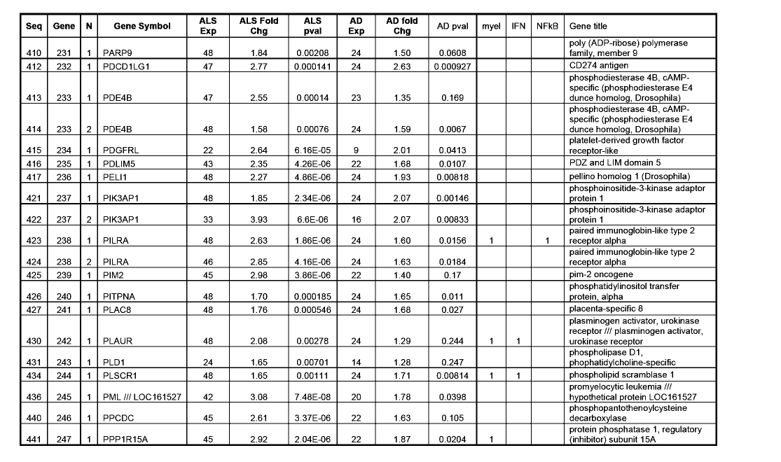

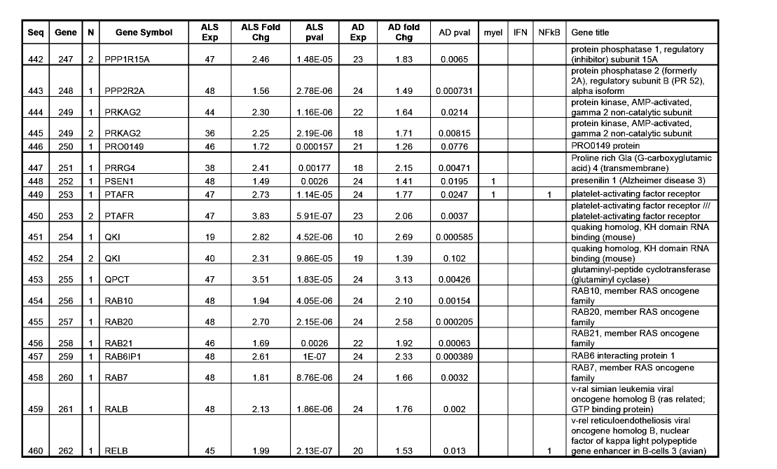

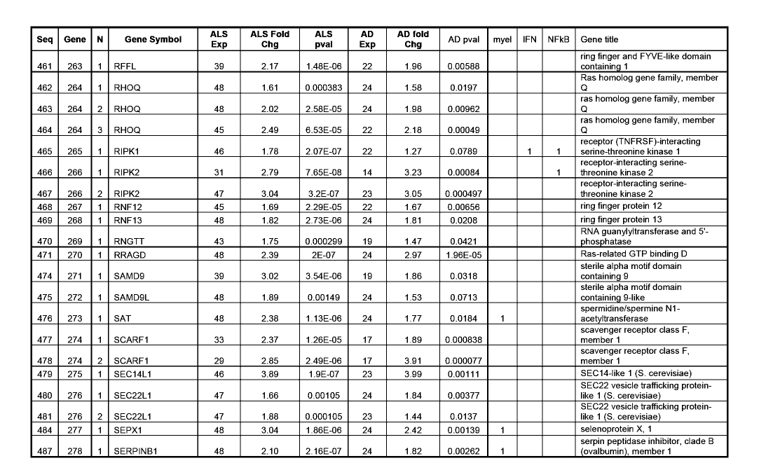

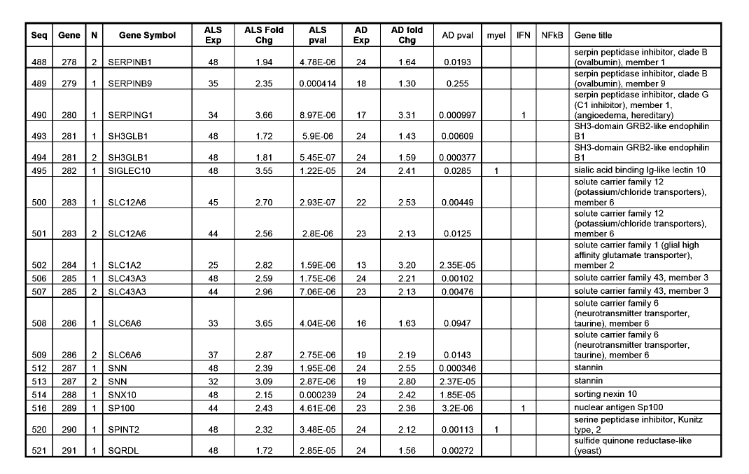

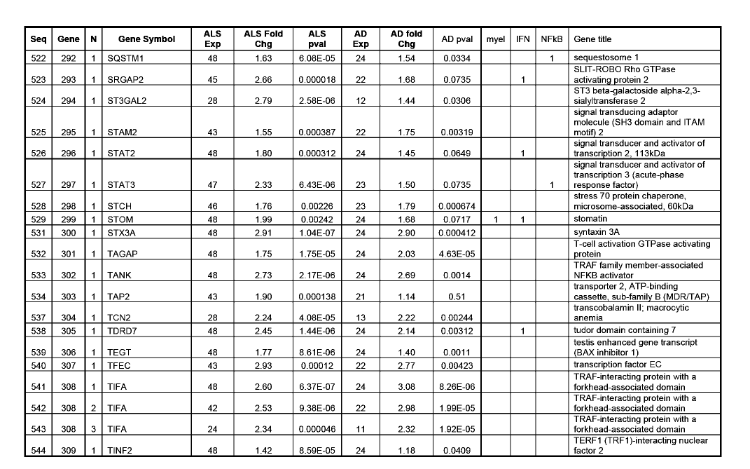

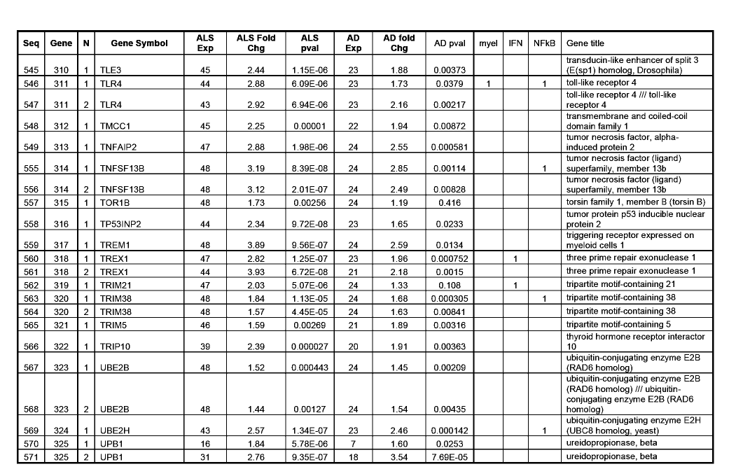

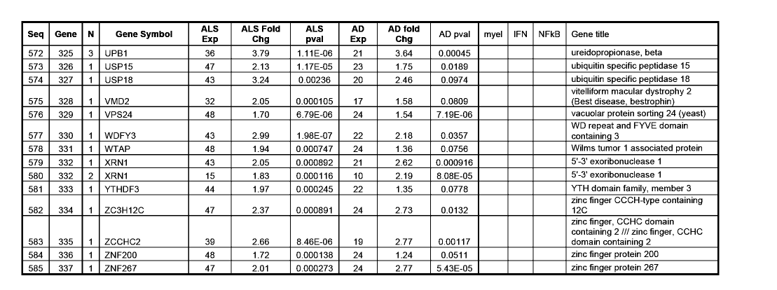

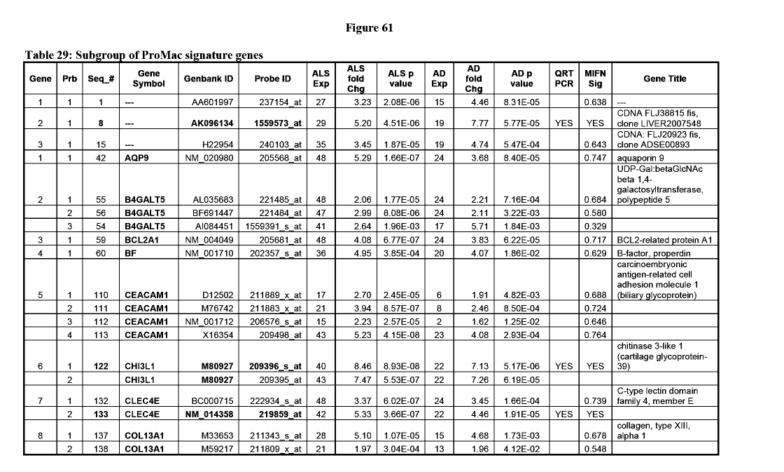

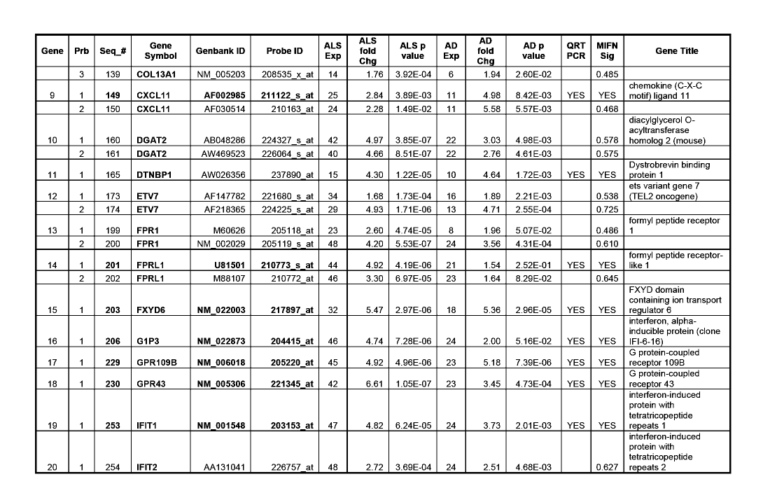

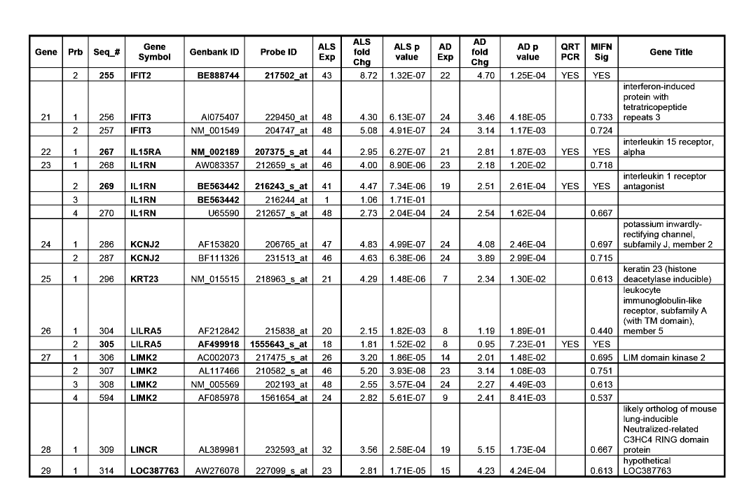

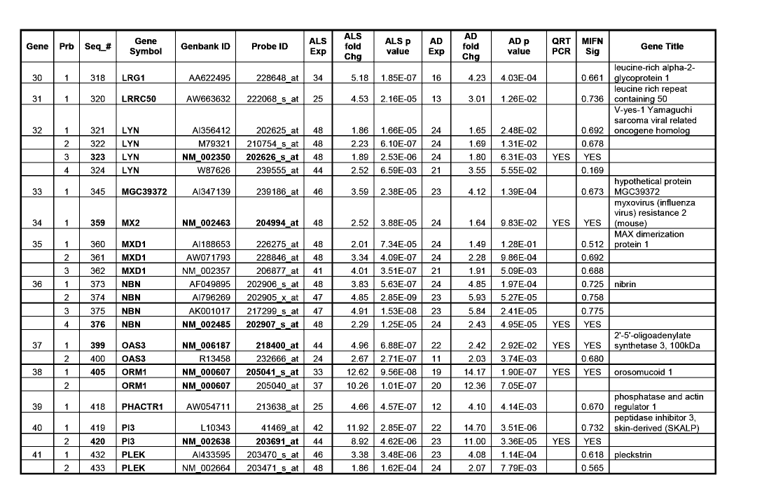

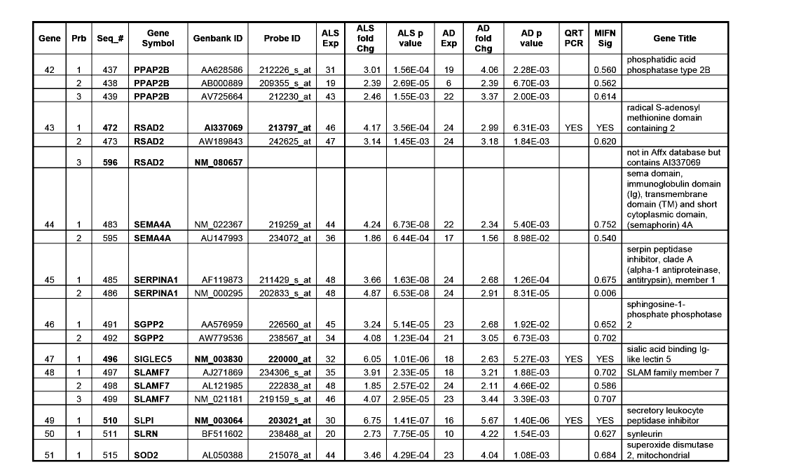

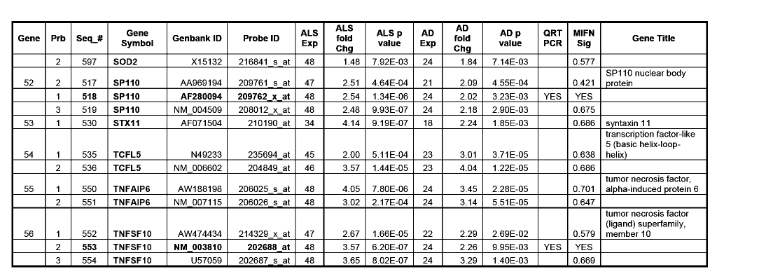

The ProMac genes or ProMac signature genes are a group of genes that are upregulated in ProMacs. In one embodiment, they can be characterized by: (1) an increased expression in individuals with ALS and AD; (2) a high degree of correlation of signals with each other; (3) a similar time course of expression; and (4) expression that is relatively poorly correlated with transcripts from other cell populations in the peripheral blood cell or from T cells. In another embodiment, they include genes that are upregulated at least 2-fold or at least 4-fold in individuals with ALS or AD. In another embodiment of the invention, the ProMac genes are the genes listed in Table 28 (FIG. 60). In another embodiment, the ProMac genes are the genes listed in Table 21 (FIG. 53). In yet another embodiment, the ProMac genes are the genes listed in Table 29 (FIG. 61).

According to the present invention, a panel of ProMac signature genes can include at least one, two, three, four, five, six, seven, or eight ProMac signature genes. In one embodiment, a panel of ProMac signature genes include at least two ProMac signature genes. In another embodiment, a panel of ProMac signature genes include at least four ProMac signature genes. In yet another embodiment, a panel of ProMac signature genes include CLEC4E, G1P3, GPR109B, IFIT2, IL1RN, MX2, NBS1, or ORM1, or combinations thereof. In yet another embodiment, a panel of ProMac signature genes include G1P3, GPR43, IFIT2, ORM1, or TNFSF10, or combinations thereof. In still another embodiment, a panel of ProMac signature genes include CHI3L1, CLEC4E, G1P3, GPR43, GPR109B, IFIT2, IL1RN, MX2, NBS1, ORM1, SIGLEC5, or SLPI, or combinations thereof.

According to another embodiment of the present invention, ProMac secondary signature genes can be used alone or in combination with ProMac signature genes. In one embodiment, ProMac secondary signature genes are genes that are not ProMac signature genes, but are up-regulated for at least 2-fold or 4-fold over the normal level in individuals with ALS or AD. In another embodiment, ProMac secondary signature genes are genes useful for distinguishing one ProMac associated disorder from another ProMac associated disorder, e.g., distinguishing cerebral neuron degeneration (e.g, AD) from motor neuron degeneration (e.g. ALS). In yet another embodiment, ProMac secondary signature genes are genes useful to be used in concert with one or more ProMac signature genes to diagnose, monitor, or prognose ProMac associated disorders. In yet another embodiment, ProMac secondary signature genes are genes useful for providing prognosis of a ProMac associate disease, e.g., ALS.

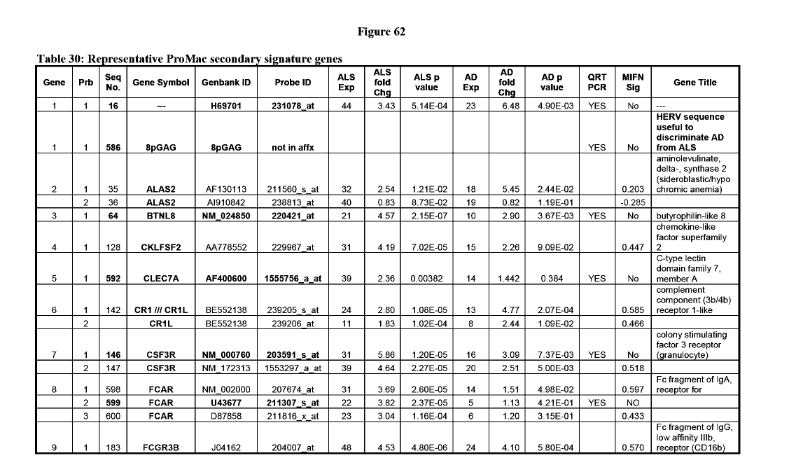

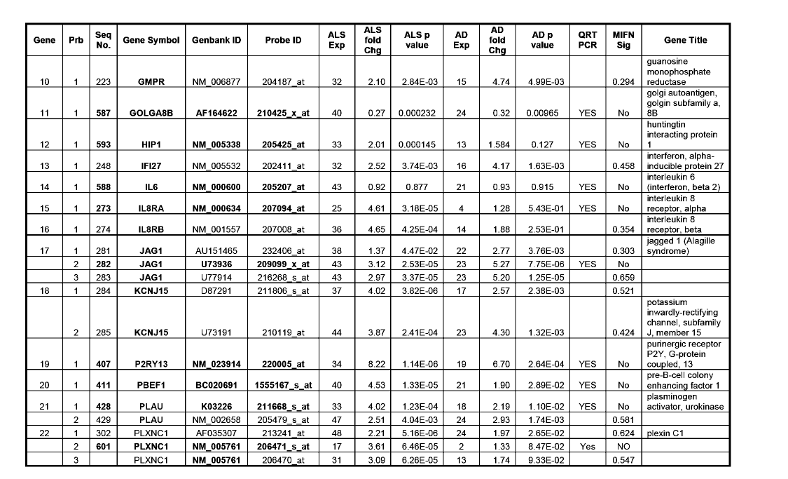

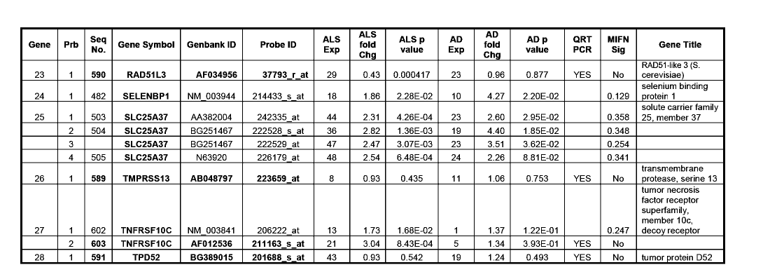

In still yet another embodiment, ProMac secondary signature genes are the genes listed in Table 30 (FIG. 62). In yet another embodiment, ProMac secondary signature genes include 8pGAG,CSF3R, GOLGIN-67, IL6, JAG1, MSP, RAD51L3, or TPD52 or combinations thereof. In still yet another embodiment, ProMac secondary signature genes include CD14, CLEC7A, FCAR, FCGR1a, GOLGIN-67, GPR86, HIP1, RAD51L3, or 8pGAG or combinations thereof. In still yet another embodiment, ProMac secondary signature genes include ALAS2, BTNL8, CKLFSF2, CR1L, CSF3R, FCAR, FCGR3B, GMPB, IF127, IL8RA, IL8RB, JAG1, KCNJ15, P2RY13, PBEF1, PLAU, PLXNC1, SLENBP1, SLC25A37, and TNFRSF10C, or combinations thereof.

3. Modulating ProMac Activity

ProMac modulators are agents that increase or decrease a biological activity of ProMacs. This invention provides methods for identifying a modulator that binds to and/or modulates the biological activity of ProMacs. More specifically, one method includes screening for ProMac modulators that are suitable for treating or preventing a ProMac associated disease through contacting a candidate modulator with ProMacs under conditions where binding can occur, and detecting if binding to the ProMac does occur. Another method involves identifying an agent capable of modulating ProMac biological activity and comprises contacting the candidate modulator with ProMacs and detecting whether the biological activity of ProMacs changes. The invention also provides methods of screening patients for the administration of these therapeutic ProMac modulators to determine, for example, individual effectiveness of the modulating agent.

For purposes of this invention, the preferred activity to modulate is macrophage proliferation. A modulator may decrease the rate of macrophage proliferation by at least 25%, preferably by at least 50%, more preferably by at least 75%, and even more preferably by at least 90%.

In some embodiments, an individual suitable for administration of a ProMac modulator, or candidate ProMac modulator is one who has been diagnosed with a ProMac associated disease or who is adjudged to be at risk for developing such a disease. Such an individual does not need to display any symptoms. An “at risk” patient may have at least one risk factor, including, but not limited to, hereditary predisposition, lack of appropriate chemical markers, deleterious environmental conditions, or retroviral infection.

a. Candidate Modulators

Candidate modulators encompass numerous chemical classes, though typically they are organic molecules, preferably small organic compounds having a molecular weight of more than 50 and less than about 2,500 daltons. Candidate modulators may comprise functional groups necessary for structural interaction with gene products, particularly hydrogen bonding, and typically include at least an amine, carbonyl, hydroxyl or carboxyl group, preferably at least two of the functional chemical groups. Candidate modulators are also found among biomolecules including, but not limited to: peptides, saccharides, fatty acids, steroids, purines, pyrimidines, derivatives, structural analogs or combinations thereof.

Candidate modulators may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting differentially expressed gene products) are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Exemplary candidate ProMac modulators include, but are not limited to, antibodies, soluble receptors, polyamine analogs, antisense oligonucleotides, RNAi polynucleotides, ribozymes, and the like. Antibodies and soluble receptors are of particular interest as candidate agents where the target differentially expressed gene product is secreted or accessible at the cell-surface.

Preferred modulators are suitable for treating or preventing a ProMac associated disease.

i. Antibody Modulators

ProMac modulators include antibodies and functional equivalents thereof that specifically bind to ProMacs and alter their biological activity. “Immunoglobulin” and “antibody” are used interchangeably and in their broadest sense herein. Thus, they encompass intact monoclonal antibodies, polyclonal antibodies, multispecific antibodies (e.g., bispecific antibodies) formed from at least two intact antibodies, and antibody fragments, so long as they exhibit the desired biological activity.

The variable domains of the heavy and light chain of an antibody recognize or bind to a particular epitope of a cognate antigen. The term “epitope” is used to refer to the specific binding sites or antigenic determinant on an antigen that the variable end of the immunoglobulin binds. Epitopes can be linear, i.e., be composed of a sequence of amino acid residues, conformational, such that an immunoglobulin recognizes a 3-D structure, or a combination thereof. Further, carbohydrate portions of a molecule, as expressed by the cell surface receptors of ProMacs can also be epitopes.

(1) Monoclonal and Polyclonal Antibodies

Immunoglobulins of the invention may be polyclonal or monoclonal, and may be produced by any of the well known methods in this art.

Polyclonal antibodies are preferably raised in animals by multiple subcutaneous (sc), intraperitoneal (ip) or intramuscular (im) injections of the relevant antigen and an adjuvant. It may be useful to conjugate the relevant antigen to a protein that is immunogenic in the species to be immunized, In addition, aggregating agents such as alum are suitably used to enhance the immune response.

The term “monoclonal antibody” refers to an antibody obtained from a population of substantially homogeneous antibodies. Monoclonal antibodies are highly specific, being directed against a single antigenic site. Furthermore, in contrast to polyclonal antibody preparations that typically include different antibodies directed against different determinants, each monoclonal antibody is directed against a single determinant on the antigen.

In addition to their specificity, monoclonal antibodies are advantageous in that they may be synthesized while uncontaminated by other immunoglobulins. For example, monoclonal antibodies may be produced by the hybridoma method or by recombinant DNA methods. Monoclonal antibodies also may be isolated from phage antibody libraries.

(2) Antibody Fragments

“Antibody fragments” comprise a portion of an intact antibody, preferably the antigen-binding or variable region thereof. Examples of antibody fragments include Fab, Fab′, F(ab′)², Fv fragments, diabodies, linear antibodies, single-chain antibody molecules, and multispecific antibodies formed from antibody fragments.

Various techniques have been developed for the production of antibody fragments. Traditionally, these fragments were derived via proteolytic digestion of intact antibodies. Two digestion methodologies that are well known in the art include papain digestion and pepsin treatment. Antibody fragments may now additionally be produced directly by recombinant host cells.

(3) Bispecific Antibodies

Bispecific antibodies of the invention are small antibody fragments with two antigen-binding sites. Each fragment comprises a heavy-chain variable domain connected to a light-chain variable domain in the same polypeptide chain. By using a linker that is too short to allow pairing between the two domains on the same chain, the domains are forced to pair with the complementary domains of another chain and create two antigen binding sites.

Methods for making bispecific antibodies are well known in the art. Traditional production of full length bispecific antibodies is based on the coexpression of two immunoglobulin heavy chain-light chain pairs, where the two chains have different specificities. Bispecific antibodies, however, may also be produced using leucine zippers.

ii. Antisense Oligonucleotides

Polynucleotide ProMac modulators may comprise one or more antisense oligonucleotides. In the context of this invention, “oligonucleotide” refers to an oligomer or polymer of RNA or DNA or variants thereof. Oligonucleotides may comprise naturally occurring nucleotides, sugars and covalent internucleoside (backbone) linkages as well as oligonucleotides having non-naturally occurring portions that function similarly. Such modified or substituted oligonucleotides possess desirable properties such as, for example, enhanced cellular uptake, enhanced affinity for polynucleotide target and increased stability in the presence of nucleases.

In general, antisense oligonucleotides specifically hybridize with one or more polynucleotides encoding a ProMac signature gene product and interfere with the normal function of the polynucleotides. An antisense oligonucleotide ProMac modulator may target DNA encoding signature genes and interfere with their replication and/or transcription, or the modulator may hybridize with RNA, including pre-mRNA and mRNA, and affect processes such as translocation of the RNA to the site of protein translation, translation of protein from the RNA, splicing of the RNA to yield one or more mRNA species, and catalytic activity that may be engaged in or facilitated by the RNA. By interfering with such processes, the antisense oligonucleotides can have the overall effect of modulating the expression of the target ProMac signature polynucleotides.

In designing an antisense oligonucleotide ProMac modulator, there are several sites within polynucleotides that may are generally desirable as hybridization regions. Two such regions are the translation initiation codon, or start codon, and the translation termination codon, or stop codon, of the open reading frame. The open reading frame itself may also be targeted effectively, as may the 5′ or 3′ untranslated regions of mRNA. Intron-exon junctions may also serve as useful target sites.

With these various target sites in mind, antisense oligonucleotide ProMac modulators that are sufficiently complementary to the target signature polynucleotides must be chosen. There must be a sufficient degree of complementarity or precise pairing such that stable and specific binding occurs between the oligonucleotide and the ProMac signature target. The level of complementarity, however, need not be 100%. Sufficiency of complementarity is attained when binding of the antisense oligonucleotide interferes with the function of the target ProMac signature polynucleotide, and non-specific binding is avoidable.

Antisense oligonucletotide ProMac modulators may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

iii. Ribozymes

Other polynucleotide molecules, such as ribozymes, could also act as modulators of ProMacs, by inhibiting expression of genes in the ProMac signature. Ribozymes are RNA molecules having an enzymatic activity that is able to repeatedly cleave other separate RNA molecules in a nucleotide base sequence-specific manner. Such enzymatic RNA molecules may be targeted to virtually any RNA transcript, and efficient cleavage achieved in vitro. See generally Kim et al., 84 PROC. NATL. ACAD. SCI. USA 8788 (1987); Haseloff & Gerlach, 334 NATURE 585 (1988); Cech, 260 JAMA 3030 (1988); Jefferies et al., 17 NUCL. ACIDS RES. 1371 (1989).

The enzymatic nature of a ribozyme may be advantageous over other technologies, such as antisense technology (where a polynucleotide molecule simply binds to a polynucleotide target to block its translation) because the effective concentration of ribozyme necessary to effect a therapeutic treatment is lower than that of an antisense oligonucleotide. This advantage reflects the ability of the ribozyme to act enzymatically. Thus, a single ribozyme molecule is able to cleave many molecules of target RNA.

A ribozyme sufficient to act as ProMac modulators is an enzymatic polynucleotide molecule that has a specific substrate binding site which is complementary to one or more of the target signature gene RNA regions, and that it has nucleotide sequences within or surrounding that substrate binding site which impart an RNA cleaving activity to the molecule.

Ribozyme modulators may be delivered to ProMacs in vivo. Delivery may involve transfection, using a DNA construct which encodes the ribozyme modulator under the control of a strong constitutive promoter. Ribozyme modulators may also be administered to cells by a variety of methods known to those familiar to the art, including, but not restricted to, encapsulation in liposomes, by ionophoresis, or by incorporation into other vehicles, such as hydrogels, cyclodextrins, biodegradable nanocapsules, and bioadhesive microspheres. Alternatively, the RNA/vehicle combination may be locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. See generally WO 94/02595; WO 93/23569.

iv. RNAi Modulators

For methods that involve RNAi (RNA interference), a double stranded RNA (dsRNA) molecule is usually used as the modulating agent. The dsRNA is prepared to be substantially identical to at least a segment of a subject polynucleotide (e.g. a cDNA or gene). In general, the dsRNA is selected to have at least 70%, 75%, 80%, 85% or 90% sequence identity with the subject polynucleotide over at least a segment of the candidate gene. In other instances, the sequence identity is even higher, such as 95%, 97% or 99%, and in still other instances, there is 100% sequence identity with the subject polynucleotide over at least a segment of the subject polynucleotide. The size of the segment over which there is sequence identity can vary depending upon the size of the subject polynucleotide. In general, however, there is substantial sequence identity over at least 15, 20, 25, 30, 35, 40 or 50 nucleotides. In other instances, there is substantial sequence identity over at least 100, 200, 300, 400, 500 or 1000 nucleotides; in still other instances, there is substantial sequence identity over the entire length of the subject polynucleotide, i.e., the coding and non-coding region of the candidate gene.

Because only substantial sequence similarity between the subject polynucleotide and the dsRNA is necessary, sequence variations between these two species arising from genetic mutations, evolutionary divergence and polymorphisms can be tolerated. Moreover, as described further infra, the dsRNA can include various modified or nucleotide analogs.

Usually the dsRNA consists of two separate complementary RNA strands. However, in some instances, the dsRNA may be formed by a single strand of RNA that is self-complementary, such that the strand loops back upon itself to form a hairpin loop. Regardless of form, RNA duplex formation can occur inside or outside of a cell.

The size of the dsRNA that is utilized varies according to the size of the subject polynucleotide whose expression is to be suppressed and is sufficiently long to be effective in reducing expression of the subject polynucleotide in a cell. Generally, the dsRNA is at least 10-15 nucleotides long. In certain applications, the dsRNA is less than 20, 21, 22, 23, 24 or 25 nucleotides in length. In other instances, the dsRNA is at least 50, 100, 150 or 200 nucleotides in length. The dsRNA can be longer still in certain other applications, such as at least 300, 400, 500 or 600 nucleotides. Typically, the dsRNA is not longer than 3000 nucleotides. The optimal size for any particular subject polynucleotide can be determined by one of ordinary skill in the art without undue experimentation by varying the size of the dsRNA in a systematic fashion and determining whether the size selected is effective in interfering with expression of the subject polynucleotide.

Double-stranded RNA can be prepared according to any of a number of methods that are known in the art, including in vitro and in vivo methods, as well as by synthetic chemistry approaches.

In vitro methods generally involve inserting the segment corresponding to the candidate gene that is to be transcribed between a promoter or pair of promoters that are oriented to drive transcription of the inserted segment and then utilizing an appropriate RNA polymerase to carry out transcription. One such arrangement involves positioning a DNA fragment corresponding to the candidate gene or segment thereof into a vector such that it is flanked by two opposable polymerase-specific promoters that can be same or different. Transcription from such promoters produces two complementary RNA strands that can subsequently anneal to form the desired dsRNA. Exemplary plasmids for use in such systems include the plasmid (PCR 4.0 TOPO) (available from Invitrogen). Another example is the vector pGEM-T (Promega, Madison, Wis.) in which the oppositely oriented promoters are T7 and SP6; the T3 promoter can also be utilized.

In a second arrangement, DNA fragments corresponding to the segment of the subject polynucleotide that is to be transcribed is inserted both in the sense and antisense orientation downstream of a single promoter. In this system, the sense and antisense fragments are cotranscribed to generate a single RNA strand that is self-complementary and thus can form dsRNA.

Various other in vitro methods have been described. Examples of such methods include, but are not limited to, the methods described by Sadher et al. (14 BIOCHEM. INT. 1015 (1987)); by Bhattacharyya (343 NATURE 484 (1990)); and by Livache, et al. (U.S. Pat. No. 5,795,715), each of which is incorporated herein by reference in its entirety.

Single-stranded RNA can also be produced using a combination of enzymatic and organic synthesis or by total organic synthesis. The use of synthetic chemical methods enable one to introduce desired modified nucleotides or nucleotide analogs into the dsRNA.

Double-stranded RNA can also be prepared in vivo according to a number of established methods (see, e.g., Sambrook, et al. (1989) MOLECULAR CLONING: A LABORATORY MANUAL, 2^{nd} ed.; TRANSCRIPTION AND TRANSLATION (B. D. Hames, and S. J. Higgins, Eds., 1984); DNA CLONING, volumes I and II (D. N. Glover, Ed., 1985); and OLIGONUCLEOTIDE SYNTHESIS (M. J. Gait, Ed., 1984). Each method is incorporated herein by reference in its entirety.

Once the single-stranded RNA has been formed, the complementary strands are allowed to anneal to form duplex RNA. Transcripts are typically treated with DNAase and further purified according to established protocols to remove proteins. Usually such purification methods are not conducted with phenol:chloroform. The resulting purified transcripts are subsequently dissolved in RNAase free water or a buffer of suitable composition.

Double-stranded RNA is generated by annealing the sense and anti-sense RNA in vitro. Generally, the strands are initially denatured to keep them separate and to avoid self-annealing. During the annealing process, typically certain ratios of the sense and antisense strands are combined to facilitate the annealing process. In some instances, a molar ratio of sense to antisense strands of 3:7 is used; in other instances, a ratio of 4:6 is utilized; and in yet other instances, the ratio is 1:1.

The buffer composition utilized during the annealing process can in some instances affect the efficacy of the annealing process and subsequent transfection procedure. While some have indicated that the buffered solution used to carry out the annealing process should include a potassium salt such as potassium chloride (e.g. at a concentration of about 80 mM). In some embodiments, the buffer is substantially potassium free. Once single-stranded RNA has annealed to form duplex RNA, typically any single-strand overhangs are removed using an enzyme that specifically cleaves such overhangs (e.g., RNAase A or RNAase T).

A number of options can be utilized to deliver the dsRNA into a cell or population of cells such as in a biological sample, cell culture, or tissue. For instance, RNA can be directly introduced intracellularly. Various physical methods are generally utilized in such instances, such as administration by microinjection (see, e.g., Zernicka-Goetz, et al., 124 DEVELOPMENT 1133 (1997); and Wianny, et al., 107 CHROMOSOMA 430 (1998)). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

If the dsRNA is to be introduced into an organism or tissue, gene gun technology is an option that can be employed. This generally involves immobilizing the dsRNA on a gold particle which is subsequently fired into the desired tissue. Research has also shown that mammalian cells have transport mechanisms for taking in dsRNA (see, e.g., Asher, et al., 223 NATURE 715 (1969)). Consequently, another delivery option is to administer the dsRNA extracellularly into a body cavity, interstitial space or into the blood system of the mammal for subsequent uptake by such transport processes. The blood and lymph systems and the cerebrospinal fluid are potential sites for injecting dsRNA. Oral, topical, parenteral, rectal and intraperitoneal administration are also possible modes of administration.

The composition introduced can also include various other agents in addition to the dsRNA. Examples of such agents include, but are not limited to, those that stabilize the dsRNA, enhance cellular uptake and/or increase the extent of interference. Typically, the dsRNA is introduced in a buffer that is compatible with the composition of the cell into which the RNA is introduced to prevent the cell from being shocked. The minimum size of the dsRNA that effectively achieves gene silencing can also influence the choice of delivery system and solution composition.

Sufficient dsRNA is introduced into the tissue to cause a detectable change in expression of a target gene (assuming the candidate gene is in fact being expressed in the cell into which the dsRNA is introduced) using available detection methodologies. The amount of dsRNA required for such modulation depends upon various factors such as the mode of administration utilized, the size of the dsRNA, the number of cells into which dsRNA is administered, and the age and size of an animal if dsRNA is introduced into an animal. An appropriate amount can be determined by those of ordinary skill in the art by initially administering dsRNA at several different concentrations for example, for example.

A number of options are available to detect interference of candidate gene expression (i.e., to detect candidate gene silencing). In general, inhibition in expression is detected by detecting a decrease in the level of the protein encoded by the candidate gene, determining the level of mRNA transcribed from the gene and/or detecting a change in phenotype associated with candidate gene expression.

v. Polyamine Analogs

Modulation of ProMac biological activity, specifically proliferation, may also be accomplished though the use of polyamine analogs, their stereoisomers, salts, and protected derivatives.

Among polyamine analogs preferred for use in this invention are those compounds with a demonstrated ability to modulate naturally occurring polyamine levels in cells. Without intending to be limited by theory, possible mechanisms include competition in the polyamine synthesis pathway; upregulation of polyamine catabolizers, such as SSAT; and affecting polyamine metabolism.

A polyamine analog, stereoisomer, salt or protected derivative (or a composition comprising an effective amount of any polyamine analog, or stereoisomer, salt or protected derivative) may be used in vitro or in vivo. In vitro, a suitable biological sample (such as a blood sample, which may or may not be enriched for the macrophage population) is contacted with the candidate modulator. In vivo, a composition of the invention is generally administered according to the manufacturer's/supplier's instructions. Generally, polyamine analogs are administered by subcutaneous or intravenous injection. They may also be administered orally.

The amount of a polyamine analog administered will depend on several variables, such as the particular analog (or stereoisomer, salt or protective derivative) used, the time course of administration, how many doses will be administered, and whether any other substances are being administered. Generally, the amount used will be as recommended by the manufacturer and/or based on empirical studies. In the case of polyamine analogs (or stereoisomer, salt, or protected derivative thereof), the amount will generally be between about 1 to about 300 mg/m²/day, possibly between about 15 to about 150 mg/m²/day. Administration is generally intermittent, meaning that analog (or stereoisomer, salt, or protected derivative thereof) is administered per a period of at least one to two days and then not administered for a period of at least one to two days, with the cycle repeated as indicated.

Polyamine analogs may be administered through a suitable pharmaceutical excipient. Pharmaceutical excipients are known in the art and are set forth in Remington's' Pharmaceutical Sciences, 18th edition, Mack Publishing (1990). The polyamine analog may also be associated with another substance that facilitates agent delivery to macrophages, or increases specificity of the agent to macrophages. For example, an agent may be associated into liposomes. Liposomes are known in the art. The liposomes in turn may be conjugated with targeting substance(s), such as IgGFc receptors. Substances that increase macrophage phagocytosis such as zymosan or tetrachlorodecaoxygen (TCDO) and/or activation such as MCSF, GMCSF or IL-3 may be used to increase uptake of anti-proliferative agent(s).

A polyamine analog (or stereoisomer, salt or protected derivative) may be administered alone, or in conjunction with other substances and/or therapies, depending on the context of administration (i.e., desired end result, condition of the individual, and indications). “In conjunction with” means that an agent is administered prior to, concurrently, or after other substance or therapy. Examples of substances that might be administered in conjunction with an agent include, but are not limited to, brain neurochemical modulators (in the context of macrophage-associated dementias), and classic anti-neoplastic agents and/or anti-lymphocytic agents such as steroids and cyclosporine derivatives. For example, a polyamine analog (or a stereoisomer, salt or protected derivative thereof) can be administered in conjunction with mitoguazone dihydrochloride.

vi. Other Modulators of ProMac Activity

Any agent that interferes with the proliferation of macrophages would be a suitable modulator. Such molecules may include, but are not limited to, cytokines, anti-proliferative chemical compounds, and other small molecules.

b. Screening for ProMac Modulators

Screening assays for ProMac modulators can be based upon any of a variety of techniques readily available and known to one of ordinary skill in the art. In general, the screening assays involve contacting ProMacs with at least one candidate modulator, and assessing the effect upon ProMac biological activity. Modulation of biological activity, or lack thereof, can be determined by, for example, detecting the expression level of a ProMac signature gene product (e.g., a decrease in mRNA transcript or polypeptide levels would in turn cause a decrease in biological activity of the gene product). Alternatively or in addition, the effect of the candidate modulator can be assessed by examining through a functional assay. For example, the effect upon cell proliferative activity can be assessed. Modulators of primary interest are those that decrease ProMac activity (e.g., proliferation).

Assays described herein can be readily adapted in the screening assay embodiments of the invention. Exemplary assays useful in screening candidate modulators include, but are not limited to, hybridization-based assays (e.g., use of nucleic acid probes or primers to assess expression levels), antibody-based assays (e.g., to assess levels of polypeptide gene products), binding assays (e.g., to detect interaction of a candidate agent with a differentially expressed polypeptide, which assays may be competitive assays where a natural or synthetic ligand for the polypeptide is available), and the like. Additional exemplary assays include, but are not necessarily limited to, cell proliferation assays, antisense knockout assays, assays to detect inhibition of cell cycle, assays of induction of cell death/apoptosis, and the like. Generally such assays are conducted in vitro, but many assays can be adapted for in vivo analyses, e.g., in an animal model of the cancer.

i. Screening for Antibody Modulators

In most embodiments, the method for detecting antibody binding to a ProMac and modulation of ProMac biological activity resulting from the binding will employ an antibody specific for a ProMac signature gene product. Specifically, methods for detecting binding may comprise contacting the ProMacs, either purified or in a biological sample, with an antibody and then detecting antibody binding to the ProMacs. More specifically, the antibody may be labeled so as to produce a detectable signal using compounds including, but not limited to, a radiolabel, an enzyme, a chromophore and a fluorophore. As is familiar to those of ordinary skill in the art, such antibody-based assays can also be used to measure the expression level of a ProMac signature gene product, therefore also providing a method to detect modulation of ProMac biological activity.

Detecting the binding of an antibody, or a functional equivalent thereof to a ProMac, when compared to a suitable control, indicates that the antibody may be capable of modulating ProMac biological activity. Suitable controls include a sample known not to contain ProMacs and a ProMac sample contacted with a non-specific antibody. A variety of methods to detect specific antibody-antigen interactions are known in the art and may be used in the method, including, but not limited to, standard immunohistological methods, immunoprecipitation, an enzyme immunoassay, and a radioimmunoassay. In general, the specific antibody will be detectably labeled, either directly or indirectly. Direct labels include radioisotopes; enzymes whose products are detectable (e.g., luciferase, 3-galactosidase, and the like); fluorescent labels (e.g., fluorescein isothiocyanate, rhodamine, phycoerythrin, and the like); fluorescence emitting metals (e.g., 112Eu, or others of the lanthaide series, attached to the antibody through metal chelating groups such as EDTA); chemiluminescent compounds (e.g., luminol, isoluminol, acridinium salts, and the like); bioluminescent compounds (e.g., luciferin, aequorin (green fluorescent protein), and the like). The antibody may be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The biological sample may be brought into contact with and immobilized on a solid support or carrier, such as nitrocellulose, that is capable of immobilizing cells. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls and to appropriate standards.

In screening for modulators, it may also be desirable to assess the binding affinity of an antibody capable of binding ProMacs. Binding affinity can be readily determined by one of ordinary skill in the art, for example, by Scatchard analysis (Scatchard, 51 ANN. NY ACAD. SCI. 660 (1949)).

4. Promacs and their Implications

Macrophages serve as the first line of defense for humans against the wide range of pathogenic organisms with which man is exposed to daily. Macrophages can ingest or phagocytose foreign bacteria or proteins, initiate an immune response, or inhibit an existing immune response depending on the various cytokines, chemokines, and other proteins the macrophage secretes. In healthy individuals these various functions of the macrophage are tightly regulated and the actions of macrophages are beneficial to the host organism. Recently, however, it has become apparent that macrophages can also be deleterious and actually promote tumor formation and spread^{1,2}. A pathogenic role for macrophages has also been described for a wide range of chronic inflammatory conditions, neurodegenerative disorders, vascular disorders, immunological disorders, including without any limitation, amyotrophic lateral sclerosis (ALS), Alzheimer's disease (AD), HIV associated dementia (HAD), macular degeneration (MacDgn), scleroderma, and arteriosclerosis, to name but a few³⁻⁷. See the list below for additional diseases associated with macrophages.

Macrophage Associated Diseases

| | Disease | Prevalence | Ref(s) |
|---|---|---|---|
| 1 | Alzheimer's Disease | 4 × 10⁶ | Casal et al Clin Biochem 36: 553-556, 2003 |
| | | | Zhang et al J Neuroimmunol 159: 215-224, 2005 |
| 2 | Amyotrophic Lateral | ˜30,000 | Reviewed in McGeer et al. Muscle Nerve 26: 459-470, 2002 |
| | Sclerosis | | Zhang et al J Neuroimmunol 159: 215-224, 2005 |
| 3 | Asthma | ˜17 × 10⁶ | Peters-Golden M. Am J Respir Cell Mol Biol 31: 3-7, 2004 |
| | | | Douwes et al. Thorax 57: 643-648, 2002 |
| 4 | Atherosclerosis | ˜4.7 × 10⁶ | Hansson G K. NEJM 352: 1685-1695, 2005 |
| 5 | Autism | 1 in 250 | Vargas et al. Ann Neurol 57: 67-81, 2005 |
| | | ˜1 × 10⁶ | |
| 6 | Dermatomyositis | 1 in 20,000 | Greenberg et al. Ann Neurol 57: 664-678, 2005 |
| | | ˜13,000 | |
| 7 | Diabetes | ˜7.4 × 10⁶ | IDDM Jun et al J Exp Med 189: 347-358, 1999 |
| | | IDDM 15% | NIDDM Wellen & Hotamisligil J Clin Invest 115: 1111: |
| | | NIDDM 83% | 1119, 2005 |
| | | Other 2% | |
| 8 | Frailty | ˜7% of >65 yrs | Walston et al Arch Intern Med 162: 2333-2341 |
| | | ˜2 × 10⁶ | |
| | | & increasing | |
| 9 | HIV associated | ˜120,000 | Anderson et al JAIDS 31: S43-S54, 2002 |
| | Dementia | | |
| 10 | Inflammatory Bowel | Crohn's ˜500,000 | Kanke et al Dig Liver Dis 36: 811-817, 2004 |
| | Disease | U colitis ˜500,000 | Boone & Ma J Clin Invest 111: 1284-1286, 2003 |
| 11 | Kidney Disease | Diabetes ˜138,000 | Erwig et al. Nephrol Dial Trans 18: 1962-1965, 2003 |
| | | Hyperten ˜91,636 | Wilson et al. Curr Opin Nephrol 13: 285-290, 2004 |
| | | Glomerul ˜60,888 | |
| 12 | Lupus | 1.5 × 10⁶ | Baechler et al PNAS 100: 2610-2615, 2003 |
| 13 | Macular Degeneration | 10 × 10⁶ | Espinosa-Hiedmann et al. Invest Ophthalmol Vis Sci. |
| | | | 44: 3586-3592, 2003 |
| 14 | Multiple Sclerosis | ˜390,000 | Carson M J Glia 40: 218-231, 2002 |
| | | | Minagar et al. J Neurol Sci 202: 13-23, 2002 |
| 15 | Obesity | 30% Obese | Weisberg et al J Clin Invest. 112: 1796-1808, 2003 |
| | | ˜5% severely | |
| | | obese | |
| | | 75 to 13 million | |
| 16 | Parkinson's Disease | ˜1 × 10⁶ | McGeer & McGeer Parkinsonims Relat Disord 10: S3-S7, |
| | | | 2004 |
| 17 | Psoriasis | ˜5.5 × 10⁶ | Nestle et al J Exp Med 202: 135-143, 2005 & refs within |
| 18 | Primary Biliary | ˜9,200 | Mathew et al Histopathology 25: 65-70, 1994 |
| | Cirrhosis | | Takii et al Lab Invest 85: 908-920, 2005 |
| 19 | Primary sclerosing | ˜7500 | Cameron R G et al Clin Biochem 34: 195-201, 2001 |
| | cholangitis | | |
| 20 | Post-Radiation syndrome | Treatment for | Veeraasarn et al Radiother Oncol 73: 179-185, 2004 |
| | | multiple cancers | |
| 21 | Rheumatoid Arthritis | 2.5 × 10⁶ | Kinne et al Arthritis Res 2: 189-202, 2000 |
| 22 | Sarcoidosis | ˜50,000 | Martin et al Am J Respir Criti Care Med 170: 567-571, 2004 |
| 23 | Schizophrenia | ˜2.2 × 10⁶ | Nikkila et al Am J Psychiatry 156: 1725-1729, 1999 |
| 24 | Scleroderma w/wo | ˜250,000 | Atamas & White, Cytokine & Growth factors reviews 14: |
| | pulmonary fibrosis | | 537-550, 2003 |

These deleterious macrophages often are seen to have elevated levels of proteins associated with cell division (such as Proliferating Cell Nuclear Antigen or PCNA) and are referred to as ProMacs for Proliferating Macrophages. Thus it would be advantageous to have a means of easily detecting and quantitating the level of ProMacs in any given individual as a means of diagnosing the onset of inflammatory conditions that are currently difficult to unambiguously diagnose (i.e. ALS or AD). Additionally a straightforward molecular assay for ProMacs would be useful for the identification and optimization of small molecule therapeutics that target the ProMac population, such as polyamine analogs. Finally definition of the cell surface receptors most highly associated with proliferating macrophages would allow for the development of antibody-based therapeutics that significantly reduce the ProMac levels of patients with minimal side affects.

This invention describes a relatively large group of coordinately regulated cellular transcripts which can be used to unambiguously identify the presence and relative levels of ProMacs in the peripheral blood of patients. The transcripts in this ProMac molecular signature share the properties of (1) being primarily expressed in macrophages; (2) having expression that is highly correlated with other transcripts in the ProMac signature; and (3) being upregulated to greater or lesser extents in individuals with disease. The genes that comprise the ProMac signature include genes involved in the response to type I interferons (α/β) and genes involved in the nuclear factor of kappa light polypeptide gene enhancer in B-cells (NFKβ) mediated regulation of transcription, as well as a large number of other genes that have been implicated in other cellular processes such as cellular remodeling and apoptosis.

The assays for the detection of ProMacs can be applied to any previously described nucleic acid amplification and quantitation format. In particular the assays described are well suited to so-called gene chip systems (Affymetrix, Agilent, Quantum Dot, Celera others); quantitative reverse transcription-polymerase chain reaction (RT-PCR) methodologies (Real time PCR systems); or other quantitative or semi-quantitative amplification technologies including Branched DNA methodologies, RRR, GenProbe-like and associated methods, ligase chain amplification, amongst others. The assays can involve as many different members of the ProMac signature as is necessary or useful and a relatively well optimized prototype assay involving use of various panels of ProMac signature genes and Light-cycler methods is described in detail below. Additionally assays can be run by flow cytometry using selected cell surface markers, or by ELISA for cell-associated or secreted proteins. Detection methods for ProMac signature genes can be combined with assays for the detection of other genes that may provide useful clinical information such that

The assays for the detection of ProMacs are shown to be useful for the detection of both Alzheimer's disease (AD) and amyotrophic lateral sclerosis (ALS). A prototype assay involving a related set of various genes is described that can differentiate ALS from AD. The assays can be useful for any human disease that is primarily driven by a chronic inflammatory state. In addition the described assays are shown to be valuable for the identification of drugs that have the ability to alter ProMac function and or kill ProMacs. Once candidate drugs for the treatment of ProMacs are identified the same assays can be employed to pre-screen potential patients to identify those likely to benefit from drug administration. Finally results from an ongoing Phase I clinical trail are presented that directly link administration of a drug known to kill ProMac in vitro to normalization of a ProMac signature in the patient. This signature normalization was associated with remission of the patients T cell lymphoma for a period of over 2 months.

### EXAMPLE 1

### Microarray Analysis of PBMCs from ALS and AD Patients

Amyotrophic Lateral Sclerosis

The signature was originally discovered while studying amyotrophic lateral sclerosis (ALS). ALS is a debilitating neurological disorder which occurs at a prevalence of 4-6 cases per 100,000 individuals with an incidence of 1 to 2 cases per 100,000. ALS manifests as progressive muscle weakness and spasticity^{4,8}. The paralysis is caused by neuronal degradation primarily in the anterior horn region of the spinal cord. The spasticity results from corticospinal degeneration. The disease is uniformly fatal, and relentlessly progressive with death ensuing, for most patients, within one to five years of the onset of symptoms. Death is generally related to respiratory failure due to paralysis^{4,8}. To this point pharmacotherapy has resulted in minimal extension of the disease course⁹.

The causes of ALS are unknown, but approximately 10% of cases are familial (reviewed in¹⁰). Approximately 25% of individuals with familial ALS have mutations within the superoxide-dismutase-1 (SOD1) gene. Transgenic mice with mutated SOD1 proteins develop a progressive muscle weakness and paralysis with cellular features very reminiscent of ALS^{11,12}. The remaining 90% of ALS cases are described as sporadic, in that there is no obvious family history of ALS. The causes of sporadic ALS are completely unknown. Various theories have been advanced that include glutamate excitotoxicity, oxidative stress, impaired axonal transport, protein misfolding, viral infection, mutation of other yet to be characterized genes, and exposure to toxic agents^{4,8,13}. However, compelling evidence for any single explanation for ALS is lacking.

Both familial and sporadic ALS are characterized by high levels of immune activation of the microglial cells of the spinal cord and cerebellum (reviewed in^{4,10}). In particular, large numbers of reactive microglia are found throughout the diseased regions and not in unaffected tissue^{4,12,14}. Other pathological observations in the anterior horn of the spinal cord include the presence of dendritic cells, significant deposits of endogenous IgG and spheroid bodies, which are composed of various classes of neurofilament proteins^{4,14}. Infiltrations of T cells are less common. Additionally, various groups have found elevated levels of monocyte chemoattractant protein-1 (MCP-1,^{15,16}), or prostaglandin PGE2¹⁷ in ALS patient cerebral spinal fluid. Use of the SOD1 mouse model has confirmed that activation of the microglial cells preceded overt hind limb paralysis and increased with progressive weakness¹². Also, expression of mutant SOD1 only in motor neurons did not induce disease¹⁸. Another study in the murine SOD1 model determined that chronic stimulation of the innate immune system with lipopolysacharide hastened the disease course¹⁹. Thus activation of spinal cord microglia may be the primary cause of neuronal degradation in ALS. The initial cause of the microglial activation is unknown.

Recently we found that the immune activation of the spinal cord in ALS patients was mirrored by increased levels of activated macrophages in the peripheral blood²⁰. The same study also found a significant relationship between activation of macrophages (as measured by mean HLA-DR levels on CD14 cells) and the rate of decline in ALSFRS values. Previous evidence for systemic involvement in ALS includes evidence for hypermetabolism^{21,22} and alterations in the skin of ALS patients^{23,24}. Additionally, increasing evidence suggests that a significant fraction of ALS patients also exhibit symptoms of frontotemporal dementia²⁵. These findings are consistent with ALS being a disease that affects multiple organ systems even though death is a direct result of motor neuron damage. In that context, systemic inflammation would be expected.

The finding of high levels of activated macrophages in peripheral blood of ALS and AD patients²⁰ led us to undertake a study of gene expression in peripheral blood cells in patients with ALS, AD, and controls using microarray technology. The clinical and demographic characteristics of the study participants are summarized in Table 1 (FIG. 33). Overall, the 3 groups were reasonably well matched with AD patients and the healthy controls tending to include more females than the ALS patients. Peripheral blood mononuclear cells were isolated via Percoll gradient centrifugation. The PBMCs were then resuspended in culture media and incubated for ˜16 hours at 37° C. under non-adherent conditions. This allowed for recovery from the isolation procedure. Total RNA was then prepared and the quantity and integrity of the total RNA was verified using an Agilent 2100 Bioanalyzer. The RNAs were then prepared for microarray hybridization according to Affymetrix protocols²⁶ and hybridized to HGU133plus2.0 microarrays. The microarrays were read using a GeneChip Scanner 3000 and raw data converted to probe specific signals employing MAS 5.0 protocols as implemented in the GCOS software package (Affymetrix, Santa Clara Calif.). Signal normalization was performed using the median signal from a set of 100 probes validated as being relatively invariant in multiple cell types²⁷. Normalized probe signals were subsequently evaluated using the Excel²⁸ and GeneSpring²⁹ software packages.

For all 3 groups and controls, the percentage of probe sets that had a signal above background was approximately 42%, c.f. Table 2 (FIG. 34). In an attempt to age match, the signals obtained from patients with AD were compared to controls who were >60 (N=11), and signals obtained with ALS patients were compared to all controls. Overall, 24,148 to 28,544 probe sets exhibited a signal of 50 or more (range 0-22,011) in at least 3 of the samples. Thus a similar fraction of the total genes was expressed in each experimental group. The total number of probe sets downregulated 2 fold or more relative to controls was ˜400 for each group. 883 probe sets were upregulated in ALS patients and 477 upregulated in AD patients.

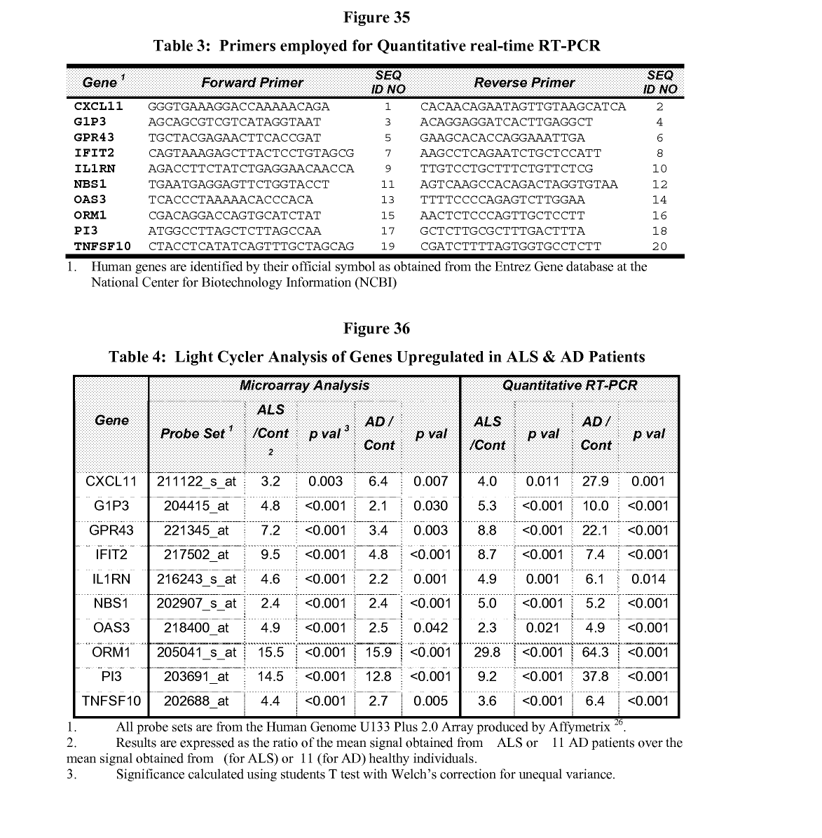

For genes upregulated in ALS and AD patients, a significant fraction of the probe sets (361 of 883, 41% for ALS patients: 361 of 477, 76% for AD patients) were also upregulated in the other patient group. The 361 probe sets encompassed 280 known genes or transcripts associated with various functions including the α/β interferon response (gene symbol ═IFIT2, IFI27), signal transduction (IRAK3, TNFAIP6) and the immune response (CD80, FCGR2A). Only 12 of the 280 common genes were also upregulated in a study of incubation-dependent changes in gene expression in PBMCs from healthy individuals³³. Thus, the changes observed in all three populations are not primarily a response to in vitro incubation. Not unexpectedly, there was also little overlap in the genes found upregulated in this study of PBMCs and the gene upregulated in previous analyses of gene transcription in post-mortem spinal cord tissue obtained from ALS patients^{34,35}. These results suggest that individuals with neurodegenerative disease had a common transcriptional response in their PBMCs not seen in controls. To confirm the results obtained in the microarray analysis, primers were designed using the sequences of 10 of the genes that appeared to be significantly upregulated in both patient groups. Sequence of oligonucleotide primers provided in Table 3 (FIG. 35). Aliquots of the RNA preparations were then RT-PCR amplified using quantitative real time methods employing a Light-Cycler³⁶. The signals obtained were normalized against the signal obtained with primers for β-actin according to the formula Ct_{actin}−Ct_{gene}=NSig where Ct=the threshold cycle for the indicated gene and NSig=the normalized signal. In general, results obtained by quantitative real-time RT-PCR were in good agreement with results obtained by microarray analysis, confirming that the majority of genes predicted to be upregulated by the microarray results were, in fact, significantly upregulated.

### EXAMPLE 2

### A Quantitative RT-PCR Assay for Detection of ALS and AD Patients

The existence of significant changes in the gene expression of PBMCs of ALS patients relative to controls raised the possibility that these changes could be used to assist in the diagnosis of ALS. Therefore, we constructed a “weighted voting” scheme using the methods described by Golub and co-workers³⁷ for 10 of the most promising primer sets. ALS and control patients were divided into training and test sets and the actin normalized signal for each sample with each primer set was determined as described above. Then the voting weights (modified Rs) were calculated using the test samples according to the formula
Mod R=(Mean ALS−Mean CON)/(StDev ALS+StDev CON)
calculated using 13 ALS samples & 11 controls.

Next the inflection point or the mid point between the ALS and control samples is calculated according to
MID=(Mean_{ALS}+Mean_{CON})/2

using the same samples as for the Mod R.

Then the signal for the individual primer (ICV) is calculated according to
ICV=Mod R(Mean NSig−Mid)

where Mean NSig refers to the mean normalized signal from however many individual assays were run.

The predictive power of the various primers was then evaluated using the test set of samples. Performance of the individual primers for the individual genes at identifying PBMCs from ALS patients was variable. Accordingly, results with 5 of the primer pairs with good performance (G1P3, GPR43, IFIT2, ORM1, TNFSF10) were added together to generate a classification index (LC5-CI) according to the formula
LC5-CI═ICV_{GIP3}+ICV_{GPR43}+ICV_{IFIT2}+ICV_{ORM1}+ICV_{TNFSF10}

The prediction strength for each sample was also calculated according to the formula
PS=LC5-CI/(|ICV_{G1P3}|+|ICV_{GPR43}|+|ICV_{IFIT2}|+|ICV_{ORM1}|+|ICV_{TNFSF10}|)

where |ICV_{gene}|=absolute value of ICV_{gene}

The PS ranges from 1.0, indicating 100% of the results obtained were positive (e,g voted for ALS), to −1.0 indicating that 100% of the results obtained were negative.

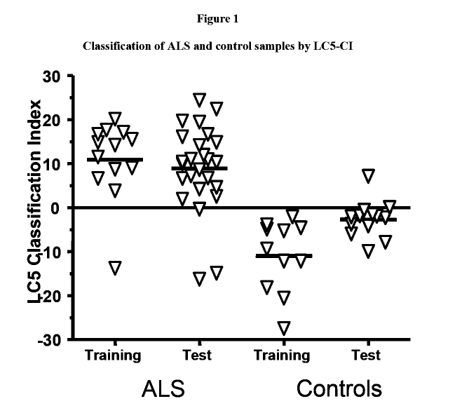

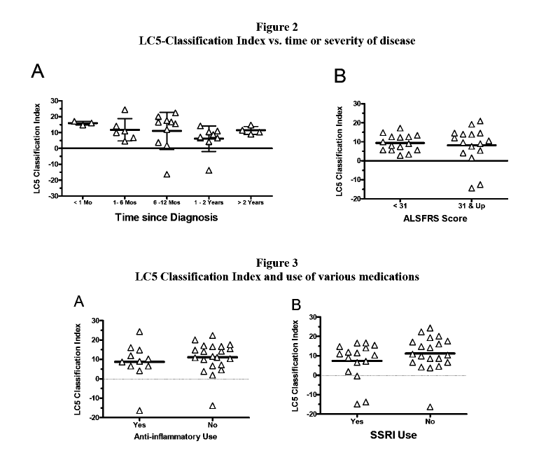

The LC5-CI scores obtained for ALS and control patient samples (both training and test) are presented in FIG. 1. The mean LC5-CI obtained with either the ALS training or test set was significantly higher (p<0.001, One way with ANOVA with Bonferroni's correction for multiple comparisons) than the mean values obtained with either of the control sets. Similarly 22 of 24 controls (92%) were correctly classified as healthy. Three controls and one ALS patient had PS values between −0.3 to 0.3 which are classified as indeterminate, meaning that the prediction obtained was made with a margin of 30% or less. Thus, if the cutoff for declaring a sample to be from an ALS patient is an LC5-CI>0 with a PS>0.3, then the LC5-CI based assay was able to identify ALS patients with a sensitivity of 89% (34 out of 38 for both training & test sets) and a specificity of 96% (one healthy individual classified as ALS with a PS>0.3).

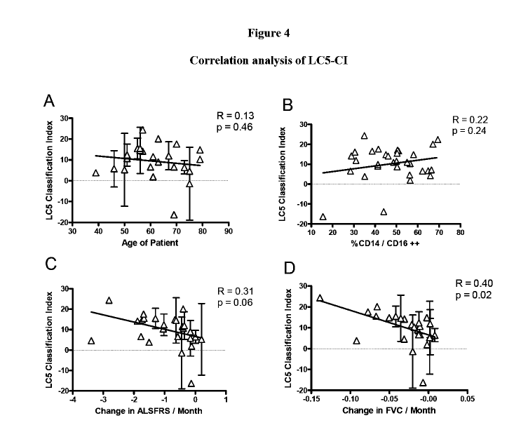

To be a useful diagnostic for ALS, the LC5-CI would have to measure transcriptional changes that were already present in individuals at the time when they become symptomatic. Alternatively, the transcriptional changes measured by the LC5-CI might be a consequence of the motor neuron destruction. If that were the case, one would expect that LC5 CI values would rise as ALS progresses and greater motor neuron damage occurs. To address this question, patients were stratified by the time since diagnosis and the mean LC5-CI values determined for each group (FIG. 2A). No significant differences between the mean LC5-CI values obtained were observed in patients whose samples were collected within 1 month of diagnosis up to more than 2 years past diagnosis. One of the more widely used measures of the extent of disease in ALS is the ALS functional rating score or ALSFRS^{30,38}. The ALSFRS is scored from 0 (worst possible score) to 48 (best possible score) and is relatively straightforward to determine. Therefore, we grouped patients by whether their ALSFRS score when the sample was obtained was above or below the median for the study population (FIG. 2B). No significant difference in the LC5-CI signals obtained from individuals with lower or higher ALSFRS scores was observed. Thus, to the extent that individuals with high ALSFRS values were earlier in their disease course, this did not raise or lower the observed LC5-CI score. Altogether, the data suggest that the transcriptional changes measured by the LC5-CI in ALS PBMCs are present when symptoms become apparent and are maintained for long periods of time. Thus, the LC5-CI should be useful diagnostically.

Next possible confounders of the LC5-CI signal were evaluated. The transcriptional state of peripheral blood cells might be expected to be altered by anti-inflammatory use. However, when the LC5-CI of ALS patients who were on anti-inflammatory medications (NSAIDs, non-prescription pain relievers) was compared to those who were not taking anti-inflammatory medications, no significant difference was observed (FIG. 3A). Nor were any differences observed in the mean LC5-CIs obtained from ALS patients who were taking statins or selective serotonin reuptake inhibitors (FIG. 3B). Finally, Riluzole is the only approved medication for the treatment of ALS⁹ and a majority (21 of 25) of the patients with ALS in this cohort were treated with Riluzole. The mean LC5-CI scores for patients taking or not taking Riluzole were also not significantly different (data not shown). Thus, although the numbers are small, there is no evidence to this point that use of medications commonly seen in ALS patients has a significant effect on the LC5-CI values obtained.

It is also possible that variations in the LC5-CI observed reflected the underlying clinical or demographic state of the ALS or control patients in this sample set. Therefore, the LC5-CI was evaluated for an association with the age and sex of the patients. The mean LC5-CI of the male and female patients were not significantly different (Males=11.1, Females 9.1, p=ns, Students T test) nor was there a significant relationship between the age of the ALS patient and the LC5-CI (FIG. 4A). Nor did the mean LC5-CI vary significantly according to the site of onset of ALS (lower limb=7.3; upper limb=13.2; bulbar=10.3, p=ns for one way ANOVA). Two of the study participants had familial ALS and both of these patients had positive LC5-CI values with high prediction strengths. Thus it appears that the transcriptional signatures of patients with familial and sporadic ALS are similar, although more samples with familial ALS are required to confirm this. As alluded to in FIG. 2B, above there was no significant relationship between the LC5-Cland the ALSFRS or FVC at the time the sample was obtained.

Next we looked at whether there was a relationship in ALS patients between the percentage of CD14/16++ cells detected by flow cytometry and the LC5-CI value obtained (FIG. 4B). Once again no significant correlation of LC5-CI with CD14/16++ percentages was observed. Nor were the LC5-CI values of healthy controls correlated with CD 14-16++ levels (data not shown). This precludes the LC5-CI as being simply a RT-PCR based detection of CD14/16++ cells. Finally we evaluated whether there was a correlation between the mean LC5-CI obtained for the ALS patients (multiple determinations for 9 of the 25 patients) and the rate of change in the ALSFRS or FVC scores of the ALS patients (FIG. 4C-D). For both the rate of change in the ALSFRS and the FVC there was a weak relationship (p=0.06 for LC5-Cland change in ALSFRS: p=0.02 for LC5-Cland change FVC) with greater decreases in ALSFRS or FVC per month associated with higher LC5-CI values. Analysis of LC5-CI as a predictor of survival was precluded by the small number of patients (four) who have died to this point. Thus a peripheral blood based expression assay similar to the LC5-CI may have some ability to predict progression.

Having validated that the LC5-CI was relatively efficient at differentiating ALS patients from controls, it was of interest to determine what sort of LC5-CI values would be obtained from samples with other neurodegenerative or inflammatory disorders.

Accordingly total RNA derived from PBMCs from individuals with HIV infection, HIV-associated neurological disease, Alzheimer's disease, and macular degeneration were prepared and tested with the LC5-CI primers analogously to the testing of the ALS patients and controls, above. PBMCs from patients with AD were tested because of the large number of shared upregulated genes identified in the microarray analyses above. PBMCs from HIV infected individuals with and without neurological dysfunction were assayed because the presence of activated macrophages in the blood of patients with HIV associated neurological disease has been noted previously³. Similary, with the macular degeneration samples, evidence for an elevation in activated macrophages in peripheral blood had been obtained³⁹. The results obtained with the various samples using the LC5-Clare presented in FIG. 5. It can be appreciated that the mean LC5-CI of the ALS or AD patients are significantly higher (p<0.001, One way ANOVA with Bonferroni correction for multiple comparison) than those obtained from healthy controls or macular degeneration patients. Additionally, the mean LC5-CI values from a group of HIV infected individuals with HIV-associated neurocognitive disorders were significantly higher than those obtained from healthy controls (p<0.001). The patients with HIV neurocognitive disorder included one patient with full-blown HIV associated dementia (the HIV neuro with the highest value, LC5-CI=13.7), five with possible or probable minor cognitive motor disorder (MCMD), and three that were classified as sub-syndromic. Overall, the LC5-CI appears to be much higher in patients with neurocognitive disorders than in healthy individuals, or in individuals with some elevation in CD 14/16++ macrophages, such as those with macular degeneration. Thus, the LC5-CI should be useful for the diagnosis of severe neurodegenerative diseases.

### EXAMPLE 3

### Differentiation of ALS from AD

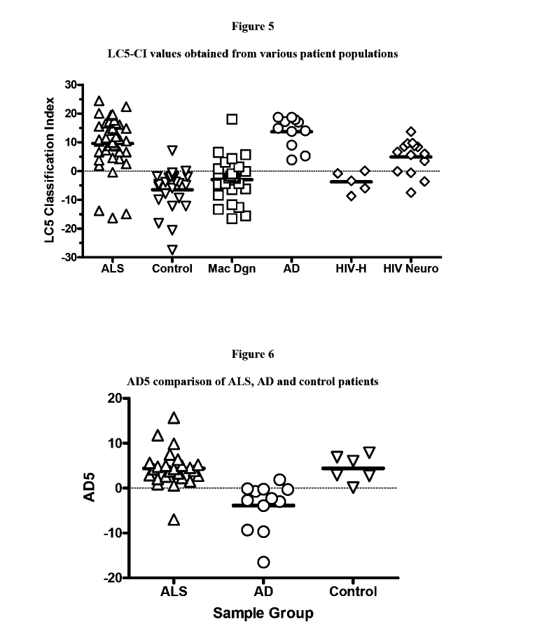

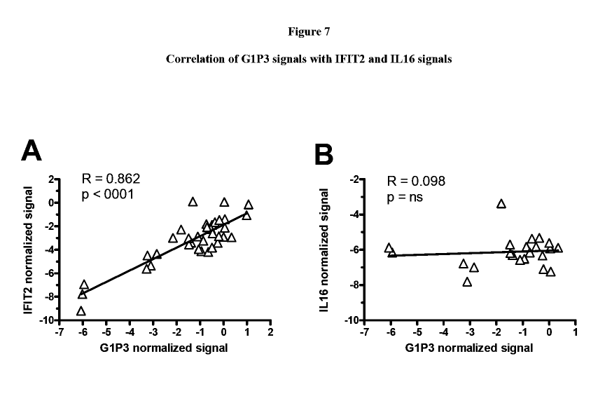

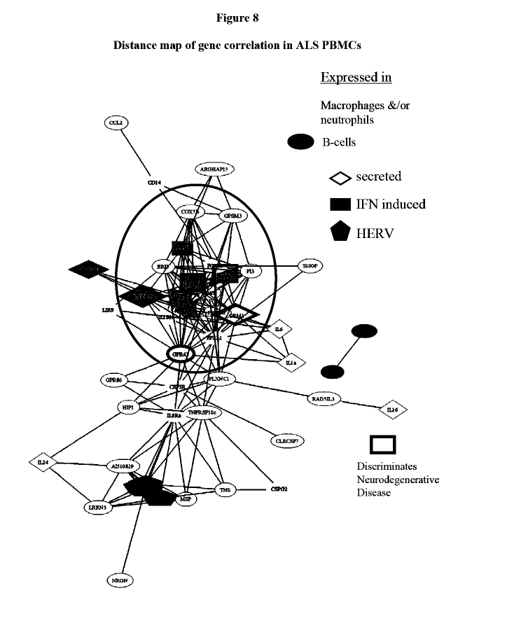

As detailed above, the LC5-CI was useful for identifying PBMCs derived from individuals with ALS and AD. However, one would also like to be able to differentiate ALS from AD in some circumstances. Therefore the microarray data (see above) was evaluated for genes that had the potential to differentiate ALS from AD and candidate genes were chosen and evaluated by quantitative RT-PCR. The results of the light-cycler RT-PCR were evaluated using a weighted voting scheme analogous to that used to generate the LC5-CI. Values obtained with 10 representative genes are presented in Table 5 (FIG. 37). Five genes, CXCL11, MSP, PI3, RAD51L3, and TPD52, four of which had modified R values of between −0.5 and −1.0, were selected to make an index for differentiating ALS from AD, designated the AD-5. As with the LC5-CI system, results obtained with the individual primers were added together with positive values “voting” for ALS and negative values voting for Alzheimer's disease. The results obtained with the training set of 25 ALS patients, 12 AD patients, and 6 age matched controls are presented in FIG. 6. All but one of the ALS samples were scored as positive and of the 24 positive (“ALS-Like”) samples 24 had good prediction strength values. Eleven of 12 AD patients were scored as negative (AD like) with variable prediction strengths. Still there was a highly significant difference in the mean AD5 values obtained with the ALS and AD patients (p<0.001). The six control samples with a mean age of 82 had AD5 values that were not significantly different from the ALS patients. Thus, use of the AD5 or a similar system will be able to efficiently differentiate AD from other diseases that generate a positive LC5-CI.

### EXAMPLE 4

### Definition of a Neurodegenerative Disease Signature

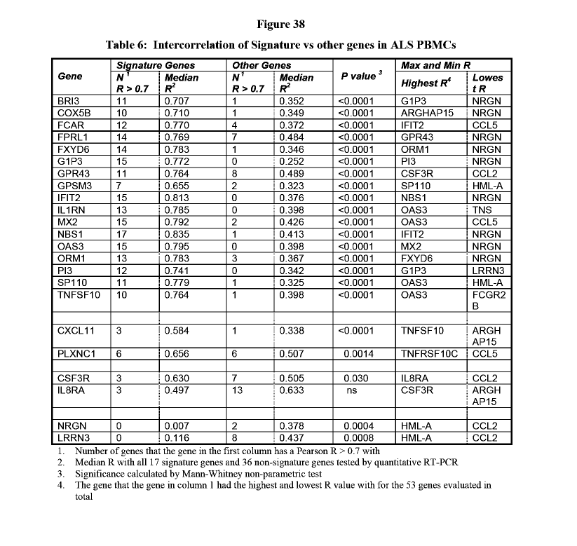

Another aspect of the LC5-CI that became apparent was that the transcription of all 5 genes used to calculate this value showed high degrees of correlation. As an example the actin normalized signals obtained with the LC5-CI genes G1P3 and IFIT2 from ALS patients PBMCs are plotted (FIG. 7A). The Pearson correlation coefficient for these two genes was R=0.862 which was highly significant. A comparison of the signals obtained with G1P3 and interleukin 16, from the same samples yielded an R=0.098, which was not significant (FIG. 7B). Accordingly, the signals obtained from each of the genes used in the RT-PCR analysis of ALS patient PBMCs were employed to determine a Pearson correlation coefficient for each of the gene pairs. This analysis identified a group of 17 genes whose expression was highly correlated. See Table 6 (FIG. 38). The genes BRI3, COX5B, FCAR, FPRL1, FXDY6, G1P3, GPR43, GPSM3, IFIT2, IL1RN, MX2, NBS1, OAS3, ORM1, PI3, SP110, and TNFSF 10 all exhibit very high median R values with each other, much lower median R values with the other 36 genes they were evaluated against. The two genes CXCL11 and PLXNC1 present a more nuanced case in that although they have higher median R values with the signature genes than with the other genes, they do not appear to be as tightly intercorrelated as the others. CSF3R and IL8Ra are two genes that, although there transcription is correlated with a portion of the signature genes, have higher degree of intercorrelation with genes outside of the signature and are not included in it. The final two genes of the table, NRGN and LRRN3, are representative of a group of genes that form an anti-signature because their transcription is the opposite of that seen with the signature genes. In particular NRGN stands out in as it is the gene with the lowest R of all 53 tested with 9 of the signature group.

The relationships between the various genes tested by RT PCR to date can be visualized using an interconnectivity map (FIG. 8). In this representation the correlation coefficients between each gene pair are converted to distances (1-R) and if the distance is <0.3 it is graphed as a line between the two genes with the length of the line roughly equivalent to the distance. In this view the signature is the large group of highly interconnected genes (circled) towards the top of the graph. Also as expected NRGN is located at the very bottom of the graph quite distant from the signature group. The genes closest to the signature group include the aforementioned CXCL11 and PLXNC1 as well as LIR9, CSF3R, and IL6. The simplest interpretation is that the signature includes transcripts from a particular cell present in the peripheral blood of patients with neurodegenerative at significantly elevated levels.

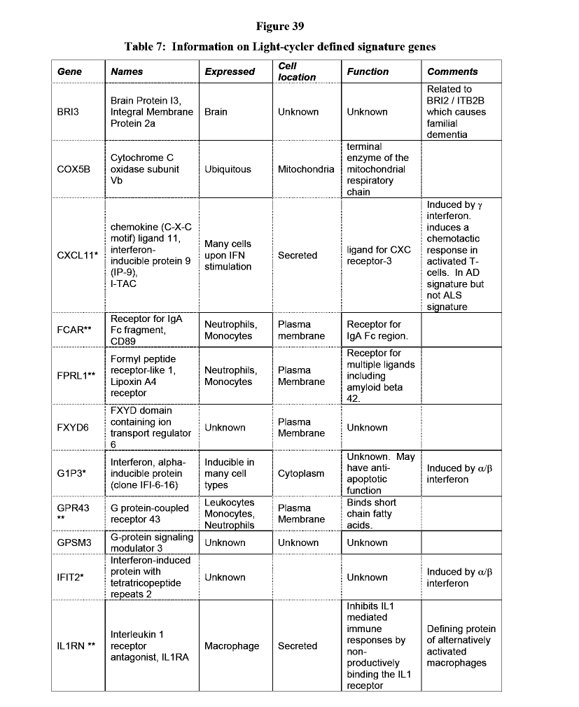

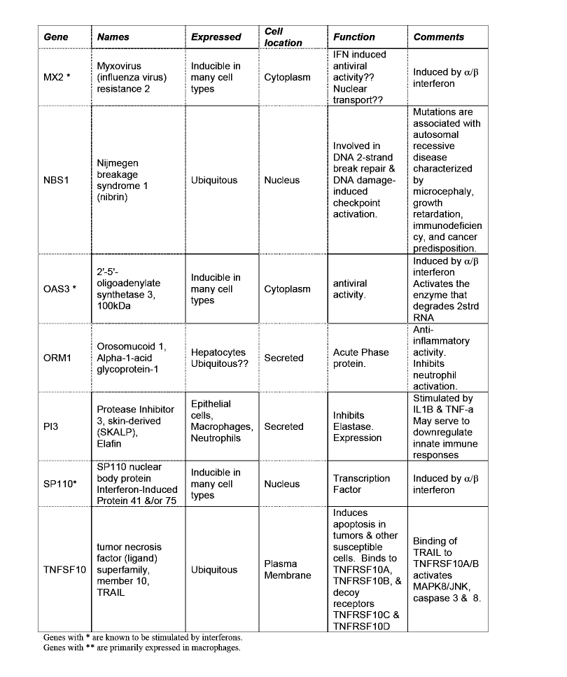

A summary of the information that can be obtained about the 17 proteins of the signature and several of the nearest neighbors are summarized in Table 7 (FIG. 39). The 17 proteins fall into 3 major groups. The first group is proteins known to be involved in the response to interferons. This group includes CXCL11, G1P3, IFIT2, IL1RN, MX2, OAS3, SPI10, and TNFSF10⁴⁰⁻⁴⁴. If the PBMCs are undergoing α/β interferon-mediated stimulation, the correlated appearance of these transcripts would not be surprising. The second group of proteins is proteins that are transcribed primarily or at significant levels in cells of myeloid lineage including, monocytes, macrophage, neutrophils, and granulocytes. These proteins include FCAR CD89), FPRL1, GPR43, and IL1RN⁴⁵⁻⁴⁸. However, it is important to note that a number of other genes that are primarily expressed in myeloid cells including, CD14 (the definitive marker for monocytes), CSF3R, IL8RA, FCGR2b, and CSPG2 are not included in the signature (Table 6 and data not shown). Thus not all monocytes/macrophages produce a signature. Whether the division between signature associated cells and other myeloid cells can be explained using known cell types (e.g. macrophages have the signature/neurtrophils do not) requires further study. Nevertheless the association of the ProMac signature gene with multiple known macrophage proteins confirms the signature bearing cell is a type of macrophage. The rest of the 17 signature genes are those with either unknown or varied function that have not generally before been described in cicrculating macrophages. These proteins include ORM1, PI3, BRI3, NBS1, and GPSM3. In particular BRI3 was thought to be a brain specific protein and PI3 was thought to be skin associated^{49,50}. Overall the signature appears to describe a macrophage-like cell (with FCAR, TNFSF10, and GPR43 on its surface, amongst others) undergoing a classic a interferon response.

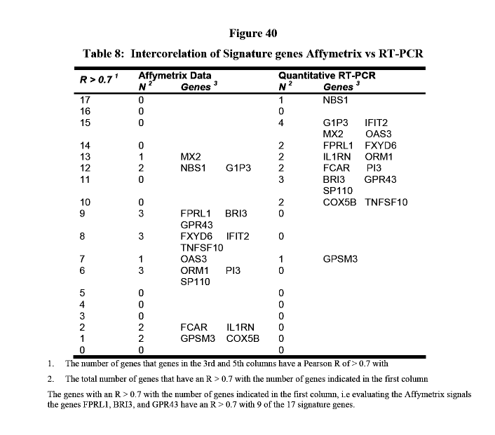

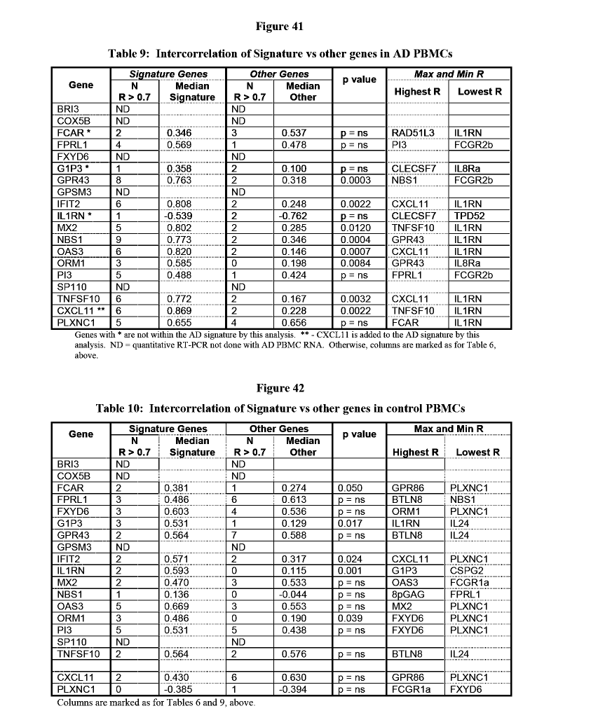

As mentioned above, microarray analysis identified a minimum of 280 genes that were significantly upregulated in both ALS and AD patients. Only a fraction of this total group have been analyzed in depth by quantitative RT-PCR. It is therefore of interest to determine which of the genes that were potentially useful for neurodegenerative disease diagnosis were subject to coordinated transcriptional regulation and could be included in a neurodegenerative signature, similar to the gene transcription signatures that have been identified in multiple cancers^{51,52}. One obvious place to start is the Affymetrix microarray data obtained with the ALS PBMC RNAs. Having now defined 17 signature genes by quantitative RT-PCR (see Table 6 (FIG. 38)) one can use these genes to pull out all other probe sets in the Affymetrix data that also are correlated to a high number of signature genes. Using only those genes that reach a signal of 50 or more in 8 out of 22 ALS samples that met acceptable quality control metrics yields 21,573 (˜39%) human probe sets under analysis. 7,456 of these sets do not have an R>0.7 with any of the 17 signature genes, leaving 14,117 with an R>0.7 with at least one. Thus it is more common for a probe set to have a high R value with at least one signature gene than it is for a probe set not to. Next we evaluated the number of genes in the signature that each individual signature gene had an R>0.7 with for both the RT-PCR data and the Affymetrix derived data. See Table 8 (FIG. 40). The first conclusion that can be drawn is that quantitative RT-PCR is a much more sensitive means of detecting correlations in gene expression in as the number of signature genes that an individual gene has high correlation coefficients with is higher using the RT-PCR data as compared to the gene chip data. Still, 13 of the 17 signature defining probes had a Pearson R>0.7 with 6 or more signature genes using the Affymetrix expression data. The remaining four genes of the RT-PCR determined signature had R>0.7 with one (GPSM3, and COX5B) or two (FCAR and IL1RN) signature genes. For the entire set of 21,573 Probe sets, 6,386 had an R>0.7 with 6 or more signature genes and would be candidates for inclusion in the total set of ALS signature genes. Another observation about signature genes is that they tend to have very poor correlation with the gene neurogranin (NRGN, see Table 6 (FIG. 38)). An evaluation of the 6386 probe sets R with the NRGN probe set indicated that the median R of the Affymetrix signature set was −0.147 and none of the probes had an R>0.7. So the Affymetrix and RT-PCR derived signature sets are similar in that inclusion in the signature excludes a high correlation with the NRGN gene.

The total set of signature genes includes a subset that are upregulated in ALS patient PBMCs. If one takes the 6,386 probe sets with R>0.7 with 6 or more signature genes and asks which of these probes are upregulated 2 or more of fold in ALS patients at a significance level of 0.05 (Students T test with Welch's correction for unequal variance), one is left with a list of 700 probes representing 516 genes (a gene can have many different probe sets) and/or transcripts that appear to be within the neurodegenerative signature and have potential utility at diagnosing ALS. Of the original 13 signature definers included in the list of 6,386, the gene BRI3 fails to reach a 2 fold differential between ALS and control samples (ALS/Control=1.67, p=0.005). The other 12 are included in the 700 up-regulated probe sets. Thus one can define the set of signature genes useful for the diagnosis of ALS as any of the 700 probes/516 genes and transcripts that can differentiate ALS from control patients and that have an R of >0.7 with 6 or more signature genes.

### EXAMPLE 5

### The ProMac Signature in AD Patients and Controls

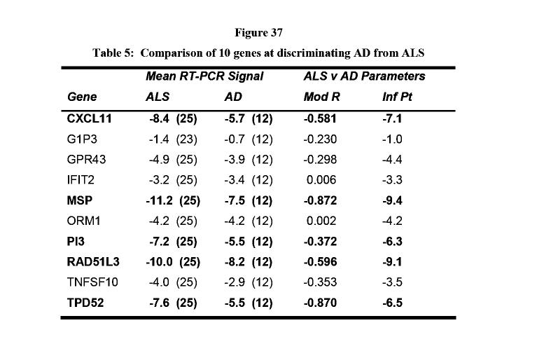

One can also ask how stable the signature is across different neurodegenerative diseases. Accordingly we evaluated the Pearson correlation coefficients obtained from the light cycler RT-PCR data obtained from PBMCs from 12 patients with Alzheimer's disease. The results are presented in Table 9 (FIG. 41). The genes GPR43, IFIT2, MX2, NBS1, OAS3, and TNFSF10 remain highly intercorrelated with each other. The genes FPRL1, PI3, and to a lesser extent ORM1 still exhibit higher correlations with signature genes than with other genes, but the overall strength of the correlation with the signature genes is dropping. The genes FCAR, G1P3 and IL1RN have little or no evidence for co-correlated transcription with the ALS signature. In particular IL1RN exhibits negative correlations with most of the signature genes. In contrast, CXCL11, which was only peripherally related to the ALS signature is now clearly correlated much more strongly with signature genes than with the 14 other genes it was tested against. Thus different neurodegenerative diseases do in fact alter the genes that fall within the signature. This alteration can occur even if there remains a strong differential between signal levels in AD patients and controls, as is the case for the gene G1P3 (see Table 4 (FIG. 36)).

Next, the AD signature analysis was extended to include the Affymetrix gene chip data. To do that we used added CXCL11 to the signature, due to its highly correlated transcription with the other signature genes tested by quantitative RT PCR. The ALS signature genes FCAR, G1P3 and IL1RN were excluded because they did not exhibit correlated transcription with the other genes by quantitative RT PCR analysis. Overall, for 22,458 genes present in 3 or more AD samples with a signal of 50 or more a total of 11,154 genes did not exhibit an R of 0.7 and higher with any of the 14 evaluated signature genes. The maximum number of signature genes any one probe set had R>0.7 was 8 of 14 (1 probe set). Of the 7 genes seen as highly intercorrelated by RT-PCR 3 had R>0.7 with 6 genes (IFIT2, NBS1, and GPR43), 3 had an R>0.7 with 5 genes (BRI3, MX2, and FXYD6) and 3 had R>0.7 with 3 genes (SP110, GPSM3, TNFSF10). CXCL11 had an R>0.7 with 2 signature genes. The three more weakly intercorrelated genes had R>0.7 only with themselves, as did OAS3. The other 3 genes that were not in the AD signature by quantitative RT-PCR (FCAR, G1P3, and IL1RN) also did not have an R>0.7 with any of the 14 evaluated genes using the Affymetrix data. Thus their exclusion from the AD signature was supported.

Since R>0.7 with 3 or more signature genes included 6 of the 7 highly intercorrelated genes we used that as our cutoff for saying a gene was potentially in the Affymetrix AD signature. This standard included 2900 of the Probe sets. The first question examined was what fraction of the 2900 probe sets exhibited R>0.7 with the weakly intercorrelated genes FPRL1, ORM1, or PI3. Overall, 139, 0, and 878 probe sets had an R>0.7 with FPRL1, ORM1, or PI3 and another gene, respectively. Of these 1017 probe sets, 480 had R>0.7 with 4 or more genes, and 83 had R>0.7 with 5 or more signature genes. Thus it was fairly certain that inclusion of FPRL1 and PI3 within the set of AD signature genes was warranted. Of the 2900 probe sets, 997 were also found in the list of ALS probe sets with R>0.7 with 6 or more signature genes. Thus, a significant fraction of the highly correlated genes remained so in both ALS and AD.

The next question was how may of the 2900 probe sets exhibited co-correlation with signature genes in AD patients and would constitute the diagnostically useful set of signature genes. As indicated in Table 2 (FIG. 34), 477 of the probe sets were upregulated at least 2 fold in AD patients relative to healthy elderly controls. Of these, 147 had an R>0.7 with 3 or more signature genes. The most upregulated potential signature genes included GPR86 (AD/Old=8.3, p=0.016, N>0.7=3), COL13A1 (AD/Old=6.8, p=0.048, N>0.7=3), and IFIT2 (AD/Old=5.6, p=0.018, N>0.7=6). As mentioned above, G1P3, PI3 and ORM1, despite their high utility at diagnosing AD were not included in the predicted AD signature due to their poor correlations with the remaining signature genes.

Therefore we combined the list of diagnostically useful signature genes from ALS and AD patients to generate the list of genes useful for the diagnosis of neurodegenerative disease. The list contains 742 probes and 542 genes or transcripts. This list does not contain the signature genes BRI3, COX5B, FCAR, CXCL11, or GPSM3 since these genes either failed to have a large enough difference between patients and controls and/or a high R value with enough other signature genes. The list does include all 5 genes used in the LC5-CI, namely G1P3, GPR43, IFIT2, ORM1, and TNFSF10.

The full set of useful signature genes that are described in this application are assembled as follows:

1. The 17 genes confirmed to be in the ALS signature by quantitative real time PCR

| | |
|---|---|
| BRI3 | COX5B |
| FCAR | FPRL1 |
| FXYD6 | G1P3 |
| GPR43 | GPSM3 |
| IFIT2 | IL1RN |
| MX2 | NBS1 |
| OAS3 | ORM1 |
| PI3 | SP110 |
| TNFSF10 | |

2. An additional gene confirmed to be in the AD signature by quantitative real time PCR CXCL11
3. All genes with an R>0.7 with 6 or more of the ALS signature genes and a mean signal in ALS patients that was at least 2 fold more at significance level of 0.05 than the signal obtained from a group of healthy controls (data as of June 2005) 687 additional probe sets representing 503 genes.
4. All genes with an R>0.7 with 3 or more of the AD signature genes (see below) and a mean signal in AD patients that was at least 2 fold more at a significance level of 0.05 than the signal obtained from a group of age-matched healthy controls

AD Signature included these 14 genes

| | |
|---|---|
| BRI3 | CXCL11 |
| FPRL1 | FXYD6 |
| GPR43 | GPSM3 |
| IFIT2 | MX2 |
| NBS1 | OAS3 |
| ORM1 | PI3 |
| SP110 | TNFSF10 |

55 additional probe sets representing 39 genes
5. All genes not included in the above lists that had an R>0.7 with 6 or more ALS signature genes and an R>0.7 with 3 or more AD signature genes.
   a. These probes represent the set of genes that are in the signature in multiple conditions but are not are significantly upregulated in ALS or AD
   b. Many of these genes are probably useful for the diagnosis of other ProMac diseases 746 additional probe sets representing 547 genes
6. Total group of sequences useful for signature analysis is 1493 probes representing 1123 genes or transcripts.

### EXAMPLE 6

### Signature is not Present in Healthy Controls

It was of interest to consider if the signature was maintained in healthy samples. Accordingly, the light cycler data obtained with the healthy control samples data was used to determine a Pearson correlation coefficient for each of the gene pairs, see Table 10 (FIG. 42). The genes G1P3, IL1RN, and IFIT2 all retain higher median R values with the signature genes than with other genes, however, the absolute number of signature genes with which these genes have an R>0.7 has dropped. As with AD samples, the genes FPRL1 and PI3 are not strongly correlated with other signature genes. Additionally, the genes FXYD6, GPR43, MX2, NBS1, OAS3, and TNFSF11 all have essentially equal median R values with signature and non-signature genes. Thus, the signature is a property of macrophages isolated from individuals with neurodegenerative or other inflammatory disease, and not a common feature of macrophages in general.

### EXAMPLE 7

### Conditions Required to Observe a Signature

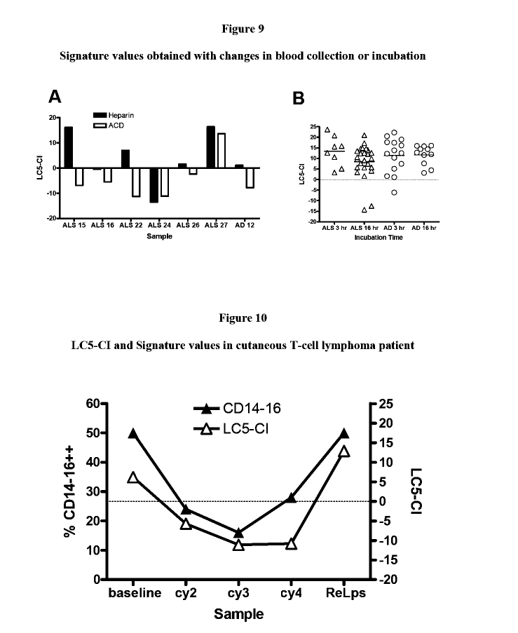

The standard protocol for detecting the ProMac signature, either using microarrays or by RT-PCR calls for drawing blood using heparin anticoagulant, isolation of PBMCs on percoll gradients, and incubating the PBMCs at 37° C. for 12-18 hours. This is essentially an in vitro procedure. Accordingly, it would be useful to define the minimum operating requirements for observing the signature. The first item looked at was the use of alternative anticoagulants. Comparison of the LC5 signals obtained from 5 ALS patients and one AD patient when there blood was collected into tubes with heparin vs acid-citrate-dextrose anticoagulant indicated that higher LC5-CI signals were routinely obtained from the blood collected into heparin anticoagulant (FIG. 9A). Next we looked at time of incubation. The LC5-CI signals from a group of 8 samples from ALS and AD patients in which blood had been collected into heparin anticoagulant tube, PBMCs purified via Percoll gradient centrifugation, and incubated for 3 hours was compared to the LC5-CI signals obtained after a 16 hour incubation (FIG. 9B). No significant differences in the mean LC5-CI values of the samples that were incubated for 3 or 16 hours was observed. Thus the signature is present after a relatively short in vitro incubation. However, efforts to detect a signature from blood collected into PAXgene tubes, which lyse cells and stabilize RNA immediately upon collection, were unsuccessful (data not shown). It is unclear whether this failure was a result of a requirement for some period of incubation or a technical failure, perhaps brought on by the high level of globin mRNA present in reticulocytes and red blood cells. Future experimentation will be required to determine the minimum period of incubation for signature development.

### EXAMPLE 8

### The Signature as a Therapeutic Index

Another use for the signature would be for the classification of patients for treatment. Currently there are two different classes of molecules that have been shown to have a positive effect on ProMacs in vivo. The first of these is a chlorite based compound, designated WF10⁵³. WF10 is approved for the treatment of hemorrhagic radiation cystitis in Thailand⁵⁴. WF10 has also been evaluated in phase III clinical trials for HIV associated disease/late stage AIDS in the United States⁵⁵. The second class of compounds that have been demonstrated to affect macrophage functionality are polyamine analogs. This encompasses several compounds including Methylglyoxal bis(guanylhydrazone) dihydrochloride hydrate (MGBG, also designated mitoguazone); N,N′-bis[3,5-bis[1(aminoiminomethyl) hydrazono]ethyl]phenyl]decanediamide tetrahydrochloride (CNI-1493 or Semapimod), and a variety of other spermine analogs including CG-47 and CG93⁵⁶⁻⁵⁹. Both MGBG and CNI-1493 have been employed in clinical trials for treatment of cancer^{60,61} and in the case of CNI-1493 other inflammatory conditions such as Crohn's disease⁶². Overall, the compounds exhibit some level of efficacy against cancer, however, a response is not universal and neither drug has yet been approved by the FDA for use in any clinical condition

As part of the clinical development of CG-47, Pathologica performed analysis on blood samples from a subset of patients currently enrolled in a Phase I trial of the safety of the investigational polyamine CG-47. The trial recruited patients who have failed conventional therapy for various lymphomas. The patients received increasing amounts of CG47 for 5 days every three weeks by infusion. The trial was a dose escalation trial in which the first 3 patients received the lowest dose, the next three a higher dose etc. One patient, a 53 Yr Old male with subcutaneous T cell lymphoma, received 4 doses of 50 mg/m² of CG-47 over a 12 week period. The patients experienced a very significant remission in his lymphoma which began after administration of the first dose of CG-47 and lasted approximately 10 weeks. Relapse of the lymphoma then occurred and a final sample was obtained. Levels of CD14-16++macrophages and the LC5-CI values obtained from the baseline and various subsequent samples are presented in FIG. 10. At presentation the patient had elevated CD14-16% s (˜50%) and a strongly positive LC5-CI score (6.2). After the first cycle of CG-47 the patient entered a clinical remission of his cancer that lasted for the next four cycles of CG-47. During his clinical remission both the CD14-16++ levels and the LC5-CI scores normalized. After the 4th cycle of CG-47 the patient experienced a relapse in his cancer and in the post relapse samples CD14-16++ levels were again elevated (50%) and his LC5-CI value had reverted to a positive score (12.9). Thus the LC5-CI scores from this patient correlated very well with his overall clinical status. This implies that one could use the LC5-CI both to pre-screen patients, to restrict treatment to those patients with elevated levels of ProMacs in their blood. The efficacy of treatment could then be followed by monitoring the LC5-CI values of the patient over the course of the trial. Although in cancer, treatment failure or response can be quite obvious, the same is not true in neurodegenerative disease, or many other inflammatory conditions. Thus clinical monitoring with the LC5-CI or similar ProMac assay may prove to be very valuable.

### EXAMPLE 9

### Detection of ProMacs Via Monitoring of Signature Proteins

The LC5-CI is a reverse-transcriptase PCR assay. This particular assay is run on a quantitative real-time PCR machine. These assays have a high level of accuracy and reproducibility, but assays are relatively labor intensive. Additionally quantitative RT-PCR assays require trained operators and familiar with contamination-control procedures. Accordingly, it would be advantageous if an assay based on protein detection via immunological or other techniques could be formulated. One assay target would be the population of cell surface receptors that are highly upregulated in ALS patients. These could be assessed by flow cytometry or similar methods. Flow cytometry could be performed both on unincubated whole blood and on isolated PBMCs after overnight incubation at 37° C. Aliquots of whole blood (usually 100 μl/test) or purified PBMCs will be combined with test antibody with or without antibody to CD14. Staining for CD14 is performed to determine if any or all of the proteins under evaluation are preferentially expressed in the monocyte/macrophage population since a significant fraction of the 1129 genes or transcripts of the ProMac signature are preferentially expressed in cells of myeloid lineage (e.g. FPRL1, GPR43, IL1RN). Control antibodies will include isotype matched IgG FITC or IgG-PE. After incubation for 15 minutes at room temperature red blood cells are lysed with FACS Lysing Solution (Becton-Dickinson) for 10 minutes. Isolated PBMCs are incubated with antibody for 15 minutes without any lysis step. Cells are then washed with phosphate buffered saline (PBS) and fixed with 1 ml of 1% paraformaldehyde in PBS. Staining data will be acquired on a FACScan flow cytometer with Cell-quest software counting at least 20,000 cells. Fluorescent signal cutoffs for individual proteins will be based on results with negative controls. Results are expressed as the percentage of total cells and CD14+ cells that have signals above the cutoff. For some analysis we will also make use of the median fluorescent signal of a sample with a given antibody.

Proteins of the signature can also be detected using antigen capture or competition assays. The proteins may be secreted and present either in the serum or plasma of individuals with ALS, or possibly in the media of the PBMCs after incubation. Alternatively the proteins could be intracellular. Both direct “sandwich” assays and indirect competition assays can be employed. The exact format of assay to use will depend on the number and types of antibodies available for a given secreted protein. Antibodies can be obtained from commercial sources, produced by peptide immunization of a suitable animal (e.g rabbits) or by the generation of monoclonal antibodies in mice or other suitable animals using standard techniques⁶³. In the sandwich assay format, 96 well microtiter plates are coated with ˜1 g of a monoclonal antibody specific for the protein of interest and blocked by incubation with BLOTTO (PBS plus 0.1% tween-20, 2.5% normal goat sera, 2.5%) or other suitable blocking agent. Aliquots of the sample to be evaluated, which can be plasma or other bodily fluids, tissue culture media, or cell lysates, are diluted in PBS or BLOTTO. Typically, samples would be tested over a range of 4-8 dilutions to provide a more accurate final estimate. Aliquots of the fluid to be tested are then added to blocked wells and allowed to bind to antibody for 90 minutes at room temperature with gentle rocking. Wells are then washed and a predetermined dilution of detection antibody is added. The detection antibody is generally biotinylated or otherwise conjugated. After incubation for one hour at room temperature with gentle agitation wells are washed and streptavidin-conjugated alkaline phosphatase or other detection reagent is added. After incubation for a suitable period, the wells are washed and para-nitrophenyl-phospahte (PNPP) or other appropriate substrate is added. After incubation for 20-30 minutes at room temperature the optical density (O.D.) in each well is read. Positive controls include wells in which various concentrations of in vitro expressed and purified protein of interest. Negative controls include samples with no added plasma or media. Results can be expressed as the dilution of sample that results in optical density of 1.0

For some signature proteins use of a competition assay to detect protein expression will be preferable. Wells are coated with ˜1 g of purified in-vitro expressed protein of interest. In vitro expression of the protein can be accomplished using either prokaryotic or eukaryotic expression systems using techniques well known in the art. After washing and blocking various dilutions of the test sample will be combined with the antibody specific for the protein of interest and incubated for at least 15 minutes. Between 4-8 dilutions of each sample will be employed. The mixtures will then be added to triplicate wells and allowed to incubate for an additional 60 minutes. Wells are then washed and alkaline phosphatase conjugated anti murine IgG or other appropriate detection reagent is added. After incubation for 60 minutes wells are washed and bound antibody detected by addition of an appropriate substrate. Positive control wells will have increasing amounts of purified in vitro expressed protein added. Negative control wells will contain only buffer. Results can be expressed as the volume of plasma, media, or extract that results in 50% inhibition of signal.

### EXAMPLE 10

This example demonstrates the use of microarray analysis of the gene transcription of peripheral blood cells to define the cells and pathways associated with systemic immune activation in ALS and AD patients and to identify genes upregulated in neurodegenerative diseases such as ALS and AD.

Amyotrophic lateral sclerosis (ALS) and Alzheimer's disease (AD) are debilitating neurological disorders in which neurodegeneration occurs in concert with an ongoing inflammatory process. The causes of ALS and AD are unknown. Approximately 5-10% of cases are familial in either disease and the remaining 90% of ALS and AD cases are described as sporadic, in that there is no obvious family history of disease. For both diseases existing therapies (e.g., riluzole for ALS) result in only modest slowing of disease progression. Both ALS and AD are characterized by evidence of systemic immune activation, in addition to local activation within focal neuropathology.

Patients and Controls

Thirty one patients with sporadic ALS, (diagnosed by El Escorial criteria, Ross et al., 1998) and 12 patients with suspected Alzheimer's disease, seen at the Forbes Norris MDA/ALS Research Center (San Francisco, Calif., USA) provided informed consent in accordance with guidelines established by the California Pacific Medical Center and University of California San Francisco (UCSF) committees on human research, coordinated by the UCSF AIDS and Cancer Specimen Resource (ACSR) program. Functional testing of ALS and AD patients was performed using the Revised-ALS Functional Rating Scale (ALSFRS-R), scored 0-48 (ALS CNTF treatment study phase I-II study group 1996) or Mini-Mental-State-exam (MMSE, Folstein et al., 1975), respectively. Patients were evaluated within one month of donating samples. Healthy controls consisted of 29 individuals who had provided informed consent and blood samples to the ACSR. All healthy controls were from the San Francisco bay area and met criteria similar to that required for standard blood donation.

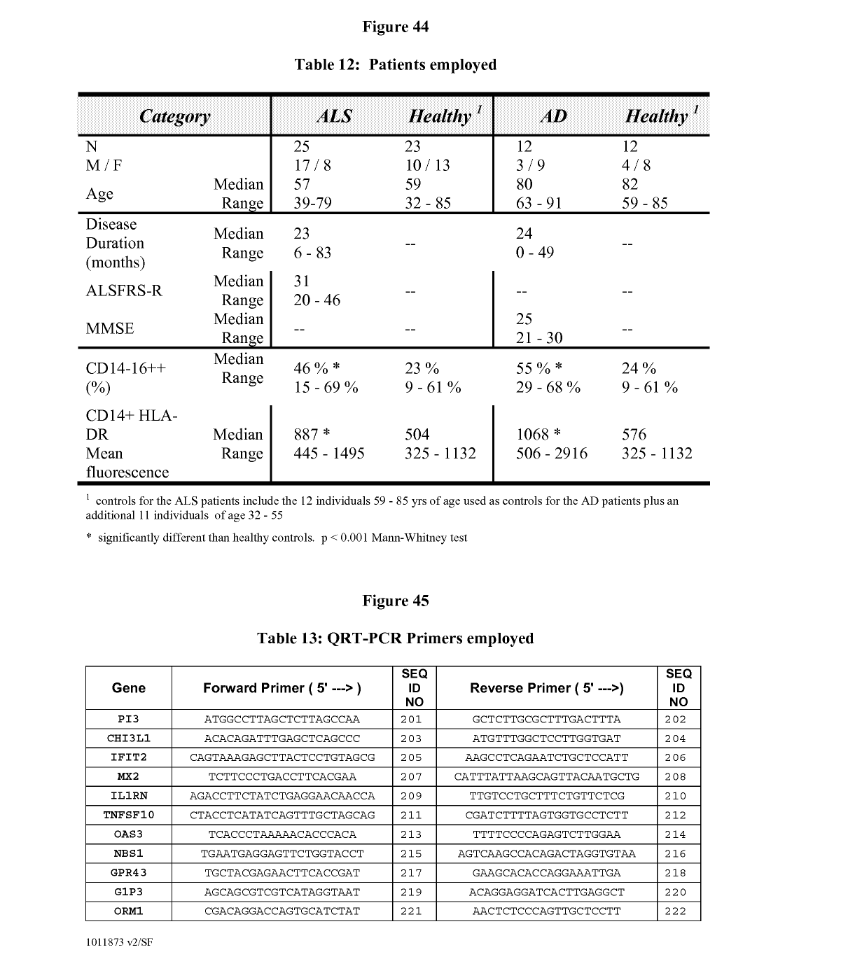

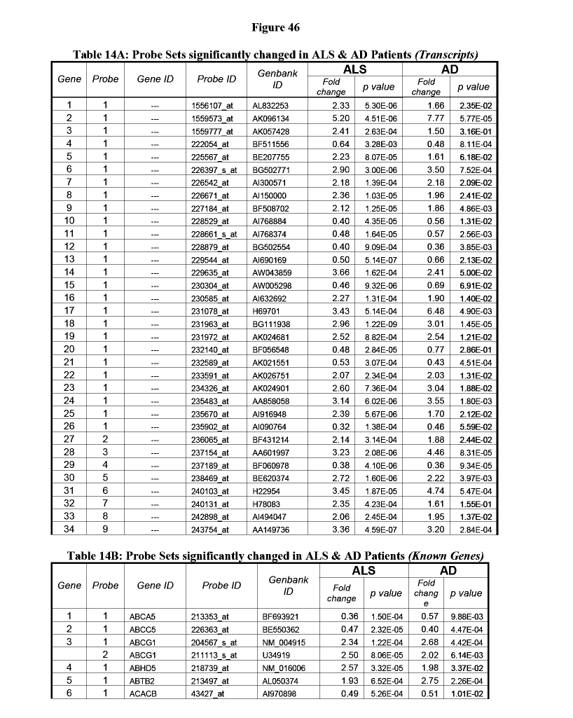

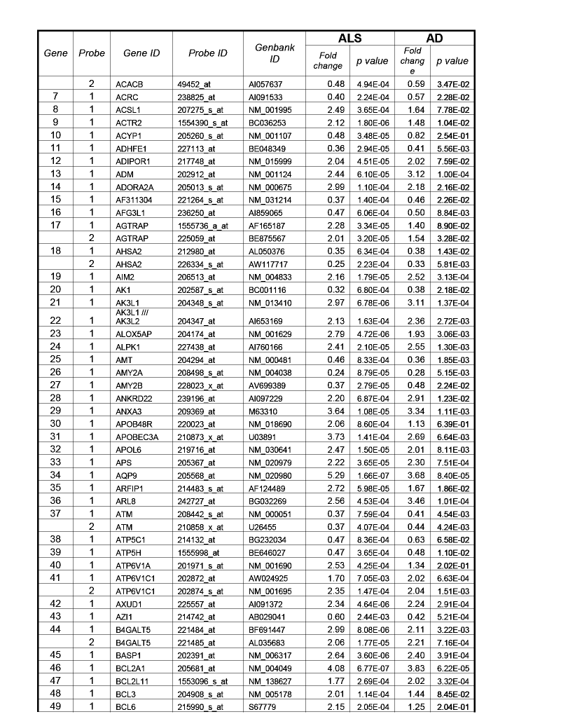

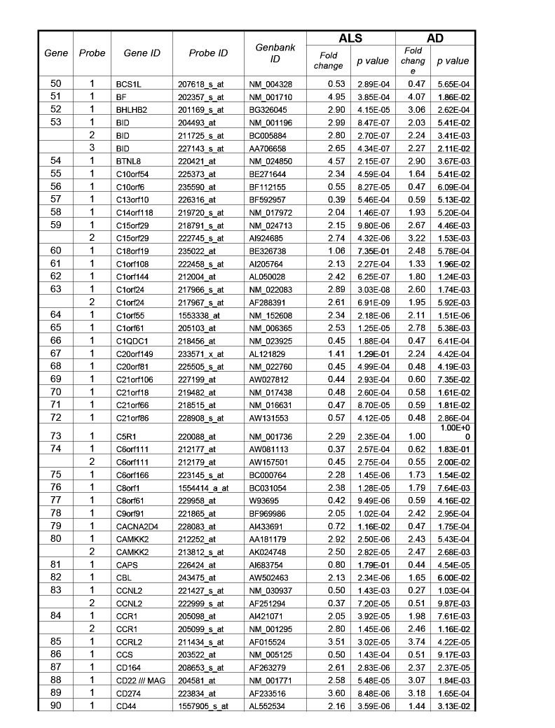

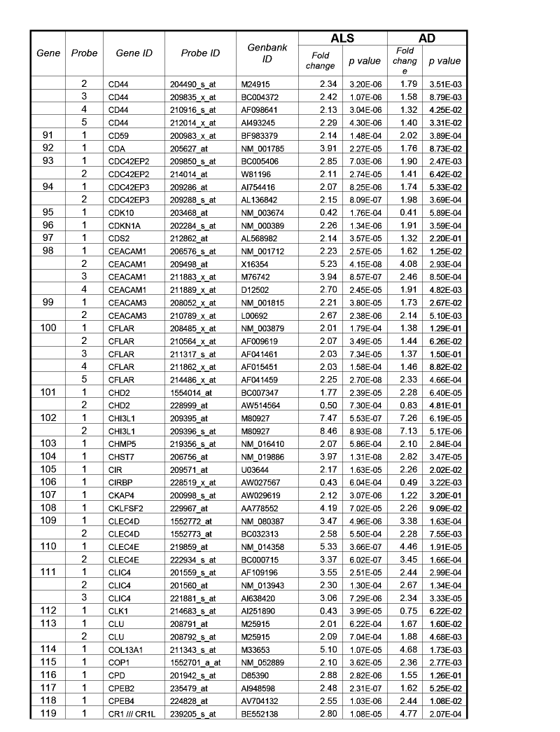

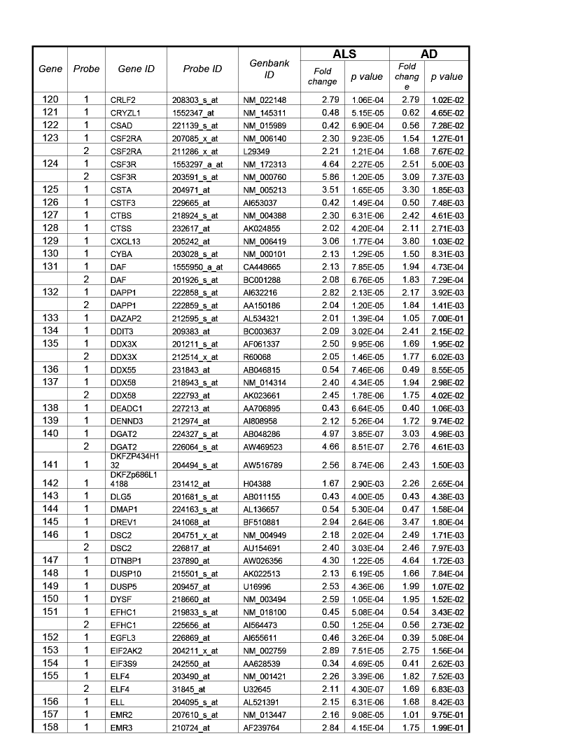

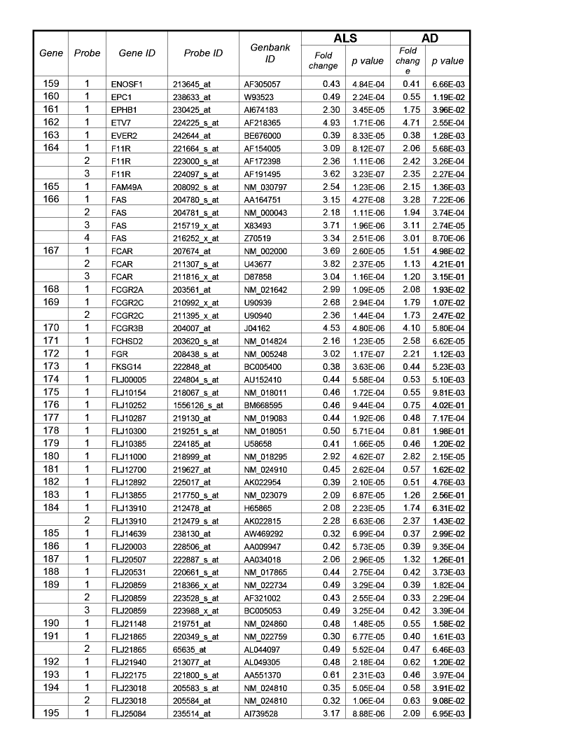

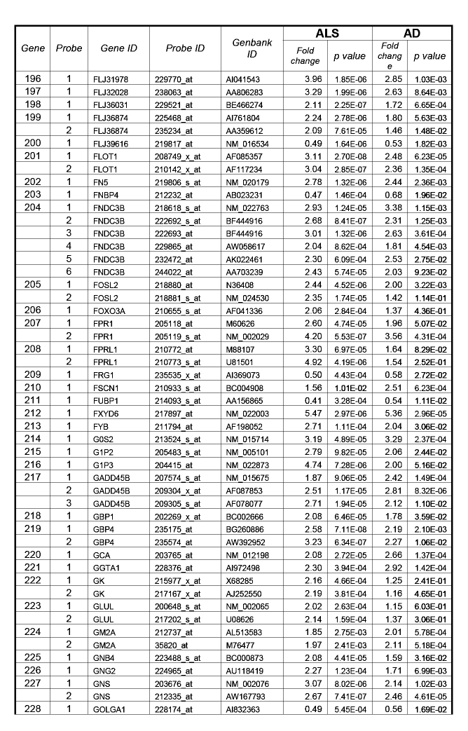

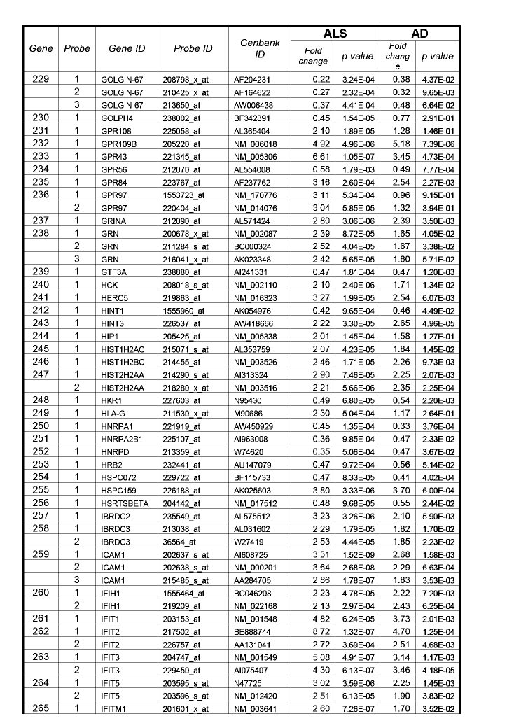

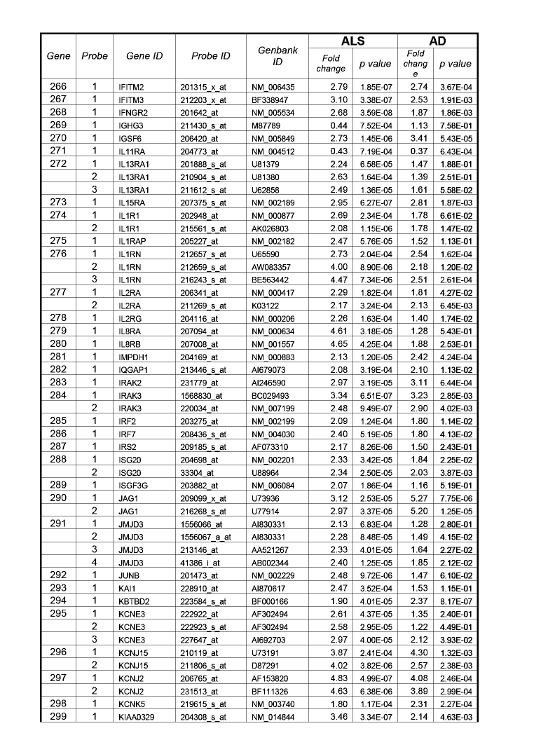

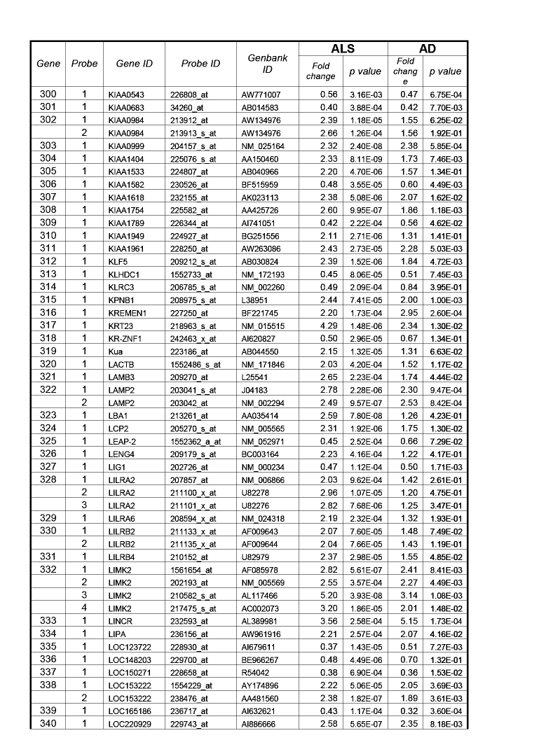

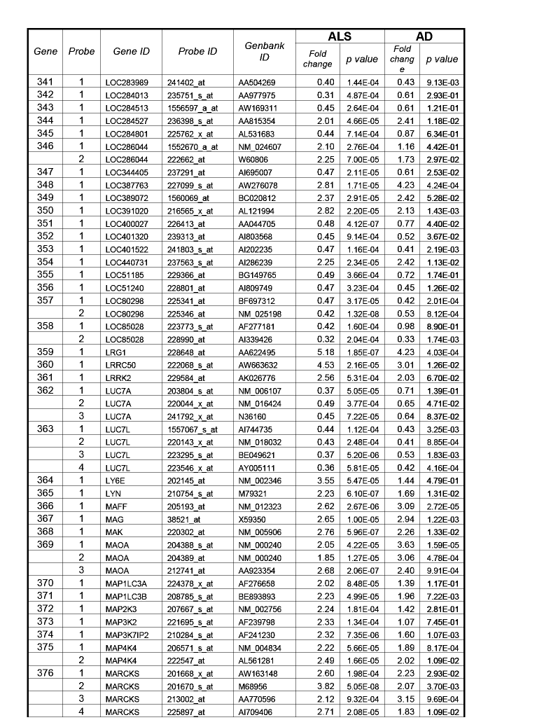

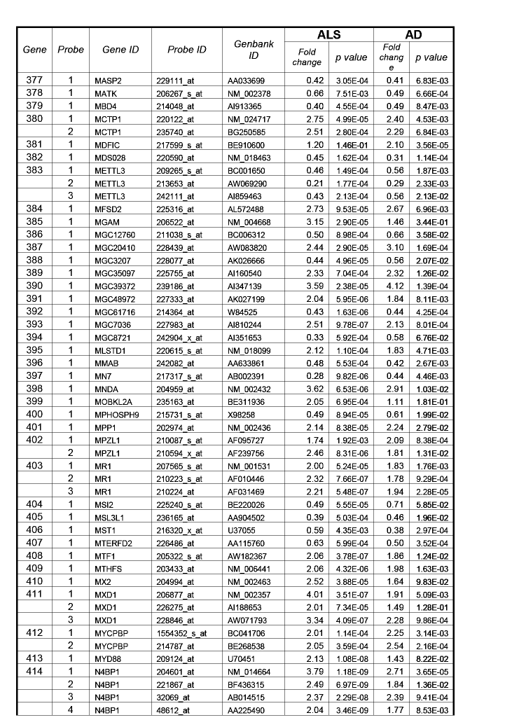

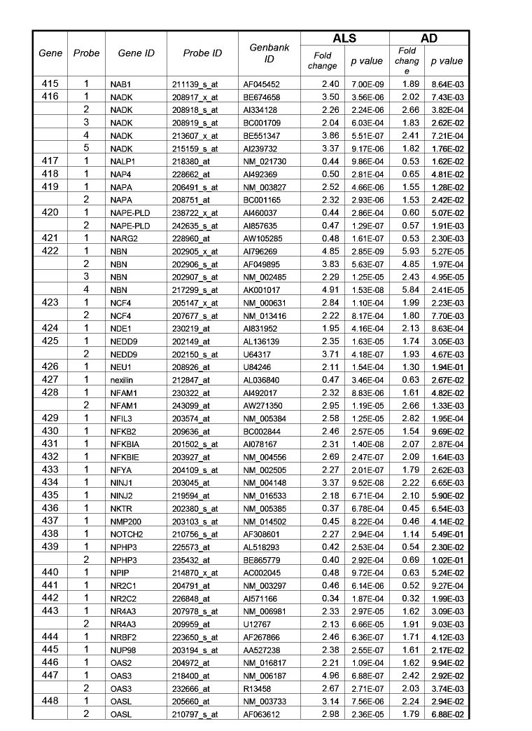

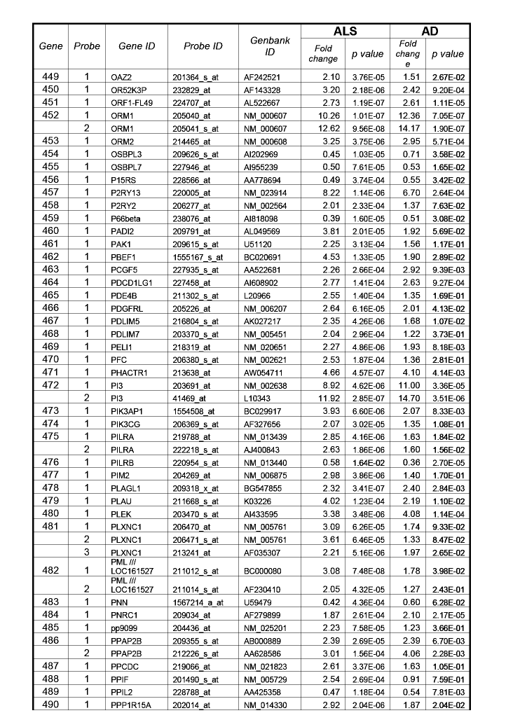

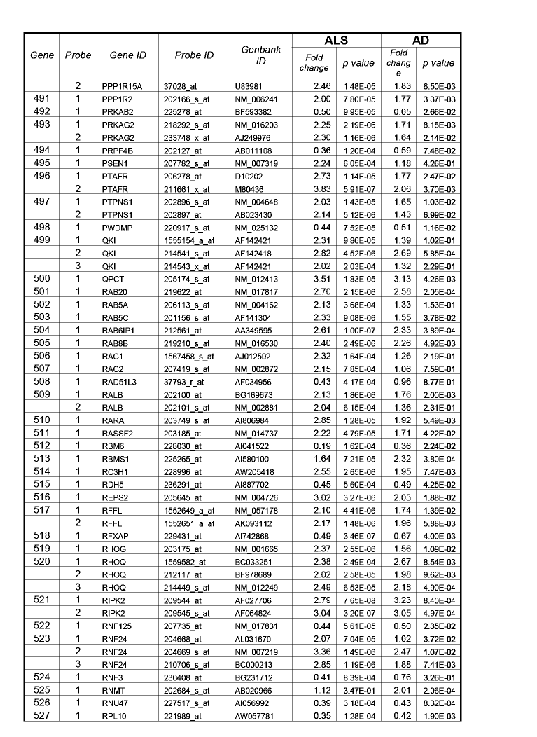

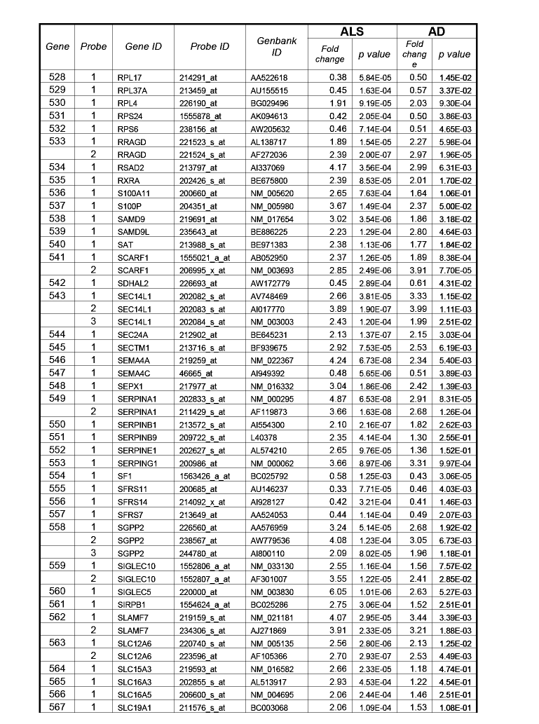

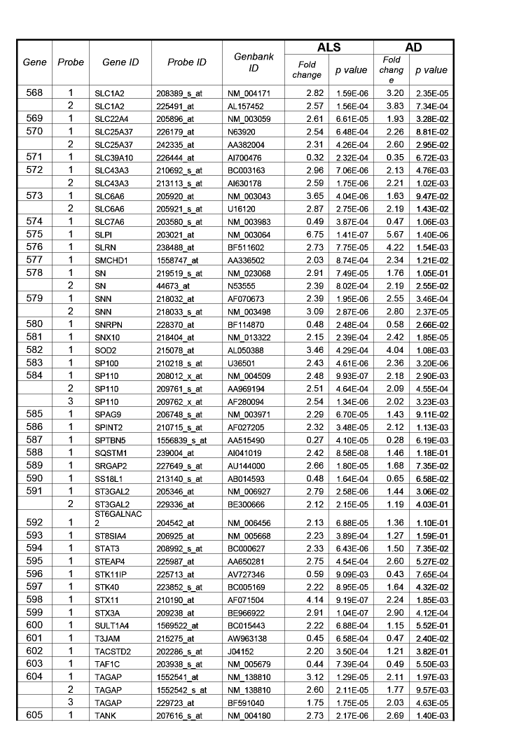

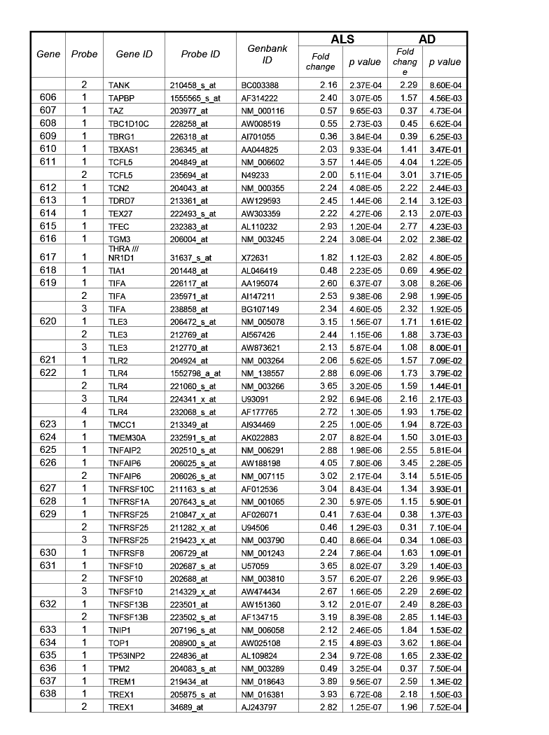

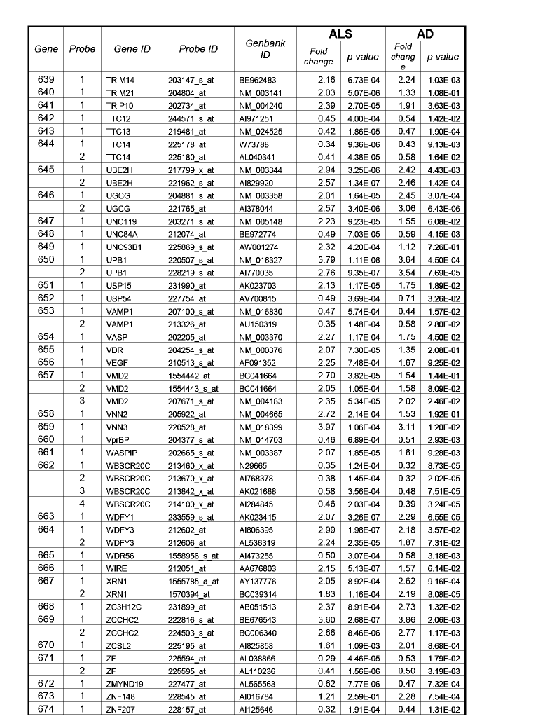

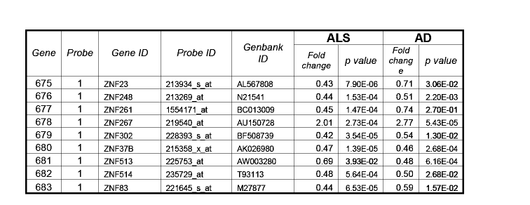

Basic demographic and clinical information about the patients and the healthy control samples are provided in Table 12 (FIG. 44). Controls for AD patients included 12 samples from healthy individuals of ages 59 to 85. Controls for ALS included these 12 samples plus an additional 11 samples from individuals of ages 32 to 55. The sporadic ALS patients studied included approximately 2 times as many men as women and ranged in age from 39 to 79 with a median age of 57. The median time since onset of symptoms (disease duration) for the ALS patients was 23 months and ALSFRS-R scores of the patients ranged from 20 to 46 with a median of 31. The AD patients studied were significantly older than the ALS patients (median age 80 vs 57) and were predominantly female. The AD patients were all early in their disease course with a median disease duration of 24 months and MMSE scores of between 20-30 (median 25). The majority of the ALS patients were taking riluzole (21 of 25) and 9 of the AD patients were taking either NMDA receptor antagonists and/or acetylcholinesterase inhibitors. The number and demographics of patients and healthy individuals listed in Table 12 (FIG. 44) reflect those whose RNA was used in the Affymetrix assay described below (see “Microarray Analysis”). Other individuals, both patients and controls, were added to the study at the time the timing of the MIFN signature induction was carried out (see below).

Flow cytometric analysis of monocytes and activated macrophages was performed on whole blood according to the procedure described in Example 18. Heparinised blood was mixed with an equal volume of sterile isotonic phosphate-buffered solution (PBS, Ca++, Mg++ free) and layered over Percoll (Amersham Biosciences, Piscataway, N.J.) at 1.087 g/ml. The cells were centrifuged and the mononuclear cell layer was collected. For some experiments, mononuclear cells were obtained via resuspension of whole blood in erythrocyte lysis buffer (155 mM NH₄Cl; ˜10 mM KHCO₃ and 0.1 mM EDTA) followed by centrifugation. After isolation, mononuclear cells were washed with PBS and resuspended at ˜10⁶/mL in RPMI 1640 supplemented with 10% fetal bovine serum (HyClone, Logan, Utah) and 110 μg/ml sodium pyruvate. Mononuclear cells were cultured for 20 hours (unless otherwise noted) at 37° C. in a humidified, 5% CO₂ incubator. Cells were then pelleted by centrifugation, washed one time with PBS and resuspended in TRIZOL (InVitrogen Corp, Carlsbad, Calif.).

Microarray Analysis

Total RNA was extracted by using Absolute RNA RT-PCR Miniprep Kit (Stratagene, La Jolla, Calif.). The quality of RNA was determined by 2100 Bioanalyzer RNA LabChip (Agilent Technologies, Palo Alto, Calif.). 100 ng of high-quality total RNA was subjected to Affymetrix 2-cycle synthesis amplification, fluorescent labeling and hybridization to Affymetrix HG-U133_Plus_{—}2 human genome array according to manufacturers protocols (Affymetrix, Santa Clara Calif.). Expression data was obtained using a Affymetrix GSC3000 scanner and processed by GCOS software (Affymetrix, Santa Clara, Calif.). Gene Spring software (Agilent Technologies, Palo Alto Calif.) was used for downstream analysis of GCOS processed data. Signals from all probe sets were normalized using Human Genome U133 Plus 2.0 Array Normalization Controls ((Affymetrix, Santa Clara Calif.).

QRT-PCR

Approximately 150 ng of total RNA from each sample was converted to cDNA using the First Strand cDNA Synthesis Kit for RT-PCR [AMV] kit (Roche Applied Diagnostics, Indianapolis Ind.) according to manufacturer's instructions. After first-strand synthesis the reverse transcriptase was denatured by incubation at 99° C. for 5 minutes followed by quick cooling. DNA was stored at −20° C. until use. PCR was performed on a LightCycler (Roche Applied Diagnostics, Indianapolis Ind.) using the LightCycler FastStart DNA Master SYBR Green I kit and ˜4 ng cDNA sample. Amplifications included one cycle of template denaturation at 95° C. for 10 minutes followed by 45 cycles of 95° C. for 10 seconds, 68° C. for 10 seconds, and 72° C. for 16 seconds. The presence of a single amplified product was confirmed by DNA melting point analysis. Threshold cycles (Ct) for each amplification reaction were determined using LightCycler Software version 3.5 (Roche Applied Diagnostics, Indianapolis Ind.). All samples were also amplified with the human β-actin LightCycler-Primer Set (Roche Applied Diagnostics, Indianapolis Ind.). The sequences of gene-specific primers employed are provided in Table 13 (FIG. 45). Results with gene-specific primers for individual samples were normalized to signals obtained with β-actin from the same sample.

Immunoassays for Secreted Proteins.

Secreted proteins were quantified by ELISA for elafin (Cell Sciences; Canton, Mass.) and interleukin 1 receptor antagonist (RayBiotech, Inc.; Norcross, Ga.). Peripheral mononuclear cells from 6 healthy and 5 ALS individuals were cultured in RPMI+10% FCS at 37° C. Cell culture supernatants collected at 3 and 24 hour time points were tested in duplicates by ELISA according to manufacturer's instructions.

Similar Gene Expression Changes are Induced in Both ALS and AD

Peripheral blood mononuclear cells were isolated from the patients and total RNA prepared as described above. The RNA was then subjected to quality assessment, amplified, and hybridized to HGU 133plus2.0 human genome microarrays which contain 54,675 probe sets. For each patient group approximately 24,000 probe sets did not exhibit a minimum signal (50) in one RNA sample and so were discarded from subsequent analyses. A probe set was declared to exhibit significantly changed expression if it exhibited a 2 fold change in signal at a p value of less than or equal to 0.001. This would limit the number of probe sets included via a Type I error to approximately 30.

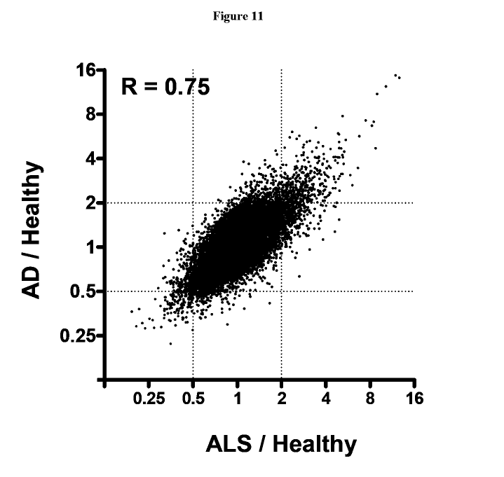

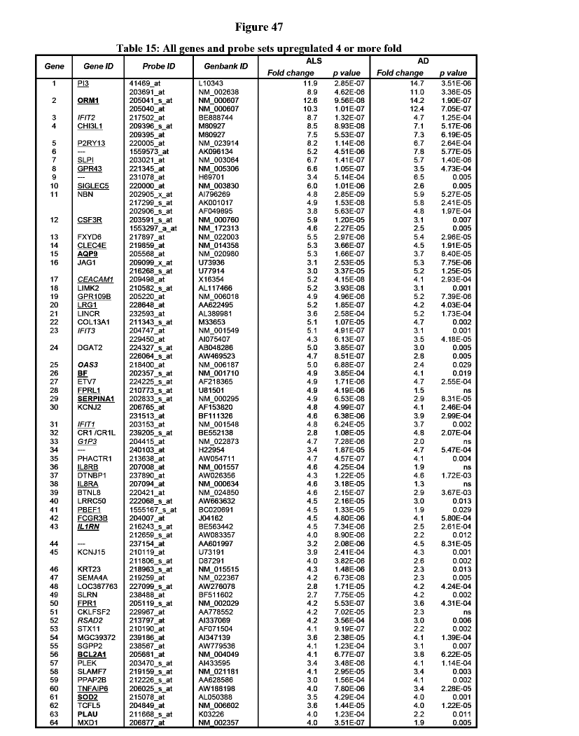

Using the above criteria, 944 probe sets representing 683 known genes and 34 transcribed sequences exhibited significantly changed transcription levels in ALS or AD PBMC RNA relative to healthy control RNA (see Tables 14A and 14B (FIG. 47)) with similar changes in both ALS and AD patients. Direct comparison of the fold change in signals between ALS patients and controls versus the fold change in signals between AD patients and controls for all expressed probe sets (see FIG. 11) resulted in a Pearson correlation coefficient of 0.75, which was highly significant (p<0.001). FIG. 11 shows that the transcriptional profiles of peripheral blood cells from ALS and AD patients are highly similar despite the different clinical presentations of the disorders. It shows a plot of the fold change for all expressed probe sets (N=28,935) for ALS patients/healthy controls (x axis) versus AD patients/healthy controls (y axis). The Pearson correlation for the entire data set is given.

Gene Ontology (GO) analysis (Gene Ontology Consortium 2000) was used to classify the genes with significantly changed RNA levels. Upregulated genes in both ALS and AD were associated with the GO terms immune response (Z score for ALS=13.1, Z-AD=7.7); defense response (Z-ALS=12.1, Z-AD=7.2); and response to biotic stimulus (Z-ALS=11.9, Z-AD=7.1). In contrast GO analysis of the down-regulated genes in ALS or AD RNA did not identify any terms that were common to both analyses (data not shown).

Upregulated Genes in ALS and AD are Dominated by Myeloid-Associated Genes

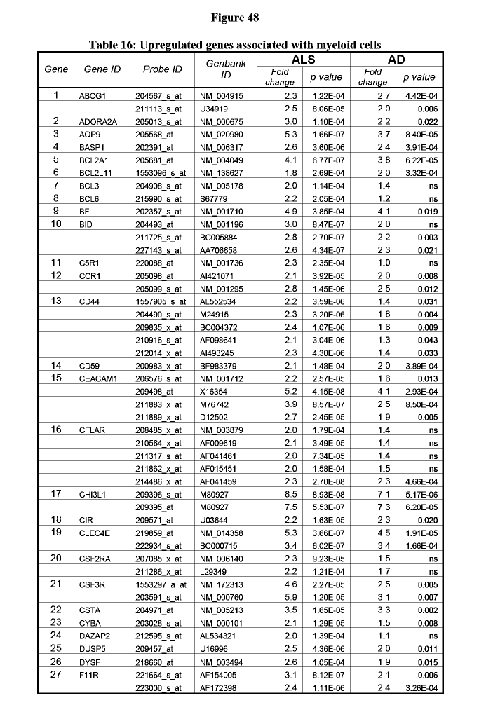

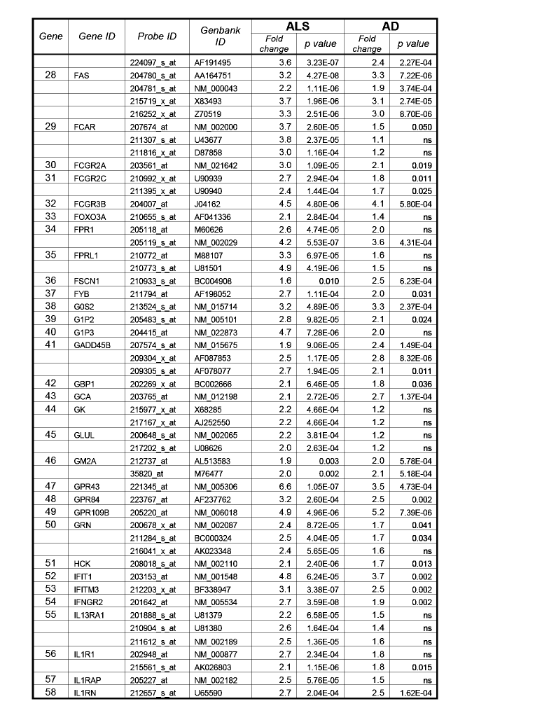

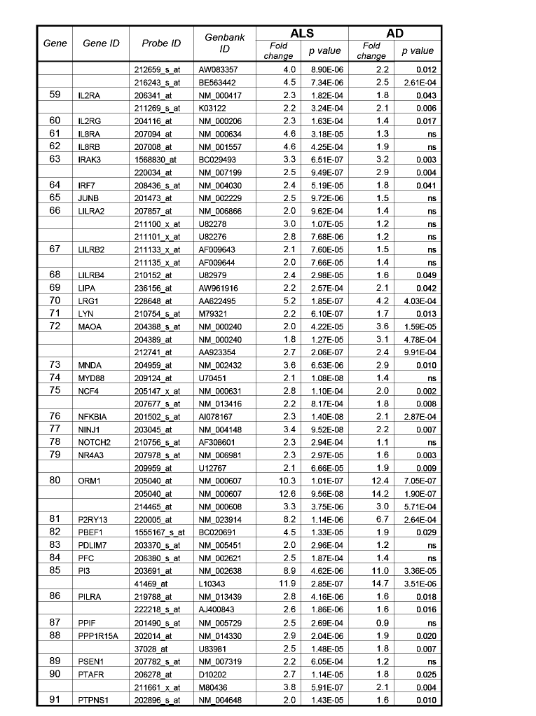

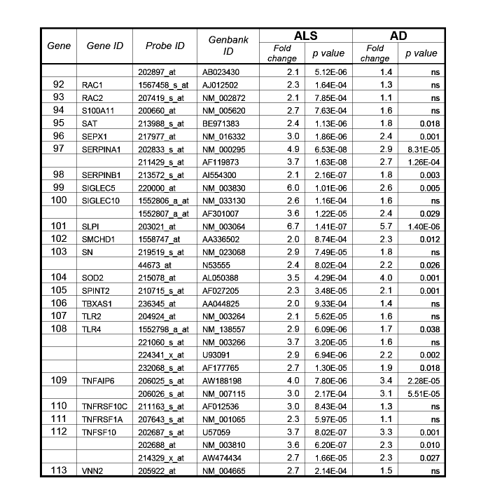

The 64 genes and transcripts showing a 4-fold or greater increase in mean signal in ALS and AD patients are listed in Table 15 (FIG. 47). The prevalence of myeloid-associated genes within the set of genes with increased signal in ALS and AD patients was determined (see Table 16 (FIG. 48)). Overall 113 genes previously associated with myeloid cells were significantly upregulated in ALS and/or AD patients, which was ˜25% of all significantly upregulated probe sets and a significant enrichment over the overall fraction of the probe sets to myeloid associated genes (25% vs 4.3% of 28,935, p<0.0001).

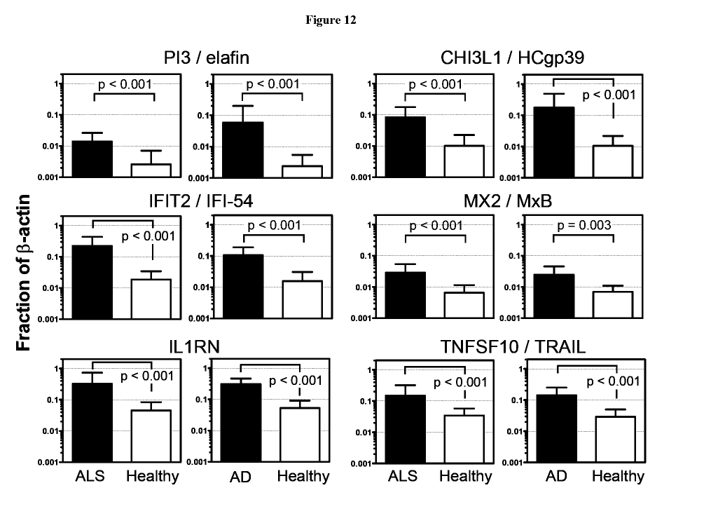

The elevated myeloid-associated genes included genes found in mature granulocytes, such as formyl peptide receptor 1 (FPR1), the interleukin 8 receptors (IL8RA and IL8RB), or colony stimulating factor 3 receptor (CSF3R) as well as other genes, such as cartilage glycoprotein-39 (CHI3L1), Nuclear receptor 4A (NR4A3), and interleukin 1 receptor antagonist (IL1RN) which are more closely associated with differentiated macrophages (Krause et al., 1996; Svensson et al., 2004; Theilgaard-Monch et al., 2005b). Genes encoding proteins associated with monocytes or dendritic cells were less common but also upregulated. QRT-PCR analysis of upregulated genes, PI3, CHI3L1, IL1RN, and TNFSF10 confirmed that the mean signals of all four genes in ALS and AD patient PBMCs were significantly higher in than in PBMCs from age-matched healthy individuals (see FIG. 12). In FIG. 12 the bars indicate mean signal obtained from QRT-PCR of total RNA samples from ALS patients (black bars), AD patients (grey bars) or age matched healthy controls (white bars) for the indicated genes (above graphs). Error bars indicate one standard deviation from the mean. Values are expressed relative to values obtained from β-actin from the same samples. P values for the comparisons are given.

By comparison, RNA levels of control gene β-actin were similar in all groups (data not shown). This shows that patients with ALS and AD show significant up-regulation of genes associated with peripheral blood myeloid cells, consistent with the increased levels of activated macrophages detectable by flow cytometry.

ALS and AD Upregulated Genes also Consist of α/β Interferon and NFκB Stimulated Genes

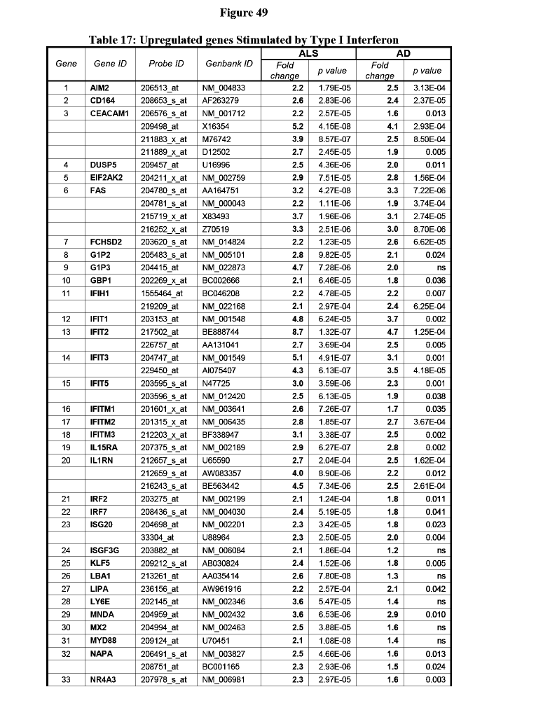

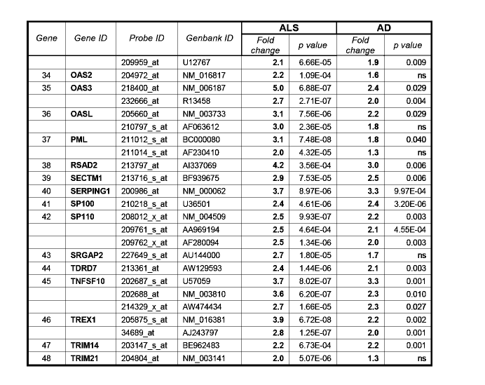

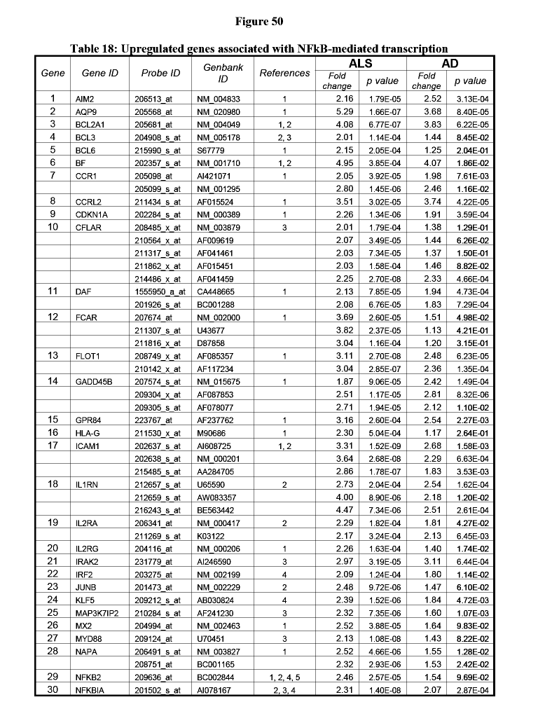

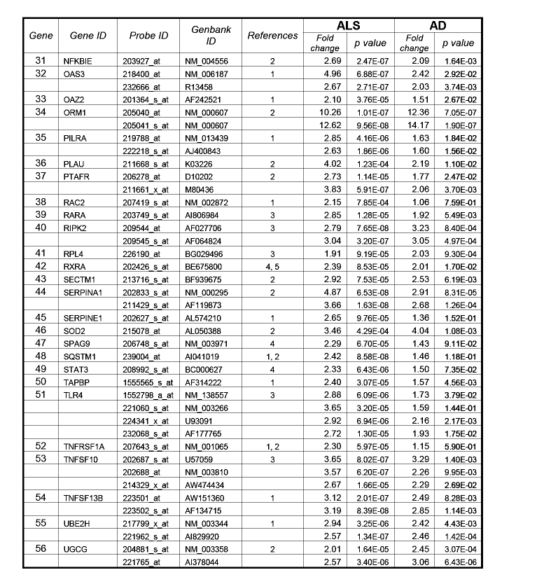

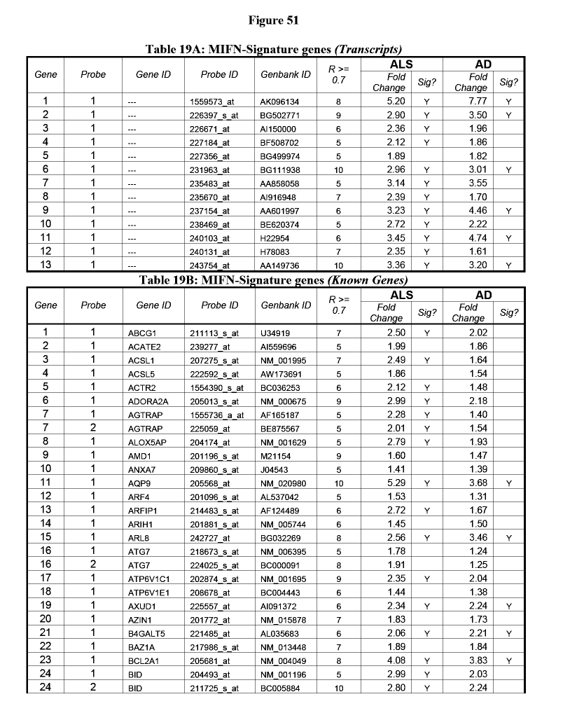

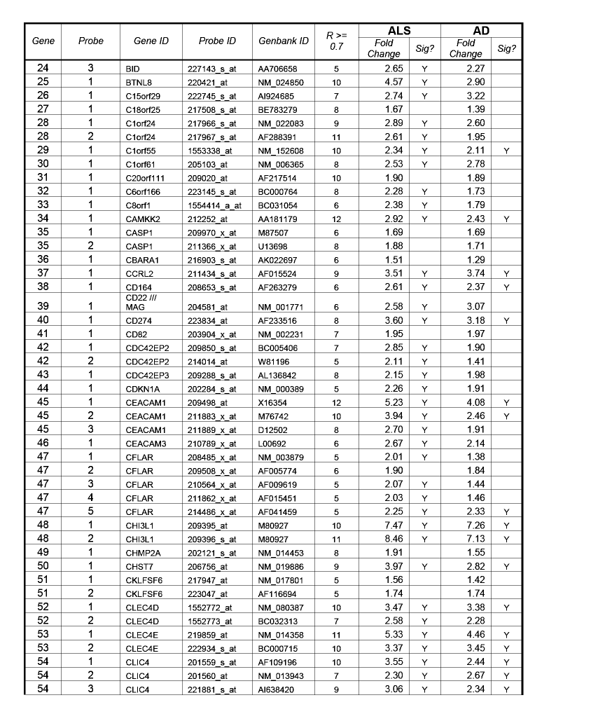

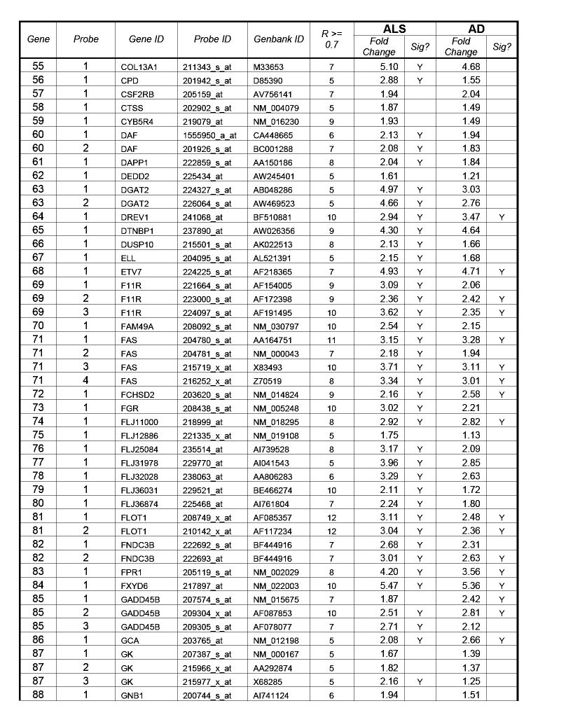

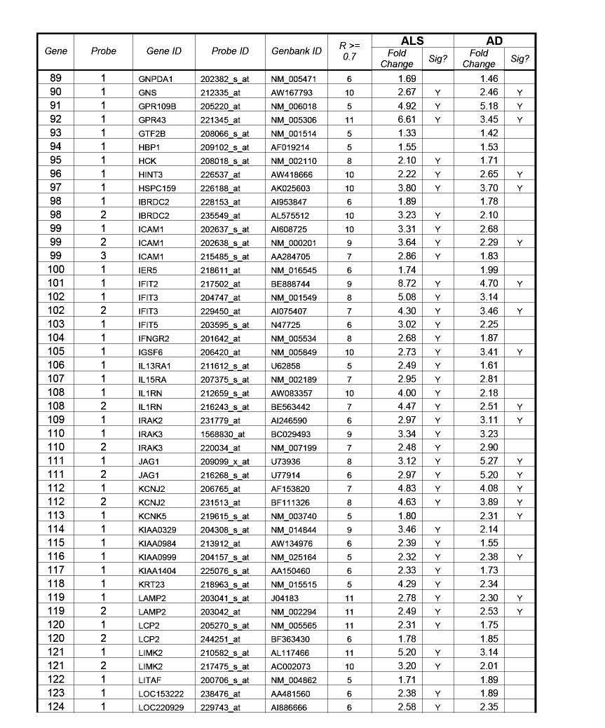

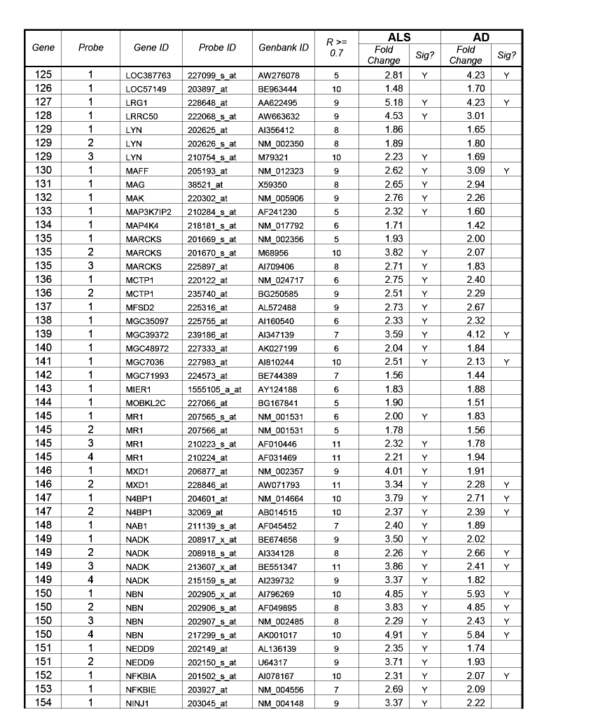

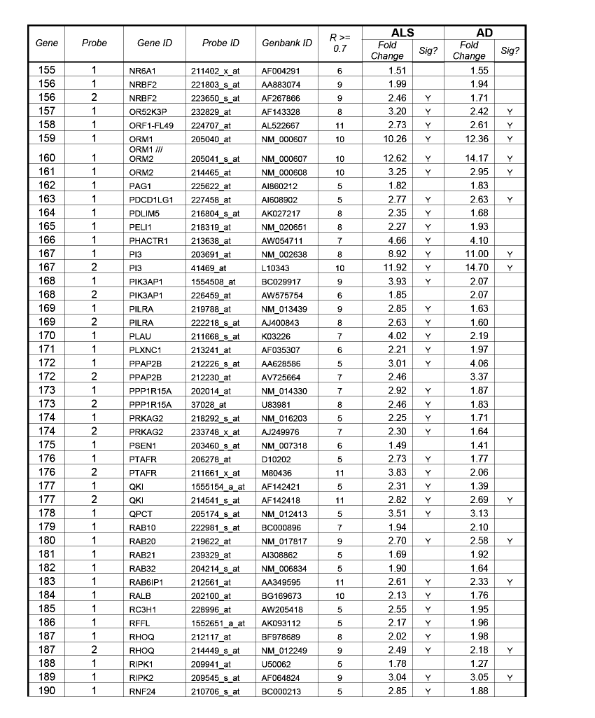

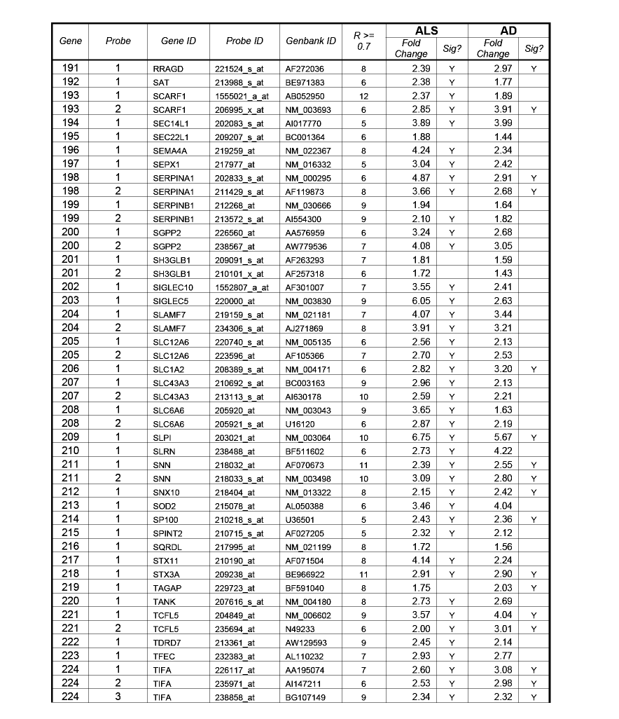

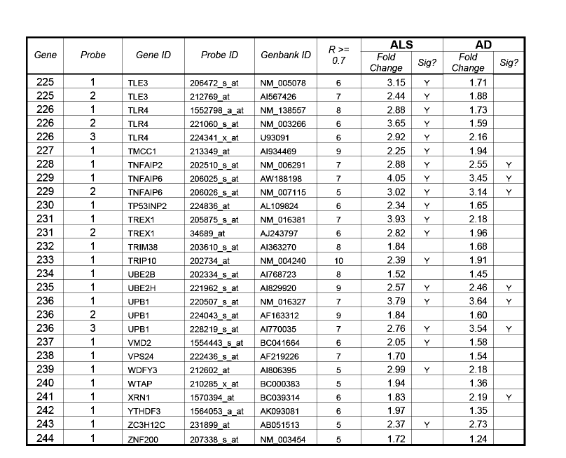

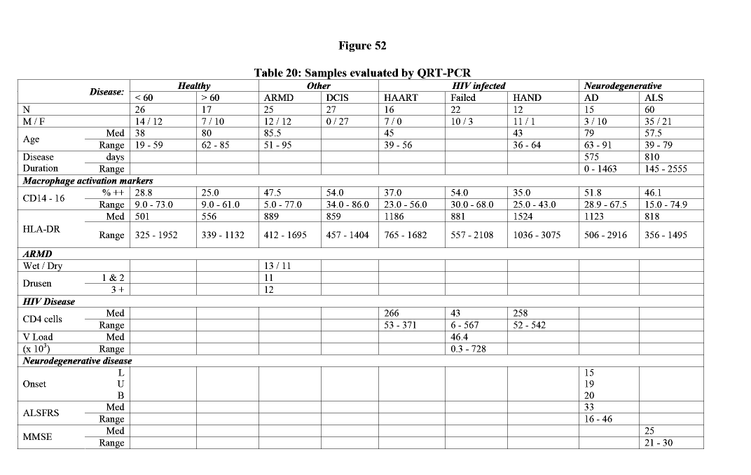

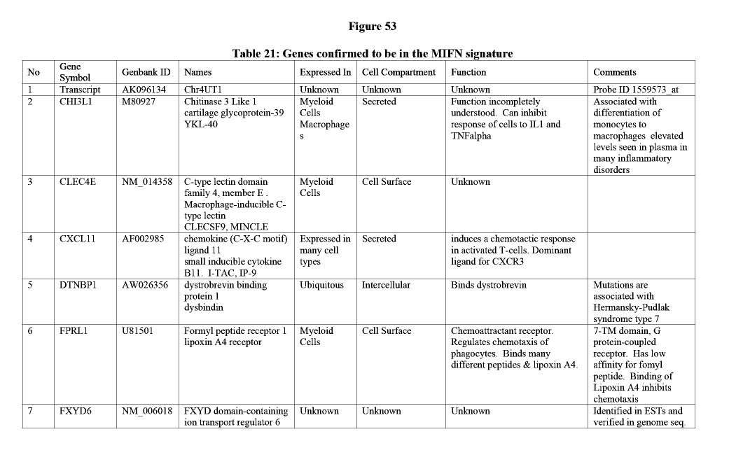

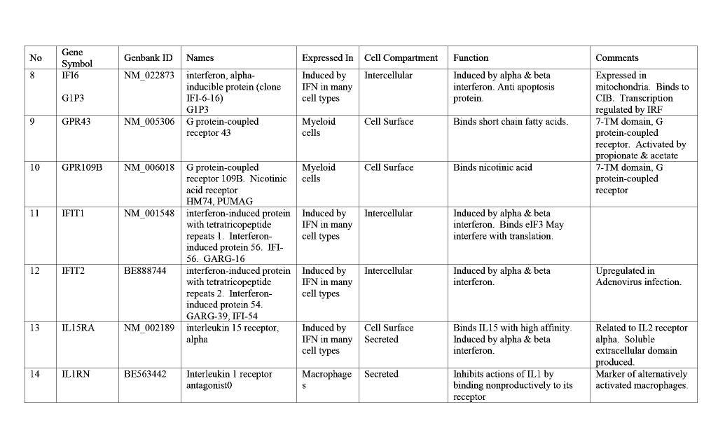

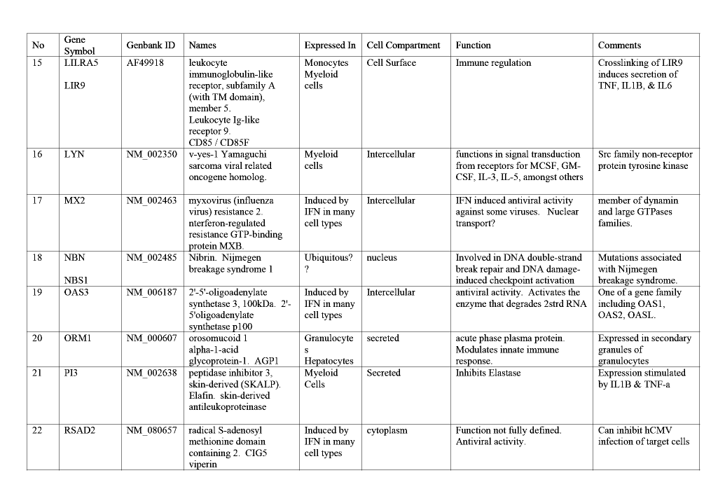

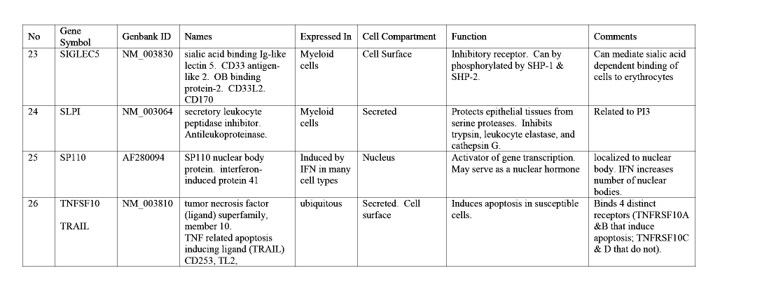

The 64 genes with 4 fold or greater elevations in mean RNA levels (see Table 15 (FIG. 47)) also included genes such as interferon-induced protein with tetratricopeptide repeats 2 (IFIT2) and 2′-5′-oligoadenylate synthetase 3 (OAS3) that are induced by type I interferons (Der et al., 1998; Baechler et al., 2003). Overall, a total of 71 probe sets to 48 genes represented (11% of all significantly increased probe sets) were known to be induced by type I (α/β) interferons (Table 15 (FIG. 47) and Table 17 (FIG. 49)). To confirm the microarray results, several of the interferon-induced genes including IFIT2 and myxovirus (influenza virus) resistance 2 (MX2) proteins were evaluated by QRT-PCR (see FIG. 12). As seen in see FIG. 12, mean levels of IFIT2 and MX2 RNA were confirmed to be significantly higher in ALS and AD PBMCs than in PBMCs from age-matched healthy individuals.

Both the interferon-induced genes and the macrophage-associated genes contained a number of genes such as interleukin 1 receptor antagonist (IL1RN) or TRAIL (TNFSF10) that also are known to impact nuclear factor kappa-B (NFκB) dimerization or whose promoters are activated by NFκB (Pahl, 1999). Overall 87 Probe sets (12% of total) to 56 genes that either bind to or are induced by NFκB proteins (see Table 18 (FIG. 50)) were significantly upregulated in ALS or AD patients. QRT-PCR analyses of NFκB-induced genes IL1RN, and TNFSF10 confirmed that both the ALS and AD patients had elevated median signals compared to healthy individuals (FIG. 12). This demonstrates that induction of the NFκB mediated transcription is one mechanism by which myeloid cells from ALS and AD patients alter their transcription relative to healthy controls.

Signals of Myeloid-Associated and Interferon-Stimulated Genes are Highly Correlated.

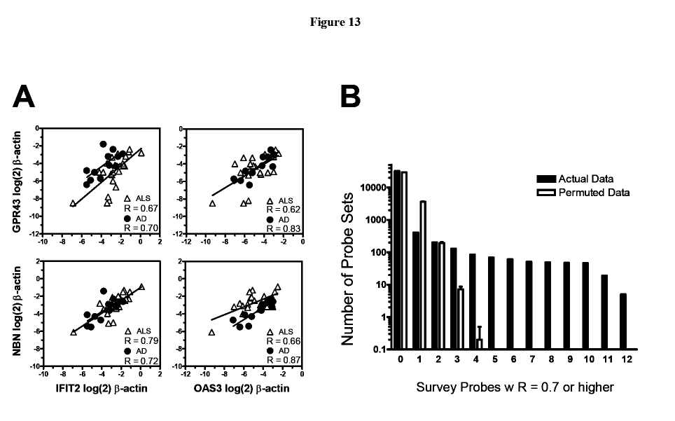

Several of the genes with a significant increase in signal in ALS and AD patients, such as nibrin (BN) and FXYD domain containing ion transport regulator 6 (FXDY6) have not been associated with myeloid origin, type I interferon, or NFκB. In order to determine if a high correlation also existed between the signals obtained from interferon-induced genes and the myeloid/NFκB genes, QRT-PCR signals of G protein coupled receptor 43 (GPR43, a myeloid-associated gene, LePoul et al., 2003) and nibrin (BN, involved in DNA damage repair, Varon et al., 1998) were compared to signals obtained from the interferon-induced genes IFIT2 and OAS3. As seen in FIG. 13, comparison of the β-actin normalized signals obtained from individual ALS (A) and AD patients ( [symbol] ) between the indicated genes (x and y axes) shows a high degree of correlation in expression of the upregulated genes. The best fit linear regression line for the ALS patients (black line) and AD patients (grey line) are also indicated as are the Pearson correlation coefficients for the comparisons. All correlations were significant at a level of at least p<0.01.

As seen in FIG. 13A, GPR43 expression was significantly correlated with the expression of IFIT2 and OAS3 in both ALS and AD patients with R values of 0.67-0.79. Similarly NBN expression significantly correlated with IFIT2 and OAS3 expression in ALS and AD patients with R values of 0.62-0.87. This shows that many of the genes with significantly increased signal in ALS and AD patients are expressed in a coordinated fashion and that ALS and AD patient PBMCs exhibit a common-myeloid cell based transcriptional signature of interferon-induced and myeloid-associated genes (the MIFN signature) not seen in healthy individuals.

The high correlation in the transcription of the highly upregulated genes in ALS and AD patients also provided a means to identify other genes that may be in the MIFN signature. To do this it was assumed that many of the most highly upregulated genes were MIFN signature members. Accordingly 12 of the probe sets with the greatest increase in signals in ALS and AD patients (the survey probes) were employed to identify other probe sets whose signals were highly correlated with the survey probes. Results obtained were compared to results obtained using a scrambled dataset.

Seen in FIG. 13B is a histogram of number of Probe sets that have a Pearson correlations coefficient of greater or equal to 0.70 with the indicated number of survey probes. The 12 survey probe sets were 41469_at (PI3), 205041_s_at (ORM1), 217502_at (IFIT2), 209396_at (CHI3L1), 220005_at (P2RY13), 1559573_at (AK096134), 203021_at (SLPI), 221345_at (GPR43), 220000_at (SIGLEC5), 203591_S_at (CSF3R), 202905_x_at (BN), and 217897 at (FXYD6). The black bar numbers were derived using actual data while the white bars indicate mean number of probe sets with R>=0.7 from 10 random permutations of the actual data set. Error bars indicate one standard deviation from the mean.

Greater than 85% of the expressed probe sets did not have a Pearson R of greater than or equal to 0.7 with any of the survey probe sets using either the actual or randomly permuted data. This shows that this means of identifying MIFN-signature members has high specificity. Using scrambled data, no probe sets would be expected to have a Pearson correlation coefficient of 0.7 or higher with 5 or more survey probe sets. In actuality, 347 probe sets to 244 genes had an R of 0.7 or higher with 5 or more of the 12 survey probe sets (see Table 19A and 19B (FIG. 51)).

This group of 244 genes represents the minimal set of myeloid/Interferon-induced (MIFN) signature genes and included 10 of the 12 test genes and 20 of 25 most highly upregulated gene and transcripts. The two test genes excluded by this analysis, P2RY13 and CSF3R, had an R>0.7 with three and one of the other survey probe sets, respectively. The remaining 10 survey probe sets had an R>0.7 with at least 8 of the other survey probes validating the initial assumption. Overall, the MIFN signature included 195 of the 484 genes that had a significantly increased signal in ALS and/or AD patients and 46 additional genes that did not meet the criteria for being significantly upregulated in ALS or AD patients. Thus the MIFN-signature genes make up a significant fraction of the upregulated genes in ALS and AD patients.

MIFN Signature Expression Correlates with Monocyte Activation In Vivo

In order to determine if the expression of the MIFN signature was related to the extent of monocyte/macrophage activation observable in vivo, the signals obtained via the QRT-PCR of genes of the MIFN signature in incubated PBMCs from ALS patients, AD patients, and controls were compared to the protein expression of the monocyte activation markers CD16 and HLA-DR as determined by flow cytometry.

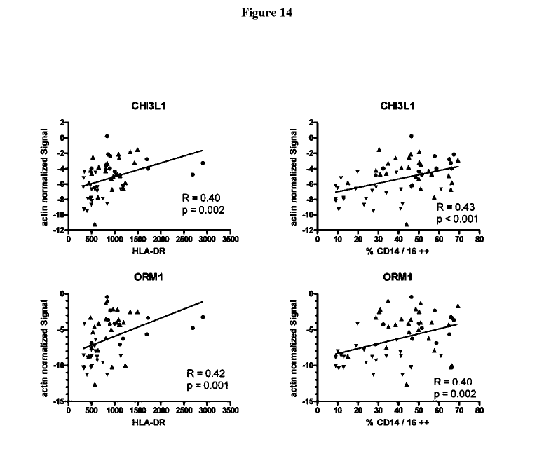

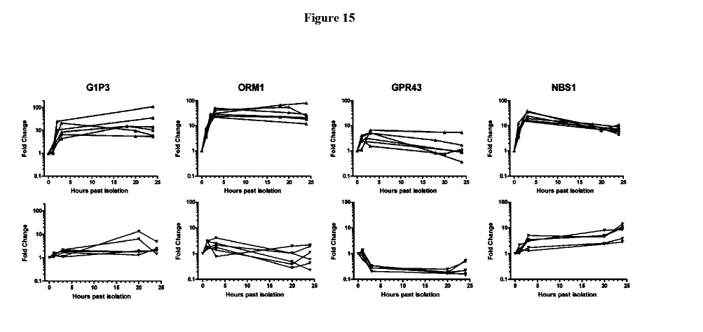

As seen in FIG. 14, multiple myeloid associated genes including α-acid glycoprotein and cartilage glycoprotein-39 were moderately (Pearson R of ˜0.4) but significantly correlated with the expression of HLA-DR and CD16 on CD14+ monocytes. FIG. 14 shows the comparison of β-actin normalized signals (y axis) for the MIFN signature genes CHI3L1 (top graphs) and ORM1 (bottom graphs) in mononuclear cells from ALS patients (▴), AD patients (●), and healthy individuals (▾) to the mean HLA-DR staining of CD14+ monocytes (left column) or the percentage of CD14+ monocytes that also expressed CD16 (right column). HLA-DR and CD16 staining was determined by flow cytometry. Mononuclear cells were incubated overnight in culture media prior to isolation of total RNA. The best fit linear regression line for the ALS patients (grey line) are indicated, as are the Pearson correlation coefficients and p values for the correlations.

This demonstrates that the expression of multiple genes of the MIFN signature can be related to the degree of macrophage activation before any in vitro cultivation.

MIFN Signature is Induced after Cell Isolation

In order to determine the timing of MIFN signature induction, cells from 6 additional ALS patients and 6 additional healthy controls were isolated and incubated for various lengths of time at 37 C post isolation. The demographic and clinical characteristics of these additional ALS patients and controls were not significantly different than those presented in Table 12 (FIG. 44, data not shown). Total RNA was prepared and the signals of four genes of the MIFN signature (G1P3, IFIT2, GPR43 and NBN) were determined.

RNA expression for 4 of the MIFN signature genes (indicated above graphs) in mononuclear cells from 6 ALS patients (top panels, black lines) and 6 controls (bottom panels, grey lines) after increasing amounts of time in culture at 37° C. is shown in FIG. 15. The mononuclear cells were prepared by ammonium chloride mediated-lysis of red blood cells. Cells were grown under non-adherent conditions as described above. The values in the figure are expressed as the fold change observed relative to the time 0 (immediately after red cell lysis) time point.

In the 6 ALS patients all 4 genes exhibited a rapid increase in signal with maximums generally reached after 3 hours of incubation. In contrast, incubation of control PBMCs led either to decreases in signal (GPR43) or minimal increases in signal that reached maximum levels at 20 to 24 hours post isolation. This shows that the MIFN signature was induced after blood cell isolation.

To confirm that the increases in transcription after blood cell isolation were accompanied by increases in protein expression, the tissue culture media from PBMC cultures from ALS patients and controls for two MIFN-signature proteins, elafin (PI3) and interleukin 1 receptor antagonist (IL1RN), that are typically secreted by myeloid cells were also evaluated. Equivalent levels of PI3 and IL1RN were detected in PBMC culture media from ALS patients and healthy individuals after one hour incubation.

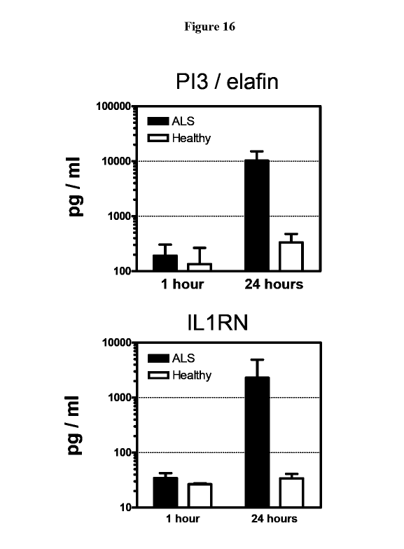

Mean levels of elafin (PI3, top panel) and interleukin 1 receptor antagonist (IL1RN, bottom panel) from cultures of mononuclear cells from 5 ALS patients (black bars) and 6 healthy individuals (white bars) after the indicated amount of time in culture at 37° C. are shown in FIG. 16. Error bars indicate one standard deviation from the mean. Mononuclear cells were prepared by ammonium chloride mediated-lysis of red blood cells. Cells were grown under non-adherent conditions as described in materials and methods.

After 24 hours mean levels of PI3 and IL1RN were increased approximately 60 fold in ALS Patient PBMC culture media but less than 3 fold in media from healthy control PBMCs. This indicates that the increased transcription of the MIFN signature in ALS PBMCs is accompanied by increased protein expression.

The current study showed that after short term cultivation peripheral blood myeloid cells from ALS and AD patients induce the coordinated expression of a very similar transcriptional program, designated the MIFN signature. This signature, includes genes associated with myeloid cells, genes induced by type I interferons, and genes that regulate or are regulated by NFκB. It also confirmed increases in protein expression for representative members of the MIFN signature and correlated the extent of MIFN-signature induction to the level of macrophage activation in vivo (without short term cultivation). Thus peripheral myeloid cells in individuals with ALS and AD appear to be primed to enter a pro-inflammatory program prior to even leaving the bloodstream. It is to be expected that some of the activated macrophages present in the blood will eventually migrate into the spinal cord or motor cortex in ALS or the cerebral cortex in Alzheimer's disease. This pathogenic model predicts that outside of the circulatory system these cells will enter the transcriptional program described in this study.

The presence of genes commonly upregulated in peripheral blood myeloid cells and the CNS of ALS and AD patients can be explained by infiltration of MIFN signature expressing monocyte/macrophages into regions of active disease.

### EXAMPLE 11

This example demonstrates that there is a high level of inter-correlation of transcription of the MIFN-Interferon signature across all samples.

Samples Employed

Relevant clinical and demographic information for all patients and controls used in this study are included in Table 20 (FIG. 52). The number of ALS patients evaluated is significantly expanded from those used in Example 10 above. Additionally, blood samples from individuals with age related macular degeneration (ARMD) are tested as they serve as a patient group with activated macrophages but without neurodegeneration. Also included are HIV infected individuals with controlled disease (HAART group), HIV infected individuals with neurological dysfunction (HAND), and HIV infected individuals with plasma viremia (“FAILED”—these are HIV-infected individuals resistant to HAART). In addition, some of the genes have been evaluated against women with ductal carcinoma in situ as women with DCIS also have high levels of activated macrophages in their blood.

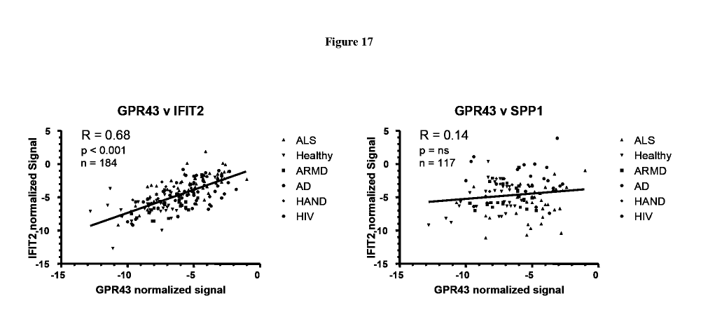

GPR43, a macrophage associated gene of the signature was compared to the transcription of IFIT2, an interferon stimulated gene. Comparing signals obtained from 184 samples (FIG. 17), including individuals with ALS, AD, HIV associated neurological disease (HAND) and other HIV infected individuals, GPR43 and IFIT2 had a Pearson correlation coefficient (R) of 0.68 (p<0.0001). In contrast, comparison of GPR43 to another gene associated with macrophages, osteopontin (SPP1, Krause et al., 1996) generated an R=0.14 which was not significant (FIG. 17).

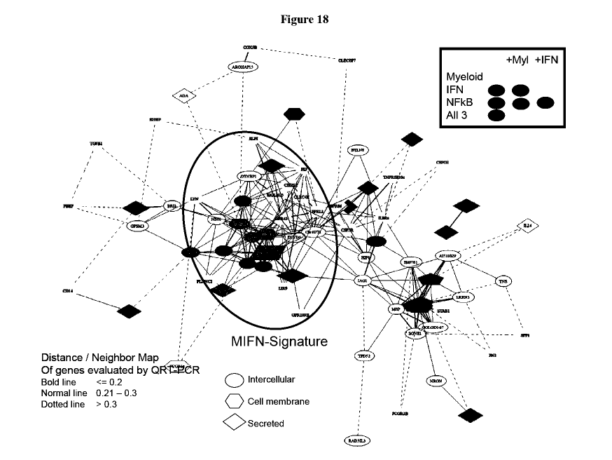

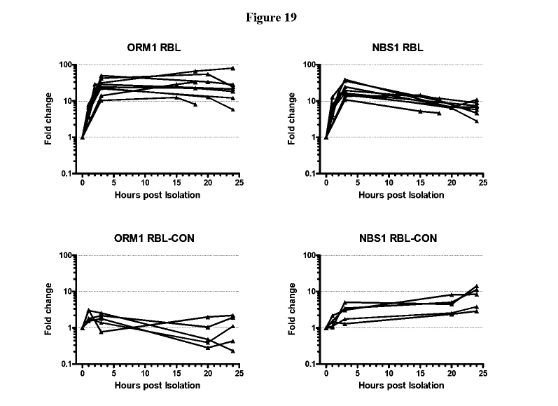

By comparing the signals obtained by QRT-PCR in a manner similar to that used in Example 10, a map of how well correlated the transcription of the various genes are to each other was generated (FIG. 18). As seen in FIG. 18, genes are connected to other genes with which they have an R>=0.7 (solid lines) or 0.8 (bold lines). Those genes that do not have an R of 0.7 or higher with any of the 75 genes analyzed are connected to the map to the two genes with which they are most closely correlated (dotted lines). In this analysis the MIFN signature (which includes GPR43 and IFIT2) is visible as a group of 26 genes that show extensive inter-correlation (circled). The MIFN-signature includes interferon stimulated genes, and cell surface receptors (hexagons) and secreted proteins (diamonds) known to be produced by monocytes, macrophages, and granulocytes.

### EXAMPLE 12

This example demonstrates that the MIFN-signature genes are induced post isolation. The same sample set was used as described in Example 10.

One of the features that distinguishes genes of the MIFN-signature is their induction in short term mononuclear cell cultures. Typical results are shown in FIG. 19. For both the secreted acute phase protein alpha 1 acid glycoprotein (ORM1) and the DNA repair protein nibrin (NBS1) signal are induced between 10-100 fold in ALS patients after culture of mononuclear cells for 3 hours at 37 C. In healthy controls however, there is no apparent induction of RNA (ORM1) or a very slow increase over 24 hours (NBS1). These characteristic short and fast induction characteristics have been confirmed for many MIFN signature proteins including the interferon stimulated genes G1P3, IFIT2, MX2, and TNFSF10; the cell surface receptors GPR43, GPR109, and SIGLEC5; and the secreted factors PI3, IL1RN and CHI3L1 (data not shown).

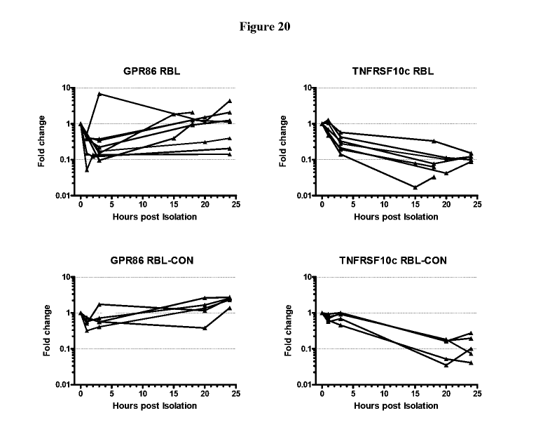

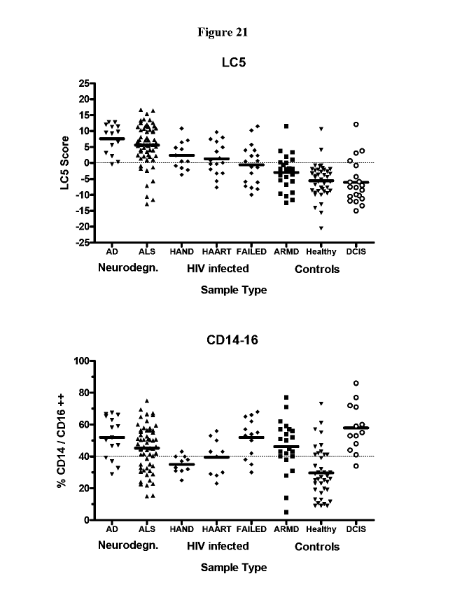

In contrast, other genes associated with macrophages and granulocytes that are upregulated in ALS patients show alternative gene expression patterns (FIG. 20). GPR86 (also designated P2RY13) which exhibits 7 fold higher expression in ALS and AD patient (see Table 15 (FIG. 47)) generally falls somewhat during the first 3 hours of mononuclear cultures from ALS patients and then may return to baseline. Trail decoy receptor (TNFRSF10c) which is 3.8 fold upregulated in ALS patients has its signals steadily fall in mononuclear cell cultures from both ALS patients and controls. This shows that MIFN signature genes can be distinguished from other genes upregulated in ALS and AD patients by the kinetics of RNA expression in culture.

### EXAMPLE 13

This example describes the method by which the MIFN signature was determined. The same sample set was used as described in Example 11.

Putting together all of the relevant QRT-PCR data obtained with the samples described in Table 20 (FIG. 52) with over 80 tested primer sets, as well as data on the incubation period required for maximum expression, and the intergene correlations depicted in FIGS. 8 and 18, a list of 26 QRT-PCR confirmed members of the MIFN signature was obtained (see Table 21 (FIG. 53)).

However, it is clear that these 26 genes do not represent the full extent of the MIFN signature, in as they represent only a small sub fraction of the genes significantly upregulated in ALS and AD patients. In an effort to define the full extent of the MIFN signature, the Probe Sets that correspond to each of the 26 confirmed members of the MIFN-signatures were employed to identify additional members of the MIFN signature using the Affymetrix probe set signals obtained with the 60 samples (25 ALS, 12 AD, and 23 healthy individuals) described in Example 10 above.

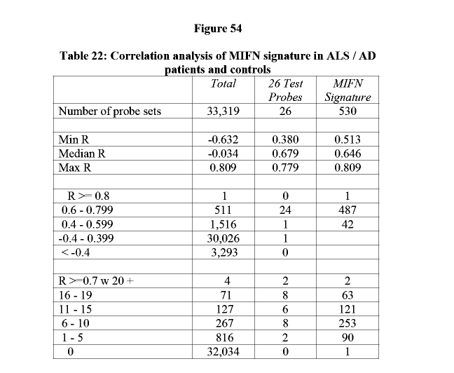

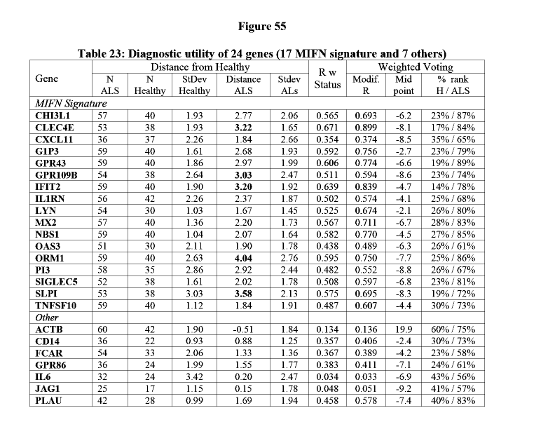

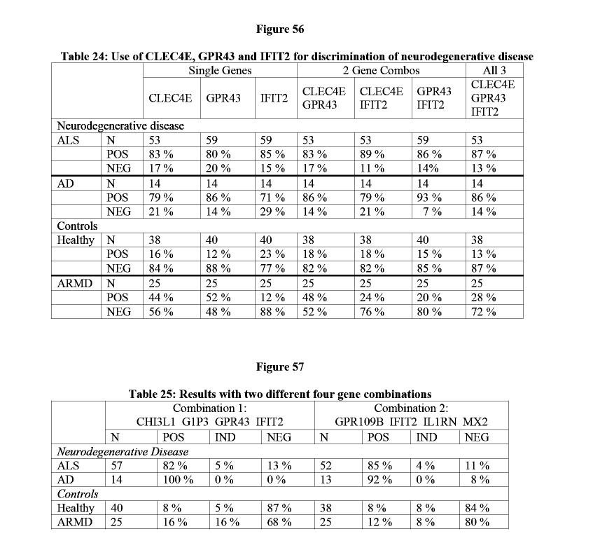

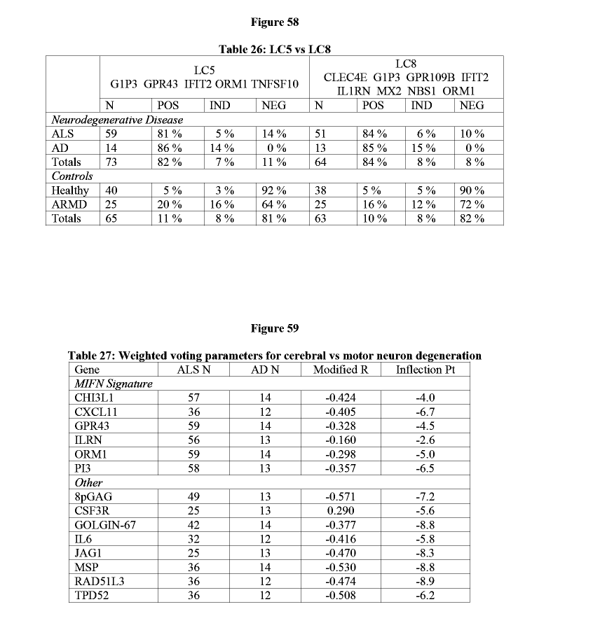

The microarray signals obtained with each expressed Probe Set for all 60 samples were compared to the signals obtained with each of the 26 Probe sets used to aid PCR primer design of the QRT-PCR confirmed members of the MIFN signature to generate a Pearson correlation coefficient for each comparison. Then the median correlation coefficient of the 26 R values was derived. Additionally, the number of the 26 confirmed Probe sets with which the Probe set under consideration had an R>=0.7 was determined. Basic statistics associated with this analysis are presented in Table 22 (FIG. 54). Over 90% of the probes had a median R between −0.4 and 0.4 which is not significant with an N of 26. Also 96% of the Probe sets did not have an R of 0.7 or higher with any of the test probe sets. Thus using the confirmed MIFN signature members as test probes to identify additional MIFN signature members has high specificity. In contrast, 24 of the 26 confirmed MIFN signature probe sets had a median R of greater than 0.6, indicating that over 90% of the MIFN signature test genes were correctly identified as MIFN signature members.

All Probe Sets with a median R of 0.6 or higher with the 26 QRT-PCR confirmed members of the MIFN signature were included as members of the full MIFN signature (N=488). To this list was added those probe sets that had an R of 0.7 or higher with 6 or more QRT-PCR confirmed members of the MIFN signature and a median R with all QRT-PCR confirmed members of the MIFN signature of 0.5 or higher (42 additional probe sets). Adding the 26 test probe sets to this list results in a final list of 556 Probe Sets representing, 549 sequences in Genbank, 393 (337+56) known genes, and 17 transcripts/Genbank sequences not definitively assigned to a gene. These 393 known genes and 17 transcripts represent the collection of genes most useful for the identification of disease inducing macrophages and the diagnosis of neurodegenerative diseases. The sequences, genes and the probe sets obtained through this analysis are listed in Tables 28 (Genbank ID table) and 29 (FIGS. 60 and 61), respectively. Note that table 29 contains an additional 13 Genbank sequences that are to genes that are in the MIFN signature, but whose probe sets did not meet the strict criteria mentioned above (549+13+41=603, the full number of Genbank sequences).

However, as seen in Table 21 (FIG. 53) as well as some of the examples below, there are genes that are not in the MIFN signature that are useful for the diagnosis of neurodegenerative disease (e.g., P2RY13/GPR86 or CSF3R/colony stimulating factor 3 receptor), differentiation of ALS from AD (e.g., RAD51 L3 or transmembrane protease, serine 13/TMPRSS13/MSP), or clinical monitoring (e.g., Golgin-67/GOLGA8B or 8pGAG). These genes of the invention are listed in Table 30 (FIG. 62).

### EXAMPLE 14

This example describes the development of a robust molecular assay to detect neurodegenerative disease (e.g., ALS and AD) using the MIFN signature genes. The same sample set was used as described in Example 11.

In order to identify which genes would most likely have diagnostic utility the signals from patients with ALS were evaluated and compared to those from healthy individuals. Three methods to assess the utility of individual genes were used—(1) distance from healthy individuals; (2) correlation with disease status; and (3) weighted voting.

In the first method, the distance from healthy individuals, the difference, i.e., how much greater or lower, between the signals observed in diseased individuals and the average signal of healthy individuals was measured. A desirable diagnostic gene would have a large separation in signals between diseased and healthy individuals with minimal variation (i.e. low standard deviation) in intragroup (disease or healthy) signals.

In the correlation with disease method, it is postulated that a perfect gene would have a high signal in individuals with disease (i.e., a value of 1 for positive) and no signal in a healthy individual (i.e., 0). Accordingly individuals with ALS are assigned the value 1 and healthy individuals are assigned the value 0. The Pearson correlation coefficient of the signals obtained with the actual gene relative to the theoretical case was determined. The higher the R obtained the closer to a perfect diagnostic gene the test gene is.

In the weighted voting method a weight was assigned to each gene based on the distance between the averages of the diseased and healthy populations and the size of the standard deviations (see Example 2, above). The advantage of this method is that weights are not limited to the range of 1 to −1 (as is the case with the correlation with disease method).

The results obtained with 24 genes are presented in Table 23 (FIG. 55). Analysis of signals from approximately 100 samples indicated that most MIFN signature genes had an average of an 4-8 fold higher signals in ALS patients (average distance of ˜2.5) with a standard deviation of around 4 fold (˜2.0). This indicated that the separations between the signals obtained from samples with ALS were not sufficiently great enough to allow for any one gene to distinguish the two populations. The gene with the best performance individually was CLEC4E which was separated by an average of 9.3 fold from healthy individuals with a standard deviation of ˜3 fold. Other genes with good separation included IFIT2 and SLPI. Control genes, such as beta actin and CD14, in contrast, had minimal separation with much greater standard deviations.

Correlation with disease status gave similar results with MIFN signature disease correlations ranging between 0.67 (CLEC4E, p val˜10⁻¹³) to 0.354 (CXCL11, p val=0.002). Control genes such as beta actin had a disease correlation near 0. Other genes not in the signature but upregulated in ALS (FCAR, GPR86, PLAU) had disease correlations of 0.367 to 0.458, as did the gene for CD14 which had a correlation with disease of 0.357 (p value=0.006). As above, CLEC4E and IFIT2 were the genes with the highest correlation with disease, but other genes such as GPR43, NBS1, and MX2, which were not as highly separated from healthy controls as some others (e.g., ORM1, GPR109B) also had relatively high correlations with disease status. Results using the modified R calculations were similar to those obtained with the correlation with disease approach.

The major difference was that the modified Rs were higher than the corresponding disease correlation value (Table 23 (FIG. 55)).

The weighted voting method requires use of the modified R value, which gives greater weight to the values obtained with the best discriminators and a midpoint value, which was set as the average of the values obtained with all healthy and diseased training samples (see Example 2, above). This midpoint or inflection point is the signal value above which samples will said to be likely associated with disease (positive values) and below which samples will said to be healthy (negative values). Assessment of where the inflection points fell in the distribution of values from healthy and diseased samples is provided in Table 23 (FIG. 55). In general the midpoints fell at around the 70-80^{th} percentile for healthy controls and the 20-30^{th} percentile for ALS patients. This reconfirmed the fact that no one gene could reliably discriminate ALS patients from healthy patients and the combinations of multiple genes was required.

Starting with 2 gene combinations, results obtained with the MIFN-signature genes CLEC4E, GPR43, and IFIT2 can be combined together and the fraction of ALS patients and controls that are called positive (likely disease) or negative (healthy) determined. To evaluate the combinations in samples not used to generate the modified R values, the predictive power of the various combinations with AD patients and individuals with age-related macular degeneration (ARMD) was determined (Table 24 (FIG. 56)). All 3 genes gave equivalent performance on ALS patients with approximately 80% of the samples correctly identified as diseased. With AD patients a wider range of values was obtained with GPR43 calling 86% of the AD patients positive and IFIT2 calling only 71% positive. For healthy controls IFIT2 was the worst of the three genes miscalling 23% of the normals tested and GPR43 was the best (miscalling 12%). With ARMD patient samples, IFIT2 became the best performing single gene, indicating that 88% of the patients were negative and 12% were positive.

For the two gene combinations all three called approximately 80% of the healthy individuals as negative and between 83-89% of the ALS patients positive. The best performing 2-gene combination was GPR43 and IFIT2 which called 13 or 14 AD patients positive (93%) and only 5 of 25 ARMD patients positive (20%). The worst combination was CLEC4E and GPR43 which called approximately half of the ARMD patients positive. When all three genes were used to discriminate patients the results were comparable to those obtained with GPR43 and IFIT2. Minor changes were seen in the number of healthy controls miscalled (15% for GR43/IFIT2 vs 13% for all 3) and the number of ARMD patients miscalled rose slightly (20% vs 28%).

As larger numbers of genes were included, a predication strength calculation to assess the robustness of the result obtained was added. The Prediction Strength (PS) is the score obtained by adding up the votes of all of the genes of the combination divided by the absolute value of all of the added votes (see Example 2 above). Accordingly a PS of 1.0 means that all of the genes voted for diseased, a PS of −1.0 means that all of the genes voted for healthy and a PS of 0.5 means that 50% more genes voted for diseased over healthy. PS values of 0.3 to −0.3 were designated indeterminate in as approximately equal amounts of genes vote for positive and negative.

The evaluation for two different four-gene combinations is shown in Table 25 (FIG. 57). It should be noted that only one gene (IFIT2) is shared between the two combinations. Despite that their performance is quite comparable, with both combinations correctly identifying 82-85% of the ALS patients, 92-100% of the AD patients while misclassifying 8% of the healthy controls and 12-16% of the ARMD patients. This demonstrates that many of the MIFN signature genes are useful diagnostically and many equally valid combinations exist for the diagnosis of neurodegenerative diseases.

### EXAMPLE 15

This example identifies useful combinations of MIFN signature genes. The same sample set was used as described in Example 11.

The LC5 and LC8 Assay

The LC5 assay uses the 5 genes G1P3, GPR43, IFIT2, ORM1 and TNFSF11 (see Example 2, above). The LC8 assay, conducted similar to the LC5 assay, uses the 8 genes CLEC4E, G1P3, GPR109B, IFIT2, IL1RN, MX2, NBS1 and ORM1. The results obtained with the two assays is presented in Table 26 (FIG. 58). It can be seen that both assays have slightly lower misclassification of healthy samples than either of the 4 gene assays with approximately 90% of these samples called negative. A comparison of the two assays, shows that the LC8 assay had slightly better performance at calling ARMD samples negative and miscalled a slightly lower number of ALS patients as healthy. In general though, with these assays, around 85% of the neurodegenerative patients will be identified and around 90% of the controls will be called negative. This example demonstrates that these assays are very useful for identifying individuals with neurodegenerative diseases such as ALS and AD.

The LC5 assay was also tested against samples from individuals with other diseases including HIV infected individuals both with and without neurological disease. The results from these assays, along with the levels of activated macrophages as measured by percentage of CD14+ monocytes also staining for CD16 are presented in FIG. 21.

As seen in FIG. 21, the LC5 assay clearly distinguished ALS and AD patients from healthy individuals or individuals with ARMD (mean LC5 ALS=5.6, AD=7.6, Healthy=−5.6, ARMD=−3.0 p<0.001 with Bonferroni's correction for all comparisons). Individuals with HAND and controlled viremia (1 out of 12 with detectable plasma viral load) were also clearly distinguished from healthy individuals (mean LC5=2.4, p<0.01) but not individuals with neurodegenerative disease, HIV infected individuals with controlled viremia (HAART, mean LC5=1.3) or individuals with plasma viremia (FAILED, mean LC5=−0.6). Women with ductal carcinoma in situ (DCIS, mean LC5=−6.1) was the group with the lowest mean LC5 score and were not significantly different than individuals with macular degeneration or healthy individuals.

In contrast, evaluation of the level of activated macrophages by flow cytometric staining of CD14-16 cells the mean percentage of monocytes that also were positive for CD16 was significantly elevated from that seen in healthy individuals (p<0.001) in all other groups except HIV infected individuals with HAND or with controlled viremia. In particular levels of CD14-16 cells were comparable in ALS, AD, ARMD, and DCIS (mean % for ALS=45.2, AD=51.9, ARMD=46.1, DCIS=57.9, p=ns). In HIV infected individuals, the lowest levels of CD14-16 cell staining was seen in individuals with controlled viremia and HAND, and the highest level of staining was seen in individuals with high HIV plasma viral loads. This indicates that the LC5 (and by inference the LC8) are not simply measuring levels of activated macrophages, since samples with very high levels of activated macrophages (e.g. DCIS) can have very low LC5 values.

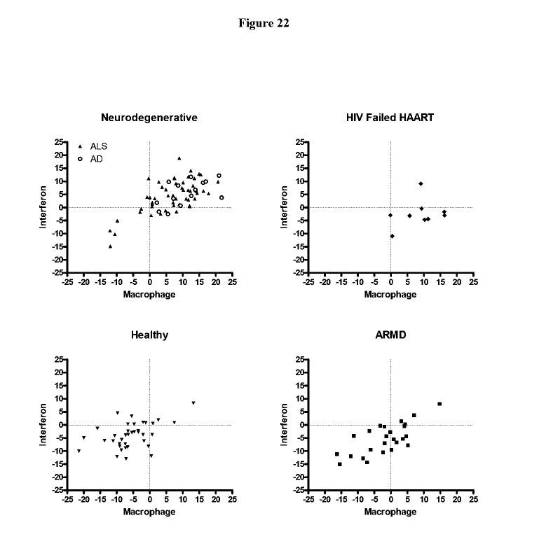

The MIFN signature is made up of a collection of genes known to be expressed in macrophages and genes induced by type I (alpha/beta) interferons. In order to quantify the expression of these two types of genes separately, an assay was performed for genes of the Interferon signature (CXCL11, G1P3, IFIT2, MX2, OAS3, TNFSF10, see FIG. 18 above) and a collection of strongly macrophage associated genes (CHI3L1, CLEC4E, GPR43, GPR109B, ORM1, PI3). This analysis provided a two dimensional plot (see FIG. 22) in which individuals with neurodegenerative disease formed a distinct cluster primarily in the positive/positive (upper right) quadrant. Healthy individuals had intermediate to low values for interferon stimulated genes and low values for macrophage associated genes and for a cluster in the negative/negative (lower left) quadrant. Individuals with ARMD in contrast had similar interferon gene values as controls but had a noticeable increase (shift to the right) in values for the macrophage associated genes (−1.8 vs −6.2). HIV infected individuals who failed HAART had relatively high values of macrophage associated genes but low to moderate interferons. This demonstrates that individual clinical groups can be evaluated according to their differential induction of macrophage associated and interferon stimulated gene of the MIFN signature. These distributions can be used to assign domains to the various groups and classify unknown samples by virtue of their distance from the center of the various domains (i.e., a novel sample located well into the upper right quadrant would be classified as neurodegenerative like and a sample in the lower right quadrant would be classified as macrophage activation w/out neurodegeneration).

### EXAMPLE 16

This example demonstrates the ability of the MIFN signature in combination with other genes of the invention to differentiate between different clinical types of neurodegeneration. The same sample set was used as described in Example 11.

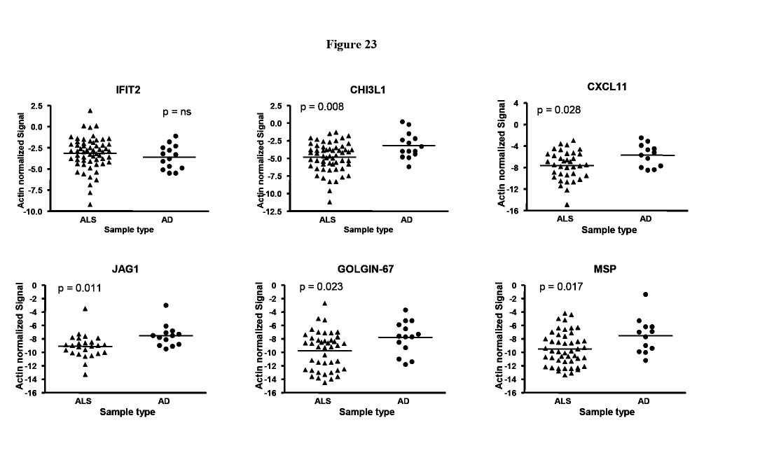

The signals obtained with six genes by QRT-PCR in patients with ALS and AD are shown in FIG. 23. Most MIFN signature genes were like IFIT2 in that there was no discernable difference in the signals obtained from ALS and AD patients. However some MIFN signature genes, such as CXCL11 and CHI3L1 showed significant increases in mean signal in AD patients relative to ALS patients. Along with some MIFN signature genes, other genes such as JAG1, GOLGIN-67, or MSP that have increased signals in AD patient samples were also seen.

Table 27 (FIG. 59) provides the modified R values and inflection points of 14 genes that have utility in differentiating ALS from AD. Most of the Modified R values are lower than those obtained for differentiating ALS from healthy individuals reflecting the smaller differences in signal being exploited. Accordingly, a collection of these genes with differential signals in the two conditions can be used to construct a weighted voting classification system, similar to that described in examples above.

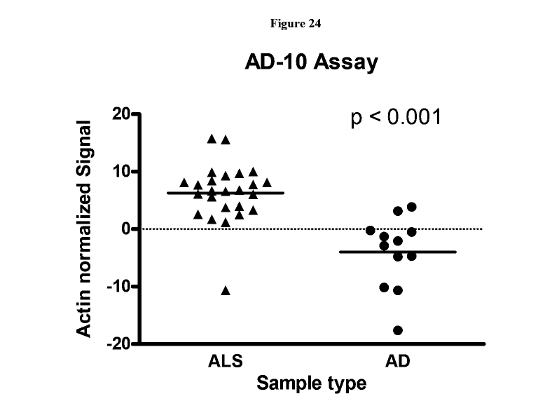

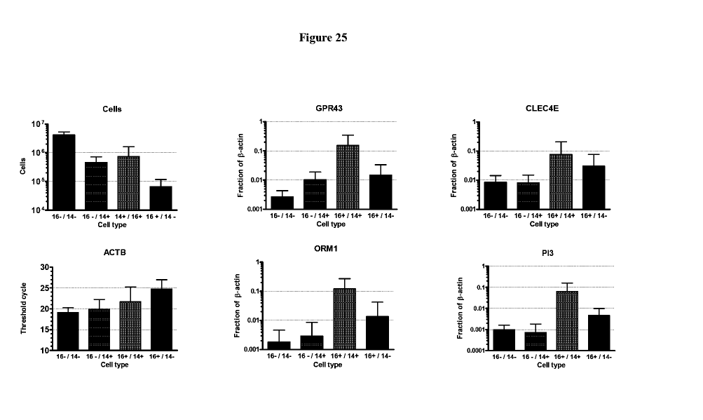

One combination of genes useful to differentiate between ALS and AD was identified as using the ten genes 8pGAG, CSF3R, GOLGIN-67, IL6, IL1RN, JAG1, MSP, PI3, RAD51 L3, and TPD52. Using this system patients with ALS received positive votes and individuals with AD received negative votes. The results obtained with a small panel of samples are provided in FIG. 24. The 25 ALS patients tested had a mean AD10 signal of 6.3 and the 12 AD patients had a mean signal of −4.0. The differences in the 2 groups were highly statistically significant (p<0.001). Additionally 10 of the 12 AD patients were classified correctly (though 2 had values between 0 and −1.0) as were 24 of 25 ALS patients. This shows that the combination of genes used can be used to further classify neurodegenerative diseases into ALS or AD.

### EXAMPLE 17

This example identifies the method and criteria used to further identify useful ProMac signature genes.

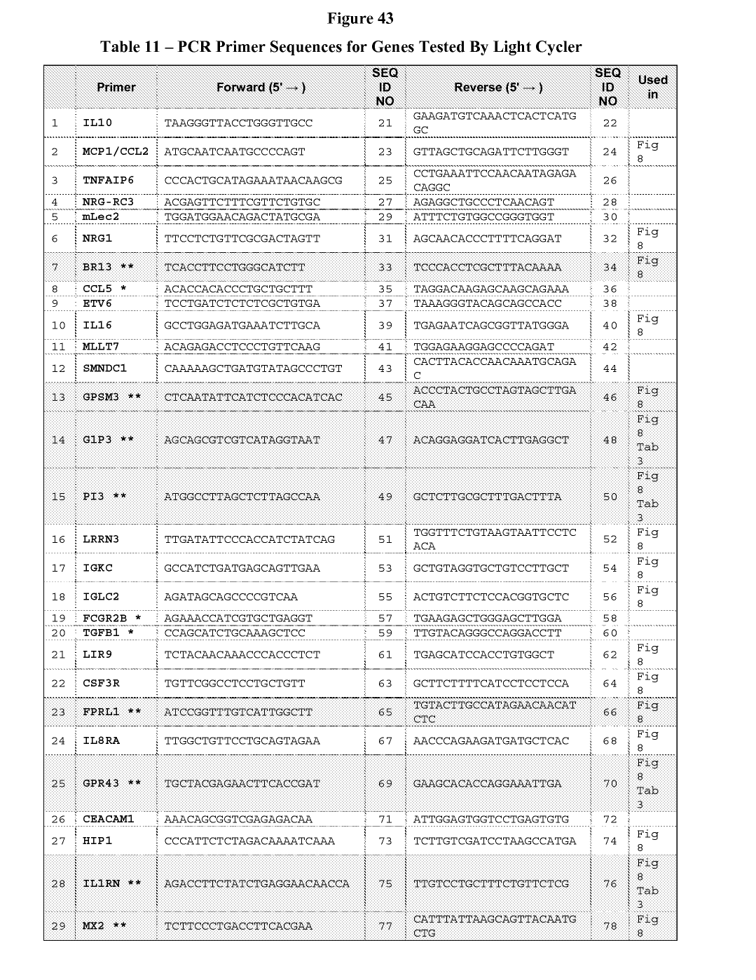

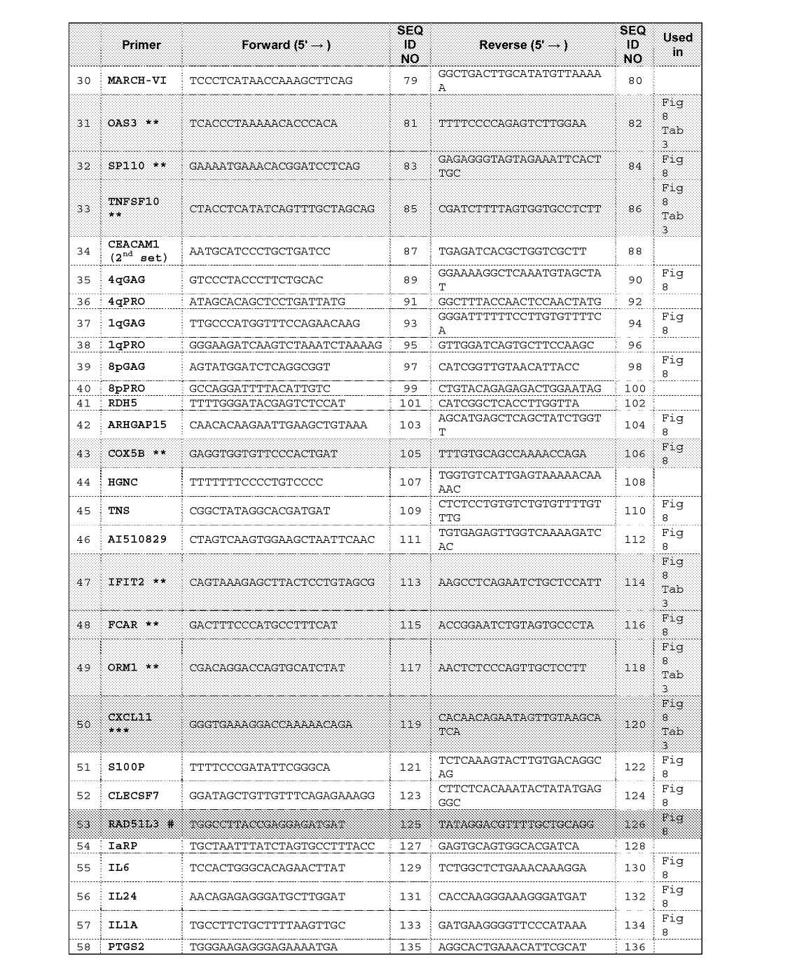

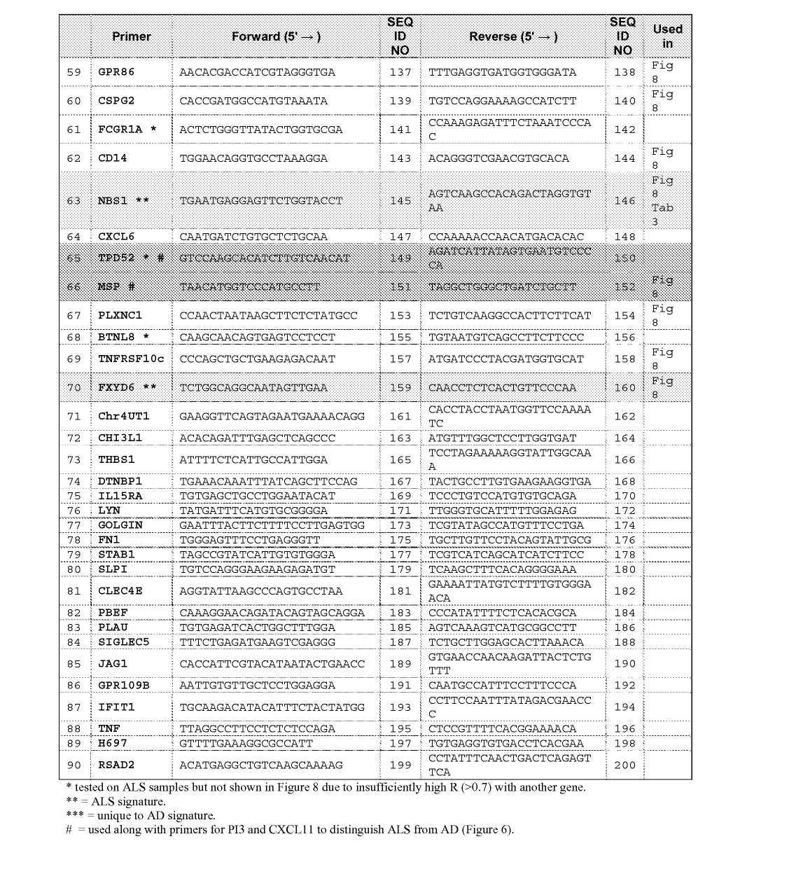

The Affymetrix data of Examples 1 and 11 were employed to further identify ProMac signature genes. This identified a set of over 80 genes, included in FIGS. 35, 43 and 45 that were evaluated by QRT-PCR.

Of these approximately 80 genes, 26 were found to be in the MIFN signature using the following criteria:

(1) they all showed an increased expression in ALS/AD;

(2) they had a high degree of correlation of signals with each other; and

(3) they showed a similar time course of expression

The probe sets used in the design of the QRT-PCR primers of the 26 genes confirmed the MIFN-signature members (see Example 13) and were then employed to identify additional MIFN signature members from the full set of Affymetrix data. This identified 368 additional genes and 16 additional transcripts as well as 25 QRT PCR confirmed genes and 1 QRT-PCR confirmed transcript; i.e., 393 known genes and 17 transcripts (the set of 556 probe sets discussed in Example 13).

Of these genes, 56 representing 108 Genbank sequences validated their use by QRT PCR (the 26 QRT-PCR confirmed members of the MIFN signature) or had a 4 fold or higher increased mean signal in ALS and/or AD patients. These genes are listed in Table 29 (FIG. 61).

### EXAMPLE 18

This example demonstrates that the type of cells that the MIFN signature is primarily expressed in are CD14/16 macrophages.

In order to determine which cell type(s) the MIFN signature is primarily expressed in, mononuclear cells were isolated via percoll gradient centrifugation from healthy individuals. The cells were then washed and incubated overnight in RPMI media plus 10% fetal bovine serum at 37 C for about 20 hours. Then the cells were incubated with antibodies to human CD16 attached to magnetic beads. The bound cells were separated from the unbound fraction using an Automacs separator (Miltenyi, Albany Calif.), which relies on magnets to retain cells bound to the CD16 beads. Both the bound and unbound cells were retained, and the bound fraction was treated with releasing reagent, which separates the cells from the beads. Then both the bound and unbound fraction were incubated with antibodies to CD14 and the bound and unbound fractions isolated using an Automacs. At the conclusion of the procedures the cells were separated into CD16+, cells double positive for CD16 and CD14, cells only positive for CD14 and cells negative for both antigens. The cells were then washed, counted, and resuspended in TRIZOL and RNA, DNA, and protein fractions obtained according to manufacturer's instructions (InVitrogen, Carlsbad, Calif.). Expression levels of actin and various MIFN signature and control genes were then determined via QRT-PCR with appropriate primers.

Mean signals obtained from separated cells of 6 healthy individuals are presented in FIG. 25. Approximately 80% (˜3-4×10⁶ cells) of the cells were negative for both CD14 and CD16. Essentially equal levels of CD14+ cells and CD14/CD16 double positive cells were isolated (8 to 10% each, though yields of CD14/16++ cells were more variable). The smallest number of cells were CD16 single positive (approximately 1.5% of the total). Results with actin represented total cell counts quite well with the lowest threshold cycles (highest signals) obtained with the double negative, cells intermediate and approximately equal threshold cycles obtained with CD14+ and CD14/CD16++ cells and the lowest signals obtained with CD16 single positive cells. In fact the correlation of Actin Ct values with cell counts was R=0.79 (p<0.0001) across all cell types, ensuring that use of beta actin for normalization was warranted.

RNA signals for 2 non signature macrophage associated genes CD14 and chondroitin sulfate proteoglycan 2 (CSPG2) were analyzed. Expression levels for both genes were lowest in the double negative cells, moderately higher in CD16+ cells and approximately equal in CD14+ monocytes and CD14/C16 double positive cells (p=ns, data not shown).

When signals from 4 macrophage associated signature genes (GPR43, ORM1, PI3, and CLEC4E, FIG. 25) were evaluated in the different cell types the lowest signals were obtained in the double negative cells and the next lowest signals were obtained in CD14 single positive cells (i.e., blood monocytes). For GPR43 signals in CD16 single positive cells and CD14 single positive cells were approximately equal. For ORM1, PI3, and CLEC4E there was a clear trend toward higher signals in CD16 single positive cells than in the CD14+ monocytes. However for all 4 MIFN signature genes the highest signals were obtained in CD14/CD16 double positive blood macrophages. In general, differences between RNA levels in CD14+ monocytes and CD16+ monocytes were greater than 10 fold and the differences were significant at p<0.05.

Flow Cytometric Analyses

To confirm the results of the cell separation studies described above, the expression of several MIFN signature proteins was verified by flow cytometry with appropriate antibodies. The following list is provided for antibodies employed and their sources:

| | |
|---|---|
| GPR43 rabbit polyclonal | Abcam Inc. |
| (epitope to the 3^{rd} | One Kendall Square, Bldg 200, 3^{rd} Floor |
| extracellular loop) | Cambridge, MA 02139 |
| | Cat# ab12571 |
| HM74 rabbit polyclonal | Abcam Inc. |
| (epitope to the N-term | One Kendall Square, Bldg 200, 3^{rd} Floor |
| extracellular) | Cambridge, MA 02139 |
| | Cat# ab12611 |
| isotype controls (FL1, | BD Biosciences |
| FL2, FL3) FL1 (FITC) | 2350 Qume Drive |
| | San Jose, CA 95131 |
| FL2 (PE) Streptavidin-PE | R&D Systems |
| | 614 McKinley Place NE |
| | Minneapolis, MN 55413 |
| FL3 (TriColor) | Invitrogen Corporation |
| | 1600 Faraday Avenue, PO Box 6482 |
| | Carlsbad, CA 92008 |
| NBS1 (mouse monoclonal | Upstate USA, Inc. |
| IgG) | 10 Old Barn Road |
| | Lake Placid, NY 12946 |
| FPRL1 (mouse monoclonal | Abcam Inc. |
| [GM1D6]) | One Kendall Square, Bldg 200, 3^{rd} Floor |
| | Cambridge, MA 02139 |
| | Cat# ab26316 |
| CD14 TriColor | Invitrogen Corporation |
| | 1600 Faraday Avenue, PO Box 6482 |
| | Carlsbad, CA 92008 |
| CD16-FITC & PE | BD Biosciences |
| | 2350 Qume Drive |
| | San Jose, CA 95131 |
| PI3 (Human | HyCult biotechnology b.v. |
| Elafin/Skalp ELISA kit) | Frontstraat 2a |
| | 5405 PB UDEN, The Netherlands |
| | c/o Cell Sciences, Inc. |
| | 480 Neponset Street, Building 12A |
| | Canton, MA 02021 |
| IL1RN (Human IL-1ra | Raybiotech, Inc. |
| ELISA Kit) | 150 Technology Parkway, |
| | Norcross, GA 30092 |
| HM74, GPR43, and FPRL1 | Sigma-Aldrich, Inc |
| (rabbit-FITC secondary | 3050 spruce Street |
| antibody, anti-rabbit | St Louis, MO 63103 |
| IgG FITC conjugate) | cat# F9887 |

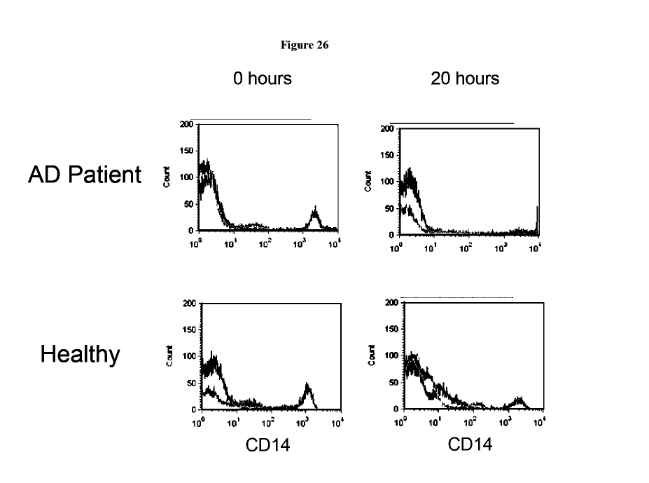

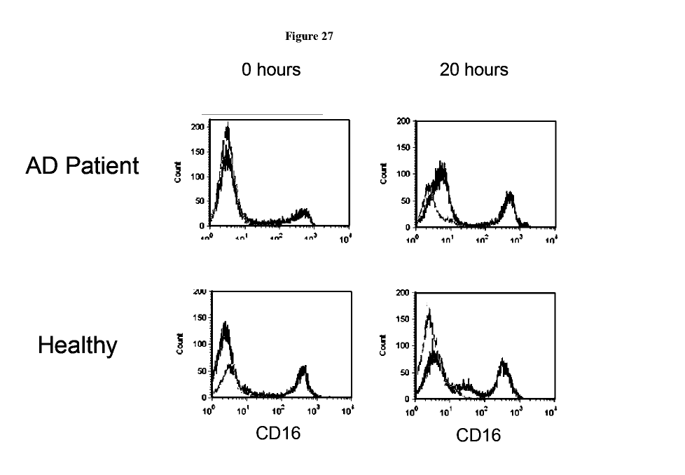

To identify cell subsets, mononuclear cells from patients with neurodegenerative diseases and healthy controls were also stained with antibodies to CD14 and CD16. Typical results obtained with an AD patient and a healthy individual are presented in FIG. 26. As seen in FIG. 26, staining of percoll purified mononuclear cells shortly after isolation with antibodies to CD14 resulted in strong labeling of 5-10% of all cells with little or no signal obtained with isotype matched control antibodies. After an overnight incubation at 37° C. under non-adherent conditions, expression of CD14 was significantly reduced in both AD patients and healthy individuals. This reduction in CD14 positive cells is associated with the differentiation of CD14 monocytes into cells more closely resembling tissue macrophages. This process initiates independent of any external stimuli, including attachment.

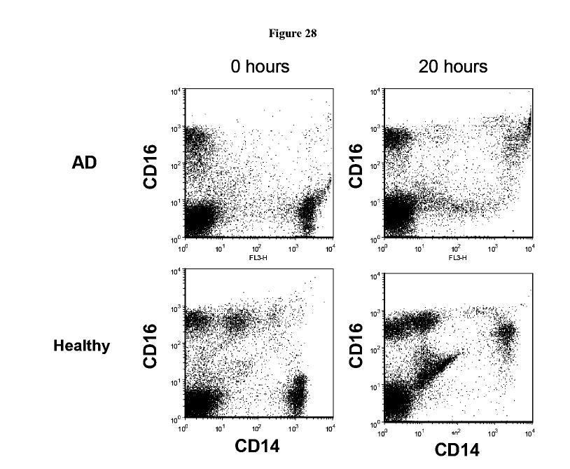

Concurrent staining of percoll purified mononuclear cells with CD16 also labeled between 10-20% of all cells, once again with little or no staining seen with an isotype matched control. In contrast to CD14 staining, the fraction of cells positive for CD16 increased after an overnight incubation (see FIG. 27). As seen from the double-stained cells in FIG. 28, low levels of expression of CD16 on CD14 monocytes immediately after isolation can be detected in both patients with neurodegenerative disease and healthy individuals. After an overnight incubation, levels of CD16 expression on CD14 monocytes increased between two- to five-fold so that up to 80% of CD14 monocytes were now also expressing CD16 (FIG. 28, 20 hour panels).

Expression of RNA of GPR43, NBS1 and other MIFN signature genes reached maximum levels at between 3 to 12 hours post isolation (see FIG. 19 or FIG. 15) and was stable or declined slowly thereafter. Similarly expression of secreted signature proteins, such as IL1RN or elafin (PI3) reached a maximum after 24 hours of incubation.

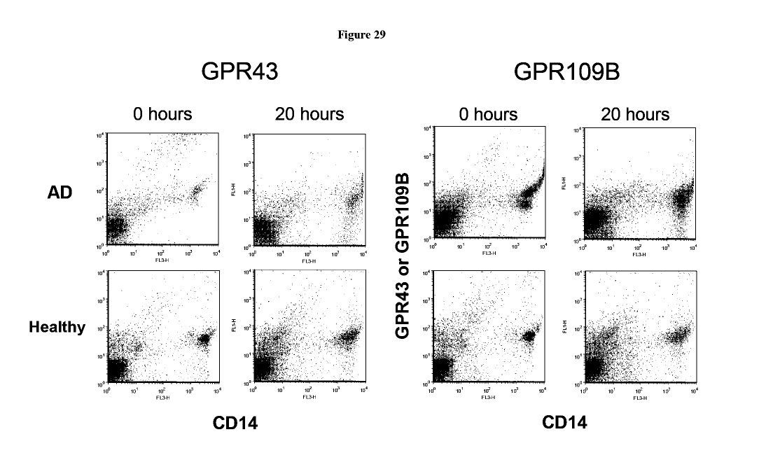

Mononuclear cells were isolated by Percoll gradient centrifugation and stained with antibodies to the cell surface receptors GPR43, GPR109B, and the intercellular protein NBS1. Typical results are presented in FIG. 29. Immediately after isolation GPR43 and GPR109B weakly but positively stain very homogenous population of cells with high CD14 expression. This shows that GPR43 and GPR109B can be detected on the surface of CD16 negative/CD14 positive monocytes. In addition GPR43 and GPR109A were expressed at higher levels on smaller numbers of CD14/CD16 double positive cells. After incubation of cells from an AD patient for 20-24 hours under non-adherent conditions a significant fraction of the GPR43 or GPR109A positive monocytes were now expressing CD16. In healthy individuals CD16+ monocytes were also seen but significantly fewer of the CD14/CD16 double positive cells also express MIFN signature proteins.

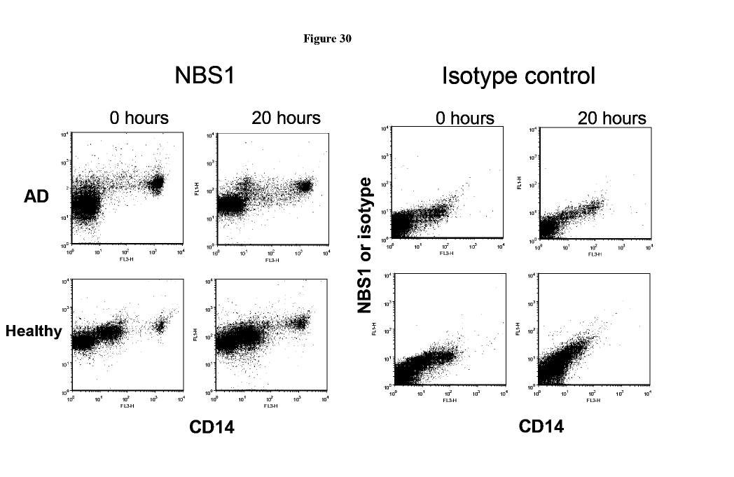

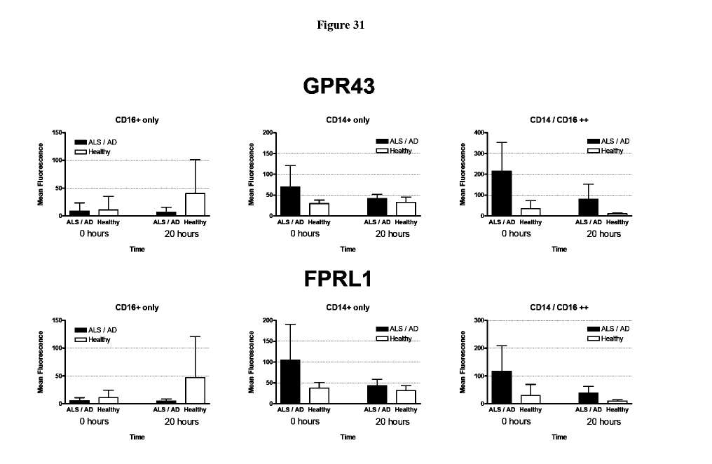

In contrast to GPR43 and GPR109B, the majority of cells of all types exhibit higher staining with antibody to the intercellular MIFN-signature protein NBS1 than seen with an isotype control (FIG. 30). At isolation, CD14+ monocytes were seen to express slightly higher levels of NBS1 than lymphocytes or other CD14 negative cell populations (FIG. 30). There was little to no staining of CD14, CD16 or double positive cells with an isotype control. So as is the case with GPR43 and GPR109B, NBS1 was expressed in the majority of CD14+ monocytes at isolation. After incubation for 20-24 hours CD14+ monocytes from healthy controls were mostly now also expressing CD16, however these double positive cells were relatively deficient of NBS1 staining. In contrast, newly formed CD14/CD16 positive monocytes from the AD patient maintained their expression of NBS1.

To confirm these observations a series of 6 ALS and AD patients and 7 healthy controls were stained with antibodies to CD14, CD16, and GPR43 and the geometric mean fluorescence of the CD14+ monocytes, CD16+ cells, and CD14/16 double positive cells were determined. The results obtained are presented in FIG. 31. It can be seen that CD16 positive cells were essentially negative for GPR43 in both neurodegenerative disease patients and controls, though some healthy controls expressed increased levels of GPR43 in CD16 cells after incubation. In CD14+ monocytes the levels of GPR43 expressed were higher than that seen in CD16+ cells and roughly equivalent, though there was a trend toward higher expression of GPR43 in neurodegenerative disease monocytes. But in CD14/16++ cells the mean fluorescence values at isolation were approximately 6 fold higher (P<0.01) and they remained 8 fold higher after overnight incubation. Similar results were obtained with the MIFN signature protein FPRL1 (although the number of samples evaluated was smaller). Overall both the data on RNA signals and the flow cytometry results indicate that the primary cell type expressing MIFN signature proteins are activated, CD14/CD16 double positive, macrophages.

### EXAMPLE 19

This example demonstrates the use the genes of the MIFN signature in concert with other genes to predict survival of ALS patients. The same sample set was used as described in Example 11.

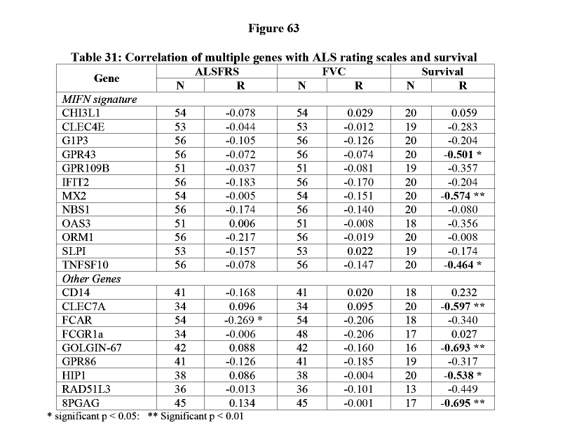

QRT-PCR signals obtained from a number of the genes of the MIFN signature, as well as some other genes associated with myeloid cells were determined and the signals obtained were evaluated for correlation with the current ALS rating scales and survival (see Table 31 (FIG. 63)). None of the MIFN signature genes were significantly correlated with either ALSFRS or FVC. Nor were a collection of other genes including CD14 FCGR1a, GPR86/P2RY13, or GOLGIN67. The one exception noted is that FCAR (also known as CD89) was very weakly anti-correlated (R=−0.269, i.e., as the ALSFRS value goes down FCAR signal increases) with ALSFRS at time of sample draw.

The situation was notably different when QRT-PCR derived signals of these same genes were compared to survival (in days) post provision of sample. For this comparison seven of the genes evaluated including 3 MIFN-signature genes (GPR43, MX2, and TNFSF10) were significantly anti-correlated with survival. Additionally the interferon stimulated gene OAS3 and the cell receptor GPR109B had R values approaching −0.4, suggesting that they were also probably anti-correlated with survival to some extent. However, a number of other MIFN-signature genes including CHI3L1, ORM1, and NBS1 had Rs˜0 and were clearly not at all related to post sample survival. The greatest anti-correlations were seen with the human endogenous retrovirus sequence 8pGAG (R=−0.695) and the intracellular gene GOLGIN-67 (R=−0.693, aka as Golgi autoantigen, golgin subfamily a, 8B). Other non MIFN-signature genes with notable anti-correlations with survival include CLEC7A, a pattern recognition receptor of macrophages and HIP1 or Huntington interacting protein 1.

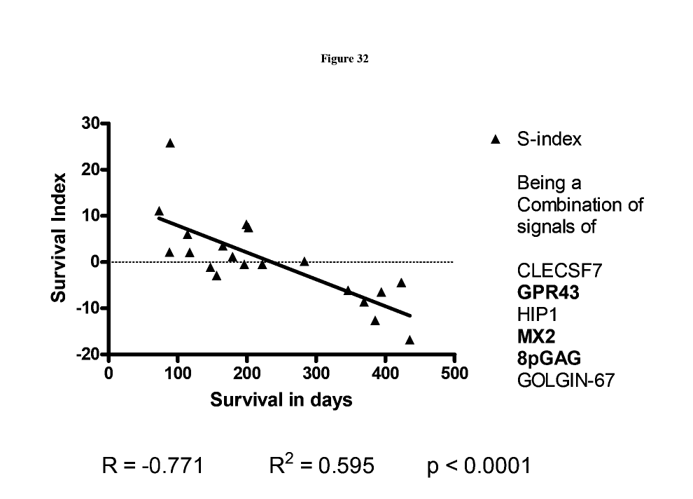

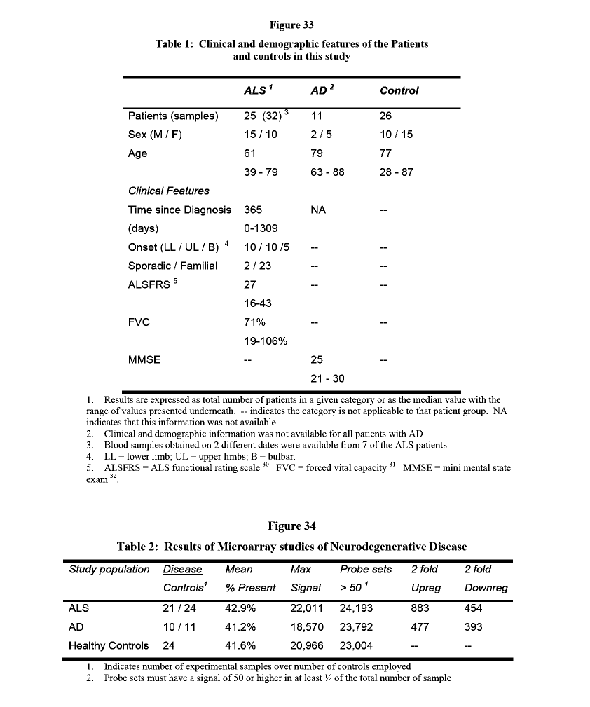

The signals obtained by QRT-PCR from the six genes (CLEC7A, GPR43, GOLGIN-67, HIP1, MX2, and 8pGAG) were combined with the highest anti-correlations with survival so as to generate a useful molecular scale. To facilitate combining signals from genes with very different expression levels (e.g. GPR43 and 8pGAG) the median actin normalized signal for each gene from all ALS patients was determined. Then the median signal was subtracted from the actin normalized signal from each individual sample (i.e., Signal of Sample—Median signal) to get a distance from median value for each sample with each gene. These were then added together to get a survival index. The results obtained are presented in FIG. 32.

As seen in FIG. 32, there is a clear inverse relationship between survival index score and survival post sample provision with a Pearson of R=−0.771 (p<0.0001). This means the lower the signal with the six genes of the index with the particular blood sample, the longer the individual was likely to survive beyond that date. This demonstrates that the genes of the MIFN signature (e.g., GPR43 and MX2, as used in this study) can be used in concert with other genes to predict survival of ALS patients. Monitoring of this type could be very useful in the performance of clinical trials on candidate drugs for ALS or for the counseling of patients.

### REFERENCES CITED

1. McGrath M. S., Shiramizu B., Herndier B. G. Clonal HIV in the pathogenesis of AIDS-related lymphoma: Sequential pathogenesis. In: Infections Causes of Cancer: Targets for intervention. James Goedert, ed. (Totowa, N.J.: Humana Press), pp. 231-242; 2000.
2. Zenger E., Abbey N. W., Weinstein M. D., Kapp L., Reis J., Gofman I., Millward C., Gascon R., Elbaggari A., Herndier B. G., McGrath M. S. Injection of human primary effusion lymphoma cells or associated macrophages into SCID mice causes murine lymphomas. Cancer Research, 62:5536-5542, 2002.
3. Minagar A, Shapshak P, Fujimura R, Ownby R, Heyes M, Eisdorfer C. The role of macrophage/microglia and astrocytes in the pathogenesis of three neurologic disorders: HIV-associated dementia, Alzheimer's disease, and multiple sclerosis. J. Neurol Sciences 202:13-23, 2002
4. McGeer P L, McGeer EG. Inflammatory processes in amyotrophic lateral sclerosis. Muscle Nerve. 26:459-470, 2002.
5. Schwartz M. Macrophages and microglia in central nervous system injury: Are they helpful or harmful? J Cerebral Blood Flow & Metab. 23:385-394, 2003.
6. Atamas S P, White B. Cytokine regulation of pulmonary fibrosis in scleroderma. Cytokine & Growth Factor Reviews. 14:537-550, 2003.
7. Cousins S W, Espinosa-Heidmann D G, Csaky K G. Monocyte activation in patients with age-related macular degeneration: a biomarker of risk for choroidal neovascularization? Arch Opthalmol. 122:1013-1018, 2004.
8. Mitchell J. D. Guidelines in motor neurone disease (MND/amyotrophic lateral sclerosis (ALS)—from diagnosis to patient care. J Neurol. 247(Suppl 6):VI/7-V/I12, 2000.
9. Miller, R., Mitchell, J. and Moore, D. Riluzole for amyotrophic lateral sclerosis (ALS)/motor neuron disease (MND) (Cochrane Review). Cochrane Library, Oxford. Issue 1, 2004. & ALS & Other Motor Neuron Disords. 2003; 191-206.
10. Hand C. K., Rouleau G. A. Familial amyotrophic lateral sclerosis. Muscle Nerve 25:135-159, 2002.
11. Tu P-H., Raju P., Robinson K. A., Gurney M. E., et al. Transgenic mice carrying a human mutant superoxide dismutase transgene develop neuronal cytoskeletal pathology resembling human amyotrophic lateral sclerosis lesions. Proc. Natl. Acad. Sci. USA 93:3155-3160, 1996.

12. Alexianu M. E., Kozovska M., Appel S. H. Immune reactivity in a mouse model of famial ALS correlates with disease progression. Neurology 57:1282-1289, 2001.
13. Bruijn L I, Miller T M, Cleveland D W. Unraveling the mechanisms involved in motor neuron degeneration in ALS. Ann Rev. Neurosci. 27:723-749, 2004.
14. Kawamata T., Akiyama H., Yamada T., McGeer P. L. Immunologic reactions in amyotrophic lateral sclerosis brain and spinal cord tissue. Am J Pathol 140:691-707, 1992.
15. Wilms H, Sievers J, Dengler R, Bufler J, Deuschl G, Lucius R. Intrathecal syntesis of monocyte chemoattractant protein-1 (MCP-1) in amyotrophic lateral sclerosis: further evidence for microglial activation in neurodegeneration. J Neuroimmunol. 144:139-142, 2003.
16. Henkel J S, Engelhardt J I, Siklos L, Simpson E P, Kim S H, Pan T, Goodman J C, Siddique T, Beers D R, Appel S H. Presence of dendritic cells, MCP-1, and activated microglia/macrophages in amyotrophic lateral sclerosis spinal cord tissue. Ann Neurol 55:221-235, 2004.
17. Almer G, Teismann P, Stevic Z, Halaschek-Wiener J, Deecke L, Kostic V, Przedborski S. Increased levels of the pro-inflammatory prostaglandin PGE2 in CSF from ALS patients. Neurology 58:1277-1279, 2002.
18. Pramatarova A, Laganiere J, Roussel J, Brisebois K, Rouleau G A. Neuron-Specific expression of mutant superoxide dismutase 1 in transgenic mice does not lead to motor impairment. J Neurosci 21:3369-3374, 2001.
19. Nguyen M D, D'Aigle T, Gowing G, Julien J-P, Rivest S. Exacerbation of motor neuron disease by chronic stimulation of innate immunity in a mouse model of amyotrophic lateral sclerosis. J. Neurosci 24:1340-1349, 2004.
20. Zhang R, Gascon R, Miller R G, Gelinas D F, Mass J, Hadlock K, Jin X, Reis J, Narvaez A, McGrath M S. Evidence for systemic immune system alterations in sporadic amyotrophic lateral sclerosis (sALS). J Neuroimmunol. 159:215-224, 2005.
21. Desport J C, Preux P M, Magy L, Boirie Y, Vallat J M, Beaufrere B, Couratier P. Factors correlated with hypermetabolism in patients with amyotrophic lateral sclerosis. Am J Clin Nutr. 74:328-334, 2001.
22. Dupuis L, Oudart H, Rene F, Aguilar J-L G, Loeffler J-P. Evidence for defective energy homeostasis in amyotrophic lateral sclerosis: Benefit of a high-energy diet in a transgenic mouse model. Proc. Natl. Acad. Sci. USA. 101:11159-11164, 2004.
23. Ono S, Toyokura Y, Mannen T, Ishibashi Y. Amyotrophic lateral sclerosis: histologic, histochemical, and ultrastructural abnormalities of skin. Neurology 36:948-56, 1986.
24. Kolde G, Bachus R, Ludolph A C. Skin involvement in amyotrophic lateral sclerosis. Lancet 347:1226-1227, 1996.
25. Lomen-Hoerth C, Murphy J, Langmore S, Kramer J H, Olnev R K, Miller B. Are amyotrophic lateral sclerosis patients cognitively normal? Neurology 60:1094-1097, 2003.
26. Affymetrix Corporation, Santa Clara, Calif. www.affymetrix.com
27. www.affymetrix.com/support/technical/mask_files.affx
28. Microsoft Corporation, Redmond Wash. www.microsoft.com
29. Agilent Technologies, Santa Clara, Calif. www.chem.agilent.com/scripts/pds.asp?1page=27881
30. Gordon P H, Miller R G, Moore D H. ALSFRS-R ALS other motor neuron disorders 5(Suppl 1):90-93, 2004
31. Lechtzin N, Rothstein J, Clawson L, Diette G B, Wiener C M. Amyotrophic lateral sclerosis: evaluation and treatment of respiratory impairment. ALS other motor neuron disorders 3:5-13, 2002
32. Malloy P F, Cummings J L, Coffey C E, Duffy J, Fink M, Lauterbach E C, Lovell M, Royall D, Salloway S. Cognitive screening instruments in neuropsychiatry: a report of the Committee on Research of the American Neuropsychiatric Association. J Neuropsychiatry Clin Neurosci. 9:189-197, 1997
33. Baechler E C, Batliwalla F M, Karypis G, Gaffney P M, Moser K, Ortmann W A, Espe K J, Balasubramanian S, Hughes K M, Chan J P, Begovich A, Chang S-Y P, Gregersen P K, Behrens T W. Expression levels for many genes in human peripheral blood cells are highly sensitive to ex vivo incubation. Genes and Immunity 5:347-353, 2004.
34. Dangond F, Hwang D, Camelo S, Pasinelli P, Frosch M P, Stephanopoulos F, Brown R H Jr, Gullans S R. Molecular signature of late-stage human ALS revealed by expression profiling of postmortem spinal cord gray matter. Physiol Genomics 16:229-239, 2004.
35. Jiang Y-M, Yamamoto M, Kobayashi Y, Yoshihara T, Liang Y, Terao S, Takeuchi H, Ishigaki S, Katsuno M, Adachi H, Niwa J-I, Tanaka F, Manabu D, Yoshida M, Hashizume Y, Sobue G. Gene expression profile of spinal motor neurons in sporadic amyotrophic lateral sclerosis. Ann Neurol 57:236-251, 2005.
36. Roche Applied Sciences Corporation, Indianapolis, Ind. www.roche-applied-science.com/index jsp
37. Golub T R, Slonim D K, Tamayo P, Huard C, Gaasenbeek M, Mesirov J P, Coller H, Loh M L, Downing J R, Caligiuri M A, Bloomfield C D, Lander E S. Molecular classification of cancer: class discovery and class prediction by gene expression monitoring. Science. 286:531-537, 1999.
38. The ALS CNTF treatment study (ACTS) phase I-II study group. The Amyotrophic Lateral Sclerosis Functional Rating Scale. Assessment of activities of daily living in patients with amyotrophic lateral sclerosis. Arch Neurol. 50:141-147, 1996.
39. S. J. Haidt, R. Gascon, M. McGrath, R. Zhang, W. Stem, M. Wieland, E. Boldrey, J. Palmer, L. Borrillo Evidence for Systemic Immunologic Activation Parameters That Distinguish Wet and Dry Forms of ARMD. ARVO 2005 annual meeting, Poster 112
40. Coclet-Ninin J, Dayer J M, Burger D. Interferon-beta not only inhibits interleukin-1 beta and tumor necrosis factor-alpha but simulates interleukin-1 receptor antagonist production in human peripheral blood mononuclear cells. Eur Cytokine Netw. 8:345-349, 1997.
41. Der S D, Zhou A, William B R G, Silverman R H. Identification of genes differentially regulated by interferon [symbol] .□, or □ using Oligonucleotide arrays. Proc Natl Acad Sci USA 95:15623-15628, 1998
42. Cole K E, Strick C A, Paradis T J, Ogborne K T, Loetscher M, Gladue R P, Lin W, Boyd J G, Moser B, Wood D E, Sahagan B G, Neote K. Interferon-inducible T cell alpha chemoattractant (1-TAC): A novel non-ELR CXC chemokine with potent activity on activated T cells through selective high affinity binding to CXCR3. J Exp Med. 187:2009-2021, 1998
43. Griffith T S, Wiley S R, Kubin M Z, Sedger L M, Maliszewski C R, Fanger N A. Monocyte-mediaged tumorcidal activity via the tumor necrosis factor related cytokine TRAIL. J Exp Med. 189:1343-1353, 1999.
44. Justesen J, Hartmann R, Kjeldgaard N O. Gene structure and function of the 2′-5′ oligoadenylate synthetase family. Cell Mol Life Sci 57:1593-1612, 2000
45. Monteiro R C, Kubagawa J, Cooper M D. Cellular distribution, regulation, and biochemical nature of an Fc□ receptor in humans. J Exp Med. 171:597-613, 1990.
46. Durstin M, Gao J L, Tiffany H L, McDermott D, Murphy P M. Differential expression of numbers of the N-formylpeptide receptor gene cluster in human phagocytes. Biochem Biophys Res Commun. 201:174-179, 1994.
47. LePoul E, Loison C, Struyf S, Springael J-Y, Lannoy V, Decobecq M-E, Bresillon S, Dupriez V, Vassart G, Damme J V, Parmentier M, Detheux M. Functional characterization of human receptors for short chain fatty acids and their role in polymorphonuclear cell activation. J Biol Chem 278:25481-25489, 2003.
48. Svensson P-A, Hagg D A, Jernas M, Englund M C O Hulten L-M, Ohlsson B G, Hulthe J, Wiklund O, Carlsson B, Fagerberg B, Carlsson L M S. Identification of genes predominantly expressed in human macrophages. Atherosclerosis 177:287-290, 2004.
49. Nonomura K, Yamanishi K, Yasuno H, Nara K, Hirose S. Up-regulation of elafin/SKALP gene expression in psoriatic epidermis. J Invest Dermatol 103:88-91, 1994
50. Vidal R, Calero M, Revesz T, Plant G, Ghiso J, Frangione B. Sequence, genomic structure, and tissue expression of human BRI3, a member of the BRI gene family. Gene 266:95-102, 2001.
51. Van't Veer L, Dai H, van de Jijver M J, He Y D, Hart A A M, Mao M, Peterse H L, Kooy K vd, Marton M J, Witteveen A T, Schreiber G J, Kerkhoven R M, Roberts C, Linsley P S, Bernards R, Friend S H. Gene expression profiling predicts clinical outcome of breast cancer. Nature 415:530-536, 2002.
52. Dave S S, Wright G, Tan B, Rosenwald A, Gascoyne R D, Chan W C, Fisher R I, Braziel R M, Rimsza L M, Grogan T M, Miller T P, LeBlanc M, Greiner T C, Weisenburger D D, Lynch J C, Vose J, Armitage J O, Smeland E B, Kvaloy S, Holte H, Delabie J, Connors J M, Lansdorp P M, Quyang Q, Lister T A, Davies A J, Norton A J, Muller-Hermelink K, Ott G, Campo E, Montserrat E, Wilson W H, Jaffe E S, Simon R, Yang L, Powell J, Zhao H, Goldschmidt N, Chiorazzi B A, Staudt L M. Prediction of survival in flollicular lymphoma based on molecular features of tumor infiltrating immune cells. N Eng J. Med 351:2159-2169, 2004.
53. McGrath M S, Kahn J O, Herndier B G. Development of WF10, a novel macrophage-regulating agent. Curr Opin Investig Drugs. 3:365-373, 2002.
54. Veerasarn V, Khorprasert C, Lorvidhaya V, Sangruchi S, Tantivatana T, Narkwong L, Kongthanarat Y, Chitapanarux I, Tesavibul C, Panichevaluk A, Puribhat S, Sangkittipaiboon S, Sookpreedee L, Lertsanguansinchai P, Phromratanapongse P, Rungpoka P, Trithratipvikul S, Lojanapiwat B, Ruangdilokrat S, Ngampanprasert P. Reduced recurrence of late hemorrhagic radiation cystitis by WF10 therapy in cervical cancer patients: a multicenter, randomized, two-arm, open-label trial. Radiother Oncol. 73:179-185, 2004.
55. Dimethaid Research Inc. Markham, Ontario, Canada. www.dimethaid.com
56. Kaczmarek L, Kaminska B, Messina L, Spampinato G, Arcidiacono A, Malaguamera L, Messina A. Inhibitors of polyamine biosynthesis block tumor necrosis factor-induced activation of macrophages. Cancer Res. 52:1891-1894, 1992.
57. Bianchi M, Bloom 0, Raabe T, Cohen P S, Chesney J, Sherry B, Schmidtmayerova H, Calandra T, Zhang X, Bukrinsky M, Ulrich P, Cerami A, Tracey K J. Suppression of proinflammatory cytokines in monocytes by a tetravalent guanylhydrazone. J Exp Med 183:927-936, 1996.
58. Reddy V. K., Valasinas A., Sarkar A., Basu H., Marton L. J., Frydman B. Conformationally restricted analogs of 1N, 12N-bisethylspermine: synthesis and growth inhibitory effects on human tumor cell lines. J Med Chem 41:4723-4732, 1998.
59. Valasinas A., Sarkar A., Reddy V. K., Marton L. J., Basu H. S., Frydman B. Conformationally restricted analogues of 1N,14N-bisethylhomospermine (BE-4-4-4): synthesis and growth inhibitory effects on human prostate cancer cells. J Med Chem 44:390-403, 2001
60. Levine A M, Tulpule A, Tessman D, Kaplan L, Giles F, Luskey B D, Scadden D T, Northfelt D W, Silverberg I, Wernz J, Espina B, Von Hoff D. Mitoguazone therapy in patients with refractory or relapsed AIDS-related lymphoma: results from a multicenter phase II trial. J Clin Oncol. 15:1094-1103, 1997.
61. Atkins M B, Redman B, Mier J, Gollob J, Weber J, Sosman J, MacPherson B L, Plasse T. A phase I study of CNI-1493, an inhibitor of cytokine release, in combination with high-dose interleukin-2 in patients with renal cancer and melanoma. Clin Cancer Res. 7:486-492, 2001
62. Lownberg M, Verhaar A, Blink B vd, Kate F T, Veventer S V, Peppelenbosch M, Hommes D. Specific inhibition of c-Raf activity by Semapimod induces clinical remission in severe Crohn's disease. J. Immunol. 175:2293-2300, 2005.
63. Harlow E. and Lane D. Using Antibodies: A laboratory manual. Cold Spring Harbor Laboratory Press. Cold Spring Harbor, N.Y., 1999.

1. A method for diagnosing a neurodegenerative disorder in a subject comprising detecting the expression of a panel of ProMac signature genes in a biological sample of the subject, wherein a higher than normal level of expression of the panel of ProMac signature genes is indicative of a neurodegenerative disorder in the subject.

2. The method of claim 1, wherein the expression of the panel of ProMac signature genes includes transcription, translation, or activation of the panel of ProMac signature genes.

3. The method of claim 1, wherein the panel of ProMac signature genes comprise at least two ProMac signature genes.

4. The method of claim 1, wherein the panel of ProMac signature genes comprise at least four ProMac signature genes.

5. The method of claim 1, wherein the panel of ProMac signature genes comprise at least five ProMac signature genes.

6. The method of claim 1, wherein the panel of ProMac signature genes comprise at least eight ProMac signature genes.

7. The method of claim 1, wherein the panel of ProMac signature genes are selected from the group consisting of the genes listed in Table 28.

8. The method of claim 1, wherein the panel of ProMac signature genes are selected from the group consisting of genes listed in Table 21.

9. The method of claim 1, wherein the panel of ProMac signature genes are selected from the group consisting of genes listed in Table 29.

10. The method of claim 1, wherein the panel of ProMac signature genes are selected from the group consisting of CLEC4E, G1P3, GPR109B, IFIT2, IL1RN, MX2, NBS1, and ORM1.

11. The method of claim 1, wherein the panel of ProMac signature genes are selected from the group consisting of G1P3, GPR43, IFIT2, ORM1, and TNFSF10.

12. The method of claim 1, further comprising detecting the expression of a panel of ProMac secondary signature genes, wherein a higher than normal level of expression of the panel of ProMac signature genes and ProMac secondary signature genes is indicative of a neurodegenerative disorder in the subject.

13. The method of claim 12, wherein the panel of ProMac secondary signature genes are selected from the group consisting of genes listed in Table 30.

14. The method of claim 12, wherein the panel of ProMac secondary signature genes comprise at least two ProMac secondary signature genes.

15. The method of claim 12, wherein the panel of ProMac secondary signature genes are selected from the group consisting of ALAS2, BTNL8, CKLFSF2, CR1L, CSF3R, FCAR, FCGR3B, GMPB, IF127, IL8RA, IL8RB, JAG1, KCNJ15, P2RY13, PBEF1, PLAU, PLXNC1, SLENBP1, SLC25A37, and TNFRSF10C.

16. The method of claim 1, wherein the neurodegenerative disorder is selected from the group consisting of amyotrophic lateral sclerosis (ALS), Charcot-Marie Tooth syndrome, Alzheimer's disease (AD), HIV-associated dementia (HAD), HIV associated neurological disorders, peripheral sensory neuropathy, diabetic neuropathy, autism, Parkinson's disease, schizophrenia, and multiple sclerosis.

17. A kit comprising one or more probes useful for detecting the expression of a panel of ProMac signature genes in a sample from a subject.

18. The kit of claim 17, wherein the probes are oligonucleotides.

19. The kit of claim 17, wherein the probes are antibodies.

20. The kit of claim 17, wherein the panel of ProMac signature genes comprise at least two ProMac signature genes.

21. The kit of claim 17, wherein the panel of ProMac signature genes comprise at least four ProMac signature genes.

22. The kit of claim 17, wherein the panel of ProMac signature genes comprise at least five ProMac signature genes.

23. The kit of claim 17, wherein the panel of ProMac signature genes comprise at least eight ProMac signature genes.

24. The kit of claim 17, wherein the panel of ProMac signature genes are selected from the group consisting of the genes listed in Table 28.

25. The kit of claim 17, wherein the panel of ProMac signature genes are selected from the group consisting of genes listed in Table 21.

26. The kit of claim 17, wherein the panel of ProMac signature genes are selected from the group consisting of genes listed in Table 29.

27. The kit of claim 17, wherein the panel of ProMac signature genes are selected from the group consisting of CLEC4E, G1P3, GPR109B, IFIT2, IL1RN, MX2, NBS1, and ORM1.

28. The kit of claim 17, wherein the panel of ProMac signature genes are selected from the group consisting of G1P3, GPR43, IFIT2, ORM1, and TNFSF10.

29. A method for distinguishing a first neurodegenerative disorder from a second neurodegenerative disorder comprising
evaluating the expression of a panel of ProMac secondary signature genesassociated with the first and the second neurodegenerative disorder in a biological sample from the subject, and
correlating the expression of the panel of ProMac secondary signature genes with the determination of the first neurodegenerative disorder or the second neurodegenerative disorder,
wherein the first neurodegenerative disorder is cerebral neuron degeneration and the second neurodegenerative disorder is motor neuron degeneration.

30. The method of claim 29, wherein the first neurodegenerative disorder is Alzheimer's disease (AD) and the second neurodegenerative disorder is amyotrophic lateral sclerosis (ALS).

31. The method of claim 29, wherein a higher than normal level of expression of the panel of ProMac secondary signature genes is indicative of the first neurodegenerative disorder.

32. The method of claim 29, wherein the panel of ProMac secondary signature genes are selected from the group consisting of 8pGAG, CSF3R, GOLGIN-67, IL6, JAG1, MSP, RAD51L3, and TPD52.

33. The method of claim 29 further comprising evaluating the expression of a panel of ProMac signature genes.

34. The method of claim 33, wherein the panel of ProMac signature genes are selected from the group consisting of CHI3L1, CXCL1L, GPR43, ILRN, ORM1, and PI3.

35. A method for monitoring the treatment of a neurodegenerative disease in a subject comprising monitoring the expression of a panel of ProMac signature genes in a biological sample from the subject, wherein the level of expression of the panel of ProMac signature genes positively correlates with the progress of the neurodegenerative disease in the subject.

36. A method for monitoring the treatment of a ProMac associated disease in a subject comprising monitoring the expression of a panel of ProMac signature genes in a biological sample from the subject, wherein the level of expression of the panel of ProMac signature genes positively correlates with the progress of the ProMac associated disease in the subject.

37. A method for monitoring the level of disease associated macrophages in a subject comprising monitoring the expression of a panel of ProMac signature genes in a biological sample from the subject, wherein the level of expression of the panel of ProMac signature genes positively correlates with the level of disease associated macrophages in the subject.

38. A method for evaluating an agent comprising contacting the agent with a macrophage and evaluating the expression of a panel of ProMac signature genes in the presence and absence of the agent, wherein a change caused by the agent is indicative of the agent as a modulator of ProMac.

39. A method for providing a prognosis of a ProMac associated disease in a subject comprising detecting the expression of a panel of ProMac signature genes in a biological sample from the subject, wherein the expression of the panel of ProMac signature genes is negatively associated with a positive outcome of the ProMac associated disease.

40. The method of claim 39, wherein the ProMac associated disease is a neurodegenerative disorder.

41. The method of claim 39 further comprising detecting the expression of a panel of ProMac secondary signature genes.

42. The method of claim 41, wherein the secondary signature genes are selected from the group consisting of CD14, CLEC7A, FCAR, FCGR1a, GOLGIN-67, GPR86, HIP1, RAD51L3, and 8PGAG.

43. The method of claim 39, wherein the panel of ProMac signature genes are selected from the group consisting of CHI3L1, CLEC4E, G1P3, GPR43, GPR109B, IFIT2, MX2, NBS1, OAS3, ORM1, SLPI, and TNFSF10.

44. A method for providing a prognosis of a ProMac associated disease in a subject comprising detecting the expression of a panel of ProMac secondary signature genes in a biological sample from the subject, wherein the expression of the panel of ProMac secondary signature genes is negatively associated with a positive outcome of the ProMac associated disease.

45. The method of claim 44, wherein the ProMac secondary signature genes are selected from the group consisting of CD14, CLEC7A, FCAR, FCGR1a, GOLGIN-67, GPR86, HIP1, RAD51L3, and 8PGAG.

46. The method of claim 44, wherein the ProMac associated disease is amyotrophic lateral sclerosis (ALS).


SEQUENCE LISTING (222 sequences)

SEQ ID NO: 1 - DNA, 22 bp, Homo sapiens;
gggtgaaaggaccaaaaacaga

SEQ ID NO: 2 - DNA, 25 bp, Homo sapiens;
cacaacagaatagttgtaagcatca

SEQ ID NO: 3 - DNA, 21 bp, Homo sapiens;
agcagcgtcgtcataggtaat

SEQ ID NO: 4 - DNA, 21 bp, Homo sapiens;
acaggaggatcacttgaggct

SEQ ID NO: 5 - DNA, 21 bp, Homo sapiens;
tgctacgagaacttcaccgat

SEQ ID NO: 6 - DNA, 20 bp, Homo sapiens;
gaagcacaccaggaaattga

SEQ ID NO: 7 - DNA, 25 bp, Homo sapiens;
cagtaaagagcttactcctgtagcg

SEQ ID NO: 8 - DNA, 22 bp, Homo sapiens;
aagcctcagaatctgctccatt

SEQ ID NO: 9 - DNA, 25 bp, Homo sapiens;
agaccttctatctgaggaacaacca

SEQ ID NO: 10 - DNA, 21 bp, Homo sapiens;
ttgtcctgctttctgttctcg

SEQ ID NO: 11 - DNA, 22 bp, Homo sapiens;
tgaatgaggagttctggtacct

SEQ ID NO: 12 - DNA, 24 bp, Homo sapiens;
agtcaagccacagactaggtgtaa

SEQ ID NO: 13 - DNA, 20 bp, Homo sapiens;
tcaccctaaaaacacccaca

SEQ ID NO: 14 - DNA, 20 bp, Homo sapiens;
ttttccccagagtcttggaa

SEQ ID NO: 15 - DNA, 21 bp, Homo sapiens;
cgacaggaccagtgcatctat

SEQ ID NO: 16 - DNA, 20 bp, Homo sapiens;
aactctcccagttgctcctt

SEQ ID NO: 17 - DNA, 21 bp, Homo sapiens;
atggccttagctcttagccaa

SEQ ID NO: 18 - DNA, 20 bp, Homo sapiens;
gctcttgcgctttgacttta

SEQ ID NO: 19 - DNA, 25 bp, Homo sapiens;
ctacctcatatcagtttgctagcag

SEQ ID NO: 20 - DNA, 22 bp, Homo sapiens;
cgatcttttagtggtgcctctt

SEQ ID NO: 21 - DNA, 20 bp, Homo sapiens;
taagggttacctgggttgcc

SEQ ID NO: 22 - DNA, 24 bp, Homo sapiens;
gaagatgtcaaactcactcatggc

SEQ ID NO: 23 - DNA, 19 bp, Homo sapiens;
atgcaatcaatgccccagt

SEQ ID NO: 24 - DNA, 21 bp, Homo sapiens;
gttagctgcagattcttgggt

SEQ ID NO: 25 - DNA, 24 bp, Homo sapiens;
cccactgcatagaaataacaagcg

SEQ ID NO: 26 - DNA, 27 bp, Homo sapiens;
cctgaaattccaacaatagagacaggc

SEQ ID NO: 27 - DNA, 21 bp, Homo sapiens;
acgagttctttcgttctgtgc

SEQ ID NO: 28 - DNA, 19 bp, Homo sapiens;
agaggctgccctcaacagt

SEQ ID NO: 29 - DNA, 21 bp, Homo sapiens;
tggatggaacagactatgcga

SEQ ID NO: 30 - DNA, 19 bp, Homo sapiens;
atttctgtggccgggtggt

SEQ ID NO: 31 - DNA, 21 bp, Homo sapiens;
ttcctctgttcgcgactagtt

SEQ ID NO: 32 - DNA, 20 bp, Homo sapiens;
agcaacacccttttcaggat

SEQ ID NO: 33 - DNA, 19 bp, Homo sapiens;
tcaccttcctgggcatctt

SEQ ID NO: 34 - DNA, 20 bp, Homo sapiens;
tcccacctcgctttacaaaa

SEQ ID NO: 35 - DNA, 20 bp, Homo sapiens;
acaccacaccctgctgcttt

SEQ ID NO: 36 - DNA, 21 bp, Homo sapiens;
taggacaagagcaagcagaaa

SEQ ID NO: 37 - DNA, 21 bp, Homo sapiens;
tcctgatctctctcgctgtga

SEQ ID NO: 38 - DNA, 20 bp, Homo sapiens;
taaagggtacagcagccacc

SEQ ID NO: 39 - DNA, 21 bp, Homo sapiens;
gcctggagatgaaatcttgca

SEQ ID NO: 40 - DNA, 21 bp, Homo sapiens;
tgagaatcagcggttatggga

SEQ ID NO: 41 - DNA, 21 bp, Homo sapiens;
acagagacctccctgttcaag

SEQ ID NO: 42 - DNA, 19 bp, Homo sapiens;
tggagaaggagccccagat

SEQ ID NO: 43 - DNA, 24 bp, Homo sapiens;
caaaaagctgatgtatagccctgt

SEQ ID NO: 44 - DNA, 23 bp, Homo sapiens;
cacttacaccaacaaatgcagac

SEQ ID NO: 45 - DNA, 24 bp, Homo sapiens;
ctcaatattcatctcccacatcac

SEQ ID NO: 46 - DNA, 25 bp, Homo sapiens;
accctactgcctagtagcttgacaa

SEQ ID NO: 47 - DNA, 21 bp, Homo sapiens;
agcagcgtcgtcataggtaat

SEQ ID NO: 48 - DNA, 21 bp, Homo sapiens;
acaggaggatcacttgaggct

SEQ ID NO: 49 - DNA, 21 bp, Homo sapiens;
atggccttagctcttagccaa

SEQ ID NO: 50 - DNA, 20 bp, Homo sapiens;
gctcttgcgctttgacttta

SEQ ID NO: 51 - DNA, 23 bp, Homo sapiens;
ttgatattcccaccatctatcag

SEQ ID NO: 52 - DNA, 25 bp, Homo sapiens;
tggtttctgtaagtaattcctcaca

SEQ ID NO: 53 - DNA, 21 bp, Homo sapiens;
gccatctgatgagcagttgaa

SEQ ID NO: 54 - DNA, 21 bp, Homo sapiens;
gctgtaggtgctgtccttgct

SEQ ID NO: 55 - DNA, 18 bp, Homo sapiens;
agatagcagccccgtcaa

SEQ ID NO: 56 - DNA, 21 bp, Homo sapiens;
actgtcttctccacggtgctc

SEQ ID NO: 57 - DNA, 20 bp, Homo sapiens;
agaaaccatcgtgctgaggt

SEQ ID NO: 58 - DNA, 20 bp, Homo sapiens;
tgaagagctgggagcttgga

SEQ ID NO: 59 - DNA, 19 bp, Homo sapiens;
ccagcatctgcaaagctcc

SEQ ID NO: 60 - DNA, 20 bp, Homo sapiens;
ttgtacagggccaggacctt

SEQ ID NO: 61 - DNA, 21 bp, Homo sapiens;
tctacaacaaacccaccctct

SEQ ID NO: 62 - DNA, 19 bp, Homo sapiens;
tgagcatccacctgtggct

SEQ ID NO: 63 - DNA, 19 bp, Homo sapiens;
tgttcggcctcctgctgtt

SEQ ID NO: 64 - DNA, 21 bp, Homo sapiens;
gcttcttttcatcctcctcca

SEQ ID NO: 65 - DNA, 20 bp, Homo sapiens;
atccggtttgtcattggctt

SEQ ID NO: 66 - DNA, 25 bp, Homo sapiens;
tgtacttgccatagaacaacatctc

SEQ ID NO: 67 - DNA, 21 bp, Homo sapiens;
ttggctgttcctgcagtagaa

SEQ ID NO: 68 - DNA, 21 bp, Homo sapiens;
aacccagaagatgatgctcac

SEQ ID NO: 69 - DNA, 21 bp, Homo sapiens;
tgctacgagaacttcaccgat

SEQ ID NO: 70 - DNA, 20 bp, Homo sapiens;
gaagcacaccaggaaattga

SEQ ID NO: 71 - DNA, 20 bp, Homo sapiens;
aaacagcggtcgagagacaa

SEQ ID NO: 72 - DNA, 21 bp, Homo sapiens;
attggagtggtcctgagtgtg

SEQ ID NO: 73 - DNA, 23 bp, Homo sapiens;
cccattctctagacaaaatcaaa

SEQ ID NO: 74 - DNA, 22 bp, Homo sapiens;
tcttgtcgatcctaagccatga

SEQ ID NO: 75 - DNA, 25 bp, Homo sapiens;
agaccttctatctgaggaacaacca

SEQ ID NO: 76 - DNA, 21 bp, Homo sapiens;
ttgtcctgctttctgttctcg

SEQ ID NO: 77 - DNA, 20 bp, Homo sapiens;
tcttccctgaccttcacgaa

SEQ ID NO: 78 - DNA, 25 bp, Homo sapiens;
catttattaagcagttacaatgctg

SEQ ID NO: 79 - DNA, 22 bp, Homo sapiens;
tccctcataaccaaagcttcag

SEQ ID NO: 80 - DNA, 23 bp, Homo sapiens;
ggctgacttgcatatgttaaaaa

SEQ ID NO: 81 - DNA, 20 bp, Homo sapiens;
tcaccctaaaaacacccaca

SEQ ID NO: 82 - DNA, 20 bp, Homo sapiens;
ttttccccagagtcttggaa

SEQ ID NO: 83 - DNA, 23 bp, Homo sapiens;
gaaaatgaaacacggatcctcag

SEQ ID NO: 84 - DNA, 25 bp, Homo sapiens;
gagagggtagtagaaattcacttgc

SEQ ID NO: 85 - DNA, 25 bp, Homo sapiens;
ctacctcatatcagtttgctagcag

SEQ ID NO: 86 - DNA, 22 bp, Homo sapiens;
cgatcttttagtggtgcctctt

SEQ ID NO: 87 - DNA, 19 bp, Homo sapiens;
aatgcatccctgctgatcc

SEQ ID NO: 88 - DNA, 20 bp, Homo sapiens;
tgagatcacgctggtcgctt

SEQ ID NO: 89 - DNA, 18 bp, Homo sapiens;
gtccctacccttctgcac

SEQ ID NO: 90 - DNA, 23 bp, Homo sapiens;
ggaaaaggctcaaatgtagctat

SEQ ID NO: 91 - DNA, 21 bp, Homo sapiens;
atagcacagctcctgattatg

SEQ ID NO: 92 - DNA, 22 bp, Homo sapiens;
ggctttaccaactccaactatg

SEQ ID NO: 93 - DNA, 23 bp, Homo sapiens;
ttgcccatggtttccagaacaag

SEQ ID NO: 94 - DNA, 23 bp, Homo sapiens;
gggattttttccttgtgttttca

SEQ ID NO: 95 - DNA, 26 bp, Homo sapiens;
gggaagatcaagtctaaatctaaaag

SEQ ID NO: 96 - DNA, 21 bp, Homo sapiens;
gttggatcagtgcttccaagc

SEQ ID NO: 97 - DNA, 19 bp, Homo sapiens;
agtatggatctcaggcggt

SEQ ID NO: 98 - DNA, 19 bp, Homo sapiens;
catcggttgtaacattacc

SEQ ID NO: 99 - DNA, 19 bp, Homo sapiens;
gccaggattttacattgtc

SEQ ID NO: 100 - DNA, 22 bp, Homo sapiens;
ctgtacagagagactggaatag

SEQ ID NO: 101 - DNA, 21 bp, Homo sapiens;
ttttgggatacgagtctccat

SEQ ID NO: 102 - DNA, 19 bp, Homo sapiens;
catcggctcaccttggtta

SEQ ID NO: 103 - DNA, 24 bp, Homo sapiens;
caacacaagaattgaagctgtaaa

SEQ ID NO: 104 - DNA, 23 bp, Homo sapiens;
agcatgagctcagctatctggtt

SEQ ID NO: 105 - DNA, 20 bp, Homo sapiens;
gaggtggtgttcccactgat

SEQ ID NO: 106 - DNA, 20 bp, Homo sapiens;
tttgtgcagccaaaaccaga

SEQ ID NO: 107 - DNA, 18 bp, Homo sapiens;
tttttttcccctgtcccc

SEQ ID NO: 108 - DNA, 25 bp, Homo sapiens;
tggtgtcattgagtaaaaacaaaac

SEQ ID NO: 109 - DNA, 19 bp, Homo sapiens;
cggctataggcacgatgat

SEQ ID NO: 110 - DNA, 25 bp, Homo sapiens;
ctctcctgtgtctgtgttttgtttg

SEQ ID NO: 111 - DNA, 25 bp, Homo sapiens;
ctagtcaagtggaagctaattcaac

SEQ ID NO: 112 - DNA, 24 bp, Homo sapiens;
tgtgagagttggtcaaaagatcac

SEQ ID NO: 113 - DNA, 25 bp, Homo sapiens;
cagtaaagagcttactcctgtagcg

SEQ ID NO: 114 - DNA, 22 bp, Homo sapiens;
aagcctcagaatctgctccatt

SEQ ID NO: 115 - DNA, 20 bp, Homo sapiens;
gactttcccatgcctttcat

SEQ ID NO: 116 - DNA, 21 bp, Homo sapiens;
accggaatctgtagtgcccta

SEQ ID NO: 117 - DNA, 21 bp, Homo sapiens;
cgacaggaccagtgcatctat

SEQ ID NO: 118 - DNA, 20 bp, Homo sapiens;
aactctcccagttgctcctt

SEQ ID NO: 119 - DNA, 22 bp, Homo sapiens;
gggtgaaaggaccaaaaacaga

SEQ ID NO: 120 - DNA, 25 bp, Homo sapiens;
cacaacagaatagttgtaagcatca

SEQ ID NO: 121 - DNA, 19 bp, Homo sapiens;
ttttcccgatattcgggca

SEQ ID NO: 122 - DNA, 24 bp, Homo sapiens;
tctcaaagtacttgtgacaggcag

SEQ ID NO: 123 - DNA, 25 bp, Homo sapiens;
ggatagctgttgtttcagagaaagg

SEQ ID NO: 124 - DNA, 25 bp, Homo sapiens;
cttctcacaaatactatatgagggc

SEQ ID NO: 125 - DNA, 21 bp, Homo sapiens;
tggccttaccgaggagatgat

SEQ ID NO: 126 - DNA, 21 bp, Homo sapiens;
tataggacgttttgctgcagg

SEQ ID NO: 127 - DNA, 25 bp, Homo sapiens;
tgctaatttatctagtgcctttacc

SEQ ID NO: 128 - DNA, 19 bp, Homo sapiens;
gagtgcagtggcacgatca

SEQ ID NO: 129 - DNA, 21 bp, Homo sapiens;
tccactgggcacagaacttat

SEQ ID NO: 130 - DNA, 20 bp, Homo sapiens;
tctggctctgaaacaaagga

SEQ ID NO: 131 - DNA, 21 bp, Homo sapiens;
aacagagagggatgcttggat

SEQ ID NO: 132 - DNA, 20 bp, Homo sapiens;
caccaagggaaagggatgat

SEQ ID NO: 133 - DNA, 21 bp, Homo sapiens;
tgccttctgcttttaagttgc

SEQ ID NO: 134 - DNA, 20 bp, Homo sapiens;
gatgaaggggttcccataaa

SEQ ID NO: 135 - DNA, 20 bp, Homo sapiens;
tgggaagagggagaaaatga

SEQ ID NO: 136 - DNA, 20 bp, Homo sapiens;
aggcactgaaacattcgcat

SEQ ID NO: 137 - DNA, 21 bp, Homo sapiens;
aacacgaccatcgtagggtga

SEQ ID NO: 138 - DNA, 20 bp, Homo sapiens;
tttgaggtgatggtgggata

SEQ ID NO: 139 - DNA, 20 bp, Homo sapiens;
caccgatggccatgtaaata

SEQ ID NO: 140 - DNA, 20 bp, Homo sapiens;
tgtccaggaaaagccatctt

SEQ ID NO: 141 - DNA, 22 bp, Homo sapiens;
actctgggttatactggtgcga

SEQ ID NO: 142 - DNA, 23 bp, Homo sapiens;
ccaaagagatttctaaatcccac

SEQ ID NO: 143 - DNA, 20 bp, Homo sapiens;
tggaacaggtgcctaaagga

SEQ ID NO: 144 - DNA, 19 bp, Homo sapiens;
acagggtcgaacgtgcaca

SEQ ID NO: 145 - DNA, 22 bp, Homo sapiens;
tgaatgaggagttctggtacct

SEQ ID NO: 146 - DNA, 24 bp, Homo sapiens;
agtcaagccacagactaggtgtaa

SEQ ID NO: 147 - DNA, 20 bp, Homo sapiens;
caatgatctgtgctctgcaa

SEQ ID NO: 148 - DNA, 21 bp, Homo sapiens;
ccaaaaaccaacatgacacac

SEQ ID NO: 149 - DNA, 23 bp, Homo sapiens;
gtccaagcacatcttgtcaacat

SEQ ID NO: 150 - DNA, 24 bp, Homo sapiens;
agatcattatagtgaatgtcccca

SEQ ID NO: 151 - DNA, 19 bp, Homo sapiens;
taacatggtcccatgcctt

SEQ ID NO: 152 - DNA, 20 bp, Homo sapiens;
taggctgggctgatctgctt

SEQ ID NO: 153 - DNA, 24 bp, Homo sapiens;
ccaactaataagcttctctatgcc

SEQ ID NO: 154 - DNA, 22 bp, Homo sapiens;
tctgtcaaggccacttcttcat

SEQ ID NO: 155 - DNA, 21 bp, Homo sapiens;
caagcaacagtgagtcctcct

SEQ ID NO: 156 - DNA, 21 bp, Homo sapiens;
tgtaatgtcagccttcttccc

SEQ ID NO: 157 - DNA, 21 bp, Homo sapiens;
cccagctgctgaagagacaat

SEQ ID NO: 158 - DNA, 21 bp, Homo sapiens;
atgatccctacgatggtgcat

SEQ ID NO: 159 - DNA, 20 bp, Homo sapiens;
tctggcaggcaatagttgaa

SEQ ID NO: 160 - DNA, 20 bp, Homo sapiens;
caacctctcactgttcccaa

SEQ ID NO: 161 - DNA, 25 bp, Homo sapiens;
gaaggttcagtagaatgaaaacagg

SEQ ID NO: 162 - DNA, 24 bp, Homo sapiens;
cacctacctaatggttccaaaatc

SEQ ID NO: 163 - DNA, 21 bp, Homo sapiens;
acacagatttgagctcagccc

SEQ ID NO: 164 - DNA, 20 bp, Homo sapiens;
atgtttggctccttggtgat

SEQ ID NO: 165 - DNA, 20 bp, Homo sapiens;
attttctcattgccattgga

SEQ ID NO: 166 - DNA, 23 bp, Homo sapiens;
tcctagaaaaaggtattggcaaa

SEQ ID NO: 167 - DNA, 24 bp, Homo sapiens;
tgaaacaaatttatcagcttccag

SEQ ID NO: 168 - DNA, 22 bp, Homo sapiens;
tactgccttgtgaagaaggtga

SEQ ID NO: 169 - DNA, 21 bp, Homo sapiens;
tgtgagctgcctggaatacat

SEQ ID NO: 170 - DNA, 20 bp, Homo sapiens;
tccctgtccatgtgtgcaga

SEQ ID NO: 171 - DNA, 20 bp, Homo sapiens;
tatgatttcatgtgcgggga

SEQ ID NO: 172 - DNA, 20 bp, Homo sapiens;
ttgggtgcatttttggagag

SEQ ID NO: 173 - DNA, 25 bp, Homo sapiens;
gaatttacttcttttccttgagtgg

SEQ ID NO: 174 - DNA, 21 bp, Homo sapiens;
tcgtatagccatgtttcctga

SEQ ID NO: 175 - DNA, 19 bp, Homo sapiens;
tgggagtttcctgagggtt

SEQ ID NO: 176 - DNA, 22 bp, Homo sapiens;
tgcttgttcctacagtattgcg

SEQ ID NO: 177 - DNA, 21 bp, Homo sapiens;
tagccgtatcattgtgtggga

SEQ ID NO: 178 - DNA, 21 bp, Homo sapiens;
tcgtcatcagcatcatcttcc

SEQ ID NO: 179 - DNA, 21 bp, Homo sapiens;
tgtccagggaagaagagatgt

SEQ ID NO: 180 - DNA, 20 bp, Homo sapiens;
tcaagctttcacaggggaaa

SEQ ID NO: 181 - DNA, 22 bp, Homo sapiens;
aggtattaagcccagtgcctaa

SEQ ID NO: 182 - DNA, 25 bp, Homo sapiens;
gaaaattatgtcttttgtgggaaca

SEQ ID NO: 183 - DNA, 25 bp, Homo sapiens;
caaaggaacagatacagtagcagga

SEQ ID NO: 184 - DNA, 20 bp, Homo sapiens;
cccatattttctcacacgca

SEQ ID NO: 185 - DNA, 21 bp, Homo sapiens;
tgtgagatcactggctttgga

SEQ ID NO: 186 - DNA, 20 bp, Homo sapiens;
agtcaaagtcatgcggcctt

SEQ ID NO: 187 - DNA, 21 bp, Homo sapiens;
tttctgagatgaagtcgaggg

SEQ ID NO: 188 - DNA, 21 bp, Homo sapiens;
tctgcttggagcacttaaaca

SEQ ID NO: 189 - DNA, 25 bp, Homo sapiens;
caccattcgtacataatactgaacc

SEQ ID NO: 190 - DNA, 25 bp, Homo sapiens;
gtgaaccaacaagattactctgttt

SEQ ID NO: 191 - DNA, 21 bp, Homo sapiens;
aattgtgttgctcctggagga

SEQ ID NO: 192 - DNA, 20 bp, Homo sapiens;
caatgccatttcctttccca

SEQ ID NO: 193 - DNA, 25 bp, Homo sapiens;
tgcaagacatacatttctactatgg

SEQ ID NO: 194 - DNA, 23 bp, Homo sapiens;
ccttccaatttatagacgaaccc

SEQ ID NO: 195 - DNA, 21 bp, Homo sapiens;
ttaggccttcctctctccaga

SEQ ID NO: 196 - DNA, 20 bp, Homo sapiens;
ctccgttttcacggaaaaca

SEQ ID NO: 197 - DNA, 18 bp, Homo sapiens;
gttttgaaaggcgccatt

SEQ ID NO: 198 - DNA, 21 bp, Homo sapiens;
tgtgaggtgtgacctcacgaa

SEQ ID NO: 199 - DNA, 22 bp, Homo sapiens;
acatgaggctgtcaagcaaaag

SEQ ID NO: 200 - DNA, 25 bp, Homo sapiens;
cctatttcaactgactcagagttca

SEQ ID NO: 201 - DNA, 21 bp, Homo sapiens;
atggccttagctcttagccaa

SEQ ID NO: 202 - DNA, 20 bp, Homo sapiens;
gctcttgcgctttgacttta

SEQ ID NO: 203 - DNA, 21 bp, Homo sapiens;
acacagatttgagctcagccc

SEQ ID NO: 204 - DNA, 20 bp, Homo sapiens;
atgtttggctccttggtgat

SEQ ID NO: 205 - DNA, 25 bp, Homo sapiens;
cagtaaagagcttactcctgtagcg

SEQ ID NO: 206 - DNA, 22 bp, Homo sapiens;
aagcctcagaatctgctccatt

SEQ ID NO: 207 - DNA, 20 bp, Homo sapiens;
tcttccctgaccttcacgaa

SEQ ID NO: 208 - DNA, 25 bp, Homo sapiens;
catttattaagcagttacaatgctg

SEQ ID NO: 209 - DNA, 25 bp, Homo sapiens;
agaccttctatctgaggaacaacca

SEQ ID NO: 210 - DNA, 21 bp, Homo sapiens;
ttgtcctgctttctgttctcg

SEQ ID NO: 211 - DNA, 25 bp, Homo sapiens;
ctacctcatatcagtttgctagcag

SEQ ID NO: 212 - DNA, 22 bp, Homo sapiens;
cgatcttttagtggtgcctctt

SEQ ID NO: 213 - DNA, 20 bp, Homo sapiens;
tcaccctaaaaacacccaca

SEQ ID NO: 214 - DNA, 20 bp, Homo sapiens;
ttttccccagagtcttggaa

SEQ ID NO: 215 - DNA, 22 bp, Homo sapiens;
tgaatgaggagttctggtacct

SEQ ID NO: 216 - DNA, 24 bp, Homo sapiens;
agtcaagccacagactaggtgtaa

SEQ ID NO: 217 - DNA, 21 bp, Homo sapiens;
tgctacgagaacttcaccgat

SEQ ID NO: 218 - DNA, 20 bp, Homo sapiens;
gaagcacaccaggaaattga

SEQ ID NO: 219 - DNA, 21 bp, Homo sapiens;
agcagcgtcgtcataggtaat

SEQ ID NO: 220 - DNA, 21 bp, Homo sapiens;
acaggaggatcacttgaggct

SEQ ID NO: 221 - DNA, 21 bp, Homo sapiens;
cgacaggaccagtgcatctat

SEQ ID NO: 222 - DNA, 20 bp, Homo sapiens;
aactctcccagttgctcctt